(19) United States
(12) Patent Application Publication
(10) Pub. No.: US 2025/0057930 A1
(43) Pub. Date: Feb. 20, 2025

(54) ANAPLASTIC LYMPHOMA KINASE (ALK) CANCER VACCINES AND METHODS OF USE THEREOF

(71) Applicant: The Children's Medical Center Corporation, Boston, MA (US)

(72) Inventors: Roberto Chiarle, Boston, MA (US); Rafael Blasco-Patino, Boston, MA (US)

(73) Assignee: The Children's Medical Center Corporation, Boston, MA (US)

(21) Appl. No.: 18/936,652

(22) Filed: Nov. 4, 2024

Related U.S. Application Data
(63) Continuation of application No. PCT/US2023/021189, filed on May 5, 2023.
(60) Provisional application No. 63/339,018, filed on May 6, 2022.

(51) Int. Cl. A61K 39/00; A61K 31/439; A61K 31/4545; A61K 31/496; A61K 31/501; A61K 31/506; A61K 31/5377; A61K 31/7084; A61K 39/395; C12N 9/12

(52) U.S. Cl. CPC A61K 39/001162; A61K 31/439; A61K 31/4545; A61K 31/496; A61K 31/501; A61K 31/506; A61K 31/5377; A61K 31/7084; A61K 39/3955; C12N 9/12; C12Y 207/10001

(57) ABSTRACT

The invention features compositions and methods for treating anaplastic lymphoma kinase (ALK)-rearranged neoplasias including Non-Small Cell Lung Cancers (NSCLCs). The methods involve administering to a subject ALK peptides and/or polynucleotides encoding the ALK peptides, optionally in combination with an immune checkpoint inhibitor (ICI) and/or an ALK tyrosine kinase inhibitor (TKI).

### CROSS-REFERENCE TO RELATED APPLICATIONS

This application is a continuation under 35 U.S.C. § 111(a) of PCT International Patent Application No. PCT/US2023/021189, filed May 5, 2023, designating the United States and published in English, which claims priority to and the benefit of U.S. Provisional Application No. 63/339,018, filed May 6, 2022, the entire contents of each of which are incorporated by reference herein.

### SEQUENCE LISTING

The present application contains a Sequence Listing which has been submitted electronically in XML format. The entire contents of the electronic XML Sequence Listing, (Date of creation: Jun. 8, 2023; Size: 374,649 bytes; Name: 167705-025101_PCT_SL.xml), is incorporated by reference herein.

### BACKGROUND OF THE INVENTION

Anaplastic lymphoma kinase (ALK) rearrangements define a distinct molecular subset of non-small cell lung cancer (NSCLC) that is initially sensitive to treatment with ALK tyrosine kinase inhibitors (TKIs). Currently, five ALK TKIs (crizotinib, alectinib, ceritinib, brigatinib, and lorlatinib) are FDA-approved for use in ALK-rearranged (or ALK⁺) NSCLCs in the United States; however long-term disease control is limited by acquired resistance commonly mediated by secondary mutations in the ALK kinase domain, bypass track activation, and other mechanisms, and no effective immunotherapies are available for refractory or relapsed tumors. Most patients receiving first-line alectinib or brigatinib will develop disease progression within three years, and the latest ALK inhibitor approved to treat such resistance, lorlatinib, will only provide an average of 7 months of disease control. Additionally, the incidence of brain metastasis is often considered a final stage of advanced illness leading to disease progression and death.

Although immune checkpoint inhibitors (ICIs) of the programmed cell death 1 (PD-1) pathway have led to improvements in overall survival for general NSCLC patients, ALK⁺ lung cancers in particular do not derive benefit from ICIs. The reasons for this unresponsiveness are still poorly understood and possibly are due to the low tumor mutational burden (TMB) of ALK⁺ NSCLC, to an unfavorable tumor microenvironment that impairs the response of tumor-infiltrating T-cells, or limited antigen presentation during ALK TKIs treatment. However, it is known that ALK protein is antigenic and can elicit spontaneous immune responses when re-expressed by tumor cells. ALK⁺ lymphoma patients spontaneously develop anti-ALK immune responses that inversely correlate with stage of disease, the amount of circulating tumor cells, and cumulative incidence of relapse. ALK-specific tumor-reactive T-cells can be detected in mononuclear cells isolated from ALK⁺ lymphoma patients peripheral blood, but not from healthy donors. Likewise, a subset of ALK⁺ NSCLC patients has high anti-ALK autoantibody levels, which correlates with improved survival. Finally, vaccination with the cytoplasmic domain of the ALK protein elicits CD8⁺ cytotoxic T-cell responses, which provide long-term protection and therapeutic benefit in mouse models of ALK⁺ lymphoma and ALK⁺ lung cancer. While there is evidence that the ALK protein is naturally immunogenic, the precise mouse and human epitopes that engage specific T cell responses are unknown and it is unclear why ALK-specific T-cell responses seen in patients are insufficient to achieve a therapeutic efficacy during ICI treatment.

Thus, there is a need for additional and improved ALK-targeted therapies in ALK-positive NSCLCs.

### SUMMARY OF THE INVENTION

As described below, the present invention features compositions and methods for treating anaplastic lymphoma kinase (ALK)-rearranged neoplasias including Non-Small Cell Lung Cancers (NSCLCs). The methods involve administering to a subject ALK peptides and/or polynucleotides encoding the ALK peptides, optionally in combination with an immune checkpoint inhibitor (ICI) and/or an ALK tyrosine kinase inhibitor (TKI).

In one aspect, the invention of the disclosure provides a method for treating a subject having an anaplastic lymphoma kinase (ALK)-rearranged and/or ALK-positive neoplasia that is resistant to ALK tyrosine kinase inhibitor therapy. The method involves administering to the subject identified as resistant to ALK tyrosine kinase inhibitor therapy, an ALK peptide and/or a polynucleotide encoding the ALK peptide, alone or in combination with a tyrosine kinase inhibitor (TKI) and/or an immune checkpoint inhibitor (ICI), thereby treating the subject.

In another aspect, the invention of the disclosure provides a method for treating metastasis or inhibiting the development of metastasis in a subject having an anaplastic lymphoma kinase (ALK)-rearranged and/or ALK-positive neoplasia. The method involves administering to the subject an ALK peptide and/or a polynucleotide encoding the ALK peptide, thereby treating metastasis in the subject.

In another aspect, the invention of the disclosure provides an isolated anaplastic lymphoma kinase (ALK) peptide capable of generating an immune response against one or more ALK-positive cancers. The ALK peptide contains a sequence with at least about 85% identity to the amino acid sequence FNHQNIVRCIGVSL (SEQ ID NO: 1).

In another aspect, the invention of the disclosure provides an isolated anaplastic lymphoma kinase (ALK) peptide capable of generating an immune response against one or more ALK-positive cancers. The ALK peptide contains a sequence with at least about 85% identity to the amino acid sequence GGDLKSFLRETRPRPSQPSSLAM (SEQ ID NO: 2).

In another aspect, the invention of the disclosure provides a polynucleotide encoding the ALK peptide of any of the above aspects, or embodiments thereof.

In another aspect, the invention of the disclosure provides a vaccine containing the polynucleotide of any of the above aspects, or embodiments thereof.

In another aspect, the invention of the disclosure provides a vaccine containing the ALK peptide of any of the above aspects, or embodiments thereof.

In another aspect, the invention of the disclosure provides an immunogenic composition containing the vaccine of any of the above aspects, or embodiments thereof, and a pharmaceutically acceptable carrier, diluent, or excipient.

In another aspect, the invention of the disclosure provides a composition containing an ALK peptide and/or a polynucleotide encoding the ALK peptide, a tyrosine kinase inhibitor (TKI), and/or an immune checkpoint inhibitor (ICI).

In another aspect, the invention of the disclosure provides a kit containing an agent for administration to a subject with one or more ALK-positive cancers. The agent contains the isolated ALK peptide, the vaccine, and/or the immunogenic composition of any of the above aspects, or embodiments thereof.

In another aspect, the invention of the disclosure provides a method for treating an HLA-B*07:02 subject having an anaplastic lymphoma kinase (ALK)-rearranged Non-Small Cell Lung Cancer (NSCLC) that is resistant to ALK tyrosine kinase inhibitor therapy. The method involves administering to the subject an ALK peptide containing a sequence selected from one or more of RPRPSQPSSL (SEQ ID NO: 3); IVRCIGVSL (SEQ ID NO: 4); VPRKNITLI (SEQ ID NO: 5); TAAEVSVRV (SEQ ID NO: 6); AMLDLLHVA (SEQ ID NO: 7); FNHQNIVRCIGVSL (SEQ ID NO: 1); and GGDLKSFLRETRPRPSQPSSLAM (SEQ ID NO: 2), and/or a polynucleotide encoding the ALK peptide, alone or in combination with lorlatinib and/or an immune checkpoint inhibitor (ICI) selected from one or more of an anti-PD1 antibody, an anti-PDL1 antibody, an anti-CTLA-4 antibody, an anti-TIM3 antibody, an anti-LAG3 antibody, an anti-TIGIT antibody, an anti-VISTA antibody, an anti-ILT2 antibody, an anti-ILT4 antibody, and an anti-KIR3DL3 antibody, thereby treating the subject.

In another aspect, the invention of the disclosure provides a method for treating metastasis in an HLA-1B*07:02 subject having an anaplastic lymphoma kinase (ALK)-rearranged Non-Small Cell Lung Cancer (NSCLC). The method involves administering to the subject an ALK peptide containing a sequence selected from one or more of RPRPSQPSSL (SEQ ID NO: 3); IVRCIGVSL (SEQ ID NO: 4); VPRKNITLI (SEQ ID NO: 5); TAAEVSVRV (SEQ ID NO: 6); AMLDLLHVA (SEQ ID NO: 7); FNHQNIVRCIGVSL (SEQ ID NO: 1); and GGDLKSFLRETRPRPSQPSSLAM (SEQ ID NO: 2), and/or a polynucleotide encoding the ALK peptide, alone or in combination with lorlatinib and/or an immune checkpoint inhibitor (ICI) selected from one or more of an anti-PD1 antibody, an anti-PDL1 antibody, an anti-CTLA-4 antibody, an anti-TIM3 antibody, an anti-LAG3 antibody, an anti-TIGIT antibody, an anti-VISTA antibody, an anti-ILT2 antibody, an anti-ILT4 antibody, and an anti-KIR3DL3 antibody, thereby treating metastasis in the subject.

In any of the above aspects, or embodiments thereof, the ALK peptide and/or polynucleotide encoding the ALK peptide, the TKI, and/or the ICI are formulated together or separately.

In any of the above aspects, or embodiments thereof, the metastasis is a central nervous system, liver, or kidney metastasis.

In any of the above aspects, or embodiments thereof, the method further involves administering to the subject a tyrosine kinase inhibitor (TKI) and/or an immune checkpoint inhibitor (ICI). In any of the above aspects, or embodiments thereof, the method further involves administering, simultaneously or sequentially, to the subject an effective amount of one or more of an ALK inhibitor, the immune checkpoint inhibitor, and/or the tyrosine kinase inhibitor (TKI).

In any of the above aspects, or embodiments thereof, the ALK inhibitor or TKI is selected from one or more of crizotinib, alectinib, ceritinib, brigatinib, ensartinib, entrectinib, and lorlatinib.

In any of the above aspects, or embodiments thereof, the neoplasia is selected from one or more of non-small cell lung cancer (NSCLC), anaplastic large cell lymphoma (ALCL), neuroblastoma, B-cell lymphoma, thyroid cancer, colon cancer, breast cancer, inflammatory myofibroblastic tumors (IMT), renal carcinoma, esophageal cancer, glioma, glioblastoma, and melanoma.

In any of the above aspects, or embodiments thereof, the immune checkpoint inhibitor is selected from one or more of a programmed cell death protein 1 (PD-1) inhibitor, a programmed death-ligand 1 (PD-L1) inhibitor, a cytotoxic T-lymphocyte-associated antigen-4 (CTLA-4) inhibitor, a T-cell immunoglobulin and mucin domain 3 (TIM3) inhibitor, a lymphocyte-activation gene 3 (LAG3) inhibitor, a T-cell immunoglobulin and ITIM domain (TIGIT) inhibitor, a V-domain immunoglobulin suppressor of T cell activation (VISTA) inhibitor, a immunoglobulin-like transcript 2 (ILT2) inhibitor, a immunoglobulin-like transcript 4 (ILT4) inhibitor, and a killer cell immunoglobulin-like receptor, three immunoglobulin domains and long cytoplasmic tail (KIR3DL3) inhibitor.

In any of the above aspects, or embodiments thereof, the peptide and/or polynucleotide administered with an adjuvant. In any of the above aspects, or embodiments thereof, the method involves administering IFN-γ or a STING agonist. In embodiments, the STING agonist contains ADU-S100.

In any of the above aspects, or embodiments thereof, the peptide contains an amino acid sequence that has at least about 95% identity to a sequence listed in any of Tables 1, 2A, 2B, 2C, and/or 7 and/or to any of the following amino acid sequences: RPRPSQPSSL (SEQ ID NO: 3); IVRCIGVSL (SEQ ID NO: 4); VPRKNITLI (SEQ ID NO: 5); TAAEVSVRV (SEQ ID NO: 6); AMLDLLHVA (SEQ ID NO: 7); FNHQNIVRCIGVSL (SEQ ID NO: 1); and GGDLKSFLRETRPRPSQPSSLAM (SEQ ID NO: 2). In any of the above aspects, or embodiments thereof, the peptide contains an amino acid sequence that has at least about 95% identity to an amino acid sequence selected from one or more of RPRPSQPSSL (SEQ ID NO: 3); IVRCIGVSL (SEQ ID NO: 4); VPRKNITLI (SEQ ID NO: 5); TAAEVSVRV (SEQ ID NO: 6); AMLDLLHVA (SEQ ID NO: 7); FNHQNIVRCIGVSL (SEQ ID NO: 1); and GGDLKSFLRETRPRPSQPSSLAM (SEQ ID NO: 2). In any of the above aspects, or embodiments thereof, the peptide contains an amino acid sequence that has at least about 95% identity to the sequence FNHQNIVRCIGVSL (SEQ ID NO: 1). In any of the above aspects, or embodiments thereof, the peptide contains an amino acid sequence that has at least about 95% identity to the sequence GGDLKSFLRETRPRPSQPSSLAM (SEQ ID NO: 2).

In any of the above aspects, or embodiments thereof, the peptide is capable of binding a human leukocyte antigen (HLA). In embodiments, the HLA is encoded by a HLA class I allele. In embodiments, the HLA class I allele is selected from one or more of HLA-A*02:01 and HLA-B*07:02. In embodiments, the subject expresses the HLA class I allele.

In any of the above aspects, or embodiments thereof, the ALK rearrangement is a nucleophosmin-ALK rearrangement (NPM-ALK) or an echinoderm microtubule-associate protein-like 4-ALK rearrangement (EML4-ALK).

In any of the above aspects, or embodiments thereof, the polynucleotide encoding the ALK peptide contains DNA and/or RNA.

In any of the above aspects, or embodiments thereof, survival of the subject is extended relative to a reference subject. In any of the above aspects, or embodiments thereof, ALK+ lung tumors are reduced in the subject relative to a reference subject. In any of the above aspects, or embodiments thereof, the method further involves generating an ALK-specific immune memory in the subject. In any of the above aspects, or embodiments thereof, the method further involves reducing metastatic spread of ALK⁺ tumor cells in the subject relative to a reference subject. In any of the above aspects, or embodiments thereof, metastatic spread to the brain is reduced in the subject relative to a reference subject. In any of the above aspects, or embodiments thereof, the method further involves inducing an immune response in the subject, where the immune response involves producing T-lymphocytes. In any of the above aspects, or embodiments thereof, the method further involves increasing the number of ALK-specific tumor-infiltrating T lymphocytes in the subject relative to a reference subject. In embodiments, the tumor-infiltrating T lymphocytes contain ALK-specific CD8+ T cells. In any of the above aspects, or embodiments thereof, tumor progression is delayed in the subject relative to a reference subject.

In any of the above aspects, or embodiments thereof, the subject is administered the peptide, lorlatinib, and an anti-CTLA-4 antibody.

In any of the above aspects, or embodiments thereof, the subject had at least one prior treatment with at least one tyrosine kinase inhibitor (TKI).

In any of the above aspects, or embodiments thereof, the method further involves administering the ALK peptide and/or polynucleotide encoding the ALK peptide, the TKI, and/or the ICI concurrently or at different times. In any of the above aspects, or embodiments thereof, the method further involves administering the ALK peptide and/or polynucleotide encoding the ALK peptide 1, 2, 3, 4, or 5 times. In any of the above aspects, or embodiments thereof, the method further involves administering the ALK peptide about every 1, 2, 3, or 4 weeks.

In any of the above aspects, or embodiments thereof, the subject is a mammal. In any of the above aspects, or embodiments thereof, the subject is a human.

In any of the above aspects, or embodiments thereof, the ALK peptide contains the amino acid sequence FNHQNIVRCIGVSL (SEQ ID NO: 1). In any of the above aspects, or embodiments thereof, the ALK peptide contains the amino acid sequence GGDLKSFLRETRPRPSQPSSLAM (SEQ ID NO: 2).

In any of the above aspects, or embodiments thereof, the composition further contains an adjuvant.

In any of the above aspects, or embodiments thereof, the vaccine contains IFN-γ or a STING agonist. In embodiments, the STING agonist contains ADU-S100. In embodiments, the adjuvant contains a synthetic complex of carboxymethylcellulose, polyinosinic-polycytidylic acid, and poly-L-lysine double-stranded RNA (poly ICLC) or CpG oligonucleotides.

In any of the above aspects, or embodiments thereof, the peptide is conjugated to an amphiphile. In any of the above aspects, or embodiments thereof, amphiphile is N-hydroxy succinimidyl ester-end-functionalized poly(ethylene glycol)-lipid (NHS-PEG2KDa-DSPE).

In any of the above aspects, or embodiments thereof, metastasis is reduced relative to an untreated control subject. In any of the above aspects, or embodiments thereof, the peptide is administered with ADU-S100. In any of the above aspects, or embodiments thereof, the ALK rearrangement is an echinoderm microtubule-associate protein-like 4-ALK rearrangement (EML4-ALK).

Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention pertains or relates. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); Benjamin Lewin, Genes V, published by Oxford University Press, 1994 (ISBN 0-19-854287-9); Kendrew et al. (eds.); The Encyclopedia of Molecular Biology, published by Blackwell Science Ltd., 1994 (ISBN 0-632-02182-9); Molecular Biology and Biotechnology: a Comprehensive Desk Reference, Robert A. Meyers (ed.), published by VCH Publishers, Inc., 1995 (ISBN 1-56081-569-8); and Hale & Marham, The Harper Collins Dictionary of Biology (1991). As used herein, the following terms have the meanings ascribed to them below, unless specified otherwise.

By “ADU-S100” is meant a compound having the structure

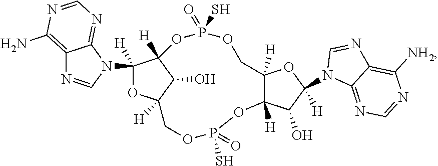 [SMILES: Nc1ncnc2c1ncn2[C@@H]1O[C@@H]2CO[P@](=O)(S)O[C@@H]3[C@H](O)[C@@H](CO[P@@](=O)(S)O[C@H]2[C@H]1O)O[C@H]3n1cnc2c(N)ncnc21]

corresponding to CAS No. 1638241-89-0, and pharmaceutically acceptable salts thereof having activity as a stimulator of interferon genes (STING).

By “alectinib” is meant a compound having the structure

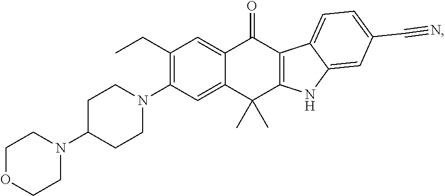 [SMILES: CCc1cc2c(cc1N1CCC(N3CCOCC3)CC1)C(C)(C)c1[nH]c3cc(C#N)ccc3c1C2=O]

corresponding to CAS No. 1256580-46-7, and pharmaceutically acceptable salts thereof having activity as a tyrosine kinase inhibitor (TKI).

By “ALK positive” is meant having detectable ALK polypeptide or polynucleotide expression. Methods for measuring ALK expression are described, for example, in Vemersson, et al. “Characterization of the expression of the ALK receptor tyrosine kinase in mice,” Gene Expr Patterns, 6:448-461 (2005) and in Dirks, et al. “Expression and functional analysis of the anaplastic lymphoma kinase (ALK) gene in tumor cell lines,” Int. J Cancer, 100:49-56 (2002), the disclosures of which are incorporated herein by reference in their entireties for all purposes. In embodiments, an ALK positive cell contains a change to the structure of the ALK gene. In some cases, an ALK positive cell expresses ALK at higher levels than a reference cell (e.g., a healthy non-neoplastic cell).

By “brigatinib” is meant a compound having the structure

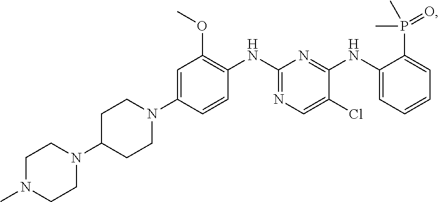 [SMILES: COc1cc(N2CCC(N3CCN(C)CC3)CC2)ccc1Nc1ncc(Cl)c(Nc2ccccc2P(C)(C)=O)n1]

corresponding to CAS No. 1197953-54-0, and pharmaceutically acceptable salts thereof having activity as a tyrosine kinase inhibitor (TKI).

By “ceritinib” is meant a compound having the structure

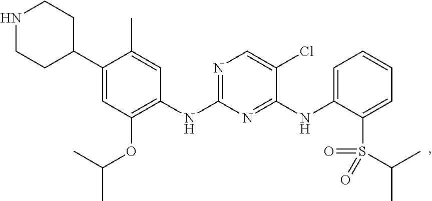 [SMILES: Cc1cc(Nc2ncc(Cl)c(Nc3ccccc3S(=O)(=O)C(C)C)n2)c(OC(C)C)cc1C1CCNCC1]

corresponding to CAS No. 1032900-25-6, and pharmaceutically acceptable salts thereof, having activity as a tyrosine kinase inhibitor (TKI).

By “crizotinib” is meant a compound having the structure

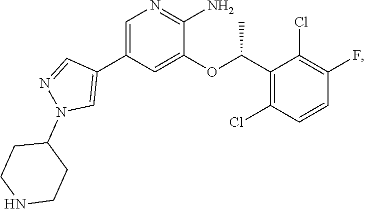 [SMILES: C[C@@H](Oc1cc(-c2cnn(C3CCNCC3)c2)cnc1N)c1c(Cl)ccc(F)c1Cl]

corresponding to CAS No. 877399-52-5, and pharmaceutically acceptable salts thereof having activity as a tyrosine kinase inhibitor (TKI).

By “ensartinib” is meant a compound having the structure

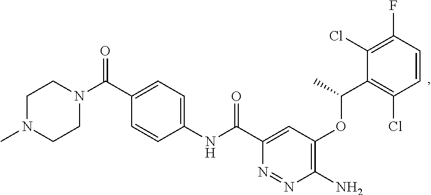 [SMILES: C[C@@H](Oc1cc(C(=O)Nc2ccc(C(=O)N3CCN(C)CC3)cc2)nnc1N)c1c(Cl)ccc(F)c1Cl]

corresponding to CAS No. 1365267-27-1, and pharmaceutically acceptable salts thereof, having activity as a tyrosine kinase inhibitor (TKI).

By “entrectinib” is meant a compound having the structure

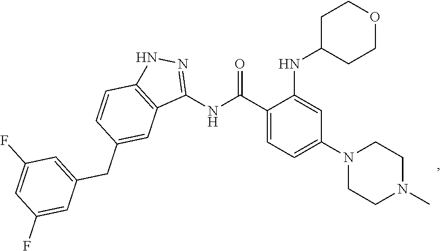 [SMILES: CN1CCN(c2ccc(C(=O)Nc3n[nH]c4ccc(Cc5cc(F)cc(F)c5)cc34)c(NC3CCOCC3)c2)CC1]

corresponding to CAS No. 1108743-60-7, and pharmaceutically acceptable salts thereof, having activity as a tyrosine kinase inhibitor (TKI).

By “lorlatinib,” “LORBRENA®,” or “LORVIQUA®” is meant a compound having the structure

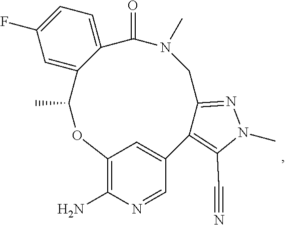 [SMILES: C[C@H]1Oc2cc(cnc2N)-c2c(nn(C)c2C#N)CN(C)C(=O)c2ccc(F)cc21]

corresponding to CAS No. 1454846-35-5, and pharmaceutically acceptable salts thereof having activity as a tyrosine kinase inhibitor (TKI).

By “adjuvant” is meant a substance or vehicle that non-specifically enhances the immune response to an antigen. Adjuvants may include a suspension of minerals (e.g., alum, aluminum hydroxide, or phosphate) on which antigen is adsorbed; or water-in-oil emulsion in which antigen solution is emulsified in mineral oil (e.g., Freund's incomplete adjuvant), sometimes with the inclusion of killed mycobacteria (Freund's complete adjuvant) to further enhance antigenicity. Immunostimulatory oligonucleotides (such as those including a CpG motif) can also be used as adjuvants (see, e.g., U.S. Pat. Nos. 6,194,388; 6,207,646; 6,214,806; 6,218,371; 6,239,116; 6,339,068; 6,406,705; and 6,429,199). Adjuvants also include biological molecules, such as costimulatory molecules. Exemplary biological adjuvants include, without limitation, interleukin-1 (IL-2), the protein memory T-cell attractant “Regulated on Activation, Normal T Expressed and Secreted” (RANTES), granulocyte-macrophage-colony stimulating factor (GM-CSF), tumor necrosis factor-alpha (TNF-α), interferon-gamma (IFN-γ), granulocyte-colony stimulation factor (G-CSF), lymphocyte function-associated antigen 3 (LFA-3, also called CD58), cluster of differentiation antigen 72 (CD72), (a negative regulator of B-cell responsiveness), peripheral membrane protein, B7-1 (B7-1, also called CD80), peripheral membrane protein, B7-2 (B7-2, also called CD86), the TNF ligand superfamily member 4 ligand (OX40L) or the type 2 transmembrane glycoprotein receptor belonging to the TNF superfamily (4-1BBL). In some embodiments, the adjuvant may be conjugated to an amphiphile as described in H. Liu et al., Structure-based programming of lymph-node targeting in molecular vaccines. Nature 507, 5199522 (2014). In some embodiments, the amphiphile conjugated to the adjuvant is N-hydroxy succinimidyl ester-end-functionalized poly(ethylene glycol)-lipid (NHS-PEG2KDa-DSPE).

By “agent” is meant any small molecule chemical compound, antibody, nucleic acid molecule, peptide, polypeptide, or fragments thereof.

By “ALK polypeptide” or “ALK peptide” is meant a protein or fragment thereof having at least 85% amino acid identity to an anaplastic lymphoma kinase (ALK) amino acid sequence associated with GenBank Accessions No.: BAD92714.1, ACY79563.1, or ACI47591.1, and that is capable of inducing an ALK-specific immune response in an immunized subject. In some embodiments, the ALK polypeptide is at least 85%, at least 90%, at least 95%, at least 98%, at least 99%, or 100% identical to the ALK protein in Homo Sapiens. In embodiments, the ALK peptide contains about, at least about, and/or nor more than about 5, 6, 7, 8, 9, 10, 11, 12, 13, 14, 15, 20, 25, 30, 35, 40, 45, or 50 amino acids. Exemplary ALK full-length amino acid sequences from Homo Sapiens are provided below (see GenBank Accessions No. BAD92714.1, ACY79563.1, and AC147591.1):

>BAD92714.1 anaplastic lymphoma kinase Ki-1 variant, partial [Homo
sapiens] (ALK cytoplasmic portion in bold font)
(SEQ ID NO: 8)
TASSGGMGAIGLLWLLPLLLSTAAVGSGMGTGQRAGSPAAGPPLQPREPLSYSRLQRKSLAVDF
VVPSLFRVYARDLLLPPSSSELKAGRPEARGSLALDCAPLLRLLGPAPGVSWTAGSPAPAEART
LSRVLKGGSVRKLRRAKQLVLELGEEAILEGCVGPPGEAAVGLLQFNLSELFSWWIRQGEGRLR
IRLMPEKKASEVGREGRLSAAIRASQPRLLFQIFGTGHSSLESPTNMPSPSPDYFTWNLTWIMK
DSFPFLSHRSRYGLECSFDFPCELEYSPPLHDLRNQSWSWRRIPSEEASQMDLLDGPGAERSKE
MPRGSFLLLNTSADSKHTILSPWMRSSSEHCTLAVSVHRHLQPSGRYIAQLLPHNEAAREILLM
PTPGKHGWTVLQGRIGRPDNPFRVALEYISSGNRSLSAVDFFALKNCSEGTSPGSKMALQSSFT
CWNGTVLQLGQACDFHQDCAQGEDESQMCRKLPVGFYCNFEDGFCGWTQGTLSPHTPQWQVRTL
KDARFQDHQDHALLLSTTDVPASESATVTSATFPAPIKSSPCELRMSWLIRGVLRGNVSLVLVE
NKTGKEQGRMVWHVAAYEGLSLWQWMVLPLLDVSDRFWLQMVAWWGQGSRAIVAFDNISISLDC
YLTISGEDKILQNTAPKSRNLFERNPNKELKPGENSPRQTPIFDPTVHWLFTTCGASGPHGPTQ
AQCNNAYQNSNLSVEVGSEGPLKGIQIWKVPATDTYSISGYGAAGGKGGKNTMMRSHGVSVLGI
FNLEKDDMLYILVGQQGEDACPSTNQLIQKVCIGENNVIEEEIRVNRSVHEWAGGGGGGGGATY
VFKMKDGVPVPLIIAAGGGGRAYGAKTDTFHPERLENNSSVLGLNGNSGAAGGGGGWNDNTSLL
WAGKSLQEGATGGHSCPQAMKKWGWETRGGFGGGGGGCSSGGGGGGYIGGNAASNNDPEMDGED
GVSFISPLGILYTPALKVMEGHGEVNIKHYLNCSHCEVDECHMDPESHKVICFCDHGTVLAEDG
VSCIVSPTPEPHLPLSLILSVVTSALVAALVLAFSGIMIVYRRKHQELQAMQMELQSPEYKLSK
LRTSTIMTDYNPNYCFAGKTSSISDLKEVPRKNITLIRGLGHGAFGEVYEGQVSGMPNDPSPLQ
VAVKTLPEVCSEQDELDFLMEALIISKFNHQNIVRCIGVSLQSLPRFILLELMAGGDLKSFLRE
TRPRPSQPSSLAMLDLLHVARDIACGCQYLEENHFIHRDIAARNCLLTCPGPGRVAKIGDFGMA
RDIYRASYYRKGGCAMLPVKWMPPEAFMEGIFTSKTDTWSFGVLLWEIFSLGYMPYPSKSNQEV
LEFVTSGGRMDPPKNCPGPVYRIMTQCWQHQPEDRPNFAIILERIEYCTQDPDVINTALPIEYG
PLVEEEEKVPVRPKDPEGVPPLLVSQQAKREEERSPAAPPPLPTTSSGKAAKKPTAAEVSVRVP
RGPAVEGGHVNMAFSQSNPPSELHRVHGSRNKPTSLWNPTYGSWFTEKPTKKNNPIAKKEPHER
GNLGLEGSCTVPPNVATGRLPGASLLLEPSSLTANMKEVPLFRLRHFPCGNVNYGYQQQGLPLE
AATAPGAGHYEDTILKSKNSMNQPGP.
>ACY79563.1 mutant anaplastic lymphoma receptor tyrosine kinase
[Homo sapiens]
(SEQ ID NO: 9)
MGAIGLLWLLPLLLSTAAVGSGMGTGQRAGSPAAGPPLQPREPLSYSRLQRKSLAVDFVVPSLF
RVYARDLLLPPSSSELKAGRPEARGSLALDCAPLLRLLGPAPGVSWTAGSPAPAEARTLSRVLK
GGSVRKLRRAKQLVLELGEEAILEGCVGPPGEAAVGLLQFNLSELFSWWIRQGEGRLRIRLMPE
KKASEVGREGRLSAAIRASQPRLLFQIFGTGHSSLESPTNMPSPSPDYFTWNLTWIMKDSFPFL
SHRSRYGLECSFDFPCELEYSPPLHDLRNQSWSWRRIPSEEASQMDLLDGPGAERSKEMPRGSF
LLLNTSADSKHTILSPWMRSSSEHCTLAVSVHRHLQPSGRYIAQLLPHNEAAREILLMPTPGKH
GWTVLQGRIGRPDNPFRVALEYISSGNRSLSAVDFFALKNCSEGTSPGSKMALQSSFTCWNGTV
LQLGQACDFHQDCAQGEDESQMCRKLPVGFYCNFEDGFCGWTQGTLSPHTPQWQVRTLKDARFQ
DHQDHALLLSTTDVPASESATVTSATFPAPIKSSPCELRMSWLIRGVLRGNVSLVLVENKTGKE
QGRMVWHVAAYEGLSLWQWMVLPLLDVSDRFWLQMVAWWGQGSRAIVAFDNISISLDCYLTISG
EDKILQNTAPKSRNLFERNPNKELKPGENSPRQTPIFDPTVHWLFTTCGASGPHGPTQAQCNNA
YQNSNLSVEVGSEGPLKGIQIWKVPATDTYSISGYGAAGGKGGKNTMMRSHGVSVLGIFNLEKD
DMLYILVGQQGEDACPSTNQLIQKVCIGENNVIEEEIRVNRSVHEWAGGGGGGGGATYVFKMKD
GVPVPLIIAAGGGGRAYGAKTDTFHPERLENNSSVLGLNGNSGAAGGGGGWNDNTSLLWAGKSL
QEGATGGHSCPQAMKKWGWETRGGFGGGGGGCSSGGGGGGYIGGNAASNNDPEMDGEDGVSFIS
PLGILYTPALKVMEGHGEVNIKHYLNCSHCEVDECHMDPESHKVICFCDHGTVLAEDGVSCIVS
PTPEPHLPLSLILSVVTSALVAALVLAFSGIMIVYRRKHQELQAMQMELQSPEYKLSKLRTSTI
MTDYNPNYCFAGKTSSISDLKEVPRKNITLIRGLGHGAFGEVYEGQVSGMPNDPSPLQVAVKTL
PEVCSEQDELDFLMEALIISKFNHQNIVRCIGVSLQSLPRFILLELMVGGDLKSFLRETRPRPS
QPSSLAMLDLLHVARDIACGCQYLEENHFIHRDIAARNCLLTCPGPGRVAKIGDFGMARDIYRA
SYYRKGGCAMLPVKWMPPEAFMEGIFTSKTDTWSFGVLLWEIFSLGYMPYPSKSNQEVLEFVTS
GGRMDPPKNCPGPVYRIMTQCWQHQPEDRPNFAIILERIEYCTQDPDVINTALPIEYGPLVEEE
EKVPVRPKDPEGVPPLLVSQQAKREEERSPAAPPPLPTTSSGKAAKKPTAAEISVRVPRGPAVE
GGHVNMAFSQSNPPSELHKVHGSRNKPTSLWNPTYGSWFTEKPTKKNNPIAKKEPHDRGNLGLE
GSCTVPPNVATGRLPGASLLLEPSSLTANMKEVPLFRLRHFPCGNVNYGYQQQGLPLEAATAPG
AGHYEDTILKSKNSMNQPGP.
>ACI47591.1 anaplastic lymphoma kinase [Homo sapiens]
(SEQ ID NO: 10)
MGAIGLLWLLPLLLSTAAVGSGMGTGQRAGSPAAGPPLQPREPLSYSRLQRKSLAVDFVVPSLF
RVYARDLLLPPSSSELKAGRPEARGSLALDCAPLLRLLGPAPGVSWTAGSPAPAEARTLSRVLK
GGSVRKLRRAKQLVLELGEEAILEGCVGPPGEAAVGLLQFNLSELFSWWIRQGEGRLRIRLMPE
KKASEVGREGRLSAAIRASQPRLLFQIFGTGHSSLESPTNMPSPSPDYFTWNLTWIMKDSFPFL
SHRSRYGLECSFDFPCELEYSPPLHDLRNQSWSWRRIPSEEASQMDLLDGPGAERSKEMPRGSF
LLLNTSADSKHTILSPWMRSSSEHCTLAVSVHRHLQPSGRYIAQLLPHNEAAREILLMPTPGKH
GWTVLQGRIGRPDNPFRVALEYISSGNRSLSAVDFFALKNCSEGTSPGSKMALQSSFTCWNGTV
LQLGQACDFHQDCAQGEDESQMCRKLPVGFYCNFEDGFCGWTQGTLSPHTPQWQVRTLKDARFQ
DHQDHALLLSTTDVPASESATVTSATFPAPIKSSPCELRMSWLIRGVLRGNVSLVLVENKTGKE
QGRMVWHVAAYEGLSLWQWMVLPLLDVSDRFWLQMVAWWGQGSRAIVAFDNISISLDCYLTISG
EDKILQNTAPKSRNLFERNPNKELKPGENSPRQTPIFDPTVHWLFTTCGASGPHGPTQAQCNNA
YQNSNLSVEVGSEGPLKGIQIWKVPATDTYSISGYGAAGGKGGKNTMMRSHGVSVLGIFNLEKD
DMLYILVGQQGEDACPSTNQLIQKVCIGENNVIEEEIRVNRSVHEWAGGGGGGGGATYVFKMKD
GVPVPLIIAAGGGGRAYGAKTDTFHPERLENNSSVLGLNGNSGAAGGGGGWNDNTSLLWAGKSL
QEGATGGHSCPQAMKKWGWETRGGFGGGGGGCSSGGGGGGYIGGNAASNNDPEMDGEDGVSFIS
PLGILYTPALKVMEGHGEVNIKHYLNCSHCEVDECHMDPESHKVICFCDHGTVLAEDGVSCIVS
PTPEPHLPLSLILSVVTSALVAALVLAFSGIMIVYRRMHQELQAMQMELQSPEYKLSKLRTSTI
MTDYNPNYCFAGKTSSISDLKEVPRKNITLIRGLGHGAFGEVYEGQVSGMPNDPSPLQVAVKTL
PEVCSEQDELDFLMEALIISKFNHQNIVRCIGVSLQSLPRFILLELMAGGDLKSFLRETRPRPS
QPSSLAMLDLLHVARDIACGCQYLEENHFIHRDIAARNCLLTCPGPGRVAKIGDFGMARDIYRA
SYYRKGGCAMLPVKWMPPEAFMEGIFTSKTDTWSFGVLLWEIFSLGYMPYPSKSNQEVLEFVTS
GGRMDPPKNCPGPVYRIMTQCWQHQPEDRPNFAIILERIEYCTQDPDVINTALPIEYGPLVEEE
EKVPVRPKDPEGVPPLLVSQQAKREEERSPAAPPPLPTTSSGKAAKKPTAAEISVRVPRGPAVE
GGHVNMAFSQSNPPSELHKVHGSRNKPTSLWNPTYGSWFTEKPTKKNNPIAKKEPHDRGNLGLE
GSCTVPPNVATGRLPGASLLLEPSSLTANMKEVPLFRLRHFPCGNVNYGYQQQGLPLEAATAPG
AGHYEDTILKSKNSMNQPGP.

Exemplary ALK peptide amino acid sequences are provided in Tables 1, 2A-2C, and/or 7.

An exemplary ALK peptide amino sequence is as follows: RPRPSQPSSL (SEQ ID NO: 3) (RPRshort).

An exemplary ALK peptide amino sequence is as follows: IVRCIGVSL (SEQ ID NO: 4) (IVRshort).

An exemplary ALK peptide amino sequence is as follows: VPRKNITLI (SEQ ID NO: 5).

An exemplary ALK peptide amino sequence is as follows: TAAEVSVRV (SEQ ID NO: 6).

An exemplary ALK peptide amino sequence is as follows: AMLDLLHVA (SEQ ID NO: 7).

An exemplary ALK peptide amino sequence is as follows:

(SEQ ID NO: 1)
FNHQNIVRCIGVSL (IVRlong).

An exemplary ALK peptide amino sequence is as follows:

(SEQ ID NO: 2)
GGDLKSFLRETRPRPSQPSSLAM (RPRlong).

By “ALK polynucleotide” is meant any nucleic acid molecule encoding an ALK polypeptide or fragment thereof. Exemplary full-length ALK nucleic acid sequences from Homo Sapiens are provided below (see GenBank Accessions No.: AB209477.4, GU128155.1, and EU788003.1):

>AB209477.4:472-5352 Homo sapiens mRNA for anaplastic lymphoma kinase
Ki-1 variantprotein, partial cds
(SEQ ID NO: 11)
ACGGCCTCCTCCGGCGGGATGGGAGCCATCGGGCTCCTGTGGCTCCTGCCGCTGCTGCTTTCCA
CGGCAGCTGTGGGCTCCGGGATGGGGACCGGCCAGCGCGCGGGCTCCCCAGCTGCGGGGCCGCC
GCTGCAGCCCCGGGAGCCACTCAGCTACTCGCGCCTGCAGAGGAAGAGTCTGGCAGTTGACTTC
GTGGTGCCCTCGCTCTTCCGTGTCTACGCCCGGGACCTACTGCTGCCACCATCCTCCTCGGAGC
TGAAGGCTGGCAGGCCCGAGGCCCGCGGCTCGCTAGCTCTGGACTGCGCCCCGCTGCTCAGGTT
GCTGGGGCCGGCGCCGGGGGTCTCCTGGACCGCCGGTTCACCAGCCCCGGCAGAGGCCCGGACG
CTGTCCAGGGTGCTGAAGGGCGGCTCCGTGCGCAAGCTCCGGCGTGCCAAGCAGTTGGTGCTGG
AGCTGGGCGAGGAGGCGATCTTGGAGGGTTGCGTCGGGCCCCCCGGGGAGGCGGCTGTGGGGCT
GCTCCAGTTCAATCTCAGCGAGCTGTTCAGTTGGTGGATTCGCCAAGGCGAAGGGCGACTGAGG
ATCCGCCTGATGCCCGAGAAGAAGGCGTCGGAAGTGGGCAGAGAGGGAAGGCTGTCCGCGGCAA
TTCGCGCCTCCCAGCCCCGCCTTCTCTTCCAGATCTTCGGGACTGGTCATAGCTCCTTGGAATC
ACCAACAAACATGCCATCTCCTTCTCCTGATTATTTTACATGGAATCTCACCTGGATAATGAAA
GACTCCTTCCCTTTCCTGTCTCATCGCAGCCGATATGGTCTGGAGTGCAGCTTTGACTTCCCCT
GTGAGCTGGAGTATTCCCCTCCACTGCATGACCTCAGGAACCAGAGCTGGTCCTGGCGCCGCAT
CCCCTCCGAGGAGGCCTCCCAGATGGACTTGCTGGATGGGCCTGGGGCAGAGCGTTCTAAGGAG
ATGCCCAGAGGCTCCTTTCTCCTTCTCAACACCTCAGCTGACTCCAAGCACACCATCCTGAGTC
CGTGGATGAGGAGCAGCAGTGAGCACTGCACACTGGCCGTCTCGGTGCACAGGCACCTGCAGCC
CTCTGGAAGGTACATTGCCCAGCTGCTGCCCCACAACGAGGCTGCAAGAGAGATCCTCCTGATG
CCCACTCCAGGGAAGCATGGTTGGACAGTGCTCCAGGGAAGAATCGGGCGTCCAGACAACCCAT
TTCGAGTGGCCCTGGAATACATCTCCAGTGGAAACCGCAGCTTGTCTGCAGTGGACTTCTTTGC
CCTGAAGAACTGCAGTGAAGGAACATCCCCAGGCTCCAAGATGGCCCTGCAGAGCTCCTTCACT
TGTTGGAATGGGACAGTCCTCCAGCTTGGGCAGGCCTGTGACTTCCACCAGGACTGTGCCCAGG
GAGAAGATGAGAGCCAGATGTGCCGGAAACTGCCTGTGGGTTTTTACTGCAACTTTGAAGATGG
CTTCTGTGGCTGGACCCAAGGCACACTGTCACCCCACACTCCTCAGTGGCAGGTCAGGACCCTA
AAGGATGCCCGGTTCCAGGACCACCAAGACCATGCTCTATTGCTCAGTACCACTGATGTCCCCG
CTTCTGAAAGTGCTACAGTGACCAGTGCTACGTTTCCTGCACCGATCAAGAGCTCTCCATGTGA
GCTCCGAATGTCCTGGCTCATTCGTGGAGTCTTGAGGGGAAACGTGTCCTTGGTGCTAGTGGAG
AACAAAACCGGGAAGGAGCAAGGCAGGATGGTCTGGCATGTCGCCGCCTATGAAGGCTTGAGCC
TGTGGCAGTGGATGGTGTTGCCTCTCCTCGATGTGTCTGACAGGTTCTGGCTGCAGATGGTCGC
ATGGTGGGGACAAGGATCCAGAGCCATCGTGGCTTTTGACAATATCTCCATCAGCCTGGACTGC
TACCTCACCATTAGCGGAGAGGACAAGATCCTGCAGAATACAGCACCCAAATCAAGAAACCTGT
TTGAGAGAAACCCAAACAAGGAGCTGAAACCCGGGGAAAATTCACCAAGACAGACCCCCATCTT
TGACCCTACAGTTCATTGGCTGTTCACCACATGTGGGGCCAGCGGGCCCCATGGCCCCACCCAG
GCACAGTGCAACAACGCCTACCAGAACTCCAACCTGAGCGTGGAGGTGGGGAGCGAGGGCCCCC
TGAAAGGCATCCAGATCTGGAAGGTGCCAGCCACCGACACCTACAGCATCTCGGGCTACGGAGC
TGCTGGCGGGAAAGGCGGGAAGAACACCATGATGCGGTCCCACGGCGTGTCTGTGCTGGGCATC
TTCAACCTGGAGAAGGATGACATGCTGTACATCCTGGTTGGGCAGCAGGGAGAGGACGCCTGCC
CCAGTACAAACCAGTTAATCCAGAAAGTCTGCATTGGAGAGAACAATGTGATAGAAGAAGAAAT
CCGTGTGAACAGAAGCGTGCATGAGTGGGCAGGAGGCGGAGGAGGAGGGGGTGGAGCCACCTAC
GTATTTAAGATGAAGGATGGAGTGCCGGTGCCCCTGATCATTGCAGCCGGAGGTGGTGGCAGGG
CCTACGGGGCCAAGACAGACACGTTCCACCCAGAGAGACTGGAGAATAACTCCTCGGTTCTAGG
GCTAAACGGCAATTCCGGAGCCGCAGGTGGTGGAGGTGGCTGGAATGATAACACTTCCTTGCTC
TGGGCCGGAAAATCTTTGCAGGAGGGTGCCACCGGAGGACATTCCTGCCCCCAGGCCATGAAGA
AGTGGGGGTGGGAGACAAGAGGGGGTTTCGGAGGGGGTGGAGGGGGGTGCTCCTCAGGTGGAGG
AGGCGGAGGATATATAGGCGGCAATGCAGCCTCAAACAATGACCCCGAAATGGATGGGGAAGAT
GGGGTTTCCTTCATCAGTCCACTGGGCATCCTGTACACCCCAGCTTTAAAAGTGATGGAAGGCC
ACGGGGAAGTGAATATTAAGCATTATCTAAACTGCAGTCACTGTGAGGTAGACGAATGTCACAT
GGACCCTGAAAGCCACAAGGTCATCTGCTTCTGTGACCACGGGACGGTGCTGGCTGAGGATGGC
GTCTCCTGCATTGTGTCACCCACCCCGGAGCCACACCTGCCACTCTCGCTGATCCTCTCTGTGG
TGACCTCTGCCCTCGTGGCCGCCCTGGTCCTGGCTTTCTCCGGCATCATGATTGTGTACCGCCG
GAAGCACCAGGAGCTGCAAGCCATGCAGATGGAGCTGCAGAGCCCTGAGTACAAGCTGAGCAAG
CTCCGCACCTCGACCATCATGACCGACTACAACCCCAACTACTGCTTTGCTGGCAAGACCTCCT
CCATCAGTGACCTGAAGGAGGTGCCGCGGAAAAACATCACCCTCATTCGGGGTCTGGGCCATGG
AGCCTTTGGGGAGGTGTATGAAGGCCAGGTGTCCGGAATGCCCAACGACCCAAGCCCCCTGCAA
GTGGCTGTGAAGACGCTGCCTGAAGTGTGCTCTGAACAGGACGAACTGGATTTCCTCATGGAAG
CCCTGATCATCAGCAAATTCAACCACCAGAACATTGTTCGCTGCATTGGGGTGAGCCTGCAATC
CCTGCCCCGGTTCATCCTGCTGGAGCTCATGGCGGGGGGAGACCTCAAGTCCTTCCTCCGAGAG
ACCCGCCCTCGCCCGAGCCAGCCCTCCTCCCTGGCCATGCTGGACCTTCTGCACGTGGCTCGGG
ACATTGCCTGTGGCTGTCAGTATTTGGAGGAAAACCACTTCATCCACCGAGACATTGCTGCCAG
AAACTGCCTCTTGACCTGTCCAGGCCCTGGAAGAGTGGCCAAGATTGGAGACTTCGGGATGGCC
CGAGACATCTACAGGGCGAGCTACTATAGAAAGGGAGGCTGTGCCATGCTGCCAGTTAAGTGGA
TGCCCCCAGAGGCCTTCATGGAAGGAATATTCACTTCTAAAACAGACACATGGTCCTTTGGAGT
GCTGCTATGGGAAATCTTTTCTCTTGGATATATGCCATACCCCAGCAAAAGCAACCAGGAAGTT
CTGGAGTTTGTCACCAGTGGAGGCCGGATGGACCCACCCAAGAACTGCCCTGGGCCTGTATACC
GGATAATGACTCAGTGCTGGCAACATCAGCCTGAAGACAGGCCCAACTTTGCCATCATTTTGGA
GAGGATTGAATACTGCACCCAGGACCCGGATGTAATCAACACCGCTTTGCCGATAGAATATGGT
CCACTTGTGGAAGAGGAAGAGAAAGTGCCTGTGAGGCCCAAGGACCCTGAGGGGGTTCCTCCTC
TCCTGGTCTCTCAACAGGCAAAACGGGAGGAGGAGCGCAGCCCAGCTGCCCCACCACCTCTGCC
TACCACCTCCTCTGGCAAGGCTGCAAAGAAACCCACAGCTGCAGAGGTCTCTGTTCGAGTCCCT
AGAGGGCCGGCCGTGGAAGGGGGACACGTGAATATGGCATTCTCTCAGTCCAACCCTCCTTCGG
AGTTGCACAGGGTCCACGGATCCAGAAACAAGCCCACCAGCTTGTGGAACCCAACGTACGGCTC
CTGGTTTACAGAGAAACCCACCAAAAAGAATAATCCTATAGCAAAGAAGGAGCCACACGAGAGG
GGTAACCTGGGGCTGGAGGGAAGCTGTACTGTCCCACCTAACGTTGCAACTGGGAGACTTCCGG
GGGCCTCACTGCTCCTAGAGCCCTCTTCGCTGACTGCCAATATGAAGGAGGTACCTCTGTTCAG
GCTACGTCACTTCCCTTGTGGGAATGTCAATTACGGCTACCAGCAACAGGGCTTGCCCTTAGAA
GCCGCTACTGCCCCTGGAGCTGGTCATTACGAGGATACCATTCTGAAAAGCAAGAATAGCATGA
ACCAGCCTGGGCCCTGA.
>GU128155.1 Homo sapiens A1200V mutant anaplastic lymphoma receptor
tyrosine kinase (ALK) mRNA, complete cds
(SEQ ID NO: 12)
ATGGGAGCCATCGGGCTCCTGTGGCTCCTGCCGCTGCTGCTTTCCACGGCAGCTGTGGGCTCCG
GGATGGGGACCGGCCAGCGCGCGGGCTCCCCAGCTGCGGGGCCGCCGCTGCAGCCCCGGGAGCC
ACTCAGCTACTCGCGCCTGCAGAGGAAGAGTCTGGCAGTTGACTTCGTGGTGCCCTCGCTCTTC
CGTGTCTACGCCCGGGACCTACTGCTGCCACCATCCTCCTCGGAGCTGAAGGCTGGCAGGCCCG
AGGCCCGCGGCTCGCTAGCTCTGGACTGCGCCCCGCTGCTCAGGTTGCTGGGGCCGGCGCCGGG
GGTCTCCTGGACCGCCGGTTCACCAGCCCCGGCAGAGGCCCGGACGCTGTCCAGGGTGCTGAAG
GGCGGCTCCGTGCGCAAGCTCCGGCGTGCCAAGCAGTTGGTGCTGGAGCTGGGCGAGGAGGCGA
TCTTGGAGGGTTGCGTCGGGCCCCCCGGGGAGGCGGCTGTGGGGCTGCTCCAGTTCAATCTCAG
CGAGCTGTTCAGTTGGTGGATTCGCCAAGGCGAAGGGCGACTGAGGATCCGCCTGATGCCCGAG
AAGAAGGCGTCGGAAGTGGGCAGAGAGGGAAGGCTGTCCGCGGCAATTCGCGCCTCCCAGCCCC
GCCTTCTCTTCCAGATCTTCGGGACTGGTCATAGCTCCTTGGAATCACCAACAAACATGCCTTC
TCCTTCTCCTGATTATTTTACATGGAATCTCACCTGGATAATGAAAGACTCCTTCCCTTTCCTG
TCTCATCGCAGCCGATATGGTCTGGAGTGCAGCTTTGACTTCCCCTGTGAGCTGGAGTATTCCC
CTCCACTGCATGACCTCAGGAACCAGAGCTGGTCCTGGCGCCGCATCCCCTCCGAGGAGGCCTC
CCAGATGGACTTGCTGGATGGGCCTGGGGCAGAGCGTTCTAAGGAGATGCCCAGAGGCTCCTTT
CTCCTTCTCAACACCTCAGCTGACTCCAAGCACACCATCCTGAGTCCGTGGATGAGGAGCAGCA
GTGAGCACTGCACACTGGCCGTCTCGGTGCACAGGCACCTGCAGCCCTCTGGAAGGTACATTGC
CCAGCTGCTGCCCCACAACGAGGCTGCAAGAGAGATCCTCCTGATGCCCACTCCAGGGAAGCAT
GGTTGGACAGTGCTCCAGGGAAGAATCGGGCGTCCAGACAACCCATTTCGAGTGGCCCTGGAAT
ACATCTCCAGTGGAAACCGCAGCTTGTCTGCAGTGGACTTCTTTGCCCTGAAGAACTGCAGTGA
AGGAACATCCCCAGGCTCCAAGATGGCCCTGCAGAGCTCCTTCACTTGTTGGAATGGGACAGTC
CTCCAGCTTGGGCAGGCCTGTGACTTCCACCAGGACTGTGCCCAGGGAGAAGATGAGAGCCAGA
TGTGCCGGAAACTGCCTGTGGGTTTTTACTGCAACTTTGAAGATGGCTTCTGTGGCTGGACCCA
AGGCACACTGTCACCCCACACTCCTCAATGGCAGGTCAGGACCCTAAAGGATGCCCGGTTCCAG
GACCACCAAGACCATGCTCTATTGCTCAGTACCACTGATGTCCCCGCTTCTGAAAGTGCTACAG
TGACCAGTGCTACGTTTCCTGCACCGATCAAGAGCTCTCCATGTGAGCTCCGAATGTCCTGGCT
CATTCGTGGAGTCTTGAGGGGAAACGTGTCCTTGGTGCTAGTGGAGAACAAAACCGGGAAGGAG
CAAGGCAGGATGGTCTGGCATGTCGCCGCCTATGAAGGCTTGAGCCTGTGGCAGTGGATGGTGT
TGCCTCTCCTCGATGTGTCTGACAGGTTCTGGCTGCAGATGGTCGCATGGTGGGGACAAGGATC
CAGAGCCATCGTGGCTTTTGACAATATCTCCATCAGCCTGGACTGCTACCTCACCATTAGCGGA
GAGGACAAGATCCTGCAGAATACAGCACCCAAATCAAGAAACCTGTTTGAGAGAAACCCAAACA
AGGAGCTGAAACCCGGGGAAAATTCACCAAGACAGACCCCCATCTTTGACCCTACAGTTCATTG
GCTGTTCACCACATGTGGGGCCAGCGGGCCCCATGGCCCCACCCAGGCACAGTGCAACAACGCC
TACCAGAACTCCAACCTGAGCGTGGAGGTGGGGAGCGAGGGCCCCCTGAAAGGCATCCAGATCT
GGAAGGTGCCAGCCACCGACACCTACAGCATCTCGGGCTACGGAGCTGCTGGCGGGAAAGGCGG
GAAGAACACCATGATGCGGTCCCACGGCGTGTCTGTGCTGGGCATCTTCAACCTGGAGAAGGAT
GACATGCTGTACATCCTGGTTGGGCAGCAGGGAGAGGACGCCTGCCCCAGTACAAACCAGTTAA
TCCAGAAAGTCTGCATTGGAGAGAACAATGTGATAGAAGAAGAAATCCGTGTGAACAGAAGCGT
GCATGAGTGGGCAGGAGGCGGAGGAGGAGGGGGTGGAGCCACCTACGTATTTAAGATGAAGGAT
GGAGTGCCGGTGCCCCTGATCATTGCAGCCGGAGGTGGTGGCAGGGCCTACGGGGCCAAGACAG
ACACGTTCCACCCAGAGAGACTGGAGAATAACTCCTCGGTTCTAGGGCTAAACGGCAATTCCGG
AGCCGCAGGTGGTGGAGGTGGCTGGAATGATAACACTTCCTTGCTCTGGGCCGGAAAATCTTTG
CAGGAGGGTGCCACCGGAGGACATTCCTGCCCCCAGGCCATGAAGAAGTGGGGGTGGGAGACAA
GAGGGGGTTTCGGAGGGGGTGGAGGGGGGTGCTCCTCAGGTGGAGGAGGCGGAGGATATATAGG
CGGCAATGCAGCCTCAAACAATGACCCCGAAATGGATGGGGAAGATGGGGTTTCCTTCATCAGT
CCACTGGGCATCCTGTACACCCCAGCTTTAAAAGTGATGGAAGGCCACGGGGAAGTGAATATTA
AGCATTATCTAAACTGCAGTCACTGTGAGGTAGACGAATGTCACATGGACCCTGAAAGCCACAA
GGTCATCTGCTTCTGTGACCACGGGACGGTGCTGGCTGAGGATGGCGTCTCCTGCATTGTGTCA
CCCACCCCGGAGCCACACCTGCCACTCTCGCTGATCCTCTCTGTGGTGACCTCTGCCCTCGTGG
CCGCCCTGGTCCTGGCTTTCTCCGGCATCATGATTGTGTACCGCCGGAAGCACCAGGAGCTGCA
AGCCATGCAGATGGAGCTGCAGAGCCCTGAGTACAAGCTGAGCAAGCTCCGCACCTCGACCATC
ATGACCGACTACAACCCCAACTACTGCTTTGCTGGCAAGACCTCCTCCATCAGTGACCTGAAGG
AGGTGCCGCGGAAAAACATCACCCTCATTCGGGGTCTGGGCCATGGCGCCTTTGGGGAGGTGTA
TGAAGGCCAGGTGTCCGGAATGCCCAACGACCCAAGCCCCCTGCAAGTGGCTGTGAAGACGCTG
CCTGAAGTGTGCTCTGAACAGGACGAACTGGATTTCCTCATGGAAGCCCTGATCATCAGCAAAT
TCAACCACCAGAACATTGTTCGCTGCATTGGGGTGAGCCTGCAATCCCTGCCCCGGTTCATCCT
GCTGGAGCTCATGGTGGGGGGAGACCTCAAGTCCTTCCTCCGAGAGACCCGCCCTCGCCCGAGC
CAGCCCTCCTCCCTGGCCATGCTGGACCTTCTGCACGTGGCTCGGGACATTGCCTGTGGCTGTC
AGTATTTGGAGGAAAACCACTTCATCCACCGAGACATTGCTGCCAGAAACTGCCTCTTGACCTG
TCCAGGCCCTGGAAGAGTGGCCAAGATTGGAGACTTCGGGATGGCCCGAGACATCTACAGGGCG
AGCTACTATAGAAAGGGAGGCTGTGCCATGCTGCCAGTTAAGTGGATGCCCCCAGAGGCCTTCA
TGGAAGGAATATTCACTTCTAAAACAGACACATGGTCCTTTGGAGTGCTGCTATGGGAAATCTT
TTCTCTTGGATATATGCCATACCCCAGCAAAAGCAACCAGGAAGTTCTGGAGTTTGTCACCAGT
GGAGGCCGGATGGACCCACCCAAGAACTGCCCTGGGCCTGTATACCGGATAATGACTCAGTGCT
GGCAACATCAGCCTGAAGACAGGCCCAACTTTGCCATCATTTTGGAGAGGATTGAATACTGCAC
CCAGGACCCGGATGTAATCAACACCGCTTTGCCGATAGAATATGGTCCACTTGTGGAAGAGGAA
GAGAAAGTGCCTGTGAGGCCCAAGGACCCTGAGGGGGTTCCTCCTCTCCTGGTCTCTCAACAGG
CAAAACGGGAGGAGGAGCGCAGCCCAGCTGCCCCACCACCTCTGCCTACCACCTCCTCTGGCAA
GGCTGCAAAGAAACCCACAGCTGCAGAGATCTCTGTTCGAGTCCCTAGAGGGCCGGCCGTGGAA
GGGGGACACGTGAATATGGCATTCTCTCAGTCCAACCCTCCTTCGGAGTTGCACAAGGTCCACG
GATCCAGAAACAAGCCCACCAGCTTGTGGAACCCAACGTACGGCTCCTGGTTTACAGAGAAACC
CACCAAAAAGAATAATCCTATAGCAAAGAAGGAGCCACACGACAGGGGTAACCTGGGGCTGGAG
GGAAGCTGTACTGTCCCACCTAACGTTGCAACTGGGAGACTTCCGGGGGCCTCACTGCTCCTAG
AGCCCTCTTCGCTGACTGCCAATATGAAGGAGGTACCTCTGTTCAGGCTACGTCACTTCCCTTG
TGGGAATGTCAATTACGGCTACCAGCAACAGGGCTTGCCCTTAGAAGCCGCTACTGCCCCTGGA
GCTGGTCATTACGAGGATACCATTCTGAAAAGCAAGAATAGCATGAACCAGCCTGGGCCCTGA.
>EU788003.1:908-5770 Homo sapiens mutant K1062M anaplastic lymphoma
kinase (ALK) mRNA, complete cds
(SEQ ID NO: 13)
ATGGGAGCCATCGGGCTCCTGTGGCTCCTGCCGCTGCTGCTTTCCACGGCAGCTGTGGGCTCCG
GGATGGGGACCGGCCAGCGCGCGGGCTCCCCAGCTGCGGGGCCGCCGCTGCAGCCCCGGGAGCC
ACTCAGCTACTCGCGCCTGCAGAGGAAGAGTCTGGCAGTTGACTTCGTGGTGCCCTCGCTCTTC
CGTGTCTACGCCCGGGACCTACTGCTGCCACCATCCTCCTCGGAGCTGAAGGCTGGCAGGCCCG
AGGCCCGCGGCTCGCTAGCTCTGGACTGCGCCCCGCTGCTCAGGTTGCTGGGGCCGGCGCCGGG
GGTCTCCTGGACCGCCGGTTCACCAGCCCCGGCAGAGGCCCGGACGCTGTCCAGGGTGCTGAAG
GGCGGCTCCGTGCGCAAGCTCCGGCGTGCCAAGCAGTTGGTGCTGGAGCTGGGCGAGGAGGCGA
TCTTGGAGGGTTGCGTCGGGCCCCCCGGGGAGGCGGCTGTGGGGCTGCTCCAGTTCAATCTCAG
CGAGCTGTTCAGTTGGTGGATTCGCCAAGGCGAAGGGCGACTGAGGATCCGCCTGATGCCCGAG
AAGAAGGCGTCGGAAGTGGGCAGAGAGGGAAGGCTGTCCGCGGCAATTCGCGCCTCCCAGCCCC
GCCTTCTCTTCCAGATCTTCGGGACTGGTCATAGCTCCTTGGAATCACCAACAAACATGCCTTC
TCCTTCTCCTGATTATTTTACATGGAATCTCACCTGGATAATGAAAGACTCCTTCCCTTTCCTG
TCTCATCGCAGCCGATATGGTCTGGAGTGCAGCTTTGACTTCCCCTGTGAGCTGGAGTATTCCC
CTCCACTGCATGACCTCAGGAACCAGAGCTGGTCCTGGCGCCGCATCCCCTCCGAGGAGGCCTC
CCAGATGGACTTGCTGGATGGGCCTGGGGCAGAGCGTTCTAAGGAGATGCCCAGAGGCTCCTTT
CTCCTTCTCAACACCTCAGCTGACTCCAAGCACACCATCCTGAGTCCGTGGATGAGGAGCAGCA
GTGAGCACTGCACACTGGCCGTCTCGGTGCACAGGCACCTGCAGCCCTCTGGAAGGTACATTGC
CCAGCTGCTGCCCCACAACGAGGCTGCAAGAGAGATCCTCCTGATGCCCACTCCAGGGAAGCAT
GGTTGGACAGTGCTCCAGGGAAGAATCGGGCGTCCAGACAACCCATTTCGAGTGGCCCTGGAAT
ACATCTCCAGTGGAAACCGCAGCTTGTCTGCAGTGGACTTCTTTGCCCTGAAGAACTGCAGTGA
AGGAACATCCCCAGGCTCCAAGATGGCCCTGCAGAGCTCCTTCACTTGTTGGAATGGGACAGTC
CTCCAGCTTGGGCAGGCCTGTGACTTCCACCAGGACTGTGCCCAGGGAGAAGATGAGAGCCAGA
TGTGCCGGAAACTGCCTGTGGGTTTTTACTGCAACTTTGAAGATGGCTTCTGTGGCTGGACCCA
AGGCACACTGTCACCCCACACTCCTCAATGGCAGGTCAGGACCCTAAAGGATGCCCGGTTCCAG
GACCACCAAGACCATGCTCTATTGCTCAGTACCACTGATGTCCCCGCTTCTGAAAGTGCTACAG
TGACCAGTGCTACGTTTCCTGCACCGATCAAGAGCTCTCCATGTGAGCTCCGAATGTCCTGGCT
CATTCGTGGAGTCTTGAGGGGAAACGTGTCCTTGGTGCTAGTGGAGAACAAAACCGGGAAGGAG
CAAGGCAGGATGGTCTGGCATGTCGCCGCCTATGAAGGCTTGAGCCTGTGGCAGTGGATGGTGT
TGCCTCTCCTCGATGTGTCTGACAGGTTCTGGCTGCAGATGGTCGCATGGTGGGGACAAGGATC
CAGAGCCATCGTGGCTTTTGACAATATCTCCATCAGCCTGGACTGCTACCTCACCATTAGCGGA
GAGGACAAGATCCTGCAGAATACAGCACCCAAATCAAGAAACCTGTTTGAGAGAAACCCAAACA
AGGAGCTGAAACCCGGGGAAAATTCACCAAGACAGACCCCCATCTTTGACCCTACAGTTCATTG
GCTGTTCACCACATGTGGGGCCAGCGGGCCCCATGGCCCCACCCAGGCACAGTGCAACAACGCC
TACCAGAACTCCAACCTGAGCGTGGAGGTGGGGAGCGAGGGCCCCCTGAAAGGCATCCAGATCT
GGAAGGTGCCAGCCACCGACACCTACAGCATCTCGGGCTACGGAGCTGCTGGCGGGAAAGGCGG
GAAGAACACCATGATGCGGTCCCACGGCGTGTCTGTGCTGGGCATCTTCAACCTGGAGAAGGAT
GACATGCTGTACATCCTGGTTGGGCAGCAGGGAGAGGACGCCTGCCCCAGTACAAACCAGTTAA
TCCAGAAAGTCTGCATTGGAGAGAACAATGTGATAGAAGAAGAAATCCGTGTGAACAGAAGCGT
GCATGAGTGGGCAGGAGGCGGAGGAGGAGGGGGTGGAGCCACCTACGTATTTAAGATGAAGGAT
GGAGTGCCGGTGCCCCTGATCATTGCAGCCGGAGGTGGTGGCAGGGCCTACGGGGCCAAGACAG
ACACGTTCCACCCAGAGAGACTGGAGAATAACTCCTCGGTTCTAGGGCTAAACGGCAATTCCGG
AGCCGCAGGTGGTGGAGGTGGCTGGAATGATAACACTTCCTTGCTCTGGGCCGGAAAATCTTTG
CAGGAGGGTGCCACCGGAGGACATTCCTGCCCCCAGGCCATGAAGAAGTGGGGGTGGGAGACAA
GAGGGGGTTTCGGAGGGGGTGGAGGGGGGTGCTCCTCAGGTGGAGGAGGCGGAGGATATATAGG
CGGCAATGCAGCCTCAAACAATGACCCCGAAATGGATGGGGAAGATGGGGTTTCCTTCATCAGT
CCACTGGGCATCCTGTACACCCCAGCTTTAAAAGTGATGGAAGGCCACGGGGAAGTGAATATTA
AGCATTATCTAAACTGCAGTCACTGTGAGGTAGACGAATGTCACATGGACCCTGAAAGCCACAA
GGTCATCTGCTTCTGTGACCACGGGACGGTGCTGGCTGAGGATGGCGTCTCCTGCATTGTGTCA
CCCACCCCGGAGCCACACCTGCCACTCTCGCTGATCCTCTCTGTGGTGACCTCTGCCCTCGTGG
CCGCCCTGGTCCTGGCTTTCTCCGGCATCATGATTGTGTACCGCCGGATGCACCAGGAGCTGCA
AGCCATGCAGATGGAGCTGCAGAGCCCTGAGTACAAGCTGAGCAAGCTCCGCACCTCGACCATC
ATGACCGACTACAACCCCAACTACTGCTTTGCTGGCAAGACCTCCTCCATCAGTGACCTGAAGG
AGGTGCCGCGGAAAAACATCACCCTCATTCGGGGTCTGGGCCATGGCGCCTTTGGGGAGGTGTA
TGAAGGCCAGGTGTCCGGAATGCCCAACGACCCAAGCCCCCTGCAAGTGGCTGTGAAGACGCTG
CCTGAAGTGTGCTCTGAACAGGACGAACTGGATTTCCTCATGGAAGCCCTGATCATCAGCAAAT
TCAACCACCAGAACATTGTTCGCTGCATTGGGGTGAGCCTGCAATCCCTGCCCCGGTTCATCCT
GCTGGAGCTCATGGCGGGGGGAGACCTCAAGTCCTTCCTCCGAGAGACCCGCCCTCGCCCGAGC
CAGCCCTCCTCCCTGGCCATGCTGGACCTTCTGCACGTGGCTCGGGACATTGCCTGTGGCTGTC
AGTATTTGGAGGAAAACCACTTCATCCACCGAGACATTGCTGCCAGAAACTGCCTCTTGACCTG
TCCAGGCCCTGGAAGAGTGGCCAAGATTGGAGACTTCGGGATGGCCCGAGACATCTACAGGGCG
AGCTACTATAGAAAGGGAGGCTGTGCCATGCTGCCAGTTAAGTGGATGCCCCCAGAGGCCTTCA
TGGAAGGAATATTCACTTCTAAAACAGACACATGGTCCTTTGGAGTGCTGCTATGGGAAATCTT
TTCTCTTGGATATATGCCATACCCCAGCAAAAGCAACCAGGAAGTTCTGGAGTTTGTCACCAGT
GGAGGCCGGATGGACCCACCCAAGAACTGCCCTGGGCCTGTATACCGGATAATGACTCAGTGCT
GGCAACATCAGCCTGAAGACAGGCCCAACTTTGCCATCATTTTGGAGAGGATTGAATACTGCAC
CCAGGACCCGGATGTAATCAACACCGCTTTGCCGATAGAATATGGTCCACTTGTGGAAGAGGAA
GAGAAAGTGCCTGTGAGGCCCAAGGACCCTGAGGGGGTTCCTCCTCTCCTGGTCTCTCAACAGG
CAAAACGGGAGGAGGAGCGCAGCCCAGCTGCCCCACCACCTCTGCCTACCACCTCCTCTGGCAA
GGCTGCAAAGAAACCCACAGCTGCAGAGATCTCTGTTCGAGTCCCTAGAGGGCCGGCCGTGGAA
GGGGGACACGTGAATATGGCATTCTCTCAGTCCAACCCTCCTTCGGAGTTGCACAAGGTCCACG
GATCCAGAAACAAGCCCACCAGCTTGTGGAACCCAACGTACGGCTCCTGGTTTACAGAGAAACC
CACCAAAAAGAATAATCCTATAGCAAAGAAGGAGCCACACGACAGGGGTAACCTGGGGCTGGAG
GGAAGCTGTACTGTCCCACCTAACGTTGCAACTGGGAGACTTCCGGGGGCCTCACTGCTCCTAG
AGCCCTCTTCGCTGACTGCCAATATGAAGGAGGTACCTCTGTTCAGGCTACGTCACTTCCCTTG
TGGGAATGTCAATTACGGCTACCAGCAACAGGGCTTGCCCTTAGAAGCCGCTACTGCCCCTGGA
GCTGGTCATTACGAGGATACCATTCTGAAAAGCAAGAATAGCATGAACCAGCCTGGGCCCTGA.

By “alteration” is meant a change in the structure, expression levels or activity of a gene or polypeptide as detected by standard art known methods such as those described herein. An alteration may be an increase or decrease. As used herein, an alteration includes a 5% change in expression levels, a 10% change in expression levels, preferably a 25% change, more preferably a 40% change, and most preferably a 50% or greater change in expression levels.

By “ameliorate” is meant decrease, reduce, delay diminish, suppress, attenuate, arrest, or stabilize the development or progression of a disease or pathological condition.

By “antibody” is meant an immunoglobulin polypeptide having immunogen binding ability. Antibodies are evoked or elicited in subjects (humans or other animals or mammals) following exposure to a specific antigen (immunogen). A subject capable of generating antibodies/immunoglobulins (i.e., an immune response) directed against a specific antigen/immunogen is said to be immunocompetent. Antibodies are characterized by reacting specifically with (e.g., binding to) an antigen or immunogen in some demonstrable way, antibody, and antigen/immunogen each being defined in terms of the other.

“Eliciting an antibody response” refers to the ability a molecule to induce the production of antibodies. Antibodies are of different classes, e.g., IgM, IgG, IgA, IgE, IgD and subtypes or subclasses, e.g., IgG1, IgG2, IgG2a, IgG2b, IgG3, IgG4. An antibody/immunoglobulin response elicited in a subject can neutralize a pathogenic (e.g., disease-causing) agent by binding to epitopes (antigenic determinants) on the agent and blocking or inhibiting the activity of the agent, and/or by forming a binding complex with the agent that is cleared from the system of the subject, e.g., via the liver.

By “amphiphile” is meant a chemical compound possessing both hydrophilic and lipophilic properties. Such a compound is called amphiphilic or amphipathic. The amphiphile may be conjugated or linked to an antigen or adjuvant cargo by a solubility-promoting polar polymer chain. In some embodiments, the amphiphile is conjugated or linked to an adjuvant. In some embodiments, the adjuvant is Freund's adjuvant. In some embodiments, the amphiphile is conjugated or linked to an ALK antigen or immunogen. In some embodiments, the amphiphile is a lipophilic albumin-binding tail. In some embodiments, the amphiphile is N-hydroxy succinimidyl ester-end-functionalized poly(ethylene glycol)-lipid (NHS-PEG2KDa-DSPE).

By “antigen” is meant an agent that can stimulate an immune response in an animal. An antigen reacts with the products of specific humoral or cellular immunity, including those induced by heterologous immunogens. In some embodiments of the disclosed compositions and methods, the antigen is an ALK protein or an antibody-binding portion thereof.

A “codon-optimized” nucleic acid (polynucleotide) refers to a nucleic acid sequence that has been altered such that the codons are optimal for expression in a particular system (such as a particular species of group of species). For example, a nucleic acid sequence can be optimized for expression in mammalian cells. Codon optimization does not alter the amino acid sequence of the encoded protein.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

“Detect” refers to identifying the presence, absence or amount of an analyte, compound, agent, or substance to be detected.

By “detectable label” is meant a composition that, when linked to a molecule of interest, renders the latter detectable, e.g., via spectroscopic, photochemical, biochemical, immunochemical, or chemical means. Nonlimiting examples of useful detectable labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition, disorder, or pathology that damages or interferes with the normal function of a cell, tissue, or organ. Examples of diseases include those caused by oncogenic ALK gene fusions, rearrangements, duplications, or mutations (e.g., ALK-positive cancers). In some embodiments, the cancer is an ALK-positive cancer. By “ALK-positive cancer” is meant a cancer or tumor that expresses the ALK protein. Nonlimiting examples of ALK-positive cancers include non-small cell lung cancer (NSCLC), anaplastic large cell lymphoma (ALCL), neuroblastoma, B-cell lymphoma, thyroid cancer, colon cancer, breast cancer, inflammatory myofibroblastic tumors (IMT), renal carcinoma, esophageal cancer, glioma, glioblastoma, and melanoma. The ALK-positive cancer may be caused by an oncogenic ALK gene that either forms a fusion gene with other genes, gains additional gene copies, or is genetically mutated. In some embodiments, the ALK-positive cancer is caused by an ALK fusion gene encoding an ALK fusion protein. In some embodiments, the ALK-positive cancer is caused by a fusion between the ALK gene and the nucleophosmin (NPM) gene encoding a NPM-ALK fusion protein. In some embodiments, the ALK-positive cancer is caused by a fusion between the ALK gene and the echinoderm microtubule-associated protein-like 4 (EML4) gene encoding an ELM4-ALK fusion protein.

By “effective amount” is meant the amount of an active therapeutic agent, composition, compound, biologic (e.g., a vaccine or therapeutic peptide, polypeptide, or polynucleotide) required to ameliorate, reduce, delay, improve, abrogate, diminish, or eliminate the symptoms and/or effects of a disease, condition, or pathology relative to an untreated patient. In some embodiments, an effective amount of an ALK peptide is the amount required to induce an ALK-specific immune response in a subject immunized with the peptide. The effective amount of an immunogen or a composition comprising an immunogen, as used to practice the methods of therapeutic treatment of a disease, condition, or pathology, varies depending upon the manner of administration, the age, body weight, and general health of the subject. Ultimately, the attending physician or veterinarian will decide the appropriate amount and dosage regimen. Such amount is referred to as an “effective” amount.

A “therapeutically effective amount” refers to a quantity of a specified agent sufficient to achieve a desired effect in a subject being treated with that agent. For example, this may be the amount of an ALK-specific antigen, immunogen, immunogenic composition, or vaccine useful for eliciting an immune response in a subject, treating and/or for preventing a disease caused by oncogenic ALK gene fusions, rearrangements, duplications, or mutations (e.g., ALK-positive cancers). Ideally, in the context of the present disclosure, a therapeutically effective amount of an ALK-specific vaccine or immunogenic composition is an amount sufficient to prevent, ameliorate, reduce, delay and/or treat a disease caused by oncogenic ALK gene fusions, rearrangements, duplications, or mutations (e.g., ALK-positive cancers) in a subject without causing a substantial cytotoxic effect in the subject. The effective amount of an ALK-specific vaccine or immunogenic composition useful for preventing, delaying, ameliorating, reducing, and/or treating a disease caused by oncogenic ALK gene fusions, rearrangements, duplications, or mutations (e.g., ALK-positive cancers) in a subject depends on, for example, the subject being treated, the manner of administration of the therapeutic composition and other factors, as noted above.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 3, 4, 5, 6, 7, 8, 9, 10, 20, 30, 40, 50, 60, 70, 80, 90, or 100, 200, 300, 400, 500, 600, 700, 800, 900, or 1000 nucleotides or amino acids. A portion or fragment of a polypeptide may be a peptide. In the case of an antibody or immunoglobulin fragment, the fragment typically binds to the target antigen.

By “fusion protein” is meant a protein generated by expression of a nucleic acid (polynucleotide) sequence engineered from nucleic acid sequences encoding at least a portion of two different (heterologous) proteins or peptides. To create a fusion protein, the nucleic acid sequences must be in the same open reading frame and contain no internal stop codons. One protein can be located at the amino-terminal (N-terminal) portion of the fusion protein or at the carboxy-terminal (C-terminal) protein thus forming an amino-terminal fusion protein or a carboxy-terminal fusion protein, respectively.

For example, a fusion protein includes an ALK protein fused to a heterologous protein. In some embodiments, the fusion protein is an ALK protein fused to a nucleophosmin (NPM) protein. In some embodiments, the NPM-ALK fusion protein is at least 85%, at least 90%, at least 95%, at least 98%, at least 99%, or 100% identical to a NPM-ALK fusion protein in Homo Sapiens. In some embodiments, the NPM-ALK fusion protein is at least 85%, at least 90%, at least 95%, at least 98%, at least 99%, or 100% identical to an exemplary NPM-ALK fusion protein amino acid sequence from Homo Sapiens as provided below (see GenBank Accession Nos. BAA08343.1, AAA58698.1):

>BAA08343.1 p80 protein [Homo sapiens](ALK cyto-
plasmic portion in bold font)
(SEQ ID NO: 14)
MEDSMDMDMSPLRPQNYLFGCELKADKDYHFKVDNDENEHQLSLRTVSLG
AGAKDELHIVEAEAMNYEGSPIKVTLATLKMSVQPTVSLGGFEITPPVVL
RLKCGSGPVHISGQHLVVYRRKHQELQAMQMELQSPEYKLSKLRTSTIMT
DYNPNYCFAGKTSSISDLKEVPRKNITLIRGLGHGAFGEVYEGQVSGMPN
DPSPLQVAVKTLPEVCSEQDELDFLMEALIISKFNHONIVRCIGVSLQSL
PRFILLELMAGGDLKSFLRETRPRPSQPSSLAMLDLLHVARDIACGCQYL
EENHFIHRDIAARNCLLTCPGPGRVAKIGDFGMARDIYRASYYRKGGCAM
LPVKWMPPEAFMEGIFTSKTDTWSFGVLLWEIFSLGYMPYPSKSNQEVLE
FVTSGGRMDPPKNCPGPVYRIMTQCWQHQPEDRPNFAIILERIEYCTQDP
DVINTALPIEYGPLVEEEEKVPVRPKDPEGVPPLLVSQQAKREEERSPAA
PPPLPTTSSGKAAKKPTAAEVSVRVPRGPAVEGGHVNMAFSQSNPPSELH
RVHGSRNKPTSLWNPTYGSWFTEKPTKKNNPIAKKEPHERGNLGLEGSCT
VPPNVATGRLPGASLLLEPSSLTANMKEVPLFRLRHFPCGNVNYGYQQQG
LPLEAATAPGAGHYEDTILKSKNSMNQPGP.
>AAA58698.1 nucleophosmin-anaplastic lymphoma
kinase fusion protein [Homo sapiens]
(SEQ ID NO: 15)
MEDSMDMDMSPLRPQNYLFGCELKADKDYHFKVDNDENEHQLSLRTVSLG
AGAKDELHIVEAEAMNYEGSPIKVTLATLKMSVQPTVSLGGFEITPPVVL
RLKCGSGPVHISGQHLVVYRRKHQELQAMQMELQSPEYKLSKLRTSTIMT
DYNPNYCFAGKTSSISDLKEVPRKNITLIRGLGHGAFGEVYEGQVSGMPN
DPSPLQVAVKTLPEVCSEQDELDFLMEALIISKFNHQNIVRCIGVSLQSL
PRFILLELMAGGDLKSFLRETRPRPSQPSSLAMLDLLHVARDIACGCQYL
EENHFIHRDIAARNCLLTCPGPGRVAKIGDFGMARDIYRASYYRKGGCAM
LPVKWMPPEAFMEGIFTSKTDTWSFGVLLWEIFSLGYMPYPSKSNQEVLE
FVTSGGRMDPPKNCPGPVYRIMTQCWQHQPEDRPNFAIILERIEYCTQDP
DVINTALPIEYGPLVEEEEKVPVRPKDPEGVPPLLVSQQAKREEERSPAA
PPPLPTTSSGKAAKKPTAAEVSVRVPRGPAVEGGHVNMAFSQSNPPSELH
KVHGSRNKPTSLWNPTYGSWFTEKPTKKNNPIAKKEPHDRGNLGLEGSCT
VPPNVATGRLPGASLLLEPSSLTANMKEVPLFRLRHFPCGNVNYGYQQQG
LPLEAATAPGAGHYEDTILKSKNSMNQPGP.

In some embodiments, the NPM-ALK fusion protein is encoded by a nucleic acid sequence that is at least 85%, at least 90%, at least 95%, at least 98%, at least 99%, or 100% identical to an exemplary nucleic acid sequence from Homo Sapiens as provided below (see GenBank Accessions No. D45915.1 and U04946.1):

>D45915.1: 90-2132 Homo sapiens mRNA for p80 protein, complete cds
(SEQ ID NO: 16)
ATGGAAGATTCGATGGACATGGACATGAGCCCCCTGAGGCCCCAGAACTATCTTTTCGGTTGTG
AACTAAAGGCCGACAAAGATTATCACTTTAAGGTGGATAATGATGAAAATGAGCACCAGTTATC
TTTAAGAACGGTCAGTTTAGGGGCTGGTGCAAAGGATGAGTTGCACATTGTTGAAGCAGAGGCA
ATGAATTACGAAGGCAGTCCAATTAAAGTAACACTGGCAACTTTGAAAATGTCTGTACAGCCAA
CGGTTTCCCTTGGGGGCTTTGAAATAACACCACCAGTGGTCTTAAGGTTGAAGTGTGGTTCAGG
GCCAGTGCATATTAGTGGACAGCACTTAGTAGTGTACCGCCGGAAGCACCAGGAGCTGCAAGCC
ATGCAGATGGAGCTGCAGAGCCCTGAGTACAAGCTGAGCAAGCTCCGCACCTCGACCATCATGA
CCGACTACAACCCCAACTACTGCTTTGCTGGCAAGACCTCCTCCATCAGTGACCTGAAGGAGGT
GCCACGGAAAAACATCACCCTCATTCGGGGTCTGGGCCATGGAGCCTTTGGGGAGGTGTATGAA
GGCCAGGTGTCCGGAATGCCCAACGACCCAAGCCCCCTGCAAGTGGCTGTGAAGACGCTGCCTG
AAGTGTGCTCTGAACAGGACGAACTGGATTTCCTCATGGAAGCCCTGATCATCAGCAAATTCAA
CCACCAGAACATTGTTCGCTGCATTGGGGTGAGCCTGCAATCCCTGCCCCGGTTCATCCTGCTG
GAGCTCATGGCGGGGGGAGACCTCAAGTCCTTCCTCCGAGAGACCCGCCCTCGCCCGAGCCAGC
CCTCCTCCCTGGCCATGCTGGACCTTCTGCACGTGGCTCGGGACATTGCCTGTGGCTGTCAGTA
TTTGGAGGAAAACCACTTCATCCACCGAGACATTGCTGCCAGAAACTGCCTCTTGACCTGTCCA
GGCCCTGGAAGAGTGGCCAAGATTGGAGACTTCGGGATGGCCCGAGACATCTACAGGGCGAGCT
ACTATAGAAAGGGAGGCTGTGCCATGCTGCCAGTTAAGTGGATGCCCCCAGAGGCCTTCATGGA
AGGAATATTCACTTCTAAAACAGACACATGGTCCTTTGGAGTGCTGCTATGGGAAATCTTTTCT
CTTGGATATATGCCATACCCCAGCAAAAGCAACCAGGAAGTTCTGGAGTTTGTCACCAGTGGAG
GCCGGATGGACCCACCCAAGAACTGCCCTGGGCCTGTATACCGGATAATGACTCAGTGCTGGCA
ACATCAGCCTGAAGACAGGCCCAACTTTGCCATCATTTTGGAGAGGATTGAATACTGCACCCAG
GACCCGGATGTAATCAACACCGCTTTGCCGATAGAATATGGTCCACTTGTGGAAGAGGAAGAGA
AAGTGCCTGTGAGGCCCAAGGACCCTGAGGGGGTTCCTCCTCTCCTGGTCTCTCAACAGGCAAA
ACGGGAGGAGGAGCGCAGCCCAGCTGCCCCACCACCTCTGCCTACCACCTCCTCTGGCAAGGCT
GCAAAGAAACCCACAGCTGCAGAGGTCTCTGTTCGAGTCCCTAGAGGGCCGGCCGTGGAAGGGG
GACACGTGAATATGGCATTCTCTCAGTCCAACCCTCCTTCGGAGTTGCACAGGGTCCACGGATC
CAGAAATAAGCCCACCAGCTTGTGGAACCCAACGTACGGCTCCTGGTTTACAGAGAAACCCACC
AAAAAGAATAATCCTATAGCAAAGAAGGAGCCACACGAGAGGGGTAACCTGGGGCTGGAGGGAA
GCTGTACTGTCCCACCTAACGTTGCAACTGGGAGACTTCCGGGGGCCTCACTGCTCCTAGAGCC
CTCTTCGCTGACTGCCAATATGAAGGAGGTACCTCTGTTCAGGCTACGTCACTTCCCTTGTGGG
AATGTCAATTACGGCTACCAGCAACAGGGCTTGCCCTTAGAAGCCGCTACTGCCCCTGGAGCTG
GTCATTACGAGGATACCATTCTGAAAAGCAAGAATAGCATGAACCAGCCTGGGCCCTGA.
>U04946.1 Human nucleophosmin-anaplastic lymphoma kinase fusion protein (NPM/ALK)
mRNA, complete cds
(SEQ ID NO: 17)
ATGGAAGATTCGATGGACATGGACATGAGCCCCCTGAGGCCCCAGAACTATCTTTTCGGTTGTG
AACTAAAGGCCGACAAAGATTATCACTTTAAGGTGGATAATGATGAAAATGAGCACCAGTTATC
TTTAAGAACGGTCAGTTTAGGGGCTGGTGCAAAGGATGAGTTGCACATTGTTGAAGCAGAGGCA
ATGAATTACGAAGGCAGTCCAATTAAAGTAACACTGGCAACTTTGAAAATGTCTGTACAGCCAA
CGGTTTCCCTTGGGGGCTTTGAAATAACACCACCAGTGGTCTTAAGGTTGAAGTGTGGTTCAGG
GCCAGTGCATATTAGTGGACAGCACTTAGTAGTGTACCGCCGGAAGCACCAGGAGCTGCAAGCC
ATGCAGATGGAGCTGCAGAGCCCTGAGTACAAGCTGAGCAAGCTCCGCACCTCGACCATCATGA
CCGACTACAACCCCAACTACTGCTTTGCTGGCAAGACCTCCTCCATCAGTGACCTGAAGGAGGT
GCCGCGGAAAAACATCACCCTCATTCGGGGTCTGGGCCATGGCGCCTTTGGGGAGGTGTATGAA
GGCCAGGTGTCCGGAATGCCCAACGACCCAAGCCCCCTGCAAGTGGCTGTGAAGACGCTGCCTG
AAGTGTGCTCTGAACAGGACGAACTGGATTTCCTCATGGAAGCCCTGATCATCAGCAAATTCAA
CCACCAGAACATTGTTCGCTGCATTGGGGTGAGCCTGCAATCCCTGCCCCGGTTCATCCTGCTG
GAGCTCATGGCGGGGGGAGACCTCAAGTCCTTCCTCCGAGAGACCCGCCCTCGCCCGAGCCAGC
CCTCCTCCCTGGCCATGCTGGACCTTCTGCACGTGGCTCGGGACATTGCCTGTGGCTGTCAGTA
TTTGGAGGAAAACCACTTCATCCACCGAGACATTGCTGCCAGAAACTGCCTCTTGACCTGTCCA
GGCCCTGGAAGAGTGGCCAAGATTGGAGACTTCGGGATGGCCCGAGACATCTACAGGGCGAGCT
ACTATAGAAAGGGAGGCTGTGCCATGCTGCCAGTTAAGTGGATGCCCCCAGAGGCCTTCATGGA
AGGAATATTCACTTCTAAAACAGACACATGGTCCTTTGGAGTGCTGCTATGGGAAATCTTTTCT
CTTGGATATATGCCATACCCCAGCAAAAGCAACCAGGAAGTTCTGGAGTTTGTCACCAGTGGAG
GCCGGATGGACCCACCCAAGAACTGCCCTGGGCCTGTATACCGGATAATGACTCAGTGCTGGCA
ACATCAGCCTGAAGACAGGCCCAACTTTGCCATCATTTTGGAGAGGATTGAATACTGCACCCAG
GACCCGGATGTAATCAACACCGCTTTGCCGATAGAATATGGTCCACTTGTGGAAGAGGAAGAGA
AAGTGCCTGTGAGGCCCAAGGACCCTGAGGGGGTTCCTCCTCTCCTGGTCTCTCAACAGGCAAA
ACGGGAGGAGGAGCGCAGCCCAGCTGCCCCACCACCTCTGCCTACCACCTCCTCTGGCAAGGCT
GCAAAGAAACCCACAGCTGCAGAGGTCTCTGTTCGAGTCCCTAGAGGGCCGGCCGTGGAAGGGG
GACACGTGAATATGGCATTCTCTCAGTCCAACCCTCCTTCGGAGTTGCACAAGGTCCACGGATC
CAGAAACAAGCCCACCAGCTTGTGGAACCCAACGTACGGCTCCTGGTTTACAGAGAAACCCACC
AAAAAGAATAATCCTATAGCAAAGAAGGAGCCACACGACAGGGGTAACCTGGGGCTGGAGGGAA
GCTGTACTGTCCCACCTAACGTTGCAACTGGGAGACTTCCGGGGGCCTCACTGCTCCTAGAGCC
CTCTTCGCTGACTGCCAATATGAAGGAGGTACCTCTGTTCAGGCTACGTCACTTCCCTTGTGGG
AATGTCAATTACGGCTACCAGCAACAGGGCTTGCCCTTAGAAGCCGCTACTGCCCCTGGAGCTG
GTCATTACGAGGATACCATTCTGAAAAGCAAGAATAGCATGAACCAGCCTGGGCCCTGA.

In some embodiments, the fusion protein is an ALK protein fused to an echinoderm microtubule-associated protein-like 4 (EML4) protein. In some embodiments, the ELM4-ALK fusion protein is at least 85%, at least 90%, at least 95%, at least 98%, at least 99%, or 100% identical to a ELM4-ALK fusion protein in Homo Sapiens or a variant thereof. In some embodiments, the ELM4-ALK fusion protein is at least 85%, at least 90%, at least 95%, at least 98%, at least 99%, or 100% identical to an exemplary ELM4-ALK fusion protein amino acid sequence from Homo Sapiens as provided below (see GenBank Accessions No. BAM37627.1 and BAF73611.1):

>BAM37627.1 EML4-ALK fusion protein [Homo sapiens]
(SEQ ID NO: 18)
MDGFAGSLDDSISAASTSDVQDRLSALESRVQQQEDEITVLKAALADVLR
RLAISEDHVASVKKSVSSKGQPSPRAVIPMSCITNGSGANRKPSHTSAVS
IAGKETLSSAAKSGTEKKKEKPQGQREKKEESHSNDQSPQIRASPSPQPS
SQPLQIHRQTPESKNATPTKSIKRPSPAEKSHNSWENSDDSRNKLSKIPS
TPKLIPKVTKTADKHKDVIINQEGEYIKMFMRGRPITMFIPSDVDNYDDI
RTELPPEKLKLEWAYGYRGKDCRANVYLLPTGEIVYFIASVVVLFNYEER
TQRHYLGHTDCVKCLAIHPDKIRIATGQIAGVDKDGRPLQPHVRVWDSVT
LSTLQIIGLGTFERGVGCLDFSKADSGVHLCVIDDSNEHMLTVWDWQRKA
KGAEIKTTNEVVLAVEFHPTDANTIITCGKSHIFFWTWSGNSLTRKQGIF
GKYEKPKFVQCLAFLGNGDVLTGDSGGVMLIWSKTTVEPTPGKGPKGVYQ
ISKQIKAHDGSVFTLCQMRNGMLLTGGGKDRKIILWDHDLNPEREIEFSA
SRARLPGHVAADHPPAVYRRKHQELQAMQMELQSPEYKLSKLRTSTIMTD
YNPNYCFAGKTSSISDLKEVPRKNITLIRGLGHGAFGEVYEGQVSGMPND
PSPLQVAVKTLPEVCSEQDELDFLMEALIISKFNHQNIVRCIGVSLQSLP
RFILLELMAGGDLKSFLRETRPRPSQPSSLAMLDLLHVARDIACGCQYLE
ENHFIHRDIAARNCLLTCPGPGRVAKIGDFGMARDIYRASYYRKGGCAML
PVKWMPPEAFMEGIFTSKTDTWSFGVLLWEIFSLGYMPYPSKSNQEVLEF
VTSGGRMDPPKNCPGPVYRIMTQCWQHQPEDRPNFAIILERIEYCTQDPD
VINTALPIEYGPLVEEEEKVPVRPKDPEGVPPLLVSQQAKREEERSPAAP
PPLPTTSSGKAAKKPTAAEISVRVPRGPAVEGGHVNMAFSQSNPPSELHK
VHGSRNKPTSLWNPTYGSWFTEKPTKKNNPIAKKEPHDRGNLGLEGSCTV
PPNVATGRLPGASLLLEPSSLTANMKEVPLFRLRHFPCGNVNYGYQQQGL
PLEAATAPGAGHYEDTILKSKNSMNQPGP.
>BAF73611.1 fusion protein EML4-ALK variant 1
[Homo sapiens]
(SEQ ID NO: 19)
MDGFAGSLDDSISAASTSDVQDRLSALESRVQQQEDEITVLKAALADVLR
RLAISEDHVASVKKSVSSKGQPSPRAVIPMSCITNGSGANRKPSHTSAVS
IAGKETLSSAAKSGTEKKKEKPQGQREKKEESHSNDQSPQIRASPSPQPS
SQPLQIHRQTPESKNATPTKSIKRPSPAEKSHNSWENSDDSRNKLSKIPS
TPKLIPKVTKTADKHKDVIINQEGEYIKMFMRGRPITMFIPSDVDNYDDI
RTELPPEKLKLEWAYGYRGKDCRANVYLLPTGEIVYFIASVVVLFNYEER
TQRHYLGHTDCVKCLAIHPDKIRIATGQIAGVDKDGRPLQPHVRVWDSVT
LSTLQIIGLGTFERGVGCLDFSKADSGVHLCVIDDSNEHMLTVWDWQKKA
KGAEIKTTNEVVLAVEFHPTDANTIITCGKSHIFFWTWSGNSLTRKQGIF
GKYEKPKFVQCLAFLGNGDVLTGDSGGVMLIWSKTTVEPTPGKGPKVYRR
KHQELQAMQMELQSPEYKLSKLRTSTIMTDYNPNYCFAGKTSSISDLKEV
PRKNITLIRGLGHGAFGEVYEGQVSGMPNDPSPLQVAVKTLPEVCSEQDE
LDFLMEALIISKFNHQNIVRCIGVSLQSLPRFILLELMAGGDLKSFLRET
RPRPSQPSSLAMLDLLHVARDIACGCQYLEENHFIHRDIAARNCLLTCPG
PGRVAKIGDFGMARDIYRASYYRKGGCAMLPVKWMPPEAFMEGIFTSKTD
TWSFGVLLWEIFSLGYMPYPSKSNQEVLEFVTSGGRMDPPKNCPGPVYRI
MTQCWQHQPEDRPNFAIILERIEYCTQDPDVINTALPIEYGPLVEEEEKV
PVRPKDPEGVPPLLVSQQAKREEERSPAAPPPLPTTSSGKAAKKPTAAEV
SVRVPRGPAVEGGHVNMAFSQSNPPSELHRVHGSRNKPTSLWNPTYGSWF
TEKPTKKNNPIAKKEPHERGNLGLEGSCTVPPNVATGRLPGASLLLEPSS
LTANMKEVPLFRLRHFPCGNVNYGYQQQGLPLEAATAPGAGHYEDTILKS
KNSMNQPGP.

In some embodiments, the ELM4-ALK fusion protein is encoded by a nucleic acid sequence that is at least 85%, at least 90%, at least 95%, at least 98%, at least 99%, or 100% identical to an exemplary nucleic acid sequence from Homo Sapiens as provided below (see GenBank Accessions No. AB663645.1 and AB274722.1):

>AB663645.1: 15-3404 Homo sapiens EML4-ALK mRNA for EML4-ALK fusion protein,
complete cds
(SEQ ID NO: 20)
ATGGACGGTTTCGCCGGCAGTCTCGATGATAGTATTTCTGCTGCAAGTACTTCTGATGTTCAAG
ATCGCCTGTCAGCTCTTGAGTCACGAGTTCAGCAACAAGAAGATGAAATCACTGTGCTAAAGGC
GGCTTTGGCTGATGTTTTGAGGCGTCTTGCAATCTCTGAAGATCATGTGGCCTCAGTGAAAAAA
TCAGTCTCAAGTAAAGGCCAACCAAGCCCTCGAGCAGTTATTCCCATGTCCTGTATAACCAATG
GAAGTGGTGCAAACAGAAAACCAAGTCATACCAGTGCTGTCTCAATTGCAGGAAAAGAAACTCT
TTCATCTGCTGCTAAAAGTGGTACAGAAAAAAAGAAAGAAAAACCACAAGGACAGAGAGAAAAA
AAAGAGGAATCTCATTCTAATGATCAAAGTCCACAAATTCGAGCATCACCTTCTCCCCAGCCCT
CTTCACAACCTCTCCAAATACACAGACAAACTCCAGAAAGCAAGAATGCTACTCCCACCAAAAG
CATAAAACGACCATCACCAGCTGAAAAGTCACATAATTCTTGGGAAAATTCAGATGATAGCCGT
AATAAATTGTCGAAAATACCTTCAACACCCAAATTAATACCAAAAGTTACCAAAACTGCAGACA
AGCATAAAGATGTCATCATCAACCAAGAAGGAGAATATATTAAAATGTTTATGCGCGGTCGGCC
AATTACCATGTTCATTCCTTCCGATGTTGACAACTATGATGACATCAGAACGGAACTGCCTCCT
GAGAAGCTCAAACTGGAGTGGGCATATGGTTATCGAGGAAAGGACTGTAGAGCTAATGTTTACC
TTCTTCCGACCGGGGAAATAGTTTATTTCATTGCATCAGTAGTAGTACTATTTAATTATGAGGA
GAGAACTCAGCGACACTACCTGGGCCATACAGACTGTGTGAAATGCCTTGCTATACATCCTGAC
AAAATTAGGATTGCAACTGGACAGATAGCTGGCGTGGATAAAGATGGAAGGCCTCTACAACCCC
ACGTCAGAGTGTGGGATTCTGTTACTCTATCCACACTGCAGATTATTGGACTTGGCACTTTTGA
GCGTGGAGTAGGATGCCTGGATTTTTCAAAAGCAGATTCAGGTGTTCATTTATGTGTTATTGAT
GACTCCAATGAGCATATGCTTACTGTATGGGACTGGCAGAGGAAAGCAAAAGGAGCAGAAATAA
AGACAACAAATGAAGTTGTTTTGGCTGTGGAGTTTCACCCAACAGATGCAAATACCATAATTAC
ATGCGGTAAATCTCATATTTTCTTCTGGACCTGGAGCGGCAATTCACTAACAAGAAAACAGGGA
ATTTTTGGGAAATATGAAAAGCCAAAATTTGTGCAGTGTTTAGCATTCTTGGGGAATGGAGATG
TTCTTACTGGAGACTCAGGTGGAGTCATGCTTATATGGAGCAAAACTACTGTAGAGCCCACACC
TGGGAAAGGACCTAAAGGTGTATATCAAATCAGCAAACAAATCAAAGCTCATGATGGCAGTGTG
TTCACACTTTGTCAGATGAGAAATGGGATGTTATTAACTGGAGGAGGGAAAGACAGAAAAATAA
TTCTGTGGGATCATGATCTGAATCCTGAAAGAGAAATAGAGTTTAGTGCTTCAAGGGCCAGGCT
GCCAGGCCATGTTGCAGCTGACCACCCACCTGCAGTGTACCGCCGGAAGCACCAGGAGCTGCAA
GCCATGCAGATGGAGCTGCAGAGCCCTGAGTACAAGCTGAGCAAGCTCCGCACCTCGACCATCA
TGACCGACTACAACCCCAACTACTGCTTTGCTGGCAAGACCTCCTCCATCAGTGACCTGAAGGA
GGTGCCGCGGAAAAACATCACCCTCATTCGGGGTCTGGGCCATGGCGCCTTTGGGGAGGTGTAT
GAAGGCCAGGTGTCCGGAATGCCCAACGACCCAAGCCCCCTGCAAGTGGCTGTGAAGACGCTGC
CTGAAGTGTGCTCTGAACAGGACGAACTGGATTTCCTCATGGAAGCCCTGATCATCAGCAAATT
CAACCACCAGAACATTGTTCGCTGCATTGGGGTGAGCCTGCAATCCCTGCCCCGGTTCATCCTG
CTGGAGCTCATGGCGGGGGGAGACCTCAAGTCCTTCCTCCGAGAGACCCGCCCTCGCCCGAGCC
AGCCCTCCTCCCTGGCCATGCTGGACCTTCTGCACGTGGCTCGGGACATTGCCTGTGGCTGTCA
GTATTTGGAGGAAAACCACTTCATCCACCGAGACATTGCTGCCAGAAACTGCCTCTTGACCTGT
CCAGGCCCTGGAAGAGTGGCCAAGATTGGAGACTTCGGGATGGCCCGAGACATCTACAGGGCGA
GCTACTATAGAAAGGGAGGCTGTGCCATGCTGCCAGTTAAGTGGATGCCCCCAGAGGCCTTCAT
GGAAGGAATATTCACTTCTAAAACAGACACATGGTCCTTTGGAGTGCTGCTATGGGAAATCTTT
TCTCTTGGATATATGCCATACCCCAGCAAAAGCAACCAGGAAGTTCTGGAGTTTGTCACCAGTG
GAGGCCGGATGGACCCACCCAAGAACTGCCCTGGGCCTGTATACCGGATAATGACTCAGTGCTG
GCAACATCAGCCTGAAGACAGGCCCAACTTTGCCATCATTTTGGAGAGGATTGAATACTGCACC
CAGGACCCGGATGTAATCAACACCGCTTTGCCGATAGAATATGGTCCACTTGTGGAAGAGGAAG
AGAAAGTGCCTGTGAGGCCCAAGGACCCTGAGGGGGTTCCTCCTCTCCTGGTCTCTCAACAGGC
AAAACGGGAGGAGGAGCGCAGCCCAGCTGCCCCACCACCTCTGCCTACCACCTCCTCTGGCAAG
GCTGCAAAGAAACCCACAGCTGCAGAGATCTCTGTTCGAGTCCCTAGAGGGCCGGCCGTGGAAG
GGGGACACGTGAATATGGCATTCTCTCAGTCCAACCCTCCTTCGGAGTTGCACAAGGTCCACGG
ATCCAGAAACAAGCCCACCAGCTTGTGGAACCCAACGTACGGCTCCTGGTTTACAGAGAAACCC
ACCAAAAAGAATAATCCTATAGCAAAGAAGGAGCCACACGACAGGGGTAACCTGGGGCTGGAGG
GAAGCTGTACTGTCCCACCTAACGTTGCAACTGGGAGACTTCCGGGGGCCTCACTGCTCCTAGA
GCCCTCTTCGCTGACTGCCAATATGAAGGAGGTACCTCTGTTCAGGCTACGTCACTTCCCTTGT
GGGAATGTCAATTACGGCTACCAGCAACAGGGCTTGCCCTTAGAAGCCGCTACTGCCCCTGGAG
CTGGTCATTACGAGGATACCATTCTGAAAAGCAAGAATAGCATGAACCAGCCTGGGCCCTGA.
>AB274722.1: 271-3450 Homo sapiens mRNA for fusion protein EML4-ALK variant 1,
complete cds
(SEQ ID NO: 21)
ATGGACGGTTTCGCCGGCAGTCTCGATGATAGTATTTCTGCTGCAAGTACTTCTGATGTTCAAG
ATCGCCTGTCAGCTCTTGAGTCACGAGTTCAGCAACAAGAAGATGAAATCACTGTGCTAAAGGC
GGCTTTGGCTGATGTTTTGAGGCGTCTTGCAATCTCTGAAGATCATGTGGCCTCAGTGAAAAAA
TCAGTCTCAAGTAAAGGCCAACCAAGCCCTCGAGCAGTTATTCCCATGTCCTGTATAACCAATG
GAAGTGGTGCAAACAGAAAACCAAGTCATACCAGTGCTGTCTCAATTGCAGGAAAAGAAACTCT
TTCATCTGCTGCTAAAAGTGGTACAGAAAAAAAGAAAGAAAAACCACAAGGACAGAGAGAAAAA
AAAGAGGAATCTCATTCTAATGATCAAAGTCCACAAATTCGAGCATCACCTTCTCCCCAGCCCT
CTTCACAACCTCTCCAAATACACAGACAAACTCCAGAAAGCAAGAATGCTACTCCCACCAAAAG
CATAAAACGACCATCACCAGCTGAAAAGTCACATAATTCTTGGGAAAATTCAGATGATAGCCGT
AATAAATTGTCGAAAATACCTTCAACACCCAAATTAATACCAAAAGTTACCAAAACTGCAGACA
AGCATAAAGATGTCATCATCAACCAAGAAGGAGAATATATTAAAATGTTTATGCGCGGTCGGCC
AATTACCATGTTCATTCCTTCCGATGTTGACAACTATGATGACATCAGAACGGAACTGCCTCCT
GAGAAGCTCAAACTGGAGTGGGCATATGGTTATCGAGGAAAGGACTGTAGAGCTAATGTTTACC
TTCTTCCGACCGGGGAAATAGTTTATTTCATTGCATCAGTAGTAGTACTATTTAATTATGAGGA
GAGAACTCAGCGACACTACCTGGGCCATACAGACTGTGTGAAATGCCTTGCTATACATCCTGAC
AAAATTAGGATTGCAACTGGACAGATAGCTGGCGTGGATAAAGATGGAAGGCCTCTACAACCCC
ACGTCAGAGTGTGGGATTCTGTTACTCTATCCACACTGCAGATTATTGGACTTGGCACTTTTGA
GCGTGGAGTAGGATGCCTGGATTTTTCAAAAGCAGATTCAGGTGTTCATTTATGTGTTATTGAT
GACTCCAATGAGCATATGCTTACTGTATGGGACTGGCAGAAGAAAGCAAAAGGAGCAGAAATAA
AGACAACAAATGAAGTTGTTTTGGCTGTGGAGTTTCACCCAACAGATGCAAATACCATAATTAC
ATGCGGTAAATCTCATATTTTCTTCTGGACCTGGAGCGGCAATTCACTAACAAGAAAACAGGGA
ATTTTTGGGAAATATGAAAAGCCAAAATTTGTGCAGTGTTTAGCATTCTTGGGGAATGGAGATG
TTCTTACTGGAGACTCAGGTGGAGTCATGCTTATATGGAGCAAAACTACTGTAGAGCCCACACC
TGGGAAAGGACCTAAAGTGTACCGCCGGAAGCACCAGGAGCTGCAAGCCATGCAGATGGAGCTG
CAGAGCCCTGAGTACAAGCTGAGCAAGCTCCGCACCTCGACCATCATGACCGACTACAACCCCA
ACTACTGCTTTGCTGGCAAGACCTCCTCCATCAGTGACCTGAAGGAGGTGCCGCGGAAAAACAT
CACCCTCATTCGGGGTCTGGGCCATGGAGCCTTTGGGGAGGTGTATGAAGGCCAGGTGTCCGGA
ATGCCCAACGACCCAAGCCCCCTGCAAGTGGCTGTGAAGACGCTGCCTGAAGTGTGCTCTGAAC
AGGACGAACTGGATTTCCTCATGGAAGCCCTGATCATCAGCAAATTCAACCACCAGAACATTGT
TCGCTGCATTGGGGTGAGCCTGCAATCCCTGCCCCGGTTCATCCTGCTGGAGCTCATGGCGGGG
GGAGACCTCAAGTCCTTCCTCCGAGAGACCCGCCCTCGCCCGAGCCAGCCCTCCTCCCTGGCCA
TGCTGGACCTTCTGCACGTGGCTCGGGACATTGCCTGTGGCTGTCAGTATTTGGAGGAAAACCA
CTTCATCCACCGAGACATTGCTGCCAGAAACTGCCTCTTGACCTGTCCAGGCCCTGGAAGAGTG
GCCAAGATTGGAGACTTCGGGATGGCCCGAGACATCTACAGGGCGAGCTACTATAGAAAGGGAG
GCTGTGCCATGCTGCCAGTTAAGTGGATGCCCCCAGAGGCCTTCATGGAAGGAATATTCACTTC
TAAAACAGACACATGGTCCTTTGGAGTGCTGCTATGGGAAATCTTTTCTCTTGGATATATGCCA
TACCCCAGCAAAAGCAACCAGGAAGTTCTGGAGTTTGTCACCAGTGGAGGCCGGATGGACCCAC
CCAAGAACTGCCCTGGGCCTGTATACCGGATAATGACTCAGTGCTGGCAACATCAGCCTGAAGA
CAGGCCCAACTTTGCCATCATTTTGGAGAGGATTGAATACTGCACCCAGGACCCGGATGTAATC
AACACCGCTTTGCCGATAGAATATGGTCCACTTGTGGAAGAGGAAGAGAAAGTGCCTGTGAGGC
CCAAGGACCCTGAGGGGGTTCCTCCTCTCCTGGTCTCTCAACAGGCAAAACGGGAGGAGGAGCG
CAGCCCAGCTGCCCCACCACCTCTGCCTACCACCTCCTCTGGCAAGGCTGCAAAGAAACCCACA
GCTGCAGAGGTCTCTGTTCGAGTCCCTAGAGGGCCGGCCGTGGAAGGGGGACACGTGAATATGG
CATTCTCTCAGTCCAACCCTCCTTCGGAGTTGCACAGGGTCCACGGATCCAGAAACAAGCCCAC
CAGCTTGTGGAACCCAACGTACGGCTCCTGGTTTACAGAGAAACCCACCAAAAAGAATAATCCT
ATAGCAAAGAAGGAGCCACACGAGAGGGGTAACCTGGGGCTGGAGGGAAGCTGTACTGTCCCAC
CTAACGTTGCAACTGGGAGACTTCCGGGGGCCTCACTGCTCCTAGAGCCCTCTTCGCTGACTGC
CAATATGAAGGAGGTACCTCTGTTCAGGCTACGTCACTTCCCTTGTGGGAATGTCAATTACGGC
TACCAGCAACAGGGCTTGCCCTTAGAAGCCGCTACTGCCCCTGGAGCTGGTCATTACGAGGATA
CCATTCTGAAAAGCAAGAATAGCATGAACCAGCCTGGGCCCTGA.

By “genetic vaccine” is meant an immunogenic composition comprising a polynucleotide encoding an antigen. In embodiments, the antigen is an ALK antigen.

“Hybridization” means hydrogen bonding, which may be Watson-Crick, Hoogsteen, or reversed Hoogsteen hydrogen bonding, between complementary nucleobases. For example, in DNA, adenine and thymine, and cytosine and guanine, are, respectively, complementary nucleobases that pair through the formation of hydrogen bonds. By “hybridize” is meant pairing to form a double-stranded molecule between complementary polynucleotide sequences (e.g., a gene), or portions thereof, under various conditions of stringency (e.g., Wahl, G. M. and S. L. Berger, (1987), Methods Enzymol., 152:399; Kimmel, A. R., (1987), Methods Enzymol. 152:507).

By way of example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “immunogen” is meant agent which is capable, under appropriate conditions, of eliciting or stimulating an immune response. In an embodiment, an immune response includes a T-cell response. As used herein, an “immunogenic composition” is a composition comprising an immunogen (such as an ALK polypeptide) or a vaccine comprising an immunogen (such as an ALK polypeptide). As will be appreciated by the skilled person in the art, if administered to a subject in need prior to the subject's contracting disease or experiencing full-blown disease, an immunogenic composition can be prophylactic and result in the subject's eliciting an immune response, e.g., a cellular immune response, to protect against disease, or to prevent more severe disease or condition, and/or the symptoms thereof. If administered to a subject in need following the subject's contracting disease, an immunogenic composition can be therapeutic and result in the subject's eliciting an immune response, e.g., a cellular immune response, to treat the disease, e.g., by reducing, diminishing, abrogating, ameliorating, or eliminating the disease, and/or the symptoms thereof. In some embodiments, the immune response is a B-cell response, which results in the production of antibodies, e.g., neutralizing antibodies, directed against the immunogen or immunogenic composition comprising the antigen or antigen sequence. In some embodiments, the immune response is a T-cell response, which results in the production of T-lymphocytes. In a manner similar to the foregoing, in some embodiments, an immunogenic composition or vaccine can be prophylactic. In some embodiments, an immunogenic composition or vaccine can be therapeutic. In some embodiments, the disease is caused by oncogenic ALK gene fusions, rearrangements, duplications, or mutations (e.g., ALK-positive cancers). In some embodiments, the cancer is an ALK-positive cancer. In some embodiments, the ALK-positive cancer is non-small cell lung cancer (NSCLC), anaplastic large cell lymphoma (ALCL), neuroblastoma, B-cell lymphoma, thyroid cancer, colon cancer, breast cancer, inflammatory myofibroblastic tumors (IMT), renal carcinoma, esophageal cancer, glioma, glioblastoma, melanoma, or a combination thereof.

The term “immune response” is meant any response mediated by an immunoresponsive cell. In one example of an immune response, leukocytes are recruited to carry out a variety of different specific functions in response to exposure to an antigen (e.g., a foreign entity). Immune responses are multifactorial processes that differ depending on the type of cells involved. Immune responses include cell-mediated responses (e.g., T-cell responses), humoral responses (B-cell/antibody responses), innate responses and combinations thereof.

By “immunogenic composition” is meant a composition that elicits an immune response in a subject. In some instances, the subject is an immunized subject.

The term “immunize” refers to the process of rendering a subject protected from a disease or pathology, or the symptoms thereof, such as by vaccination. In an embodiment, the term “immunize” relates to injecting a polypeptide comprising an oncogenic ALK gene fusions, rearrangements, duplications, or mutations (e.g., ALK-positive cancers), or fragments thereof.

By “increases” is meant a positive alteration of at least 5%, 10%, 25%, 30%, 40%, 50%, 75%, 80%, 85%, 90%, 95%, 98%, or 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid, protein, or peptide is purified if it is substantially free of cellular material, debris, non-relevant viral material, or culture medium when produced by recombinant DNA techniques, or of chemical precursors or other chemicals when chemically synthesized. Purity and homogeneity are typically determined using standard purification methods and analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified. The term “isolated” also embraces recombinant nucleic acids or proteins, as well as chemically synthesized nucleic acids or peptides.

By “isolated polynucleotide” is meant a nucleic acid molecule that is free of the genes which flank the gene, in the naturally-occurring genome of the organism from which the nucleic acid molecule of the invention is derived. In some instances the nucleic acid molecule is a DNA molecule or an RNA molecule. The term includes, for example, a recombinant DNA that is incorporated into a vector; into an autonomously replicating plasmid or virus; into the genomic DNA of a prokaryote or eukaryote; or that exists as a separate molecule independent of other sequences (for example, a cDNA or a genomic or cDNA fragment produced by PCR or restriction endonuclease digestion). In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 40%, by weight, at least 50%, by weight, at least 60%, by weight, free from the proteins and naturally-occurring organic molecules with which it is naturally associated. Preferably, an isolated polypeptide preparation is at least 75%, more preferably at least 90%, and most preferably at least 99%, by weight, free from the proteins and naturally-occurring organic molecules with which it is naturally associated. An isolated polypeptide may be obtained, for example, by extraction from a natural source; by expression of a recombinant nucleic acid encoding such a polypeptide; or by chemically synthesizing the protein. Purity can be measured by any standard, appropriate method, for example, column chromatography, polyacrylamide gel electrophoresis, or by HPLC analysis. An isolated polypeptide can refer to an ALK antigen or immunogen polypeptide generated by the methods described herein.

By “linker” is meant one or more amino acids that serve as a spacer between two polypeptides or peptides of a fusion protein.

By “marker” is meant any protein or polynucleotide having an alteration in expression level or activity that is associated with a disease, condition, pathology, or disorder.

As used herein, “obtaining” as in “obtaining an agent” includes synthesizing, isolating, purchasing, or otherwise acquiring the agent.

By “operably linked” is meant that a first polynucleotide is positioned adjacent to a second polynucleotide that directs transcription of the first polynucleotide when appropriate molecules are bound to the second polynucleotide. By way of example, a first nucleic acid sequence is operably linked to a second nucleic acid sequence when the first nucleic acid sequence is placed in a functional relationship with the second nucleic acid sequence. For instance, a promoter is operably linked to a coding sequence if the promoter affects (allows) the transcription or expression of the coding sequence. Generally, operably linked DNA sequences are contiguous and, where necessary to join two protein-coding regions, are in the same open reading frame.

The nucleic acid sequence encoding an ALK peptide (antigen peptide) generated by the described methods can be optimized for expression in mammalian cells via codon-optimization and RNA optimization (such as to increase RNA stability) using procedures and techniques practiced in the art.

The term “pharmaceutically acceptable vehicle” refers to conventional carriers and excipients that are physiologically and pharmaceutically acceptable for use, particularly in mammalian subjects. A non-limiting examples of a mammalian subject is a human subject. Pharmaceutically acceptable vehicles are known to the skilled practitioner in the pertinent art and can be readily found in Remington's Pharmaceutical Sciences, by E. W. Martin, Mack Publishing Co., Easton, Pa., 15th Edition (1975) and its updated editions, which describes compositions and formulations suitable for pharmaceutical delivery of one or more therapeutic or immunogenic compositions, such as one or more vaccines, and additional pharmaceutical agents. In general, the nature of a pharmaceutically acceptable carrier depends on the particular mode of administration being employed. For instance, parenteral formulations usually comprise injectable fluids/liquids that include pharmaceutically and physiologically acceptable fluids such as water, physiological saline, balanced salt solutions, aqueous dextrose, glycerol, or the like as a vehicle. For solid compositions (for example, powder, pill, tablet, or capsule forms), conventional non-toxic solid carriers may include, for example, pharmaceutical grades of mannitol, lactose, starch, or magnesium stearate, which typically stabilize and/or increase the half-life of a composition or drug. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

By “plasmid” is meant a circular nucleic acid molecule capable of autonomous replication in a host cell.

The terms “protein,” “peptide,” “polypeptide,” and their grammatical equivalents are used interchangeably herein, and refer to a polymer of amino acid residues linked together by peptide bonds. The terms refer to a protein, peptide, or polypeptide of any size, structure, or function. Typically, a protein, peptide, or polypeptide will be at least three (3) amino acids long. A protein, peptide, or polypeptide can refer to an individual protein or a collection of proteins. One or more of the amino acids in a protein, peptide, or polypeptide can be modified, such as glycoproteins, for example, by the addition of a chemical entity such as a carbohydrate group, a hydroxyl group, a phosphate group, a farnesyl group, an isofarnesyl group, a fatty acid group, a linker for conjugation, functionalization, or other modifications, etc. A protein, peptide, or polypeptide can also be a single molecule or can be a multi-molecular complex. A protein, peptide, or polypeptide can be just a fragment of a naturally occurring protein or peptide. A protein, peptide, or polypeptide can be naturally occurring, recombinant, or synthetic, or any combination thereof.

In some embodiments, a protein comprises a proteinaceous part, e.g., an amino acid sequence constituting a nucleic acid binding domain, and an organic compound, e.g., a compound that can act as a nucleic acid cleavage agent. In some embodiments, a protein is in a complex with, or is in association with, a nucleic acid, e.g., RNA or DNA. Any of the proteins provided herein can be produced by any method known in the art. For example, the proteins provided herein can be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

Conservative amino acid substitutions are those substitutions that, when made, least interfere with the properties of the original protein, that is, the structure and especially the function of the protein is conserved and is not significantly changed by such substitutions. Examples of conservative amino acid substitutions are known in the art, e.g., as set forth in, for example, U.S. Publication No. 2015/0030628. Conservative substitutions generally maintain (a) the structure of the polypeptide backbone in the area of the substitution, for example, as a sheet or helical conformation; (b) the charge or hydrophobicity of the molecule at the target site; and/or (c) the bulk of the side chain

The substitutions that are generally expected to produce the greatest changes in protein properties are non-conservative, for instance, changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

By “promoter” is meant a polynucleotide sufficient to direct transcription. A promoter includes necessary nucleic acid sequences near the start site of transcription. A promoter also optionally includes distal enhancer or repressor sequence elements. A “constitutive promoter” is a promoter that is continuously active and is not subject to regulation by external signals or molecules. In contrast, the activity of an “inducible promoter” is regulated by an external signal or molecule (for example, a transcription factor). By way of example, a promoter may be a CMV promoter.

As will be appreciated by the skilled practitioner in the art, the term “purified” does not require absolute purity; rather, it is intended as a relative term. Thus, for example, a purified peptide, protein, or other active compound is one that is isolated in whole or in part from naturally associated proteins and other contaminants. In certain embodiments, the term “substantially purified” refers to a peptide, protein, or other active compound that has been isolated from a cell, cell culture medium, or other crude preparation and subjected to routine methods, such as fractionation, chromatography, or electrophoresis, to remove various components of the initial preparation, such as proteins, cellular debris, and other components.

By “reduces” is meant a negative alteration of at least 5%, 10%, 25%, 30%, 40%, 50%, 75%, 80%, 85%, 90%, 95%, 98%, or 100%.

By “reference” is meant a standard or control condition. In some cases, the reference is a healthy cell or a healthy subject, or the reference is a cell or subject that does not have or is not associated with a cancer or tumor (e.g., a non-small cell lung cancer (NSCLC)). In some instances, the reference is a subject or cell prior to being administered a composition or being treated for a disease or a subject or cell that has not been administered a composition or treatment. In some instances, the reference is a subject or cell prior to a change in a treatment.

A “reference sequence” is a defined sequence used as a basis for sequence comparison. The reference sequence can be an ALK antigen nucleotide or polypeptide sequence. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a compound or antibody that recognizes and binds a polypeptide of the invention, but which does not substantially recognize and bind other molecules in a sample, for example, a biological sample, which naturally includes a polypeptide of the invention, such as an ALK polypeptide or peptide.

Nucleic acid molecules useful in the methods described herein include any nucleic acid molecule that encodes a polypeptide as described, or a fragment thereof. Such nucleic acid molecules need not be 100% identical with an endogenous nucleic acid sequence, but will typically exhibit substantial identity.

By “substantially identical” is meant a polypeptide or nucleic acid molecule exhibiting at least 50% identity to a reference amino acid sequence or nucleic acid sequence. Examples of reference amino acid sequences and nucleic acid sequences include any of those provided herein. In embodiments, such a sequence is at least 60%, or at least 80% or 85%, or at least or equal to 90%, 95%, 98% or even 99% identical at the amino acid level or nucleic acid to the sequence used for comparison. Polynucleotides having “substantial identity” to an endogenous sequence are in some instances capable of hybridizing with at least one strand of a double-stranded nucleic acid molecule.

“Sequence identity” refers to the similarity between amino acid or nucleic acid sequences that is expressed in terms of the similarity between the sequences. Sequence identity is frequently measured in terms of percentage identity (or similarity or homology); the higher the percentage, the more similar the sequences are. Homologs or variants of a given gene or protein will possess a relatively high degree of sequence identity when aligned using standard methods. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence. In addition, other programs and alignment algorithms are described in, for example, Smith and Waterman, 1981, Adv. Appl. Math. 2:482; Needleman and Wunsch, 1970, J Mol. Biol. 48:443; Pearson and Lipman, 1988, Proc. Natl. Acad. Sci. U.S.A. 85:2444; Higgins and Sharp, 1988, Gene 73:237-244; Higgins and Sharp, 1989, CABIOS 5:151-153; Corpet et al., 1988, Nucleic Acids Research 16:10881-10890; Pearson and Lipman, 1988, Proc. Natl. Acad. Sci. U.S.A. 85:2444; and Altschul et al., 1994, Nature Genet. 6:119-129. The NCBI Basic Local Alignment Search Tool (BLAST™) (Altschul et al. 1990, J. Mol. Biol. 215:403-410) is readily available from several sources, including the National Center for Biotechnology Information (NCBI, Bethesda, Md.) and on the Internet, for use in connection with the sequence analysis programs blastp, blastn, blastx, tblastn and tblastx.

By “subject” is meant an animal. Non-limiting examples of animals include a mammal, including, but not limited to, a human, a non-human primate, or a non-human mammal, such as a bovine, equine, canine, ovine, or feline mammal, or a sheep, goat, llama, camel, or a rodent (e.g., rat, mouse), gerbil, or hamster. In a nonlimiting example, a subject is one who has, is at risk of developing, or who is susceptible to a disease caused by oncogenic ALK gene fusions, rearrangements, duplications, or mutations (e.g., ALK-positive cancers). In particular aspects as described herein, the subject is a human subject, such as a patient.

Ranges provided herein are understood to be shorthand for all of the values within the range, inclusive of the first and last stated values. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or greater, consecutively, such as to 100 or greater.

As used herein, the terms “treat,” “treating,” “treatment,” and the like refer to reducing, diminishing, decreasing, delaying, abrogating, ameliorating, or eliminating, a disease, condition, disorder, or pathology, and/or symptoms associated therewith. While not intending to be limiting, “treating” typically relates to a therapeutic intervention that occurs after a disease, condition, disorder, or pathology, and/or symptoms associated therewith, have begun to develop to reduce the severity of the disease, etc., and the associated signs and symptoms. It will be appreciated that, although not precluded, treating a disorder or condition does not require that the disease, condition, disorder, pathology, or the symptoms associated therewith, be completely eliminated.

As referred to herein, a “transformed” or “transfected” cell is a cell into which a nucleic acid molecule or polynucleotide sequence has been introduced by molecular biology techniques. As used herein, the term “transfection” encompasses all techniques by which a nucleic acid molecule or polynucleotide may be introduced into such a cell, including transfection with viral vectors, transformation with plasmid vectors, and introduction of naked nucleic acid (DNA or RNA) by electroporation, lipofection, and particle gun acceleration.

By “vaccine” is meant a preparation of immunogenic material capable of eliciting an immune response. In embodiments, a vaccine is administered to a subject to treat a disease, condition, or pathology, or to prevent a disease, condition, or pathology. In some instances, the disease is caused by oncogenic ALK gene fusions, rearrangements, duplications, or mutations (e.g., ALK-positive cancers). In embodiments, the immunogenic materials is a protein or nucleic acid molecule. The immunogenic material may include, for example, antigenic proteins, peptides, or DNA derived from ALK-expressing tumors or cell lines. Vaccines may elicit a prophylactic (preventative) immune response in the subject; they may also elicit a therapeutic response immune response in a subject. As mentioned above, methods of vaccine administration vary according to the vaccine, and can include routes or means, such as inoculation (intravenous or subcutaneous injection), ingestion, inhalation, or other forms of administration. Inoculations can be delivered by any number of routes, including parenteral, such as intravenous, subcutaneous, or intramuscular. Vaccines may also be administered with an adjuvant to boost the immune response.

As used herein, a “vector” refers to a nucleic acid molecule into which foreign nucleic acid can be inserted without disrupting the ability of the vector to replicate in and/or integrate into a host cell. A vector can include nucleic acid sequences that permit it to replicate in a host cell, such as an origin of replication. An insertional vector is capable of inserting itself into a host nucleic acid. A vector can also include one or more selectable marker genes and other genetic elements. An expression vector is a vector that contains the necessary regulatory sequences to allow transcription and translation of inserted gene or genes in a host cell. In some embodiments of the present disclosure, the vector encodes an ALK protein. In some embodiments, the vector is the pTR600 expression vector (U.S. Patent Application Publication No. 2002/0106798; Ross et al., 2000, Nat Immunol. 1(2):102-103; and Green et al., 2001, Vaccine 20:242-248).

Unless specifically stated or obvious from context, as used herein, the term “or” is understood to be inclusive. Unless specifically stated or obvious from context, as used herein, the terms “a,” “an,” and “the” are understood to be singular or plural. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description.

Unless specifically stated or obvious from context, as used herein, the term “about” is understood as within a range of normal tolerance in the art, for example within 2 standard deviations of the mean. Unless otherwise clear from context, all numerical values provided herein are modified by the term about.

The recitation of a listing of chemical groups in any definition of a variable herein includes definitions of that variable as any single group or combination of listed groups. The recitation of some embodiments for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1J provide images, schematics, and bar graphs showing immune checkpoint inhibitors (ICIs) did not increase the efficacy of ALK TKIs in ALK⁺ lung cancer mouse models. (FIG. 1A) Representative H&E staining of hEML4-ALK rearranged lung tumor in hEML4-ALK Tg mice (left panel) and mEml4-Alk rearranged lung tumor in Ad-EA mice (right panel). Scale bars indicate 100 μm. (FIG. 1B) Coronal MRI lung sections of a representative Ad-EA mouse enrolled in 15 days treatment of lorlatinib combined with anti-PD-1. Arrows indicate tumor lesions. (FIGS. 1C and 1D) Schematic representation of treatment protocol in hEML4-ALK Tg mice (FIG. 1C) and Ad-EA mice (FIG. 1D). (FIGS. 1E, 1F, and 1G) Quantification of volume changes compared with baseline tumor volume (change from baseline, %±SEM) in hEML4-ALK Tg mice treated as in FIG. 1C at T0 (FIG. 1E), T4 (FIG. 1F), and T8 (FIG. 1G). (FIGS. 1H, 1I, and 1J) Quantification of volume changes compared with baseline tumor volume (change from baseline, %±SEM) of Ad-EA mice treated as in FIG. 1D at T0 (FIG. 1H), T4 (FIG. 1I), and T8 (FIG. 1J). Pre: pre-treatment; T0: end of treatment; T4: 4 weeks after treatment suspension; T8: 8 weeks after treatment suspension. Each dot represents an individual mouse. n/s, not significant. (All P values were calculated using an unpaired two-tailed Student's t test). In FIGS. 1E-1J, the bars from left-to-right correspond to Rat IgG, Anti-PD-1, Anti-PD-L1, Crizotinib+Rat IgG, Crizotinib+Anti-PD-1, Crizotinib+Anti-PD-L1, Lorlatinib+Rat IgG, Lorlatinib+Anti-PD-1, and Lorlatinib+Anti-PD-L1

FIGS. 2A-2F provide a schematic, scatter plots, images, and bar graphs showing identification of ALK immunogenic peptides in mouse models. (FIG. 2A) Schematic representation of ALK peptides screening in vivo. (FIG. 2B) IFN-γ-ELISPOT assay of splenocytes isolated as in FIG. 2A. Data from three experimental replicates are shown as average number of spots (±SEM). A cut-off value of 100 IFN-γ spot forming units (SFU) was applied to provide a threshold of responsiveness. Splenocytes isolated from NPM-ALK Tg mice and WT BALB/c mice were used respectively as positive and negative control. (FIG. 2C) Representative IFN-γ intracellular staining in CD4⁺ and CD8⁺ splenocytes isolated as in (FIG. 2A) from a mouse vaccinated with SLP7 and pulsed with 10 μg/mL of the same peptide. FIG. 2C discloses SEQ ID NO: 32. (FIG. 2D) Representative IFN-γ ELISPOT analysis of splenocytes isolated from naïve and mice vaccinated with ALK-short peptide 7 (PGPGRVAKI (SEQ ID NO: 22)). (FIG. 2E) Quantification of PGPGRVAKI (SEQ ID NO: 22)-specific CD8⁺ T-cells splenocytes (left bar) or lung tumor infiltrating T-cells (right bar) from hEML4-ALK Tg mice when 12 week-old. Cells were gated from viable CD8⁺ T-cells. Each dot represents a mouse. (FIG. 2F) Dextramer staining of PGPGRVAKI (SEQ ID NO: 22)-specific CD8⁺ T-cells isolated from naïve and PGPGRVAKI (SEQ ID NO: 22)-vaccinated mice splenocytes displayed as percentage (±SEM). Each dot represents an individual mouse (unpaired two-tailed Student's t test) ***P<0.001. FIG. 2F. discloses SEQ ID NO: 22.

FIGS. 3A-3L provide plots, images, and bar graphs showing tumor localization dictated the strength of the anti-ALK spontaneous immune response and determines the response to ICI in ALK⁺ lung tumors. (FIG. 3A) mEml4-Alk¹ subcutaneous tumor-bearing mice were treated as indicated. Data shown as average tumor volume (mm³±SEM). In FIG. 3A, Squares represent Anti-PD-1, circles represent Untreated, Triangles represent Anti-CTLA-4, and inverted triangles represent Combo. (FIG. 3B) Kaplan-Meier curves showing overall survival of mice described in FIG. 3A (Log-rank test). (FIG. 3C) Eml4-Alk^{PGPGRVAKI} (“PGPGRVAKI” disclosed as SEQ ID NO: 22) cells were injected subcutaneously in syngeneic BALB/c mice and spontaneous tumor growth was measured. Two independent experiments are shown as individual tumor volumes. FIG. 3C discloses SEQ ID NO: 22. (FIG. 3D) Eml4-Alk^{PGPGRVAKI} (“PGPGRVAKI” disclosed as SEQ ID NO: 22) cells were injected as in FIG. 3C and mice treated as indicated. Tumor growth was measured. Two independent experiments are shown as individual tumor volumes. FIG. 3D discloses SEQ ID NO: 22. (FIG. 3E) Eml4-Alk^{PGPGRVAKI} (“PGPGRVAKI” disclosed as SEQ ID NO: 22) cells were injected as in FIG. 3C and mice treated as indicated. Tumor-free mice were subcutaneously rechallenged at day 50 after initial tumor injection with either mEml4-Alk (n=5) or with Eml4-Alk^{PGPGRVAKI} (“PGPGRVAKI” disclosed as SEQ ID NO: 22) (n=5) and tumor volume monitored. FIG. 3E discloses “PGPGRVAKI” as SEQ ID NO: 22. (FIG. 3F) Eml4-Alk^{PGPGRVAKI} (“PGPGRVAKI” disclosed as SEQ ID NO: 22) cells were injected as in FIG. 3C and mice treated as indicated. Tumor-free mice were subcutaneously rechallenged at day 50 after initial tumor injection with either mEml4-Alk (n=3) or with Eml4-Alk^{PGPGRVAKI} (“PGPGRVAKI” disclosed as SEQ ID NO: 22) (n=3) and tumor volume monitored. FIG. 3F discloses “PGPGRVAKI” as SEQ ID NO: 22. (FIG. 3G) Kaplan-Meier curves showing overall survival of mice described in FIGS. 3C-3F. Rechallenged mice are not shown (Log-rank test). In FIG. 3G, from lower-left to upper-right, going counterclockwise, the curves correspond to Untreated, Anti-PD-1, Combo, and Anti-CTLA-4. FIG. 3G discloses SEQ ID NO: 22. (FIG. 3H) Kaplan-Meier curves showing overall survival of mice subjected to intravenous tumor rechallenge. After subcutaneous rechallenge with Eml4-Alk^{PGPGRVAKI} (“PGPGRVAKI” disclosed as SEQ ID NO: 22), tumor-free mice were once again rechallenged through intravenous injection with either mEml4-Alk (n=4) or Eml4-Alk^{PGPGRVAKI} (“PGPGRVAKI” disclosed as SEQ ID NO: 22) (n=4) cell lines at day 100 post initial transplant (Log-rank test). FIG. 3H discloses SEQ ID NO: 22. (FIG. 3I) Kaplan-Meier curves showing overall survival of mice injected intravenously with Eml4-Alk^{PGPGRVAKI} (“PGPGRVAKI” disclosed as SEQ ID NO: 22) cell line and treated as indicated (Log-rank test). FIG. 3I discloses SEQ ID NO: 22. (FIG. 3J) Representative H&E staining of lung adenocarcinomas from syngeneic BALB/c mice described in FIG. 3I. Black arrows indicate lung tumors. (FIG. 3K) IFN-γ-ELISPOT analysis of isolated splenocytes after 15 days post subcutaneous (subcutis) and intravenous (lung) injection of mice treated as in FIGS. 3G and 3I. Data is shown as average number of spots (±SEM). Each dot represents an individual mouse (Unpaired two-tailed Student's t test). (FIG. 3L) Dextramer staining PGPGRVAKI (SEQ ID NO: 22)-specific CD8⁺ T-cells isolated splenocytes after 15 days post subcutaneous (subcutis) and intravenous (lung) injection of mice treated as in FIGS. 3G and 3I. Data is displayed as percentage (%±SEM). Each dot represents an individual mouse (Unpaired two-tailed Student's t test) *P<0.05; **P<0.005; ****P<0.0001; n/s, not significant.

FIGS. 4A-4G provide bar graphs, a schematic, and plots showing enhancement of the anti-ALK immune responses by vaccination leads to rejection of ALK⁺ lung tumors in combination with ALK TKI. (FIG. 4A) Representative IFN-γ-ELISPOT analysis of isolated splenocytes from tumor-bearing BALB/c mice intravenously injected with mEml4-Alk (left), Eml4-Alk^{PGPGRVAKI-1} (“PGPGRVAKI” disclosed as SEQ ID NO: 22) (middle), and WT BALB/c mice vaccinated with ALK vaccine (right) (Unpaired two-tailed Student's t test). FIG. 4A discloses “PGPGRVAKI” as SEQ ID NO: 22. (FIG. 4B) Dextramer staining of PGPGRVAKI (SEQ ID NO: 22)-specific CD8⁺ T-cells isolated from lung tumor-infiltrating lymphocytes of mice treated as indicated (Unpaired two-tailed Student's t test). (FIG. 4C) PD-1 staining is displayed as average percentage (%±SEM) within the CD8⁺/Dextramer⁺ populations in c (Unpaired two-tailed Student's t test). (FIG. 4D) Schematic representation of treatment protocol. DIE, once a day. FIG. 4D discloses SEQ ID NO: 22. (FIG. 4E) Kaplan-Meier curves showing the overall survival of mice treated as indicated in FIG. 4D (Log-rank test). In order from bottom-left to top-right, going counterclockwise, the curves represented correspond to Untreated, Lorlatinib+anti-PD-1, Lorlatinib, Lorlatinib+anti-CTLA-4, Lorlatinib+ALK vax, Lorlatinib+ALK vax+anti-PD-1, and Lorlatinib+ALK vax+anti-CTLA-4 (FIG. 4F) Dextramer staining of PGPGRVAKI (SEQ ID NO: 22)-specific CD8⁺ T-cells isolated from total blood in tumor-free mice from e rechallenged intravenously with Eml4-Alk^{PGPGRVAKI-1} (“PGPGRVAKI” disclosed as SEQ ID NO: 22) cell line 250 days after initial tumor injection. Each dot represents an individual mouse and data are displayed as percentage (%±SEM). (FIG. 4G) Kaplan-Meier curves showing overall survival of mice from f after rechallenge (Log-rank test). *P<0.05; **P<0.005; ***P<0.001; ****P<0.0001; n/s, not significant.

FIGS. 5A and 5B provide images and a bar graph showing ALK vaccine in combination with ALK TKI prevents brain metastasis in mouse models. (FIG. 5A) Representative H&E staining of brain tissue of syngeneic BALB/c mice enrolled in the brain metastasis assay and treated as indicated. Scale bars=50 μm. (FIG. 5B) Incidence proportion represented as percentage of brain metastases within the indicated treatment regimes. CNS. Central Nervous System. The bars in FIG. 5B correspond to the labels provided in the legend, as listed going from left-to-right and top-to-bottom (i.e., the first bar corresponds to Untreated and the last on the right corresponds to Lorlatinib+ALK vax+anti-CTLA-4).

FIGS. 6A-6H provide images, bar graphs, plots, and histograms showing tumor escape in vaccinated mice is due to reversible MHC-I downregulation. (FIG. 6A) Representative H&E staining of lung adenocarcinomas from syngeneic BALB/c mice described in FIG. 4D. Black arrows indicate lung tumors. (FIG. 6B) H2-Dd staining of lung tumor escapers, displayed as MFI (±SEM) from mice described in FIG. 4D (Unpaired two-tailed Student's t test). (FIG. 6C) H2-Dd staining displayed as MFI of MHC-I^{low} lung tumor escapers with (+) and without (−) IFN-γ stimulation. (FIGS. 6D-6G) Two MHC-I^{high} (FIGS. 6D and 6E) and two MHC-I^{low} (FIGS. 6F and 6G) lung tumor escapers were reinjected subcutaneously into naïve syngeneic BALB/c mice and treated as indicated. Each line represents an individual mouse. (FIG. 6H) H2-Dd staining of eight MHC-I^{low} lung tumor escapers treated or not with a STING agonist (ADU-S100, 50 μM) (1-4: ex vivo cell lines generated from mice treated with lorlatinib+ALK vax; 5-8: ex vivo cell lines generated from mice treated with lorlatinib+ALK vax+anti-PD-1). ****P<0.0001. In FIG. 6H, the “Isotype” is plotted only in the upper-left plot and corresponds to the leftmost curve. FIG. 6H discloses SEQ ID NO: 22.

FIGS. 7A-7E provide images, plots, and bar graphs showing identification of immunogenic ALK peptides in ALK⁺ NSCLC patients. (FIG. 7A) Representative H&E (upper panel), ALK (middle panel), and MHC-I IHC (lower panel) of patient's lung adenocarcinoma bearing EML4-ALK fusions. Scale bars indicate 100 μm. (FIGS. 7B and 7C) LC-DIAMS from pan-HLA immunoprecipitations of the ALK⁺ cell line NCI-H2228 (FIG. 7B) and a lung ALK⁺ tumor biopsy (FIG. 7C). Precursor ions and Poisson chromatograms with detection of ALK peptides at elution marked by arrows. FIG. 7C discloses SEQ ID NOS 3 and 5, respectively, in order of appearance. (FIG. 7D) Quantification of IFN-γ ELISPOT assays from splenocytes isolated from CB6F1-B2m^{tm1Unc}; Tg(B2M)55Hpl; Tg(HLA-B*0702/H2-Kb) mice (Tg HLA B*07:02) vaccinated (FIG. 16A) with peptides IVRshort (n=6) or IVRlong (n=6) (upper panel) or RPRshort 9 (n=6) or RPRlong (n=6) (lower panel). PBS was used as a vaccination control (n=5). Each bar represents splenocytes from individual mice incubated with either peptide (IVRCIGVSL (SEQ ID NO: 4) upper panel, RPRSQPSSL (SEQ ID NO: 23) lower panel) or peptide vehicle as a negative control. Error bars mean±SD of the technical replicates. FIG. 7D discloses “IVRCIGVSL” as SEQ ID NO: 4. (FIG. 7E) IFN-γ ELISPOT assay of CD8⁺ T cells isolated from a patient and expanded in the presence of either IVRCIGVSL (SEQ ID NO: 4) or RPRSQPSSL (SEQ ID NO: 23) peptides as indicated in FIG. 16B. 25000 CD8⁺ T cells were incubated with 25000 autologous B cells pulsed with the indicated peptides. Unpulsed B cells (vehicle) and B cells pulsed CEF-MHC Class I Control Peptide Pool “plus” (CEF+) were used respectively as negative and positive controls. FIG. 7E discloses SEQ ID NOS 4, 3, 4, 3, 3, 3, 23, and 23, respectively, in order of appearance.

FIGS. 8A-8L provide plots, bar graphs, and a schematic showing immune checkpoint inhibitors (ICIs) did not increase the efficacy of ALK TKIs in ALK⁺ lung cancer mouse models. (FIGS. 8A and 8B) Kaplan-Meier curves showing overall survival of mice described in FIG. 1C (FIG. 8A) and in FIG. 1D (FIG. 8B). (FIGS. 8C-8E) Quantification of volume change compared with baseline tumor volume (change from baseline, %±SEM) in hEML4-ALK Tg mice treated with higher doses of ALK inhibitors and ICIs at T0 (FIG. 8C), T4 (FIG. 8D), and T8 (FIG. 8E). In FIGS. 8C-8E, the bars correspond to the samples as listed in the legend from left-to-right. (FIG. 8F) Kaplan-Meier curves showing overall survival of hEML4-ALK Tg mice treated with higher doses of ALK inhibitors and ICIs. (FIG. 8G) Schematic representation of long-term treatment protocol in Ad-EA mice. (FIGS. 8H-8J) Quantification of volume changes compared with baseline tumor volume (change from baseline, %±SEM) in Ad-EA mice treated as in FIG. 8G at T0 (FIG. 8H), T4 (FIG. 8I), and T8 (FIG. 8J). T0: end of treatment; T4: 4 weeks after end of treatment; T8: 8 weeks after end of treatment. Each dot represents an individual mouse. (FIGS. 8K and 8L) Kaplan-Meier curves showing the overall survival of Ad-EA mice described in FIG. 8G. (FIG. 8K) 1 month of lorlatinib treatment; (FIG. 8L) 2 months of lorlatinib treatment. *P<0.05, n/s, not significant. (All P values were calculated using an unpaired two-tailed Student's t test).

FIGS. 9A-9I provides a schematic, plots, histograms, scatter plots, and bar graphs showing identification of ALK immunogenic peptides in mouse models. (FIG. 9A) Schematic representation of ALK peptide screening. A set of 21 synthetic long peptides (SLPs) covering the entire coding region of the ALK-DNA vaccine was synthesized. Peptides A/B were synthetized to cover the cytoplasmic portion of hALK protein that is not represented in the ALK-DNA vaccine. (FIGS. 9B-9D) Benchmarking MHC-I algorithms for peptide-binding affinity prediction. NetMHC4.0 showing peptide affinity (predicted IC50 values in nM) vs. NetMHCpan4.1 showing the rank of the predicted binding score (% Rank EL). Computational predictions of peptide binding to MHC-I of SLPA (FIG. 9B). FIG. 9B discloses SEQ ID NOS 24 and 47, respectively, in order of appearance, SLP7 (FIG. 9C) and SLP20 and 21. FIG. 9C discloses SEQ ID NOS 32 and 22, respectively, in order of appearance. (FIG. 9D). FIG. 9D discloses SEQ ID NOS 45-46, and 48-49, respectively, in order of appearance. In FIGS. 9B-9D, peptides with sequences represented in light grey or medium-grey and listed in the plots were good binder candidates. (FIGS. 9E and 9F) IFN-γ intracellular staining of CD4⁺-gated (FIG. 9E) and CD8⁺-gated (FIG. 9F) splenocytes isolated from mice vaccinated with the indicated peptides and stimulated in vitro with the corresponded peptide. (FIG. 9G) Flow cytometric analysis of H2-Kd and H-2-Dd expression on ASB XIV and ASB XIV^{TAP2KO}. (FIG. 9H) H2-Dd and H2-Kd staining of ASB XIV^{TAP2KO} cells incubated with increasing concentrations of PGPGRVAKI (SEQ ID NO: 22) peptide displayed as mean fluorescence intensity (MFI) (SEM). (FIG. 9I) Representative PGPGRVAKI (SEQ ID NO: 22)-specific dextramer staining of splenocytes from naïve and mice vaccinated with ALK-short peptide 7 (PGPGRVAKI (SEQ ID NO: 22)). Cells were gated from viable CD8⁺ T-cells.

FIGS. 10A-10H provide a schematic, plots, immunoblot images, plots, and images showing generation and validation of mEml4-Alk immortalized cell line. (FIG. 10A) Schematic representation of the generation of the mEml4-Alk immortalized cell lines. (FIG. 10B) Sanger sequencing chromatogram showing the mElm4-Alk inversion. *Mouse Eml4-Alk inversion occurs in exon 14 of Eml4 and exon 20 of mouse Alk, however is displayed as in humans. FIG. 10B discloses SEQ ID NOS 372 and 372, respectively, in order of appearance. (FIG. 10C) Immunoblot analysis showing mEml4-Alk protein expression in two mEml4-Alk (mEml4-Alk¹ and mEml4-Alk²) immortalized cell lines. KP1233 cell line was used as a negative control; ALK SP8 antibody recognizes the mouse EML4-ALK (arrow); ALK D5F3 antibody recognizes the human EML4-ALK in NCI-H3122. *Indicates a non-specific band recognized by the SP8 antibody. (FIG. 10D) Dose response curves of mElm4-Alk¹ to ALK inhibitors (crizotinib, alectinib, ceritinib, brigatinib, ensartinib, entrectinib, and lorlatinib). (FIG. 10E) Immunoblot for indicated proteins in mEml4-Alk¹ cells treated with crizotinib and lorlatinib at the indicated concentrations for 6 h. (FIG. 10F) Subcutaneous tumor growth (mm³±SEM) of mEml4-Alk-1 and mEml4-Alk⁻² immortalized cell lines in NSG and syngeneic BALB/c mice. (FIG. 10G) Representative H&E of lung tumors generated in syngeneic BALB/c mice injected intravenously with mEml4-Alk. 2× magnification (left panel); 40× magnification (right panel). Scale bar=100 μm. (FIG. 10H) Quantification of volume changes compared with baseline tumor volume (change from baseline, %±SEM) in syngeneic BALB/c mice injected subcutaneously with a mEml4-Alk cell line and treated as shown. Red arrow indicates the end of treatment FIGS. 11A-11J provide annotated sequences, images, plots, chromatograms, immunoblot images, plots, images, and bar graphs showing generation and validation of Eml4-Alk^{PGPGRVAKI} (“PGPGRVAKI” disclosed as SEQ ID NO: 22) immortalized cell lines. (FIG. 11A) Schematic illustration of mouse and human ALK short peptide 7 sequence differences. H2-Dd % Rank_EL, Elution Likelihood. FIG. 11A discloses SEQ ID NOS 373, 108, 374, and 22, respectively, in order of appearance. (FIG. 11B) Schematic representation of mouse Alk peptide 7 genetic editing using CRISPR/Cas9. FIG. 11B discloses SEQ ID NOS 108 and 22, respectively, in order of appearance. (FIG. 11C) Sanger sequencing chromatogram showing the cDNA sequence of mEml4-Alk (upper panel), Eml4-Alk^{PGPGRVAKI-1} (“PGPGRVAKI” disclosed as SEQ ID NO: 22) (middle panel), and Eml4-Alk^{PGPGRVAKI-2} (“PGPGRVAKI” disclosed as SEQ ID NO: 22) (lower panel) of PGPGRVAKI peptide (SEQ ID NO: 22). FIG. 11C discloses SEQ ID NOS 375-376, 22, 377-378, 22, and 377-378, respectively, in order of appearance. (FIGS. 11D and 11E) Dose response curves of), Eml4-Alk^{PGPGRVAKI-1} (“PGPGRVAKI” disclosed as SEQ ID NO: 22) (FIG. 11D) and), Eml4-Alk^{PGPGRVAKI-2} (“PGPGRVAKI” disclosed as SEQ ID NO: 22). FIG. 11D discloses SEQ ID NO: 22. (FIG. 11E) to ALK inhibitors (crizotinib, alectinib, ceritinib, brigatinib, ensartinib, entrectinib, and lorlatinib). FIG. 11E discloses SEQ ID NO: 22. (FIG. 11F) Immunoblot for indicated proteins in Eml4-Alk^{PGPGRVAKI-1} (“PGPGRVAKI” disclosed as SEQ ID NO: 22) cells treated with lorlatinib at the indicated concentrations for 6 h. FIG. 11F discloses SEQ ID NO: 22. (FIG. 11G) Representative H&E of lung tumors generated in syngeneic BALB/c mice injected intravenously with Eml4-Alk^{PGPGRVAKI-1} (“PGPGRVAKI” disclosed as SEQ ID NO: 22). 2× magnification (left panel); 40× magnification (right panel). Scale bars=100 μm. FIG. 11G discloses SEQ ID NO: 22. (FIG. 11H) IFN-γ-ELISPOT analysis of freshly isolated splenocytes from tumor-bearing BALB/c mice 15 days after been injected subcutaneously (subcutis) with mEml4-Alk (left) and either subcutaneously (subcutis) or intravenously (lung) with Eml4-Alk^{PGPGRVAKI-1} (“PGPGRVAKI” disclosed as SEQ ID NO: 22) (middle), and Eml4-Alk^{PGPGRVAKI-2} (“PGPGRVAKI” disclosed as SEQ ID NO: 22) (right). FIG. 11H discloses “PGPGRVAKI” as SEQ ID NO: 22 (FIGS. 11I and 11J) Dextramer staining of PGPGRVAKI (SEQ ID NO: 22)-specific CD8⁺ T-cells isolated splenocytes (FIG. 11I) and lung tumor-infiltrating T-cells (FIG. 11J) at day 15 post subcutaneous (subcutis) and intravenous (lung) injection. Mice were injected with mElm4-Alk (left panel), with Eml4-Alk^{PGPGRVAKI-1} (“PGPGRVAKI” disclosed as SEQ ID NO: 22) (middle panel), and with Eml4-Alk^{PGPGRVAKI-2} (“PGPGRVAKI” disclosed as SEQ ID NO: 22) (right panel). **P<0.005; ***P<0.001; ****P<0.0001. (All P values were calculated using an unpaired two-tailed Student's t test). FIGS. 11I and 11J disclose “PGPGRVAKI” as SEQ ID NO: 22.

FIGS. 12A-12C provide bar graphs and images showing that the ALK vaccine expanded PGPGRVAKI (SEQ ID NO: 22)-specific lung tumor infiltrating CD8⁺ T-cells. (FIG. 12A) Quantitative analysis of intratumor CD8 positive cells per high power field (HPF) from mice in indicated treatment regimes. Data displayed as %±SEM (unpaired two-tailed Student's t test). (FIG. 12B) Dextramer staining of PGPGRVAKI (SEQ ID NO: 22)-specific CD8⁺ T-cells isolated from lung tumors, displayed as percentage (%±SEM). Mice were treated as indicated, and tumors collected upon death. Each dot represents an individual mouse. (FIG. 12C) Representative H&E and paired CD8 IHC staining of lung tumors from mice treated as indicated. Scale bars=50 μm. ***P<0.001.

FIGS. 13A-13C Efficacy of the ALK vaccine as monotherapy against ALK+ lung tumors in hEML4-ALK Tg mice. (FIG. 13A) Schematic representation of treatment protocol of hEML4-ALK Tg mice vaccinated either with ALK peptide pool (peptide A, 7, 20/21) or with ALK-DNA vaccine. (FIG. 13B) Cytotoxic activity of ALK-specific CD8⁺ T-cells in mice treated as in FIG. 13A. Each dot represents a mouse. (FIG. 13C) Tumor volume measured by MRI at the indicated time points in hEML4-ALK Tg mice (untreated, ALK-peptide vaccinated [peptides A, 7 and 20/21 and ALK-DNA vaccinated). *P<0.05; **P<0.005; ***P<0.001; n/s, not significant. (All P values were calculated using an unpaired two-tailed Student's t test). In FIG. 3C triangles represent ALK-DNA vax, squares represent ALK-prep vax, and circles represent Untreated.

FIGS. 14A-14L provide immunoblot images, bar graphs, and chromatograms showing tumor escape in vaccinated mice is due to reversible MHC-I downregulation. (FIG. 14A) Relative normalized expression of ALK in lung tumors escapers from each indicated treatment. (FIG. 14B) Immunoblot for indicated proteins from four MHC-I^{high} and four MHC-I^{low} tumor escaper lines. Eml4-Alk^{PGPGRVAKI-1} (“PGPGRVAKI” disclosed as SEQ ID NO: 22) parental cell line was used as control. FIG. 14B discloses SEQ ID NO: 22. (FIG. 14C) Representative Sanger sequencing chromatogram showing the PGPGRVAKI (SEQ ID NO: 22) peptide cDNA sequencing of lung tumor escapers from each indicated treatment group. mEml4-Alk represents the unedited sequence. FIG. 14C discloses SEQ ID NOS 375, 379, 377, 380, 377, 380, 377, 381, 377, and 380, respectively, in order of appearance. (FIG. 14D) PD-L1 staining of lung tumor escapers, displayed as MFI (±SEM). Mice were treated as indicated and each dot represents an individual tumor. (FIGS. 14E-14L) Relative normalized expression of LPM2 (FIG. 14E), LPM7 (FIG. 14F), TAP1 (FIG. 14G), TAP2 (FIG. 14H), β2M (FIG. 14I), TAPASIN (FIG. 14J), MECL1 (FIG. 14K), and STING (FIG. 14L) genes in lung tumor escapers from each indicated treatment group.

FIGS. 15A-15E provide a bar graph, and plots showing MHC-I expression in ALK+ NSCLC and identification of MHC-I human ALK peptides. (FIG. 15A) Provides a bar graph showing MHC-I H-scores and ALK H-scores and demonstrating that all ALK⁺ NSCLC patients had MHC-I H-scores that would imply that the patients would benefit from and/or be responsive to administration of the ALK vaccines of the present disclosure. In FIG. 15A, the bars on the left in each pair of bars represents “MHC-I H-score” and the bars on the right in each pair of bars represents “ALK H-score.” (FIG. 15B) Targeted mass spectrometry from HLA A*02:01 immunoprecipitations of the ALCL cell line DEL. Oxidized and non-oxidized methionine forms of the peptide AMLDLLHVA (SEQ ID NO: 7) were monitored. FIG. 15B discloses “AMLDLLHVA” as SEQ ID NO: 7. (FIG. 15C) Identification of ALK-specific peptides by discovery mass spectrometry from pan-HLA immunoprecipitations of the ALCL cell line KARPAS-299. FIG. 15C discloses SEQ ID NOS 5, 382-385, and 4, and 3, respectively, in order of appearance. (FIGS. 15D and 15E) Identification of ALK-specific peptides by LC-DIAMS from pan-HLA immunoprecipitations of the ALK-positive cell lines DEL (FIG. 15D) and KARPAS-299 (FIG. 15E). Precursor ions and Poisson chromatograms with detection at elution marked by an arrow. FIGS. 15D and 15E disclose SEQ ID NOS 7, 5, 3, and 4, respectively, in order of appearance.

FIGS. 16A and 16B provide schematics. (FIG. 16A) Scheme of the vaccination of CB6F1-B2m^{tm1Unc}; Tg(B2M)55Hpl; Tg(HLA-B*0702/H2-Kb) mice. (FIG. 16B) Scheme of the expansion of CD8⁺ T cells in the presence of ALK-specific peptides. The displayed alternative methods were applied for those patients with fewer PBMCs.

FIG. 17 provides a chart with shaded cells that provides a detailed group analysis corresponding to FIG. 1.

FIGS. 18A-18D provide a schematic, a line graph, a dot plot, and images illustrating the lack of detectable toxicity of the ALK vaccine. FIG. 18A is a schematic representation of HLA-A*02:01 and HLA-B*07:02 transgenic mice vaccinated with either AMLDLLHVA (SEQ ID NO: 7) or IVRCIGVSL (SEQ ID NO: 4) peptides, respectively, and CDN adjuvant. FIG. 18B show average mouse weight over time. Data are represented as mean±SEM. FIG. 18C shows representative H&E and pan-HLA immunohistochemistry staining of the hypothalamus region from HLA-A*02:01 and HLA-B*07:02 transgenic mice vaccinated as in FIG. 18A. FIG. 18C shows 40× magnification. Scale bars=100 μm. FIG. 18D shows quantitative analysis of infiltrating CD8⁺ T cells per high power field (HPF) in the hypothalamus region from HLA-A*02:01 and HLA-B*07:02 transgenic mice vaccinated as in FIG. 18A. Data are represented as %±SEM. Unpaired two-tailed Student's t test, n/s=not significant.

### DETAILED DESCRIPTION OF THE INVENTION

The invention features compositions and methods that are useful for treating anaplastic lymphoma kinase (ALK)-rearranged Non-Small Cell Lung Cancers (NSCLCs). The methods involve administering to a subject ALK peptides and/or polynucleotides encoding the ALK peptides, optionally in combination with an immune checkpoint inhibitor (ICI) and/or an ALK tyrosine kinase inhibitor (TKI).

The invention is based, at least in part, upon the discovery, as detailed in the Examples provided herein, that ALK vaccination completely prevented metastatic dissemination of ALK⁺ tumors, including brain metastasis. The ALK vaccination also impaired tumor progression and achieved complete cure in a subset of subjects. It was also found that the spontaneous systemic and intratumoral ALK-specific CD8⁺ T-cell response was lower when the same ALK⁺ cells grew as tumors in the lungs compared to tumors in the flank. Consequently, ICI induced rejection of flank ALK⁺ tumors but was infective against lung tumors, consistent with an inefficient priming of ALK-specific CD8⁺ T cells in the lung. In contrast, priming of ALK-specific CD8⁺ T cells was enhanced by single peptide vaccination leading to growth impairment and eradication of lung tumors in combination with ALK TKI therapy. ALK vaccination restored ALK-specific T cell priming against ALK+ lung cancer (ALK-rearranged NSCLC). Further, the invention is also based at least in part upon the identification of human ALK peptides that bind the HLA-A*0201 and B*0702 MHC-I alleles that are immunogenic in transgenic mice and are recognized by CD8⁺ T-cells of NSCLC patients. Not intending to be bound by theory, the data provided herein pave the way for the development of a clinical ALK vaccine to treat ALK⁺ NSCLC.

Lung cancer is the most common cause of cancer-related death worldwide, and the annual incidence of anaplastic lymphoma kinase (ALK) expressing non-small cell lung cancer (NSCLC) in the U.S. is about 8,000 cases. In these patients, treatment with ALK tyrosine kinase inhibitors (TKIs) fails to induce durable remissions. In this context, a successful ALK vaccine could lead to durable responses and greatly improve survival and quality of life for NSCLC patients. ALK represents an attractive target for vaccine development because of its oncogenicity, its immunogenicity, and its restricted expression to tumor tissue rather than healthy adult tissue. Importantly, use of a therapeutic ALK peptide vaccine could potentially be extended to many other cancer types which are driven by ALK rearrangements or activating mutations (i.e., ALK-positive cancers), such as anaplastic large cell lymphoma (ALCL), neuroblastoma, B-cell lymphoma, thyroid cancer, colon cancer, breast cancer, inflammatory myofibroblastic tumors (IMT), renal carcinoma, esophageal cancer, glioma, glioblastoma, and melanoma. Therefore, a vaccine as described herein generated against the rearranged portion of ALK can both prevent the development of ALK-positive tumors and more effectively treat patients diagnosed with ALK-positive tumors.

As described below, the present invention features isolated ALK-specific immunogenic antigens, e.g., peptide antigens, derived from ALK-positive cell lines and immune cells from patients with ALK-positive cancers. Such immunogenic antigens are also referred to as “immunogens” herein. The ALK-specific immunogenic antigens elicit a potent immune response, e.g., in the form of reactive T-lymphocytes, following administration or delivery to, or introduction into, a subject, particularly, a human subject. The isolated ALK-specific immunogenic antigens may be used in methods to treat and/or reduce disease caused by oncogenic ALK gene fusions, rearrangements, duplications, or mutations. The isolated ALK-specific immunogenic antigens may be conjugated to an amphiphilic tail in order to significantly increase T-cell expansion and greatly enhance anti-tumor efficacy.

The immunogenic ALK antigens described herein may be used in immunogenic compositions (e.g., ALK-specific vaccines) that treat ALK-positive cancers caused by oncogenic ALK gene fusions, rearrangements, duplications, or mutations in a subject, particularly a human subject, to whom the immunogenic composition or vaccine, is administered. The vaccine elicits a potent ALK protein-specific T cell response that treats and/or protects against ALK-positive cancers in a subject. The antigens, immunogens, immunogenic compositions and vaccines, and pharmaceutical compositions thereof, of the invention provide an additional treatment option for patients that have either become resistant to or have failed to respond to prior and traditional therapies for ALK-positive cancers.

### Identification of ALK Proteins

The use of computational algorithms has been successfully applied in recent studies to identify T-cell neoantigens in both human and mice (Carreno B M, et al. Cancer immunotherapy. A dendritic cell vaccine increases the breadth and diversity of melanoma neoantigen-specific T-cells. Science. 2015; 348(6236):803-808; and Gubin M M, et al. Checkpoint blockade cancer immunotherapy targets tumor-specific tumor antigens. Nature. 2014; 515(7528):577-581). However, these algorithms are almost exclusively based on the affinities of synthetic peptides, and do not necessarily consider other parameters such as antigen expression levels, intracellular processing, and the transport of the peptides prior to HLA binding. Altogether, these factors can significantly limit the accuracy of the current algorithms in predicting genuine T-cell epitopes.

By using a liquid chromatography-tandem mass spectrometry (LC-MS/MS) analysis of the peptides presented on the surface of HLA monoallelic cells, only 26% of the peptides that actually bind common HLA alleles were predicted by Immune Epitope Database (IEDB) algorithms, one of the most commonly used algorithms (www.iedb.org) (Abelin J G, et al. Mass Spectrometry Profiling of HLA-Associated Peptidomes in Mono-allelic Cells Enables More Accurate Epitope Prediction. Immunity. 2017; 46(2):315-326). The level of accuracy drops down to 0% for rare HLA alleles. Thus, current algorithms used to predict antigenic peptides are generally inaccurate and misleading as to which antigens are actually presented on tumor cell surfaces (Abelin J G, et al. Mass Spectrometry Profiling of HLA-Associated Peptidomes in Mono-allelic Cells Enables More Accurate Epitope Prediction. Immunity. 2017; 46(2):315-326).

For direct identification of ALK antigenic peptides effectively presented on the cell surface of tumor cells in the most common HLA haplotypes, the HLA monoallelic cell system and algorithms as provided in Abelin J G, et al. (Mass Spectrometry Profiling of HLA-Associated Peptidomes in Mono-allelic Cells Enables More Accurate Epitope Prediction. Immunity. 2017; 46(2):315-326), which is incorporated herein in its entirety, were adapted. To directly identify ALK peptides actually presented on the surface of ALK-expressing tumor cells, HLA-peptide complexes were pulled from ALK-expressing cell lines lysates and the HLA-bound peptides were analyzed using liquid chromatography-tandem mass spectrometry (LC-MS/MS).

The invention provides for a method for identifying the ALK-specific peptides provided herein as described in Examples 2 and 7. In some embodiments, the HLA is presented by a human ALK⁺ tumor cell line expressing an HLA class I allele. In some embodiments, the HLA class I allele is HLA A*02:01 or HLA B*07:02.

In some embodiments, ALK-expressing cell lines may be used in identifying the ALK antigenic peptides provided herein encode specific HLA-alleles (e.g., HLA class I alleles) and may express or may be transduced with a construct to express an ALK fusion protein (e.g., ELM4-ALK or NPM-ALK). In some embodiments, the ALK-expressing cells lines are generated as described in Abelin J G, et al. (Mass Spectrometry Profiling of HLA-Associated Peptidomes in Mono-allelic Cells Enables More Accurate Epitope Prediction. Immunity. 2017; 46(2):315-326), which is incorporated herein in its entirety.

In some embodiments, the ALK-expressing cell line may include the B721.221 human lymphoblastic cell line, which does not express endogenous HLA class I (A, B and C) due to gamma-ray-induced mutations in the HLA complex (Shimizu Y, DeMars R. Production of human cells expressing individual transferred HLA-A, -B, -C genes using an HLA-A, -B, -C null human cell line. J. Immunol. 1989; 142(9):3320-3328). In some embodiments, the B721.221 cell line is transduced with a construct encoding an EML4-ALK fusion protein. In some embodiments, the construct encodes EML4-ALK variant 1, the most frequent EML4-ALK fusion protein (Lin J J, et al. Impact of EML4-ALK Variant on Resistance Mechanisms and Clinical Outcomes in ALK-Positive Lung Cancer. J. Clin. Oncol. 2018:JCO2017762294.

In some embodiments, the fusion protein is an ALK protein fused to a nucleophosmin (NPM) protein (NPM-ALK). In some embodiments, ALK-expressing cell lines may include anaplastic large cell lymphoma (ALCL) cell lines encoding frequent HLA-alleles (e.g., Karpas-299, DEL, and SR-786). These ALCL cell lines express high levels of the NPM-ALK fusion protein.

### ALK Immunogenic Polypeptides

The present invention features the identification of ALK antigens and immunogenic polypeptides (immunogens) with the ability to generate an immune response so as to treat a disease and its symptoms, either prophylactically or therapeutically, caused by oncogenic ALK gene fusions, rearrangements, duplications, or mutations (e.g., ALK-positive cancers) following administration and delivery to a susceptible subject. It will also be appreciated that the isolated ALK antigen proteins as described herein and used as immunogens elicit an immune response, e.g., producing T-lymphocytes, in a subject. The ALK antigens and immunogens of the invention may be incorporated into a pharmaceutical composition, immunogenic composition, or vaccine as provided herein.

In some embodiments, the isolated ALK antigen protein elicits a protective immune response against at least one, more than one, or all types of ALK-positive cancers.

In some embodiments, the disease caused by oncogenic ALK gene fusions, rearrangements, duplications, or mutations is an ALK-positive cancer. Nonlimiting examples of ALK-positive cancers include non-small cell lung cancer (NSCLC), anaplastic large cell lymphoma (ALCL), neuroblastoma, B-cell lymphoma, thyroid cancer, colon cancer, breast cancer, inflammatory myofibroblastic tumors (IMT), renal carcinoma, esophageal cancer, glioma, glioblastoma, melanoma, or a combination thereof. In some embodiments, the ALK-positive cancer is non-small cell lung cancer (NSCLC). In some embodiments, the ALK-positive cancer is anaplastic large cell lymphoma (ALCL).

### ALK Antigens and Immunogens

The present invention provides herein ALK antigens and immunogens capable of generating an immune response against one or more diseases caused by oncogenic ALK gene fusions, rearrangements, duplications, or mutations (e.g., ALK-positive cancers). An ALK antigen or immunogen as described herein is a polypeptide, peptide, or antibody-binding portion thereof. In some embodiments, the ALK antigen or immunogen is an ALK polypeptide or fragment thereof.

In some embodiments, ALK antigen or immunogen amino acid sequence comprises an amino acid sequence that is at least 95%, at least 98%, at least 99%, or 100% identical to a sequence provided in Table 1, 2A, 2B, 2C, and/or 7 and/or to any of the following amino acid sequences: RPRPSQPSSL (SEQ ID NO: 3); IVRCIGVSL (SEQ ID NO: 4); VPRKNITLI (SEQ ID NO: 5); TAAEVSVRV (SEQ ID NO: 6); AMLDLLHVA (SEQ ID NO: 7); FNHQNIVRCIGVSL (SEQ ID NO: 1); and/or GGDLKSFLRETRPRPSQPSSLAM (SEQ ID NO: 2). In some embodiments, the ALK antigen or immunogen comprises an amino acid sequence that is at least 95%, at least 98%, at least 99%, or 100% identical to: RPRPSQPSSL (SEQ ID NO: 3). In some embodiments, the ALK antigen or immunogen comprises an amino acid sequence that is at least 95%, at least 98%, at least 99%, or 100% identical to: IVRCIGVSL (SEQ ID NO: 4). In some embodiments, the ALK antigen or immunogen comprises an amino acid sequence that is at least 95%, at least 98%, at least 99%, or 100% identical to: VPRKNITLI (SEQ ID NO: 5). In some embodiments, the ALK antigen or immunogen comprises an amino acid sequence that is at least 95%, at least 98%, at least 99%, or 100% identical to: TAAEVSVRV (SEQ ID NO: 6). In some embodiments, the ALK antigen or immunogen comprises an amino acid sequence that is at least 95%, at least 98%, at least 99%, or 100% identical to: AMLDLLHVA (SEQ ID NO: 7). In some embodiments, the ALK antigen or immunogen comprises an amino acid sequence that is at least 95%, at least 98%, at least 99%, or 100% identical to: FNHQNIVRCIGVSL (SEQ ID NO: 1). In some embodiments, the ALK antigen or immunogen comprises an amino acid sequence that is at least 95%, at least 98%, at least 99%, or 100% identical to:

(SEQ ID NO: 2)
GGDLKSFLRETRPRPSQPSSLAM.

In some embodiments, the ALK antigen or immunogen is conjugated to an amphiphile or amphiphilic tail. In some embodiments, the amphiphile is N-hydroxy succinimidyl ester-end-functionalized poly(ethylene glycol)-lipid (NHS-PEG2KDa-DSPE). ALK amph-peptides may significantly increase T-cell expansion and greatly enhance anti-tumor efficacy. ALK amph-peptides may be generated as taught in H. Liu et al., Structure-based programming of lymph-node targeting in molecular vaccines. Nature 507, 5199522 (2014), which is incorporated herein in its entirety.

In some embodiments, the ALK antigen or immunogen, optionally conjugated to an amphiphile or amphiphilic tail, comprises an amino acid sequence from Table 1, 2A, 2B, 2C, and/or 7 and/or selected from the following amino acid sequences: RPRPSQPSSL (SEQ ID NO: 3); IVRCIGVSL (SEQ ID NO: 4); VPRKNITLI (SEQ ID NO: 5); TAAEVSVRV (SEQ ID NO: 6); AMLDLLHVA (SEQ ID NO: 7); FNHQNIVRCIGVSL (SEQ ID NO: 1); and/or GGDLKSFLRETRPRPSQPSSLAM (SEQ ID NO: 2).

In some embodiments, the ALK antigen or immunogen, optionally conjugated to an amphiphile or amphiphilic tail, comprises flanking amino acid sequences. In some embodiments, the flanking amino acid sequences are on either side or on both sides of the ALK antigen or immunogen sequence. In some embodiments, the ALK antigen or immunogen a central core amino acid sequence with flanking amino acid sequences on both sides of the core. In some embodiments, the core amino acid sequence is about 9 to 10 amino acids in length. In some embodiments, the flanking amino acid sequences are between 5 to 15 amino acids. In some embodiments, the ALK antigen or immunogen conjugated to an amphiphile or amphiphilic tail comprises an amino acid sequence that is about 9 to about 30 amino acids in length.

In some embodiments, the ALK antigen or immunogen is a polynucleotide molecule. In some embodiments, the ALK antigen or immunogen has a polynucleotide sequence that encodes a polypeptide or peptide antigen or fragment thereof as described herein.

In some embodiments, ALK polynucleotide sequences encode ALK antigen or immunogen amino acid sequences that are at least 95%, at least 98%, at least 99%, or 100% identical to the sequences provided in Table 1, 2A, 2B, 2C, and/or 7 and/or to any of the following amino acid sequences: RPRPSQPSSL (SEQ ID NO: 3); IVRCIGVSL (SEQ ID NO: 4); VPRKNITLI (SEQ ID NO: 5); TAAEVSVRV (SEQ ID NO: 6); AMLDLLHVA (SEQ ID NO: 7); FNHQNIVRCIGVSL (SEQ ID NO: 1); and/or GGDLKSFLRETRPRPSQPSSLAM (SEQ ID NO: 2). In some embodiments, the ALK polynucleotide sequence encodes the ALK antigen or immunogen amino acid sequence that is at least 95%, at least 98%, at least 99%, or 100% identical to: RPRPSQPSSL (SEQ ID NO: 3). In some embodiments, the ALK polynucleotide sequence encodes the ALK antigen or immunogen amino acid sequence that is at least 95%, at least 98%, at least 99%, or 100% identical to: IVRCIGVSL (SEQ ID NO: 4). In some embodiments, the ALK polynucleotide sequence encodes the ALK antigen or immunogen amino acid sequence that is at least 95%, at least 98%, at least 99%, or 100% identical to: VPRKNITLI (SEQ ID NO: 5). In some embodiments, the ALK polynucleotide sequence encodes the ALK antigen or immunogen amino acid sequence that is at least 95%, at least 98%, at least 99%, or 100% identical to: TAAEVSVRV (SEQ ID NO: 6). In some embodiments, the ALK polynucleotide sequence encodes the ALK antigen or immunogen amino acid sequence that is at least 95%, at least 98%, at least 99%, or 100% identical to: AMLDLLHVA (SEQ ID NO: 7). In some embodiments, the ALK polynucleotide sequence encodes the ALK antigen or immunogen amino acid sequence that is at least 95%, at least 98%, at least 99%, or 100% identical to: FNHQNIVRCIGVSL (SEQ ID NO: 1). In some embodiments, the ALK polynucleotide sequence encodes the ALK antigen or immunogen amino acid sequence that is at least 95%, at least 98%, at least 99%, or 100% identical to: GGDLKSFLRETRPRPSQPSSLAM (SEQ ID NO: 2).

In some embodiments, the amino acid sequence of the antigen or immunogen, e.g., the ALK protein, is reverse translated and optimized for expression in mammalian cells. As will be appreciated by a skilled practitioner in the art, optimization of the nucleic acid sequence includes optimization of the codons for expression of a sequence in mammalian cells and RNA optimization (such as RNA stability).

In some embodiments, the ALK antigen or immunogen is isolated and/or purified. In some embodiments, the antigen or immunogen is formulated for administration to a subject in need. In some embodiments, the antigen or immunogen is administered to a subject in need thereof in an effective amount to elicit an immune response (e.g., a T-cell response) in the subject. In some embodiments, the immune response produces T-lymphocytes. In some embodiments, the immune response is prophylactic or therapeutic. In some embodiments, the immune response is associated with a reduction in metastatic dissemination of tumors.

In some embodiments, fusion proteins comprising the ALK antigen polypeptides are as described herein. In some embodiments, the ALK polypeptide can be fused to any heterologous amino acid sequence to form the fusion protein. By way of example, peptide components of ALK polypeptides may be generated independently and then fused together to produce an intact ALK polypeptide antigen, for use as an immunogen.

### ALK Immunogenic Compositions and Vaccines

The ALK antigens or immunogens may be used in immunogenic compositions or vaccines to elicit an immune response, e.g., a T-cell response, against disease caused by oncogenic ALK gene fusions, rearrangements, duplications, or mutations (e.g., ALK-positive cancers). In some embodiments, the immune response includes producing T-lymphocytes.

In particular embodiments, the ALK polypeptides of the immunogenic compositions or vaccines contain antigenic determinants that serve to elicit an immune response in a subject (e.g., the production of activated T-cells) that can treat and/or protect a subject against disease caused by oncogenic ALK gene fusions, rearrangements, duplications, or mutations (e.g., ALK-positive cancers) and symptoms thereof.

In some embodiments, such immunogenic compositions or vaccines as described herein contain at least one ALK antigen or immunogen and are effective in treating, reducing, delaying, or preventing at least one disease caused by oncogenic ALK gene fusions, rearrangements, duplications, or mutations (e.g., ALK-positive cancers). In some embodiments, such immunogenic compositions or vaccines as described herein contain two or more ALK antigens or immunogens and are effective in treating, reducing, or preventing at least one disease caused by oncogenic ALK gene fusions, rearrangements, duplications, or mutations (e.g., ALK-positive cancers). In some embodiments, the two or more ALK antigens or immunogens comprise one, two, or more amino acid sequences selected from the following: AMLDLLHVA (SEQ ID NO: 7); RPRPSQPSSL (SEQ ID NO: 3); IVRCIGVSL (SEQ ID NO: 4); VPRKNITLI (SEQ ID NO: 5); TAAEVSVRV (SEQ ID NO: 6); FNHQNIVRCIGVSL (SEQ ID NO: 1); and/or GGDLKSFLRETRPRPSQPSSLAM (SEQ ID NO: 2). In some embodiments, the two or more ALK antigens or immunogens comprise two or more amino acid sequences selected from Tables 1, 2A-2C, and/or 7. In some embodiments, the immunogenic compositions or vaccines contain at least one ALK antigen or immunogen conjugated to an amphiphile or amphiphilic tail. In some embodiments, at least one of the two or more ALK antigens or immunogens in an immunogenic composition or vaccine is conjugated to an amphiphile or amphiphilic tail. In some embodiments, the amphiphile is N-hydroxy succinimidyl ester-end-functionalized poly(ethylene glycol)-lipid (NHS-PEG2KDa-DSPE). In some embodiments, the two or more ALK antigens or immunogens are provided in equal concentration ratios in an immunogenic composition or vaccine.

Because the ALK antigens or immunogens and the sequences thereof as described herein and used as immunogenic compositions or vaccines elicit an immune response in an immunocompetent subject, they provide a superior vaccine against which an immune response (e.g., producing T-lymphocytes) is generated.

In some embodiments, an immunogenic composition or a vaccine is provided that elicits an immune response (e.g., producing T-lymphocytes) in a subject following introduction, administration, or delivery of the antigen or immunogen to the subject. The route of introduction, administration, or delivery is not limited and may include, for example, intravenous, subcutaneous, intramuscular, oral, or other routes. The immunogenic composition or vaccine may be therapeutic (e.g., administered to a subject following a symptom of disease caused by oncogenic ALK gene fusions, rearrangements, duplications, or mutations (e.g., ALK-positive cancers)) or prophylactic (e.g., administered to a subject prior to the subject having or expressing a symptom of disease, or full-blown disease, caused by oncogenic ALK gene fusions, rearrangements, duplications, or mutations (e.g., ALK-positive cancers)).

### Vectors

Vectors containing a nucleotide sequence encoding an isolated ALK polypeptide or peptide antigen are provided. In some embodiments, the vectors comprise a nucleotide sequence encoding an ALK polypeptide or peptide antigen. In some embodiments, the vectors comprise a nucleotide sequence encoding the ALK polypeptide or peptide antigen. In some embodiments, the vector further includes a promoter operably linked to the nucleotide sequence encoding the ALK polypeptide. In a particular embodiment, the promoter is a cytomegalovirus (CMV) promoter.

The vectors used to express an ALK antigen as described herein may be any suitable expression vector known and used in the art. In some embodiments, the vector is a prokaryotic or eukaryotic vector. In some embodiments, the vector is an expression vector, such as a eukaryotic (e.g., mammalian) expression vector. In another embodiment, the vector is a plasmid (prokaryotic or bacterial) vector. In another embodiment, the vector is a viral vector. In some embodiments, the vector is an RNA polynucleotide suitable for translation in a cell.

Provided are isolated, non-naturally occurring polypeptide antigens, e.g., ALK polypeptide antigens, produced by transfecting a host cell with an expression vector as known and used in the art under conditions sufficient to allow for expression of the polypeptide, e.g., an ALK polypeptide, in the cell. Isolated cells containing the vectors are also provided.

Also provided is an ALK polypeptide, as described herein, produced by transfecting a host cell with a vector containing a polynucleotide encoding the ALK polypeptide. Also provided in some embodiments is an ALK polypeptide, as described herein, produced by transfecting a host cell with a vector encoding the ALK polypeptide under conditions sufficient to allow for expression of the ALK protein. Collections of plasmids (vectors) are also contemplated. In certain embodiments, the collection of plasmids includes plasmid encoding an ALK protein as described herein.

### Compositions and Pharmaceutical Compositions for Administration

Compositions comprising at least one ALK protein, or a polynucleotide encoding at least one ALK protein, as described herein are provided. In some embodiments, the compositions further comprise a pharmaceutically acceptable carrier, diluent, excipient, or vehicle. In some embodiments, an adjuvant (a pharmacological or immunological agent that modifies or boosts an immune response, e.g., to produce more antibodies that are longer-lasting) is also employed. For example, without limitation, the adjuvant can be an inorganic compound, such as alum, aluminum hydroxide, or aluminum phosphate; mineral or paraffin oil; squalene; detergents such as Quil A; plant saponins; Freund's complete or incomplete adjuvant, a biological adjuvant (e.g., cytokines such as IL-1, IL-2, or IL-12); bacterial products such as killed Bordetella pertussis, or toxoids; or immunostimulatory oligonucleotides (such as CpG oligonucleotides). In some embodiments, the adjuvant is conjugated to an amphiphile as previously described (H. Liu et al., Structure-based programming of lymph-node targeting in molecular vaccines. Nature 507, 5199522 (2014)). In some embodiments, the amphiphile is N-hydroxy succinimidyl ester-end-functionalized poly(ethylene glycol)-lipid (NHS-PEG2KDa-DSPE)

Compositions and preparations (e.g., physiologically or pharmaceutically acceptable compositions) containing ALK polypeptides or polynucleotides for parenteral administration include, without limitation, sterile aqueous or non-aqueous solutions, suspensions, and emulsions. Nonlimiting examples of non-aqueous solvents include propylene glycol, polyethylene glycol, vegetable oils, such as olive oil and canola oil, and injectable organic esters, such as ethyl oleate. Aqueous carriers include water, alcoholic/aqueous solutions, emulsions, or suspensions, including saline and buffered media. Parenteral vehicles include, for example, sodium chloride solution, Ringer's dextrose, dextrose and sodium chloride, lactated Ringer's, or fixed oils. Intravenous vehicles include, for example, fluid and nutrient replenishers, electrolyte replenishers (such as those based on Ringer's dextrose), and the like. Preservatives and other additives may also be present in such compositions and preparations, such as, for example, antimicrobials, antioxidants, chelating agents, colorants, stabilizers, inert gases, and the like.

Some of the compositions may potentially be administered as a pharmaceutically acceptable acid- or base-addition salt, formed by reaction with inorganic acids, such as hydrochloric acid, hydrobromic acid, perchloric acid, nitric acid, thiocyanic acid, sulfuric acid, and phosphoric acid, and organic acids, such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, tri-alkyl and aryl amines and substituted ethanolamines.

Provided herein are pharmaceutical compositions which include a therapeutically effective amount of an isolated ALK polypeptide or polynucleotide antigen, alone, or in combination with a pharmaceutically acceptable carrier. Pharmaceutically acceptable carriers include, but are not limited to, saline, buffered saline, dextrose, water, glycerol, ethanol, and combinations thereof. The carrier and composition can be sterile, and the formulation suits the mode of administration. The composition can also contain minor amounts of wetting or emulsifying agents, or pH buffering agents. The composition can be a liquid or aqueous solution, suspension, emulsion, dispersion, tablet, pill, capsule, powder, or sustained release formulation. A liquid or aqueous composition can be lyophilized and reconstituted with a solution or buffer prior to use. The composition can be formulated as a suppository, with traditional binders and carriers such as triglycerides. Oral formulations can include standard carriers, such as pharmaceutical grades of mannitol, lactose, starch, magnesium stearate, sodium saccharine, cellulose, and magnesium carbonate. Any of the commonly known pharmaceutical carriers, such as sterile saline solution or sesame oil, can be used. The medium can also contain conventional pharmaceutical adjunct materials such as, for example, pharmaceutically acceptable salts to adjust the osmotic pressure, buffers, preservatives, and the like. Other media that can be used in the compositions and administration methods as described are normal saline and sesame oil.

### Methods of Treatment, Administration and Delivery

Methods of treating a disease, or symptoms thereof, caused by the oncogenic ALK gene fusions, rearrangements, duplications, or mutations (e.g., ALK-positive cancers) are provided. In embodiments, the methods treat or reduce rates of metastasis (e.g., a central nervous system metastasis) in a subject having an ALK-rearranged NLSCLC. The methods comprise administering a therapeutically effective amount of an antigen, immunogen, immunogenic composition, or vaccine, as described herein, or a pharmaceutical composition comprising the immunogen or a vaccine, as described herein, to a subject (e.g., a mammal), in particular, a human subject. The invention provides methods of treating a subject suffering from, or at risk of, or susceptible to disease, or a symptom thereof, or delaying the progression of a disease caused by oncogenic ALK gene fusions, rearrangements, duplications, or mutations (e.g., ALK-positive cancers). In some embodiments, the method includes administering to the subject (e.g., a mammalian subject), an amount or a therapeutic amount of an immunogenic composition or a vaccine comprising at least one ALK antigen polypeptide, sufficient to treat the disease, delay the growth of, or treat the symptoms thereof, caused by the oncogenic ALK gene under conditions in which the disease and/or the symptoms thereof are treated.

In some embodiments, the methods herein include administering to the subject (including a human subject identified as in need of such treatment) an effective amount of an isolated, ALK antigen or immunogen polypeptide, or an immunogenic composition or vaccine, or a pharmaceutical composition thereof, as described herein to produce such effect. The treatment methods are suitably administered to subjects, particularly humans, suffering from, susceptible to, or at risk of having a disease, or symptoms thereof, caused by oncogenic ALK gene fusions, rearrangements, duplications, or mutations, namely, ALK-positive cancers. Nonlimiting examples of ALK-positive cancers include non-small cell lung cancer (NSCLC), anaplastic large cell lymphoma (ALCL), neuroblastoma, B-cell lymphoma, thyroid cancer, colon cancer, breast cancer, inflammatory myofibroblastic tumors (IMT), renal carcinoma, esophageal cancer, glioma, glioblastoma, melanoma, or a combination thereof.

In embodiments, the methods of the present disclosure involve administering an ALK peptide and/or polynucleotide encoding the ALK peptide to a subject more than once. In some cases, the ALK peptide and/or polynucleotide encoding the ALK peptide is administered about or at least about 1, 2, 3, 4, 5, 6, 7, 8, 9, 10, 11, 12, 13, 14, 15, 16, 17, 18, 19, or 20 times. In some cases, the ALK peptide and/or polynucleotide encoding the ALK peptide is administered no more than about 2, 3, 4, 5, 6, 7, 8, 9, 10, 11, 12, 13, 14, 15, 16, 17, 18, 19, or 20 times. In some embodiments, the ALK peptide and/or polynucleotide encoding the ALK peptide is administered about or at least about every day, week, 2 weeks, 3 weeks, month, 2 months, 3 months 4 months, 5 months, 6 months, year, 2 years, 3 years, 4 years, 5 years, or 10 years. In some embodiments, the ALK peptide and/or polynucleotide encoding the ALK peptide is administered no more than about every day, week, 2 weeks, 3 weeks, month, 2 months, 3 months 4 months, 5 months, 6 months, year, 2 years, 3 years, 4 years, 5 years, or 10 years.

Identifying a subject in need of such treatment can be based on the judgment of the subject or of a health care professional and can be subjective (e.g., opinion) or objective (e.g., measurable by a test or diagnostic method). Briefly, the determination of those subjects who are in need of treatment or who are “at risk” or “susceptible” can be made by any objective or subjective determination by a diagnostic test (e.g., blood sample, biopsy, genetic test, enzyme, or protein marker assay), marker analysis, family history, and the like, including an opinion of the subject or a health care provider. In some embodiments, the subject in need of treatment can be identified by measuring ALK specific autoantibodies and ALK-specific T-cell responses in a patient sample (e.g., blood sample) or by assessing infiltrating immune cell subsets from a tumor core biopsy from a subject. The ALK antigens and immunogens, such as ALK polypeptides, immunogenic compositions and vaccines as described herein, may also be used in the treatment of any other disorders in which disease caused by oncogenic ALK gene fusions, rearrangements, duplications, or mutations may be implicated. A subject undergoing treatment can be a non-human mammal, such as a veterinary subject, or a human subject (also referred to as a “patient”).

In addition, prophylactic methods of preventing or protecting against a disease (e.g., metastatic spread of a tumor), or symptoms thereof, caused by oncogenic ALK gene fusions, rearrangements, duplications, or mutations are provided. Such methods comprise administering a therapeutically effective amount of a pharmaceutical composition comprising an ALK immunogenic composition or vaccine as described herein to a subject (e.g., a mammal, such as a human), in particular, prior to development or onset of a disease, such as ALK-positive tumors or cancers.

In another embodiment, a method of monitoring the progress of a disease caused by oncogenic ALK gene fusions, rearrangements, duplications, or mutations (e.g., ALK-positive cancers), or monitoring treatment of the disease is provided. The method includes a diagnostic measurement (e.g., CT scan, screening assay or detection assay) in a subject suffering from or susceptible to disease or symptoms thereof associated with oncogenic ALK gene fusions, rearrangements, duplications, or mutations (e.g., ALK-positive cancers), in which the subject has been administered an amount (e.g., a therapeutic amount) of an isolated ALK protein, as described herein, or an immunogenic composition or vaccine as described herein, sufficient to treat the disease or symptoms thereof. The diagnostic measurement in the method can be compared to samples from healthy, normal controls; in a pre-disease sample of the subject; or in other afflicted/diseased patients to establish the treated subject's disease status. For monitoring, a second diagnostic measurement may be obtained from the subject at a time point later than the determination of the first diagnostic measurement, and the two measurements can be compared to monitor the course of disease or the efficacy of the therapy/treatment. In certain embodiments, a pre-treatment measurement in the subject (e.g., in a sample or biopsy obtained from the subject or CT scan) is determined prior to beginning treatment as described; this measurement can then be compared to a measurement in the subject after the treatment commences and/or during the course of treatment to determine the efficacy of (monitor the efficacy of) the disease treatment. In some embodiments, efficacy of the disease treatment can be performed with antibody marker analysis and/or interferon-gamma (IFN-γ) ELISPOT assays.

The isolated ALK antigen polypeptide or polynucleotide encoding the polypeptide, or compositions thereof, can be administered to a subject by any of the routes normally used for introducing a recombinant protein or composition containing the recombinant protein into a subject. Routes and methods of administration include, without limitation, intradermal, intramuscular, intraperitoneal, intrathecal, parenteral, such as intravenous (IV) or subcutaneous (SC), vaginal, rectal, intranasal, inhalation, intraocular, intracranial, or oral. Parenteral administration, such as subcutaneous, intravenous, or intramuscular administration, is generally achieved by injection (immunization). Injectables can be prepared in conventional forms and formulations, either as liquid solutions or suspensions, solid forms (e.g., lyophilized forms) suitable for solution or suspension in liquid prior to injection, or as emulsions. Injection solutions and suspensions can be prepared from sterile powders, granules, and tablets. Administration can be systemic or local.

The isolated ALK polypeptides or polynucleotide(s) encoding the polypeptides, or compositions thereof, can be administered in any suitable manner, such as with pharmaceutically acceptable carriers, diluents, or excipients as described supra. Pharmaceutically acceptable carriers are determined in part by the particular immunogen or composition being administered, as well as by the particular method used to administer the composition. Accordingly, a pharmaceutical composition comprising the isolated ALK antigen polypeptides or compositions thereof, can be prepared using a wide variety of suitable and physiologically and pharmaceutically acceptable formulations.

Further provided is a method of eliciting or generating an immune response in a subject with a disease caused by oncogenic ALK gene fusions, rearrangements, duplications, or mutations (e.g., ALK-positive cancers) by administering to the subject an isolated ALK protein antigen or immunogen, or immunogenic composition or vaccine thereof, as described herein. In some embodiments, the ALK protein can be administered using any suitable route of administration, such as, for example, by intramuscular injection. In some embodiments, the ALK protein is administered as a composition comprising a pharmaceutically acceptable carrier. In some embodiments, the composition comprises an adjuvant selected from, for example, alum, Freund's complete or incomplete adjuvant, a biological adjuvant or immunostimulatory oligonucleotides (such as CpG oligonucleotides). In some embodiments, the adjuvant is conjugated to an amphiphile. In other embodiments, the composition may be administered in combination with one or more therapeutic agents or molecules.

Also provided is a method of immunizing a subject against disease or the symptoms thereof caused by oncogenic ALK gene fusions, rearrangements, duplications, or mutations (e.g., ALK-positive cancers), in which the method involves administering to the subject an isolated ALK protein or polynucleotide encoding the protein as described herein, or administering an immunogenic composition or vaccine thereof. In some embodiments of the method, the composition further comprises a pharmaceutically acceptable carrier, diluent, excipient, and/or an adjuvant. For example, the adjuvant can be alum, Freund's complete or incomplete adjuvant, a biological adjuvant or immunostimulatory oligonucleotides (such as CpG oligonucleotides). In some embodiments, the adjuvant is conjugated to an amphiphile. In some embodiments, the ALK peptides (or compositions thereof) are administered intramuscularly.

An advantage of the immunogens and immunogenic compositions comprising ALK antigens described herein is that an immune response is elicited against not only the ALK-expressing tumor or cell line from which the antigen was derived, but also against one or more, or all, ALK-positive cancers, e.g., non-small cell lung cancer (NSCLC), anaplastic large cell lymphoma (ALCL), neuroblastoma, B-cell lymphoma, thyroid cancer, colon cancer, breast cancer, inflammatory myofibroblastic tumors (IMT), renal carcinoma, esophageal cancer, glioma, glioblastoma, melanoma, or a combination thereof. In some embodiments, the immunogens and immunogenic compositions described herein elicit immune responses against Non-Small Cell Lung Cancer (NSCLC). In some embodiments, the immunogens and immunogenic compositions described herein elicit immune responses against ALCL. Thus, the ALK immunogens are more cost effective to produce, and beneficially elicit an immune response, thus, obviating a need to make and administer a poly- or multivalent immunogenic composition or vaccine.

Administration of the isolated ALK antigen polypeptides or compositions thereof, can be accomplished by single or multiple doses. The dose administered to a subject should be sufficient to induce a beneficial therapeutic response in a subject over time, such as to inhibit, block, reduce, ameliorate, protect against, or prevent disease caused by oncogenic ALK gene fusions, rearrangements, duplications, or mutations (e.g., ALK-positive cancers). The dose required will vary from subject to subject depending on the species, age, weight, and general condition of the subject, by the severity of the cancer being treated, by the particular composition being used and by the mode of administration. An appropriate dose can be determined by a person skilled in the art, such as a clinician or medical practitioner, using only routine experimentation. One of skill in the art is capable of determining therapeutically effective amounts of ALK antigen or immunogen, or immunogenic compositions or vaccines thereof, that provide a therapeutic effect or protection against diseases caused by ALK gene fusions, rearrangements, duplications, or mutations (e.g., ALK-positive cancers) suitable for administering to a subject in need of treatment or protection.

### Adjuvants and Combination Therapies

The ALK immunogens or immunogenic compositions or vaccines containing an ALK-specific peptide antigen can be administered alone or in combination with other therapeutic agents to enhance antigenicity or immunogenicity, e.g., to increase an immune response, such as the elicitation of specific or neutralizing antibodies, in a subject. For example, the ALK-specific peptide can be administered with an adjuvant, such as alum, Freund's incomplete adjuvant, Freund's complete adjuvant, biological adjuvant, or immunostimulatory oligonucleotides (such as CpG oligonucleotides). The adjuvant may be conjugated to an amphiphile as previously described (H. Liu et al., Structure-based programming of lymph-node targeting in molecular vaccines. Nature 507, 5199522 (2014)). In some embodiments, the amphiphile conjugated to the adjuvant is N-hydroxy succinimidyl ester-end-functionalized poly(ethylene glycol)-lipid (NHS-PEG2KDa-DSPE).

One or more cytokines, such as interleukin-1 (IL-2), interleukin-6 (IL-6), interleukin-12 (IL-12), the protein memory T-cell attractant “Regulated on Activation, Normal T Expressed and Secreted” (RANTES), granulocyte-macrophage-colony stimulating factor (GM-CSF), tumor necrosis factor-alpha (TNF-α), or interferon-gamma (IFN-γ); a stimulator of interferon genes (STING) agonist (e.g., ADU-S100); one or more growth factors, such as GM-CSF or granulocyte-colony stimulation factor (G-CSF); one or more molecules such as the TNF ligand superfamily member 4 ligand (OX40L) or the type 2 transmembrane glycoprotein receptor belonging to the TNF superfamily (4-1BBL), or combinations of these molecules, can be used as biological adjuvants, if desired or warranted (see, e.g., Salgaller et al., 1998, J Surg. Oncol. 68(2):122-38; Lotze et al., 2000, Cancer J Sci. Am. 6(Suppl 1):561-6; Cao et al., 1998, Stem Cells 16(Suppl 1):251-60; Kuiper et al., 2000, Adv. Exp. Med. Biol. 465:381-90). These molecules can be administered systemically (or locally) to a subject. These molecules and the ALK immunogens or immunogenic compositions or vaccines can be administered as the same or as separate dosage forms.

Several ways of inducing cellular responses, both in vitro and in vivo, are known and practiced in the art. Lipids have been identified as agents capable of assisting in priming cytotoxic lymphocytes (CTL) in vivo against various antigens. For example, palmitic acid residues can be attached to the alpha and epsilon amino groups of a lysine residue and then linked (for example, via one or more linking residues, such as glycine, glycine-glycine, serine, serine-serine, or the like) to an immunogenic peptide (U.S. Pat. No. 5,662,907). The lipidated peptide can then be injected directly in a micellar form, incorporated in a liposome, or emulsified in an adjuvant. As another example, E. coli lipoproteins, such as tripalmitoyl-S-glycerylcysteinlyseryl-serine can be used to prime tumor-specific CTL when covalently attached to an appropriate peptide. Moreover, the induction of neutralizing antibodies can also be primed with the same molecule conjugated to a peptide which displays an appropriate epitope, and two compositions can be combined to elicit both humoral and cell-mediated responses where such a combination is deemed desirable.

The ALK-specific peptides can also be administered as a combination therapy with one or more other therapeutic agents, such as ALK inhibitors, tyrosine kinase inhibitors (TKIs), and/or immune checkpoint inhibitors. Non-limiting examples of ALK inhibitors include lorlatinib (LORBRENA®). Non-limiting examples of checkpoint inhibitors include programmed cell death protein 1 (PD-1) inhibitors, programmed death-ligand 1 (PD-L1) inhibitors, cytotoxic T-lymphocyte-associated antigen-4 (CTLA-4) inhibitors, T-cell immunoglobulin and mucin domain 3 (TIM3) inhibitors, lymphocyte-activation gene 3 (LAG3) inhibitors, T-cell immunoglobulin and ITIM domain (TIGIT) inhibitors, V-domain immunoglobulin suppressor of T cell activation (VISTA) inhibitors, immunoglobulin-like transcript 2 (ILT2) inhibitors, immunoglobulin-like transcript 4 (ILT4) inhibitors, and killer cell immunoglobulin-like receptor, three immunoglobulin domains and long cytoplasmic tail (KIR3DL3) inhibitors. Non-limiting examples of checkpoint inhibitors include antibodies or fragments thereof. Nonlimiting examples of PD-1 inhibitors include pembrolizumab (KEYTRUDA®) and nivolumab (OPDIVO®). Non-limiting examples of PD-L1 inhibitors include atezolizumab (TECENTRIQ®), avelumab (BAVENCIO®), and durvalumab (IMFINZI®). Non-limiting examples of TIM3 inhibitors include sabatolimab and cobolimab. Non-limiting examples of LAG3 inhibitors include relatimab. Non-limiting examples of TIGIT inhibitors include vibostolimab, ociperlimab, domvanalimab, and etigilimab. Non-limiting examples of VISTA inhibitors include onvatilimab. Non-limiting examples of ILT2 inhibitors include BND-22. Non-limiting examples of ILT4 inhibitors include MK-4830 and JTX-8064. Non-limiting examples of KIR3DL3 inhibitors include NPX-267. Nonlimiting examples of CTLA-4 inhibitors include ipilimumab (YERVOY®). Non-limiting examples of TKI inhibitors include crizotinib, ceritinib, alectinib, brigatinib, ensartinib, entrectinib, and lorlatinib.

In some embodiments, one or more ALK inhibitors, immune checkpoint inhibitors, and/or TKI inhibitors is administered simultaneously or sequentially with ALK-specific peptide antigens, immunogens, or immunogenic compositions or vaccines containing an ALK-specific peptide antigen or immunogen. In some embodiments, the ALK-specific peptide antigen, immunogen, or immunogenic composition or vaccine containing an ALK-specific peptide antigen or immunogen is administered with a PD-1 inhibitor, a PD-L1 inhibitor, a CTLA-4 inhibitor, a TIM3 inhibitor, a LAG3 inhibitor, a TIGIT inhibitor, a VISTA inhibitor, an ILT2 inhibitor, an ILT4 inhibitor, and/or a KIR3DL3 inhibitor. In some embodiments, the PD-1 inhibitor is an anti-PD-1 antibody. In some embodiments, the PD-L1 inhibitor is an antibody. In some embodiments, the CTLA-4 inhibitor is an anti-CTLA-4 antibody. In some embodiments, the TIM3 inhibitor is an anti-TIM3 antibody. In some embodiments, the LAG3 inhibitor is an anti-LAG3 antibody. In some embodiments, the TIGIT inhibitor is an anti-TIGIT antibody. In some embodiments, the VISTA inhibitor is an anti-VISTA antibody. In some embodiments, the ILT2 inhibitor is an anti-ILT2 antibody. In some embodiments, the ILT4 inhibitor is an anti-ILT4 antibody. In some embodiments, the KIR3DL3 inhibitor is an anti-KIR3DL3 antibody.

In some embodiments, the ALK-specific peptide antigen, immunogen, or immunogenic composition or vaccine containing an ALK-specific peptide antigen or immunogen is administered with a TKI inhibitor. In some embodiments, the ALK-specific peptide antigen, immunogen, or immunogenic composition or vaccine containing an ALK-specific peptide antigen or immunogen is administered with an ALK inhibitor. In some embodiments, the ALK-specific peptide antigen, immunogen, or immunogenic composition or vaccine containing an ALK-specific peptide antigen or immunogen is administered with lorlatinib.

In some embodiments, the ALK-specific peptide antigen, immunogen, or immunogenic composition or vaccine containing an ALK-specific peptide antigen or immunogen is administered with a PD-1 inhibitor, a PD-L1 inhibitor, and/or a CTLA-4 inhibitor in combination with an ALK inhibitor. In some embodiments, the ALK-specific peptide antigen, immunogen, or immunogenic composition or vaccine containing an ALK-specific peptide antigen or immunogen is administered with an ALK inhibitor, optionally in combination with one or more of an PD-1 inhibitor, a PD-L1 inhibitor, a CTLA-4 inhibitor, IFN-γ, and/or a STING agonist (e.g., ADU-S100). In some embodiments, the ALK-specific peptide antigen, immunogen, or immunogenic composition or vaccine containing an ALK-specific peptide antigen or immunogen is administered with lorlatinib.

While treatment methods may involve the administration of a vaccine containing a ALK immunogenic protein as described herein, one skilled in the art will appreciate that the ALK protein itself, as a component of a pharmaceutically acceptable composition or as a fusion protein, can be administered to a subject in need thereof to elicit an immune response against an ALK-positive cancer in the subject.

### Kits

Also provided are kits containing the ALK antigen or immunogen as described, or an immunogenic composition, or a vaccine, or a pharmaceutically acceptable composition containing the antigen or immunogen and a pharmaceutically acceptable carrier, diluent, or excipient, for administering to a subject, for example. The antigen or immunogen may be in the form of an ALK protein (polypeptide) or a polynucleotide (a polynucleotide encoding an ALK polypeptide), as described herein. Kits containing one or more of the plasmids, or a collection of plasmids as described herein, are also provided. As will be appreciated by the skilled practitioner in the art, such a kit may contain one or more containers that house the antigen, immunogen, vaccine, or composition, carriers, diluents, or excipients, as necessary, and instructions for use.

The practice of the present invention employs, unless otherwise indicated, conventional techniques of molecular biology (including recombinant techniques), microbiology, cell biology, biochemistry, and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Useful techniques for particular embodiments will be discussed in the sections that follow.

The following examples are put forth to provide those of ordinary skill in the art with a complete disclosure and description of how to make and use the assay, screening, and therapeutic methods of the invention, and are not intended to limit the scope of what the inventors regard as their invention.

### EXAMPLES

The following examples are provided to illustrate certain particular features and/or embodiments. The examples should not be construed to limit the disclosure to the particular features or embodiments described.

### Example 1. Immune Checkpoint Inhibitors (ICIS) Did not Increase the Efficacy of Anaplastic Lymphoma Kinase (ALK) Treatment with Alk Tyrosine Kinase Inhibitors (TKIS) in Alk⁺ Lung Cancer Mouse Models

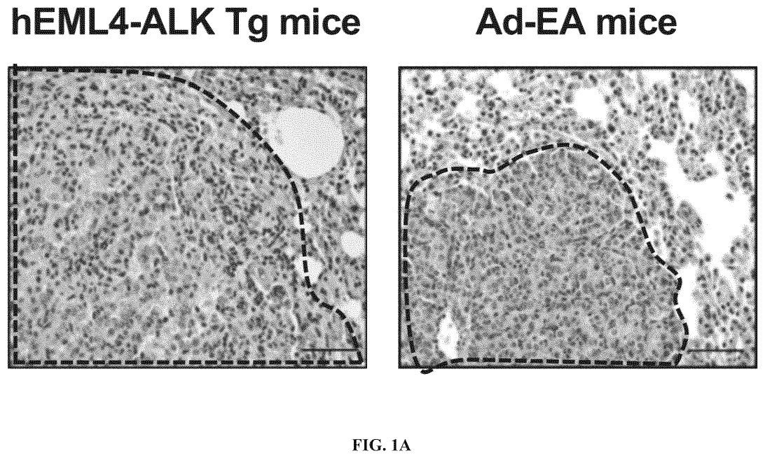

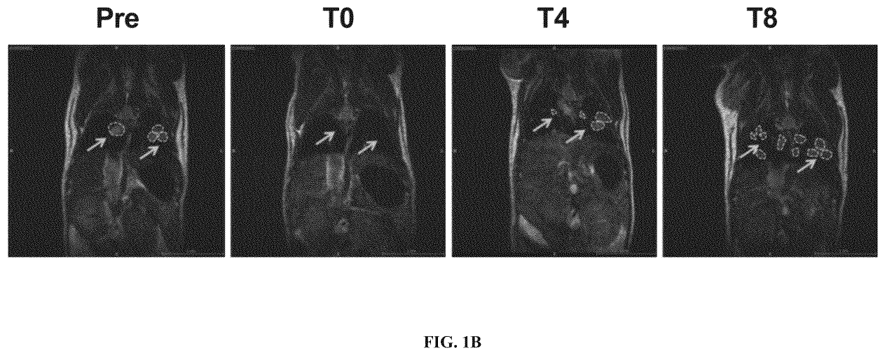

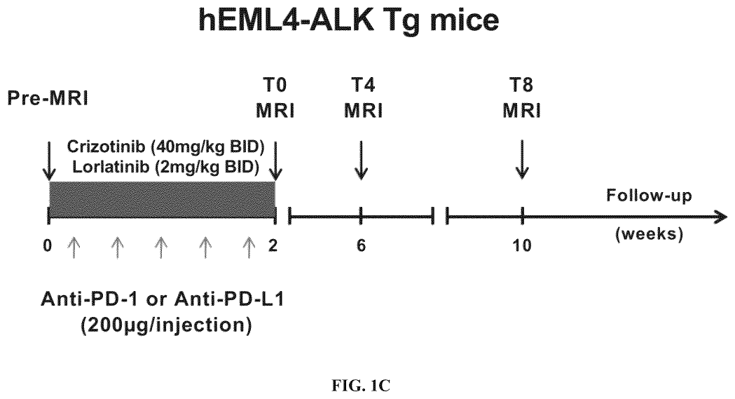

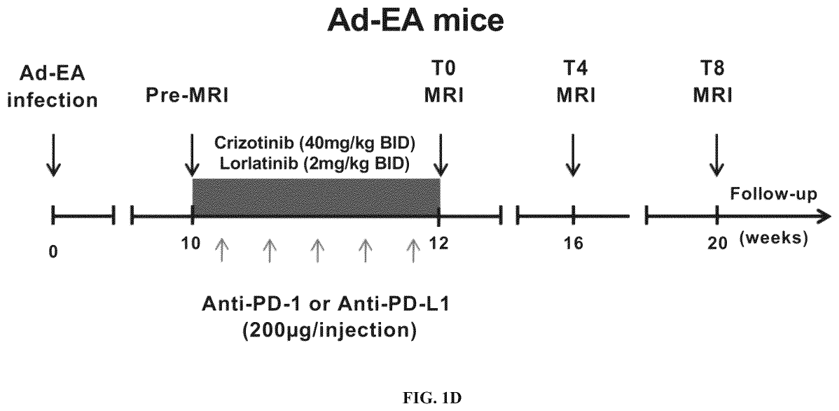

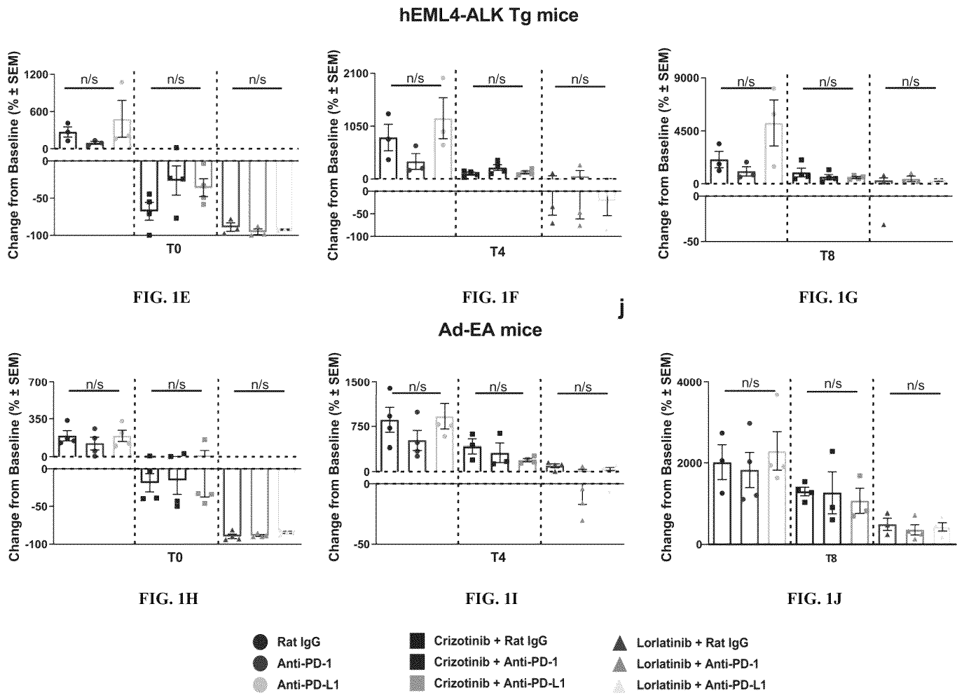

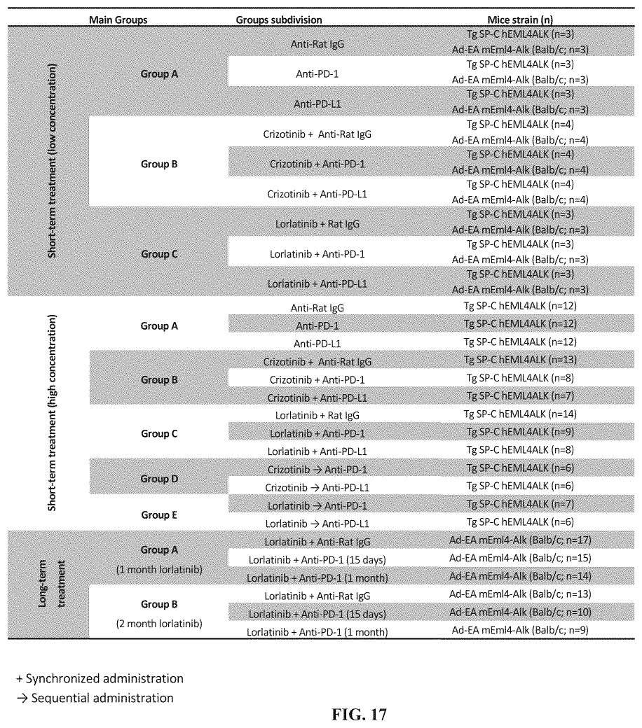

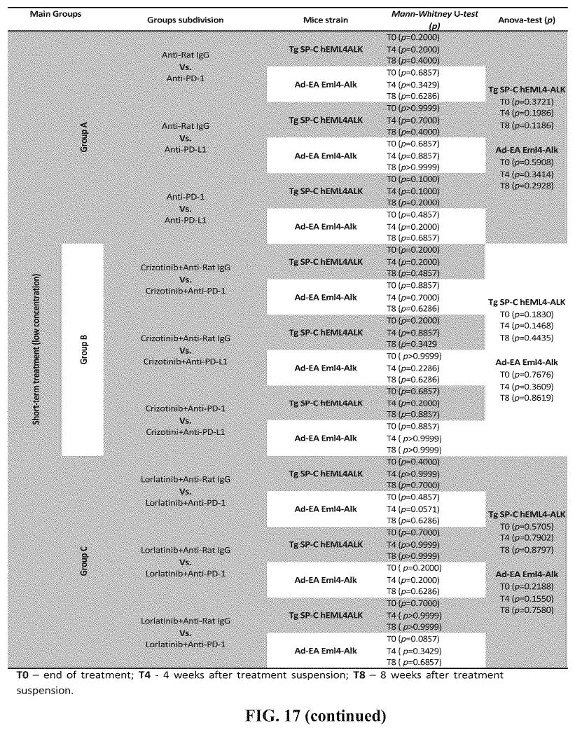

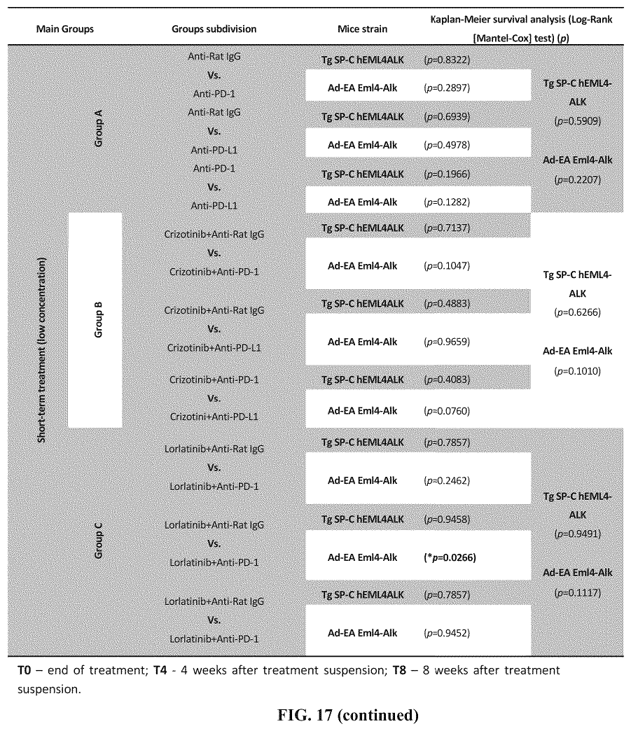

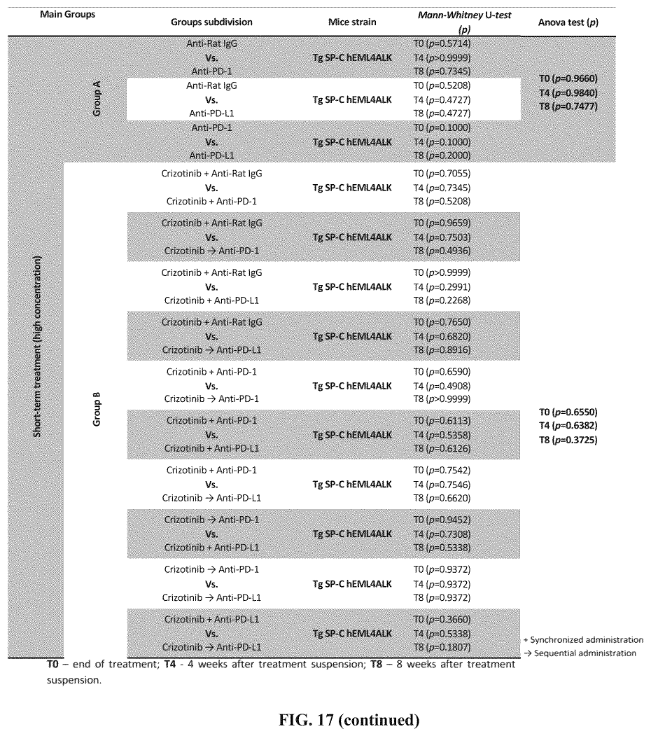

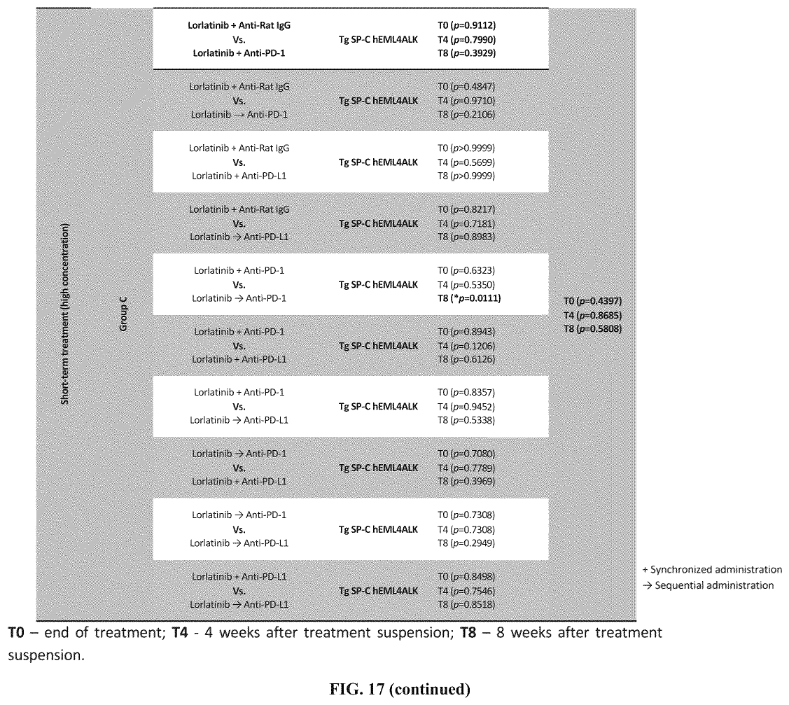

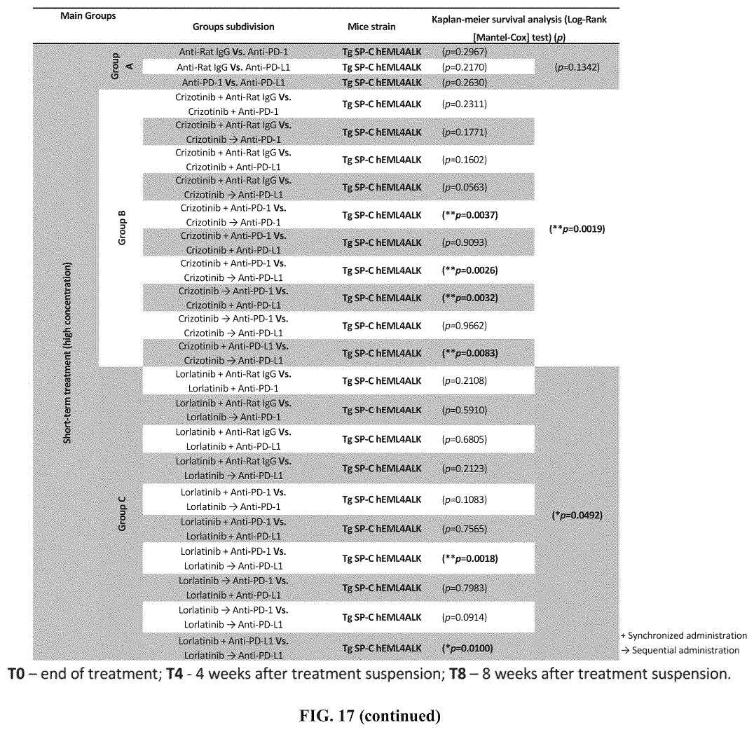

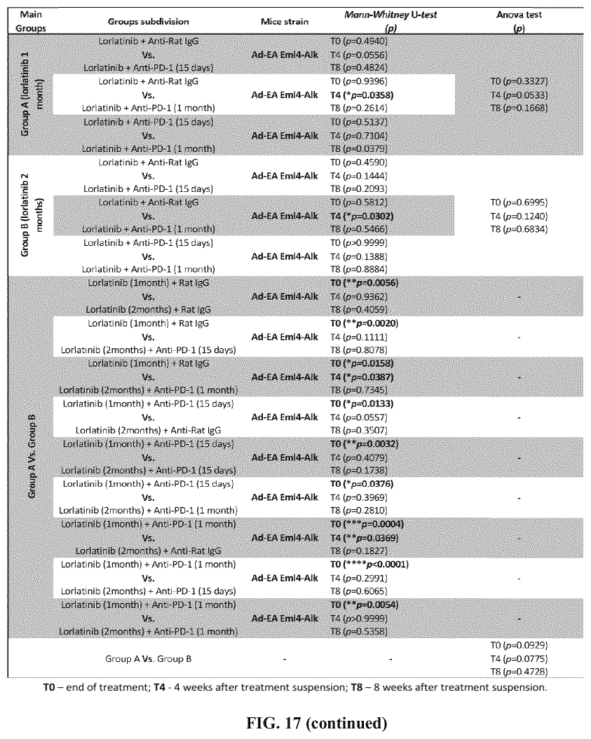

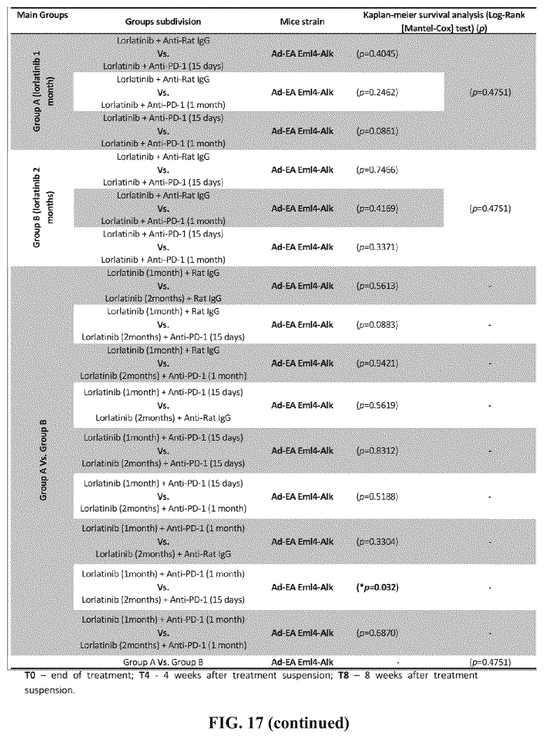

To study how to improve immunotherapy for ALK⁺ Non-Small Cell Lung Cancer (NSCLC), two mouse models previously developed were leveraged. EML4-ALK transgenic (Tg) mice, which express the human EML4-ALK (E13;A20, human variant 1) driven by the SP-C promoter (henceforth referred as hEML4-ALK Tg mice), and BALB/c mice infected intratracheally with adenovirus carrying the CRISPR/Cas9 system to induce in vivo the Eml4-Alk rearrangement (E14;A20, mouse variant 1) (henceforth referred as Ad-EA mice). Both models rapidly developed lung tumors, typically detectable at 12-weeks after birth in hEML4-ALK Tg mice or 10-weeks after adenovirus infection in Ad-EA mice, with comparable tumor morphology (FIG. 1A). In these models, magnetic resonance imaging (MRI) (FIG. 1B) allowed precise cohort stratification and tumor follow-up. Mice were divided into two major cohorts and treated with two different ALK tyrosine kinase inhibitors (TKIs) (crizotinib [40 mg/kg] or lorlatinib [2 mg/kg]) alone or in combination with ICI (anti-PD-1 or anti-PD-L1 antibodies) (FIGS. 1C-1D; FIG. 17). Since ALK TKI treatment alone induced a profound reduction of tumor burden in these models, it was evaluated whether the addition of ICI either potentiated tumor reduction at the end of treatment, or delayed tumor recurrence at 4 or 8 weeks after treatment suspension by quantifying the tumor volume change at each time point in comparison with tumor volume baseline (FIGS. 1C-1D and FIG. 17).

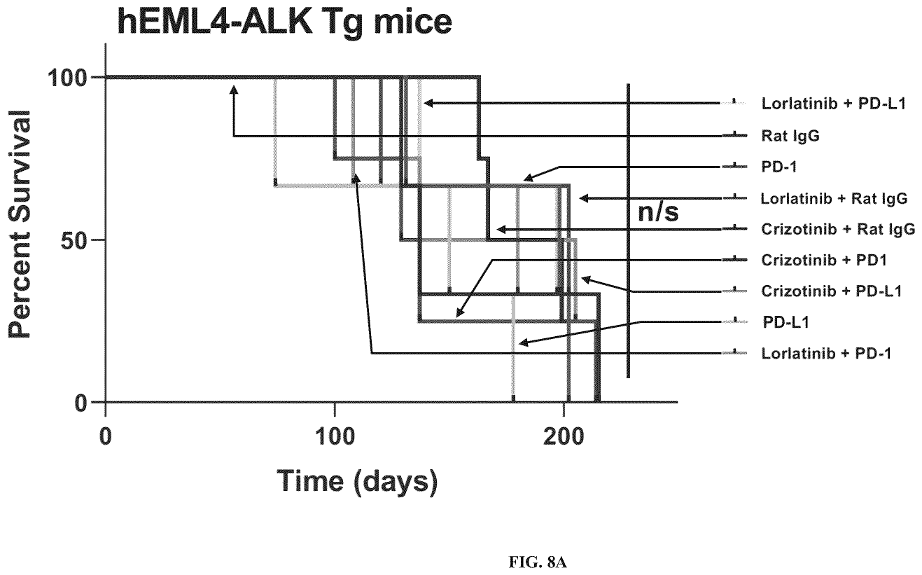

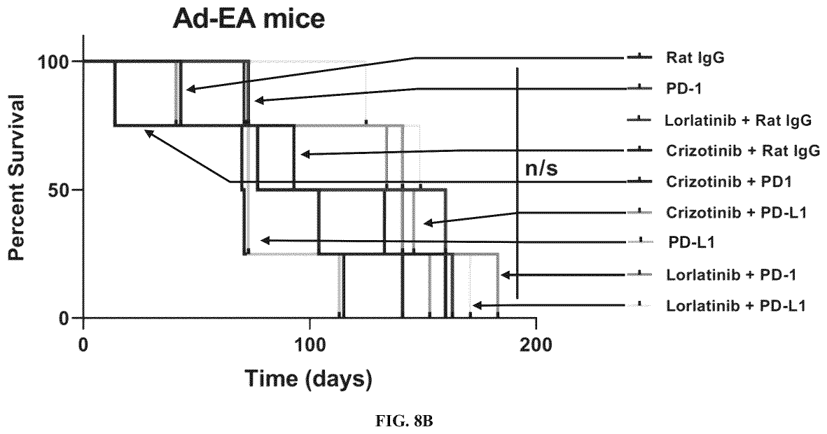

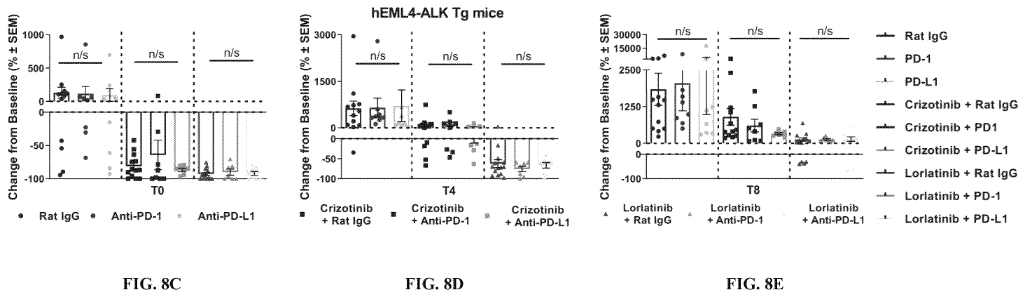

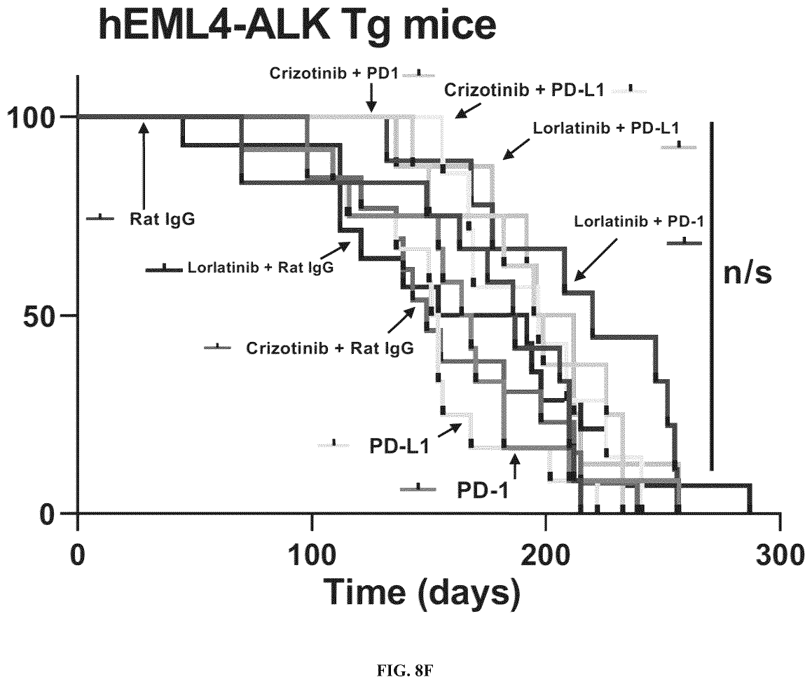

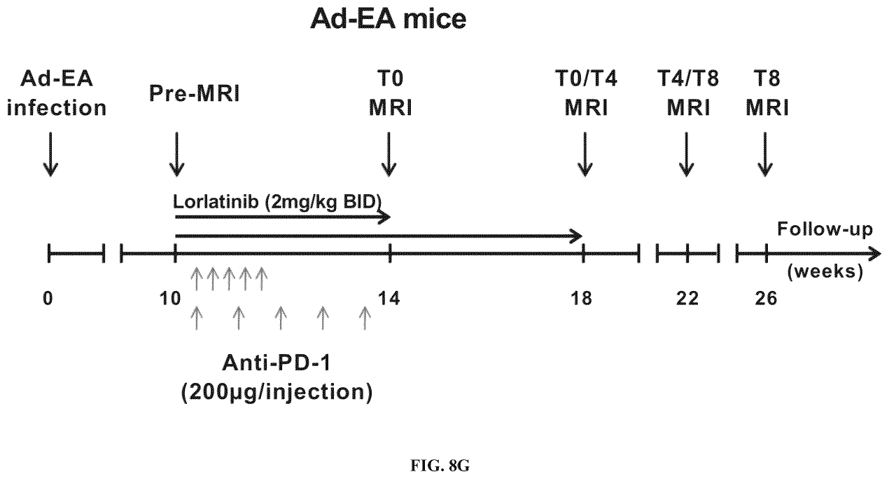

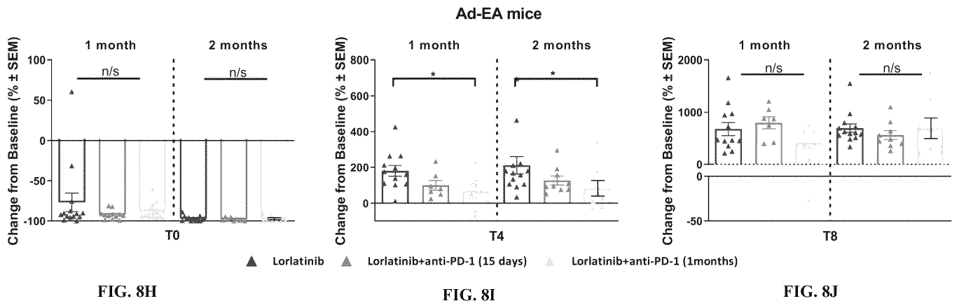

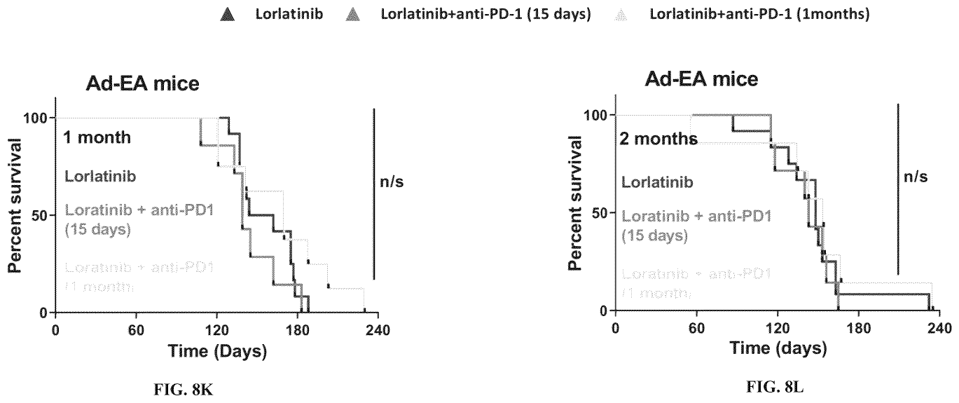

At the end of treatment lorlatinib induced almost complete regression of tumors in both hEML4-ALK Tg and Ad-EA mice, while crizotinib induced partial tumor regression in hEML4-ALK Tg and stabilized tumors in Ad-EA mice, consistent with the more potent activity of lorlatinib against ALK-driven lung cancer. Consequently, tumors relapsed faster in mice treated with crizotinib than in mice treated with lorlatinib (FIGS. 1E-1G and FIGS. 1H-1J). Anti-PD-1 or anti-PD-L1 did not induce significant tumor reduction when administered as monotherapy, did not induce greater tumor regression at the end of treatment, nor did delay tumor relapse after treatment suspension when combined with ALK TKIs (FIGS. 1E-1J). The addition of ICIs to ALK TKIs did not translate into an extension of the overall survival in both mouse models (FIGS. 8A and 8B). Similar experiments repeated with Ad-EA mice in a different mouse strain (C57BL/6 Ad-EA mice) showed similar results.

Next, different treatment protocols were tested, including higher concentrations of ALK TKIs and ICIs and prolonged treatments (FIG. 17). Higher dosages of crizotinib induced an almost complete tumor regression in hEML4-ALK Tg mice, similar to higher dosages of lorlatinib (FIG. 8C). Yet again, the addition of ICIs did not delay tumor relapses after treatment suspension (FIGS. 8D and 8E), nor did extend the overall survival of mice (FIGS. 8F and 17). For prolonged treatments, Ad-EA mice were treated with lorlatinib for 4 or 8 consecutive weeks alone or in combination with anti-PD-1 (FIGS. 8G and 17). Despite achieving a complete remission by MRI when treated with lorlatinib up to 8 weeks, tumors still relapsed upon treatment suspension (FIGS. 8H-8J). In these settings, the addition of anti-PD-1 induced a transient delay of tumor growth at 4 weeks that was eventually lost at 8 weeks after treatment suspension (FIGS. 8I and 8J) without significant improvement in overall survival (FIGS. 8K and 8L). All together, these data in mouse models of ALK-rearranged lung cancer faithfully recapitulated the absence of therapeutic benefit when ICIs is added to ALK TKIs, as observed in ALK-rearranged non-small cell lung cancer (NSCLC) patients.

### Example 2. Identification of Anaplastic Lymphoma Kinase (Alk) Immunogenic Peptides in Mouse Models

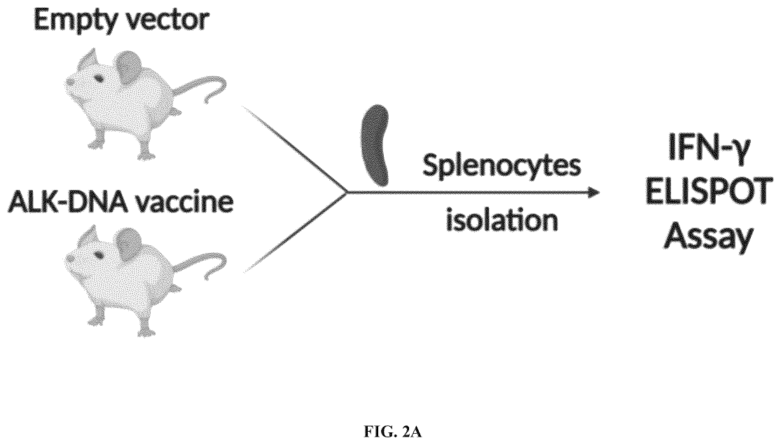

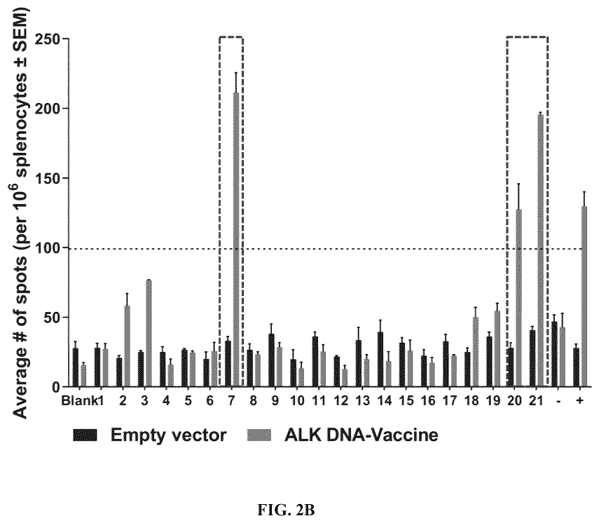

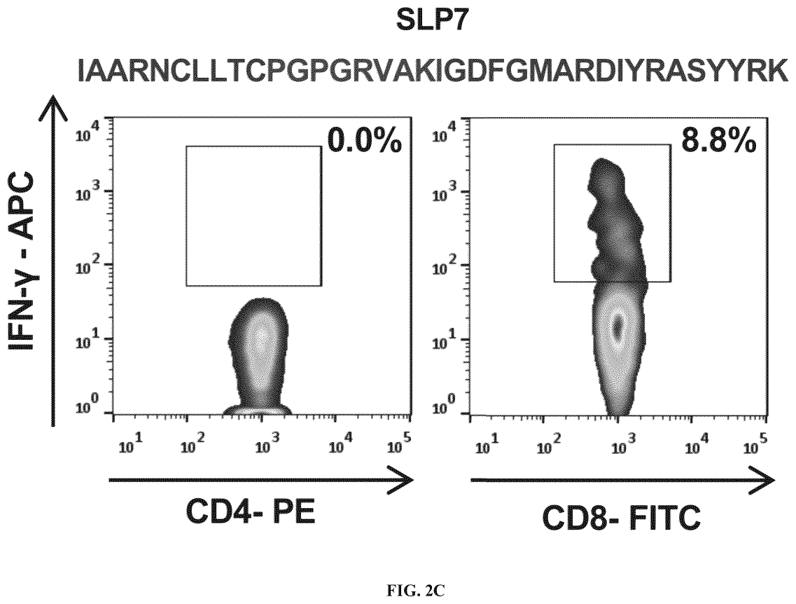

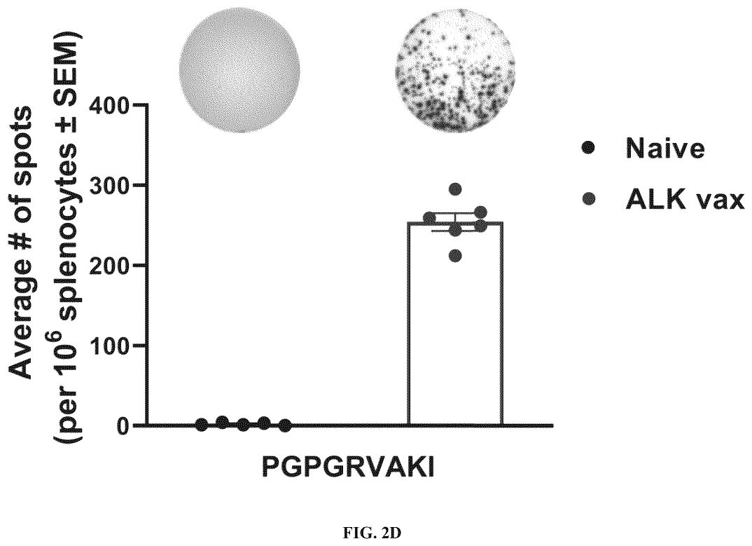

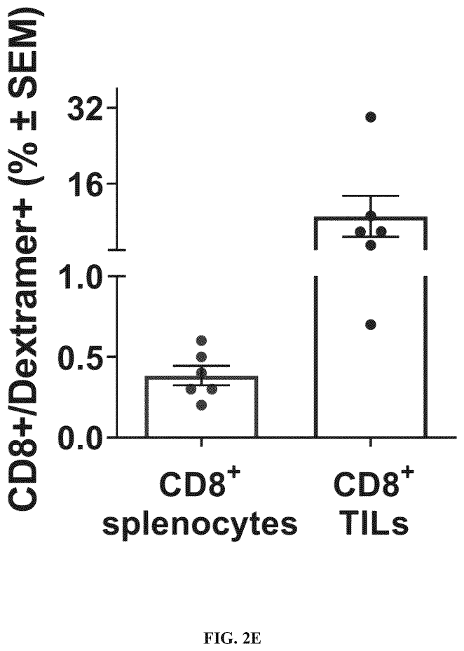

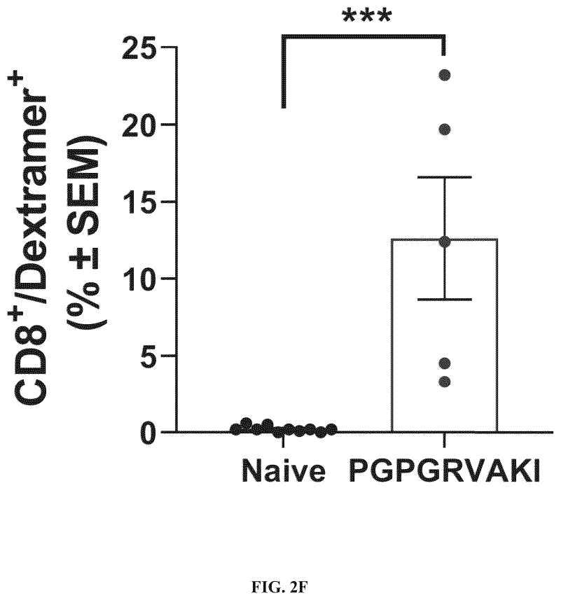

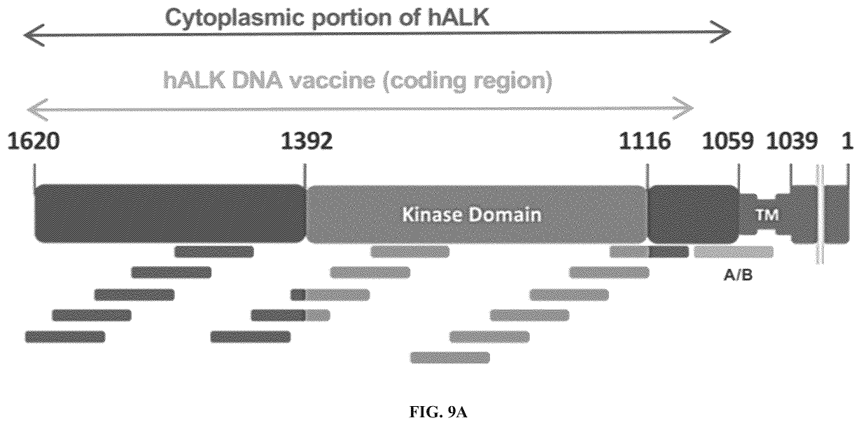

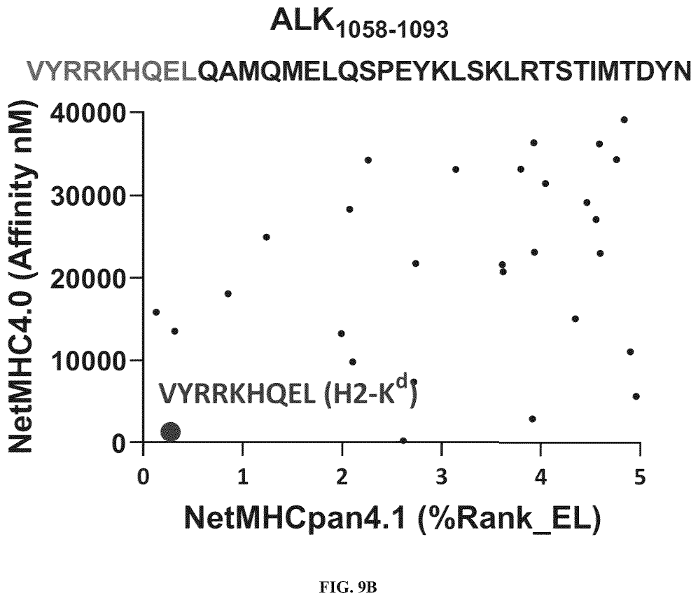

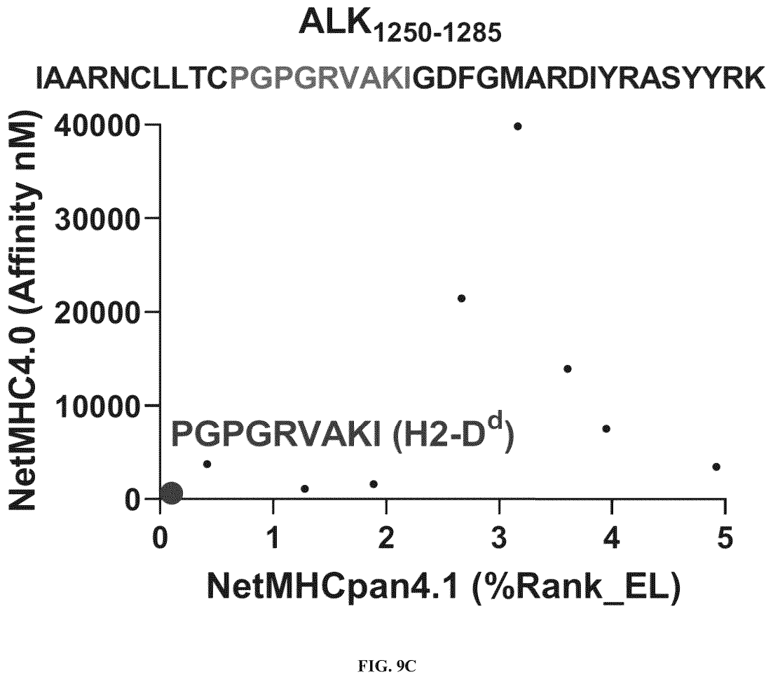

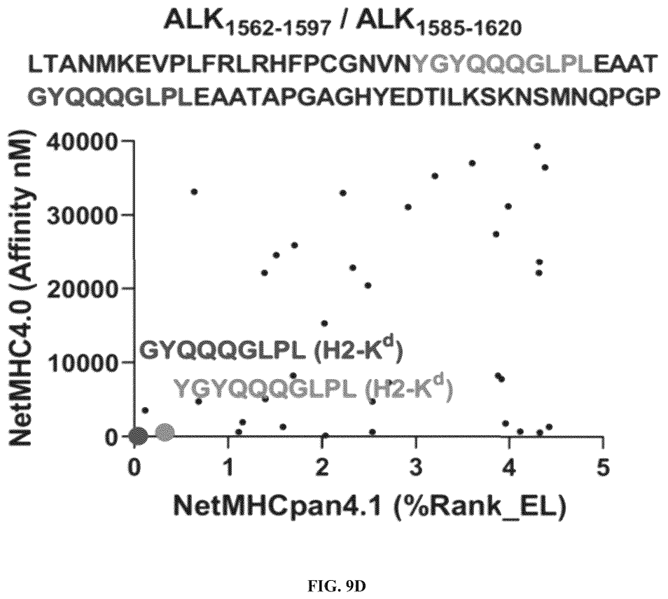

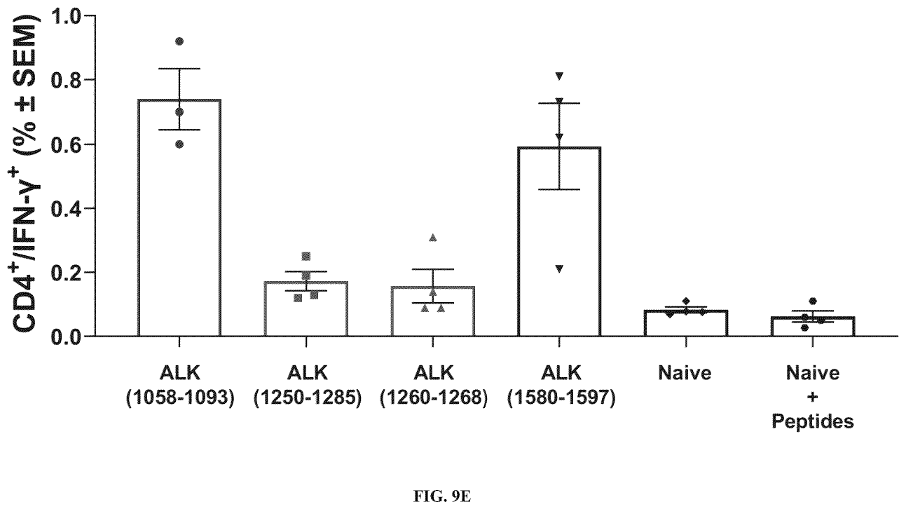

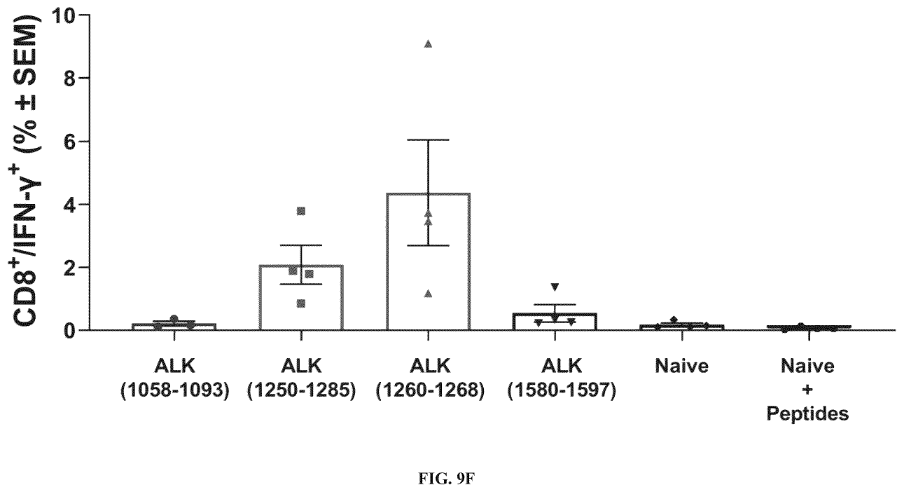

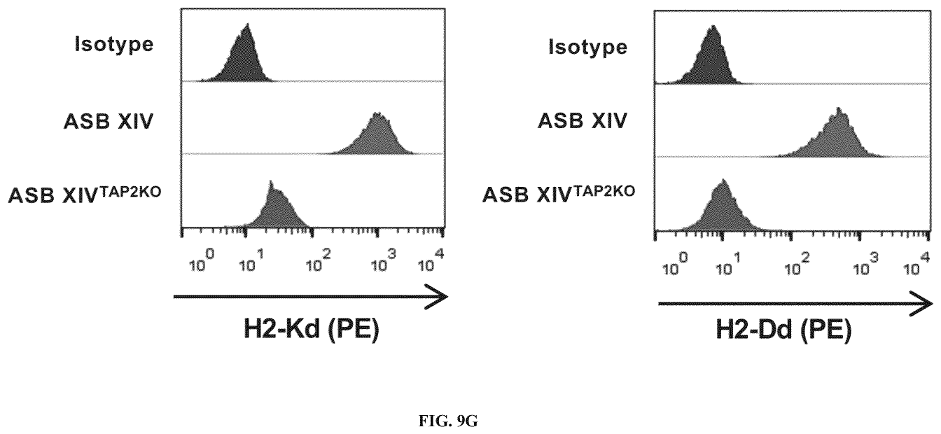

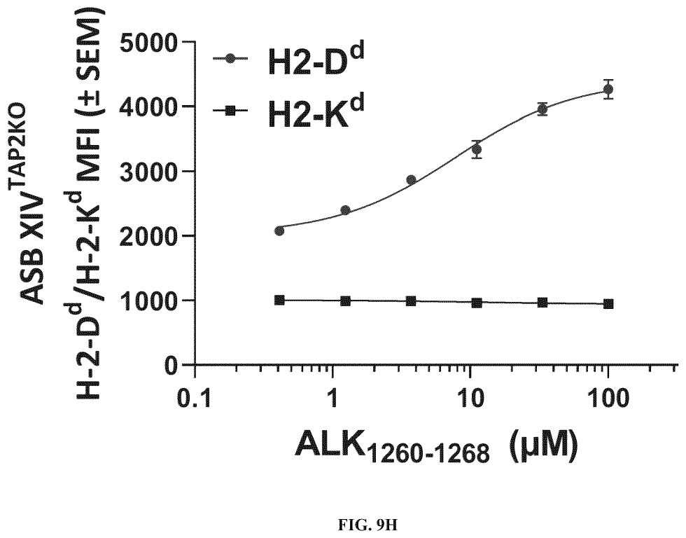

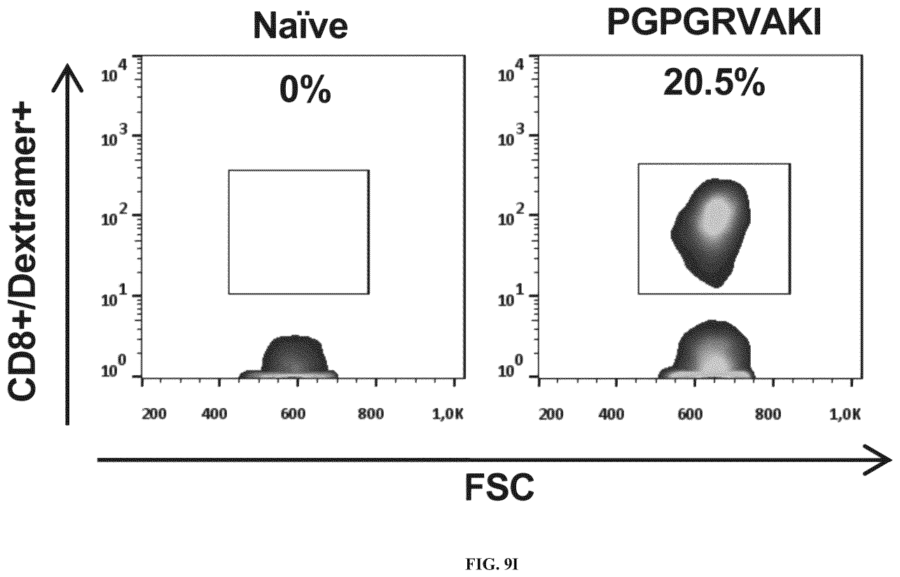

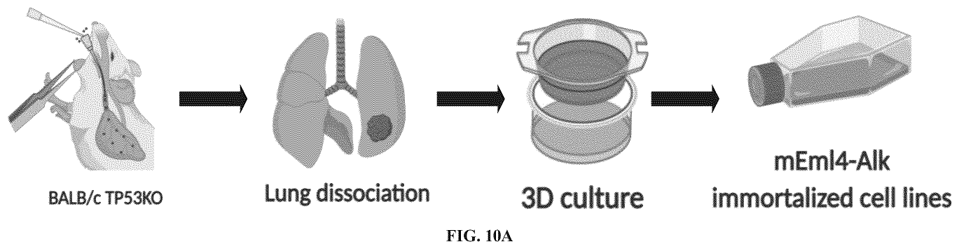

Despite the known immunogenicity of the ALK protein, the data imply that ALK⁺ mouse lung tumors induce an immune response insufficient to be activated by immune checkpoint inhibitor (ICI) treatment. Therefore, it was decided to directly investigate anti-ALK immune responses during ICI treatment by identifying the specific ALK peptides that induce T-cell mediated immune responses in mouse models. To identify the ALK-specific T-cell epitopes, an in vitro peptide screening was performed. A set of 21 overlapping synthetic long peptides (SLP), that encompassed the ALK cytoplasmic domain (FIG. 9A; Table 1), was incubated with splenocytes isolated from mice vaccinated with a vector encoding most of the ALK cytoplasmic domain (ALK-DNA vaccine) as previously described in Chiarle R, Martinengo C, Mastini C, Ambrogio C, D'Escamard V, Fori G, et al. The anaplastic lymphoma kinase is an effective oncoantigen for lymphoma vaccination. Nat Med 2008; 14(6):676-80 doi 10.1038/nm1769 and in Voena C, Menotti M, Mastini C, Di Giacomo F, Longo D L, Castella B, et al. Efficacy of a Cancer Vaccine against ALK-Rearranged Lung Tumors. Cancer immunology research 2015; 3(12):1333-43 doi 10.1158/2326-6066.CIR-15-0089, and analyzed by INF-γ ELISPOT assay (FIG. 2A). SLP7 (hALK₁₂₅₀₋₁₂₈₅) induced a 6.27-fold increase in the number of IFN-γ-spots in the ALK-DNA vaccine group compared to the control group (FIG. 2B; 203±21.6 vs. 32.3±6.6, respectively). Weaker responses to SLP20 (hALK₁₅₆₂₋₁₅₉₇) and SLP21 (hALK₁₅₈₅₋₁₆₂₀) were also observed (FIG. 2B).

TABLE 1
Synthetic Long Peptides (SLPs) overlapping the cytoplasmic portion of ALK encoded
in the hALK DNA-based vaccine

| Cytoplasmic portion of ALK - Synthetic Long Peptides (SLP | | | SEQ ID |
|---|---|---|---|
| peptides) | | Nomenclature | NO: |
| A | VYRRKHQELQAMQMELQSPEYKLSKLRTSTIMTDYN | ALK₁₀₅₈₋₁₀₉₃ | 24 |
| B | KLRTSTIMTDYNPNYCFAGKTSSISDLKEVPRKNIT | ALK₁₀₈₂₋₁₁₁₇ | 25 |
| 1 | SDLKEVPRKNITLIRGLGHGAFGEVYEGQVSGMPDN | ALK₁₁₀₆₋₁₁₄₁ | 26 |
| 2 | VYEGQVSGMPDNPSPLQVAVKTLPEVCSEQDELDFL | ALK₁₁₃₀₋₁₁₆₅ | 27 |
| 3 | EVCSEQDELDFLMEALIISKFNHQNIVRCIGVSLQS | ALK₁₁₅₄₋₁₁₈₉ | 28 |
| 4 | NIVRCIGVSLQSLPRFILLELMAGGDLKSFLRETR | ALK₁₁₇₈₋₁₂₁₂ | 29 |
| 5 | GDLKSFLRETRPRPSQPSSLAMLDLLHVARDIACGC | ALK₁₂₀₂₋₁₂₃₇ | 30 |
| 6 | LLHVARDIACGCQYLEENHFIHRDIAARNCLLTCPG | ALK₁₂₂₆₋₁₂₆₁ | 31 |
| 7 | IAARNCLLTCPGPGRVAKIGDFGMARDIYRASYYRK | ALK₁₂₅₀₋₁₂₈₅ | 32 |
| 8 | ARDIYRASYYRKGGCAMLPVKWMPPEAFMEGIFTSK | ALK₁₂₇₄₋₁₃₀₉ | 33 |
| 9 | PEAFMEGIFTSKTDTWSFGVLLWEIFSLGYMPYPSK | ALK₁₂₉₈₋₁₃₃₃ | 34 |
| 10 | IFSLGYMPYPSKSNQEVLEFVTSGGRMDPPKNCPGP | ALK₁₃₂₂₋₁₃₅₇ | 35 |
| 11 | GRMDPPKNCPGPVYRIMTQCWQHQPEDRPNFAIILE | ALK₁₃₄₆₋₁₃₈₁ | 36 |
| 12 | PEDRPNFAIILERIEYCTQDPDVINTALPIEYGPLV | ALK₁₃₇₀₋₁₄₀₅ | 37 |
| 13 | NTALPIEYGPLVEEEEKVPVRPKDPEGVPPLLVSQQ | ALK₁₃₉₄₋₁₄₂₉ | 38 |
| 14 | PEGVPPLLVSQQAKREEERSPAAPPPLPTTSSGKAA | ALK₁₄₁₈₋₁₄₅₃ | 39 |
| 15 | PPLPTTSSGKAAKKPTAAEISVRVPRGPAVEGGHVN | ALK₁₄₄₂₋₁₄₇₇ | 40 |
| 16 | PRGPAVEGGHVNMAFSQSNPPSELHKVHGSRNKPTS | ALK₁₄₆₆₋₁₅₀₁ | 41 |
| 17 | HKVHGSRNKPTSLWNPTYGSWFTEKPTKKNNPIAKK | ALK₁₄₉₀₋₁₅₂₅ | 42 |
| 18 | KPTKKNNPIAKKEPHDRGNLGLEGSCTVPPNVATGR | ALK₁₅₁₄₋₁₅₄₉ | 43 |
| 19 | SCTVPPNVATGRLPGASLLLEPSSLTANMKEVPLFR | ALK₁₅₃₈₋₁₅₇₃ | 44 |
| 20 | LTANMKEVPLFRLRHFPCGNVNYGYQQQGLPLEAAT | ALK₁₅₆₂₋₁₅₉₇ | 45 |
| 21 | GYQQQGLPLEAATAPGAGHYEDTILKSKNSMNQPGP | ALK₁₅₈₅₋₁₆₂₀ | 46 |

Analysis in silico with MHC-I epitope-binding algorithms (Tables 2A-2C) identified four ALK peptides predicted to bind to BALB/c mice MHC-I alleles: 9-mer VYRRKHQEL (SEQ ID NO: 47) (hALK₁₀₅₈₋₁₀₆₆), GYQQQGLPL (SEQ ID NO: 48) (hALK₁₅₈₅₋₁₅₉₃) and 10-mer YGYQQQGLPL (SEQ ID NO: 49) (hALK₁₅₈₄₋₁₅₉₃) were predicted to bind the H2-Kd allele, while the 9-mer PGPGRVAKI (SEQ ID NO: 22) (hALK₁₂₆₀₋₁₂₆₈) the H2-Dd allele (FIGS. 9B-9D)). To confirm the immunogenicity of these peptides and determine the type of response elicited, BALB/c mice were vaccinated with a mixture of synthetic long peptides (SLP) encompassing the predicted anaplastic lymphoma kinase (ALK) peptides and IFN-γ cytoplasmic production was tested in CD4⁺ and CD8⁺ T-cells stimulated with the corresponding peptides. Surprisingly, the three SLP peptides containing the 9- and 10-mer predicted to bind to H2-Kd allele (VYRRKHQEL (SEQ ID NO: 47), GYQQQGLPL (SEQ ID NO: 48) and YGYQQQGLPL (SEQ ID NO: 49)), only elicited CD4⁺ T-cell responses (FIGS. 9E-9F); in contrast, SLP7 (hALK₁₂₅₀₋₁₂₈₅) and the corresponding 9-mer PGPGRVAKI (SEQ ID NO: 22) (hALK₁₂₆₀₋₁₂₆₈) elicited specific CD8⁺ T-cell responses (FIGS. 2C, 9E, and 9F).

TABLE 2A
Prediction of ALK binding peptides
VYRRKHQELQAMQMELQSPEYKLSKLRTSTIMTDYN (SEQ ID
NO: 24)

| | SEQ | | NetMHC4.0 |
|---|---|---|---|
| | ID | NetMHCpan4.1 | (Affinity |
| Peptide (Allele) | NO: | (% Rank_EL) | nM) |
| QSPEYKLSKL (Dd) | 50 | 0.133 | 15836.26 |
| LSKLRTSTI (Dd) | 51 | 0.853 | 18083.63 |
| VYRRKHQEL (Dd) | 47 | 1.239 | 24936.52 |
| VYRRKHQEL (Kd) | 47 | 0.277 | 1316.68 |
| SPEYKLSKL (Ld) | 52 | 0.319 | 13555.91 |
| ELQAMQMEL (Ld) | 53 | 1.993 | 13258.68 |
| SPEYKLSKL (Dd) | 52 | 2.0776 | 28307.28 |
| KHQELQAMQM (Kd) | 54 | 2.107 | 9824.29 |
| QSPEYKLSK (Dd) | 55 | 2.2617 | 34280.82 |
| EYKLSKLRTSTI | 56 | 2.614 | 236.41 |
| (Kd) | | | |
| KHQELQAM (Kd) | 57 | 2.722 | 7388.94 |
| LQSPEYKLSKL (Dd) | 58 | 2.7409 | 21746.72 |
| QELQAMQMEL (Ld) | 59 | 3.143 | 33162.61 |
| SPEYKLSKL (Kd) | 52 | 3.615 | 21612.32 |
| MQMELQSPEYKL | 60 | 3.624 | 20737.47 |
| (Ld) | | | |
| ELQSPEYKL (Ld) | 61 | 3.802 | 33196.01 |
| KHQELQAMQMEL | 62 | 3.919 | 2920.44 |
| (Kd) | | | |
| QSPEYKLS (Dd) | 63 | 3.9315 | 36394.17 |
| QSPEYKLSKLR (Dd) | 64 | 3.9378 | 23109.6 |
| MELQSPEYKL (Ld) | 65 | 4.049 | 31435.26 |
| LSKLRTSTI (Kd) | 51 | 4.348 | 15049.61 |
| LQSPEYKLSKL (Ld) | 58 | 4.464 | 29145.16 |
| SKLRTSTI (Dd) | 66 | 4.5583 | 27098.91 |
| QSPEYKLSKL (Ld) | 50 | 4.59 | 36238.16 |
| KLSKLRTSTI (Dd) | 67 | 4.5979 | 22995.36 |
| YRRKHQEL (Dd) | 68 | 4.7616 | 34355.08 |
| VYRRKHQEL (Ld) | 47 | 4.839 | 39107.59 |
| ELQAMQMEL (Kd) | 53 | 4.9 | 11046.96 |
| KLSKLRTSTI (Kd) | 67 | 4.96 | 5636.56 |

TABLE 2B
Prediction of ALK binding peptides
IAARNCLLTCPGPGRVAKIGDFGMARDIYRASYYRK (SEQ
ID NO: 32)

| | SEQ | | NetMHC4.0 |
|---|---|---|---|
| | ID | NetMHCpan4.1 | (Affinity |
| Peptide (Allele) | NO: | (% Rank_EL) | nM) |
| PGPGRVAKI (Dd) | 22 | 0.104 | 602.96 |
| TCPGPGRVAKI (Dd) | 69 | 0.417 | 3732.8 |
| GPGRVAKI (Dd) | 70 | 1.282 | 1092.72 |
| VPGPGRVAKI (Dd) | 71 | 1.89 | 1570.72 |
| IAARNCLL (Dd) | 72 | 2.668 | 21463.17 |
| CPGPGRVAKI (Ld) | 73 | 3.166 | 39811.61 |
| GPGRVAKIGDF (Ld) | 74 | 3.609 | 13907.56 |
| CPGPGRVAKIGDF (Ld) | 75 | 3.952 | 7503.83 |
| LTCPGPGRVAKI (Dd) | 76 | 4.925 | 3466.63 |

TABLE 2C
Prediction of ALK binding peptides
LTANMKEVPLFRLRHFPCGNVNYGYQQQGLPLEAAT
(SEQ ID NO: 45)
GYQQQGLPLEAATAPGAGHYEDTILKSKNSMNQPGP
(SEQ ID NO: 46)

| | SEQ | | NetMHC4.0 |
|---|---|---|---|
| | ID | NetMHCpan4.1 | (Affinity |
| Peptide (Allele) | NO: | (% Rank_EL) | nM) |
| GYQQQGLPL (Kd) | 48 | 0.041 | 82.34 |
| VPLFRLRHF (Ld) | 77 | 0.114 | 3569.44 |
| YGYQQQGLPL (Kd) | 49 | 0.326 | 587.8 |
| ANMKEVPLF (Dd) | 78 | 0.643 | 33142.17 |
| NYGYQQQGL (Kd) | 79 | 0.685 | 4779.92 |
| NYGYQQQGLPL (Kd) | 80 | 1.1118 | 660.04 |
| APGAGHYEDTIL (Ld) | 81 | 1.154 | 1977.22 |
| YGYQQQGL (Dd) | 82 | 1.388 | 22106.61 |
| APGAGHYEDTI (Ld) | 83 | 1.395 | 5117.54 |
| KEVPLFRLRHF (Ld) | 84 | 1.518 | 24575.97 |
| YQQQGLPL (Kd) | 85 | 1.585 | 1345.6 |
| TAPGAGHYEDTI (Dd) | 86 | 1.694 | 8203.29 |
| VPLFRLRHF (Dd) | 77 | 1.712 | 25910.71 |
| ANMKEVPL (Dd) | 87 | 2.0276 | 15267.25 |
| TILKSKNSM (Ld) | 88 | 2.0383 | 134.07 |
| HYEDTILKSKNSM | 89 | 2.2284 | 32995.82 |
| (Kd) | | | |
| ANMKEVPLF (Ld) | 78 | 2.3305 | 22814.46 |
| TANMKEVPL (Dd) | 90 | 2.4907 | 20402.74 |
| AGHYDTIL (Dd) | 91 | 2.5365 | 4779.92 |
| TANMKEVPLD (Ld) | 92 | 2.5379 | 660.04 |
| VNYGYQQQGLPL (kd) | 93 | 2.7103 | 7290.94 |
| MKEVPLFRLRHF (Ld) | 94 | 2.9242 | 31070.08 |
| FPCGNVNYGY (Ld) | 95 | 3.21 | 35291.7 |
| GYQQQGLPLEA (Kd) | 96 | 3.6081 | 37008.05 |
| RHFPCGNV (Kd) | 97 | 3.8519 | 44906 |
| YGYQQQWGLPL (Ld) | 98 | 3.8631 | 27418.62 |
| TILKSKNSM (Kd) | 88 | 3.8793 | 8203.29 |
| GHYEDTIL (Kd) | 99 | 3.9174 | 7738.4 |
| TAPGAGHY (Dd) | 100 | 3.9612 | 1825.75 |
| YGYQQQGLPL (Dd) | 49 | 3.9972 | 31180.86 |
| TAPGAGHYEDTIL | 101 | 4.1176 | 702.5 |
| (Dd) | | | |
| RHFPCGNVNY (Kd) | 102 | 4.1222 | 40562.06 |
| EVPLFRLRHF (Ld) | 103 | 4.3045 | 39303.13 |
| KEVPLFRL (Ld) | 104 | 4.3175 | 22106.6 |
| HYEDTILKS (Kd) | 105 | 4.3242 | 23620.24 |
| ANMKEVPLF (Kd) | 78 | 4.3275 | 587.8 |
| HFPCGNVNY (Kd) | 106 | 4.3886 | 36441.45 |
| TANMKEVPLF (Ld) | 107 | 4.4268 | 1345.6 |

In order to confirm that the 9-mer PGPGRVAKI (SEQ ID NO: 22) directly bound H-2-Dd, ALK-negative BALB/c-syngeneic lung cancer cell line ASB-XIV was edited to generate a cell line knockout for the Tap2 gene (ASB-XIV^{TAP2KO}). In the absence of TAP2, peptide-MHC-I complexes are not formed resulting in a low MHC-I surface expression that can be increased when exogenous peptides bind, and thereby stabilize the peptide-MHC-I complexes on the cell surface. ASB-XIV^{TAP2KO} cells showed low H2-Kd and H2-Dd surface expression (FIG. 9G). H2-Dd, but not H2-Kd, was stabilized on the surface of ASB-XIV^{TAP2KO} upon incubation with increasing concentrations of PGPGRVAKI (SEQ ID NO: 22) (FIG. 9H), confirming its specific binding to H2-Dd allele. Vaccination of naïve BALB/c mice with the PGPGRVAKI (SEQ ID NO: 22) peptide (henceforth referred as ALK vaccine) induced specific CD8⁺ T-cell responses detected by IFN-γ ELISPOT assay (FIG. 2D) and by a PGPGRVAKI (SEQ ID NO: 22)-H2-Dd dextramers (ALK dextramer) in all vaccinated mice, but not in control mice (FIGS. 2E and 9I). Overall, these data identified PGPGRVAKI (SEQ ID NO: 22) as an ALK immunogenic peptide that binds the H2-Dd allele in BALB/C mice and induces specific CD8⁺ T-cell responses.

Next, it was asked whether spontaneous anaplastic lymphoma kinase (ALK)-specific CD8⁺ tumor-infiltrating T lymphocytes (TILs) were present in mice bearing ALK⁺ tumors in the lungs. ALK dextramer⁺ T-cells represented an average of 9% of total CD8⁺ lung TILs in hEML4-ALK Tg mice, but only 0.4% of CD8⁺ splenocytes (FIG. 2F), likely due to an intratumoral enrichment of ALK-specific CD8⁺ T-cells caused by the presence of the ALK antigen. Yet, the presence of ALK-specific CD8⁺ TILs in the lung tumors was not sufficient to impair tumor growth during treatment with ICIs (FIG. 1E-1G), which suggested that spontaneous ALK immune responses were either non-functioning or not effectively reactivated by immune checkpoint inhibitors (ICIs). Thus, while spontaneous ALK-specific T-cell responses were generated in mice with ALK⁺ lung tumors, they were not sufficient to achieve an effective anti-tumor response, nor were they enhanced by ICI. In contrast, newly generated ALK-specific CD8⁺ T-cell responses through vaccination efficiently targeted tumor cells.

### Example 3. Tumor Localization Determined the Robustness of Anti-Anaplastic Lymphoma Kinase (Alk) Spontaneous Immune

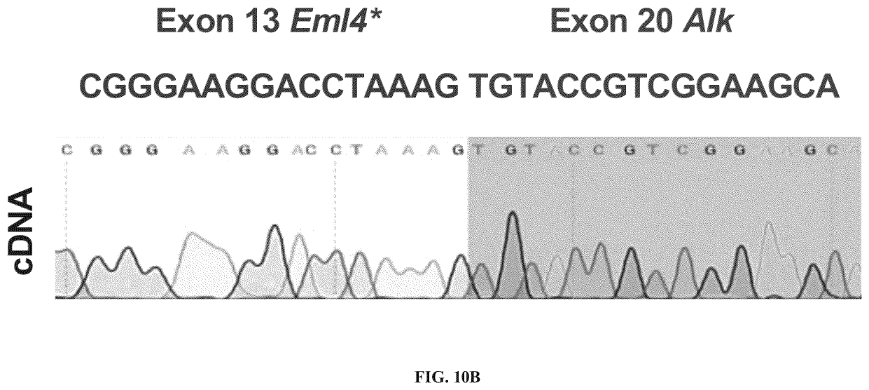

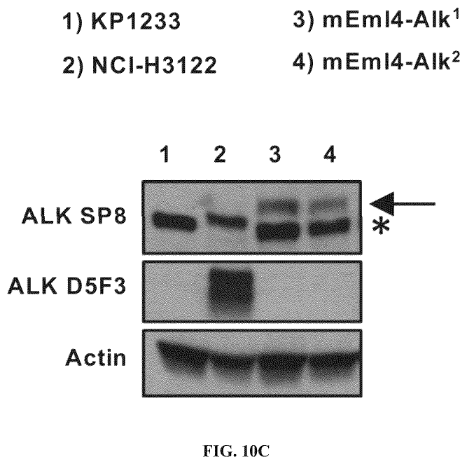

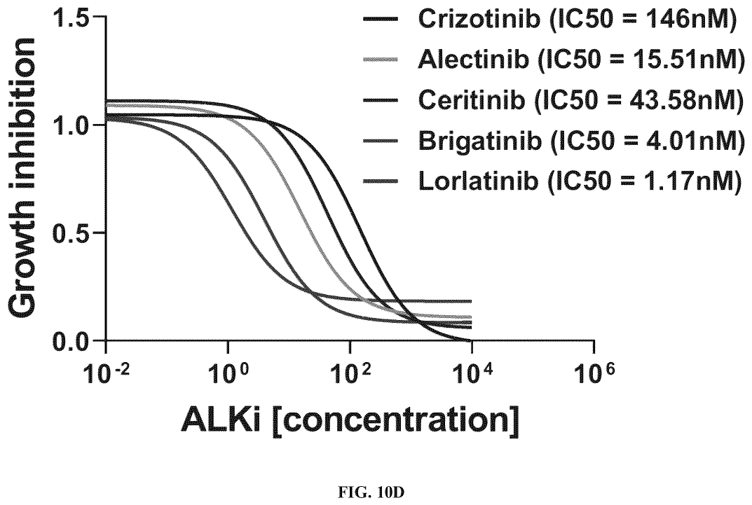

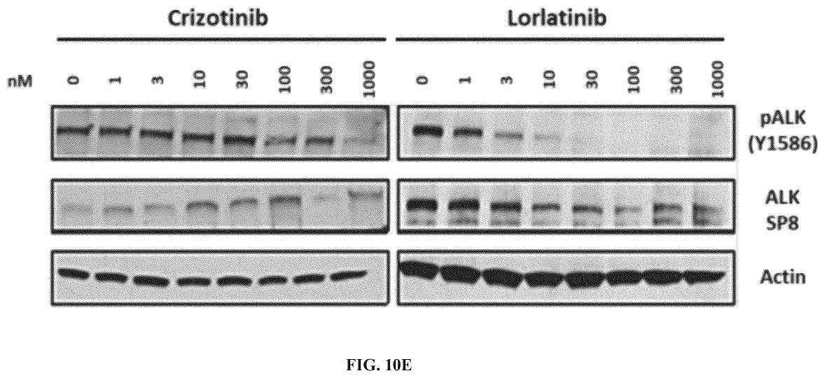

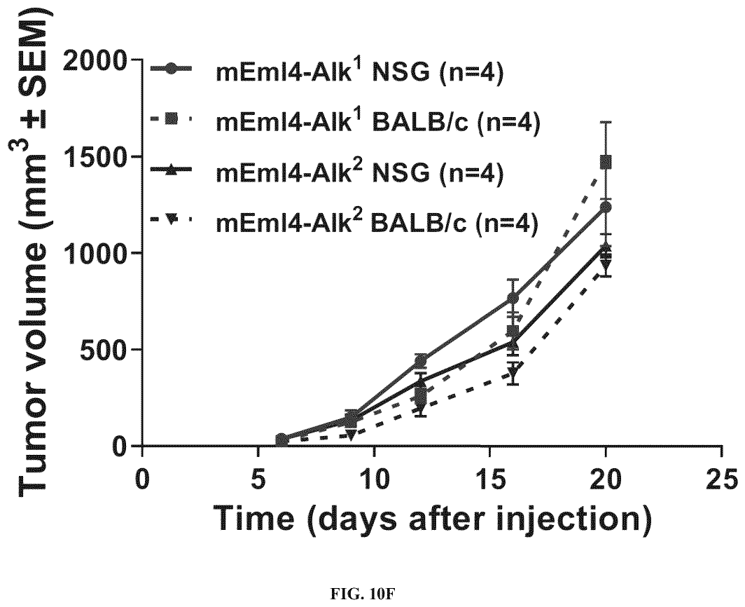

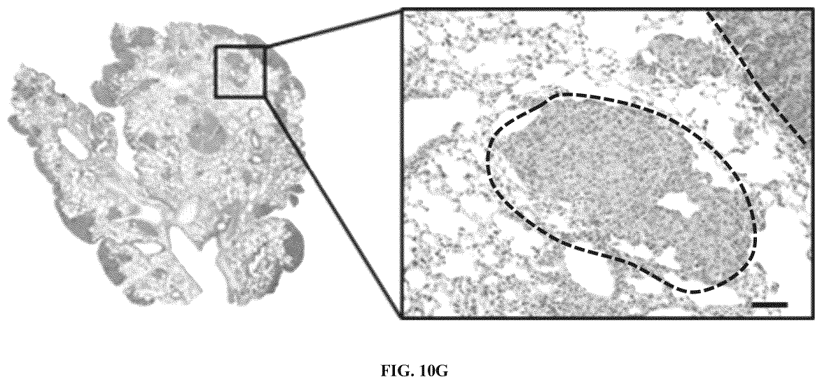

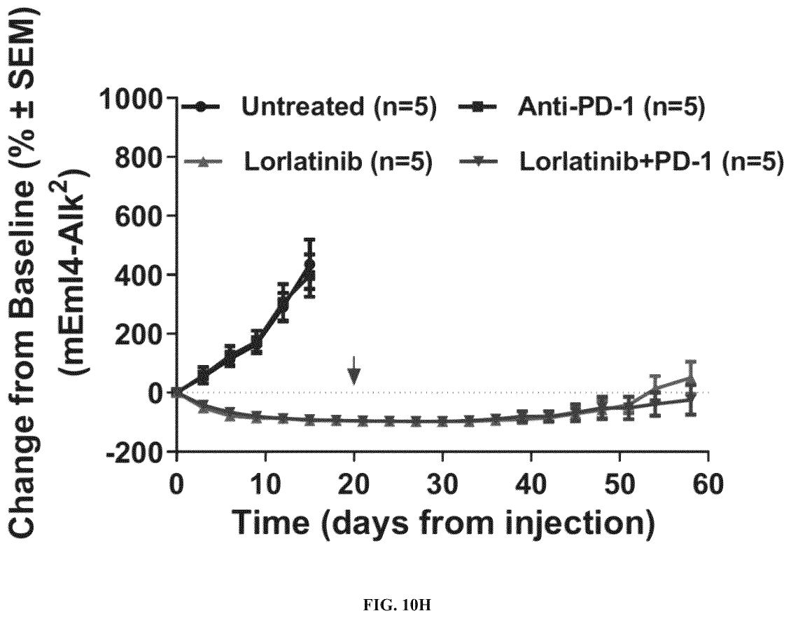

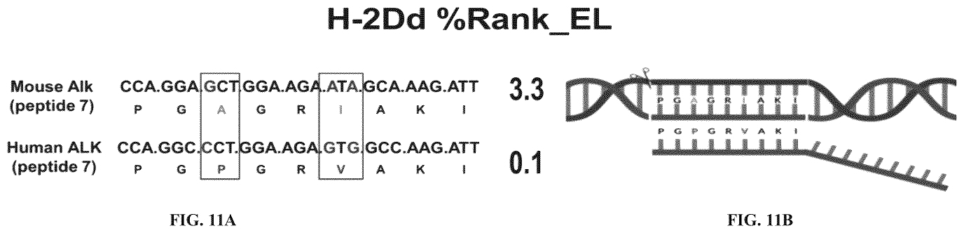

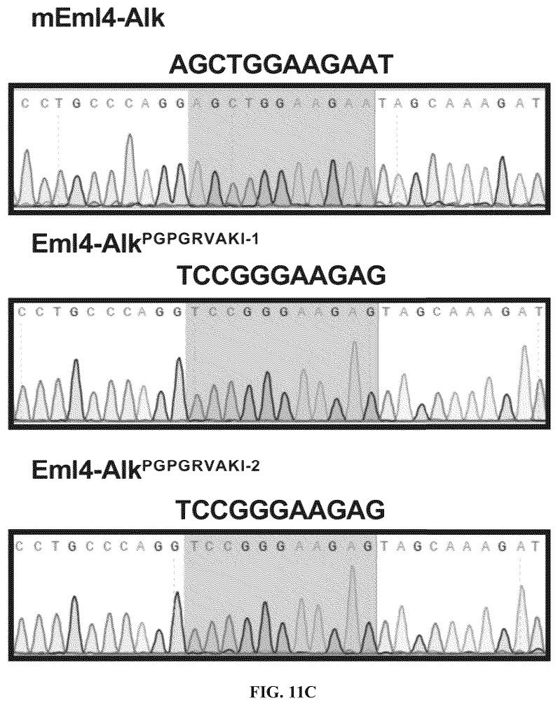

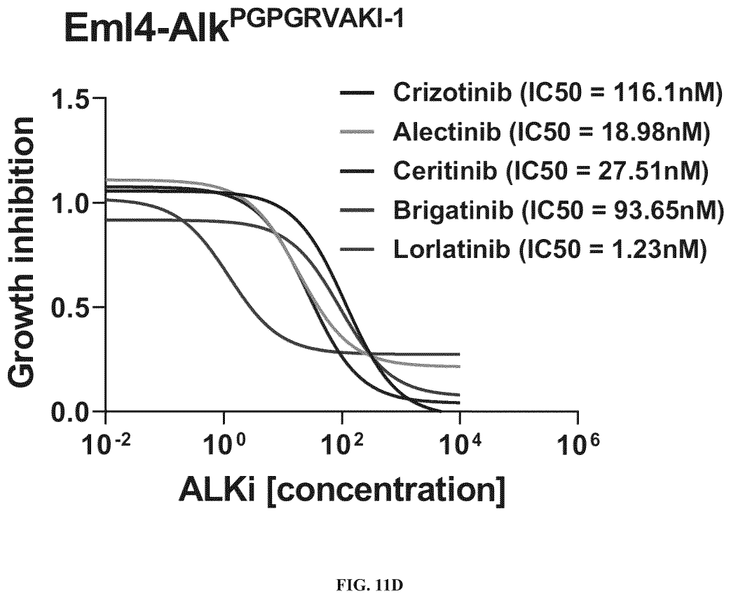

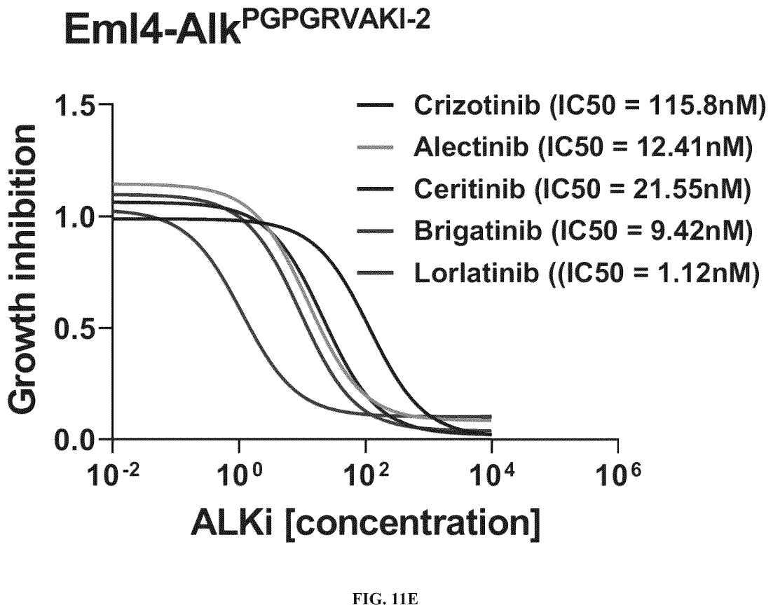

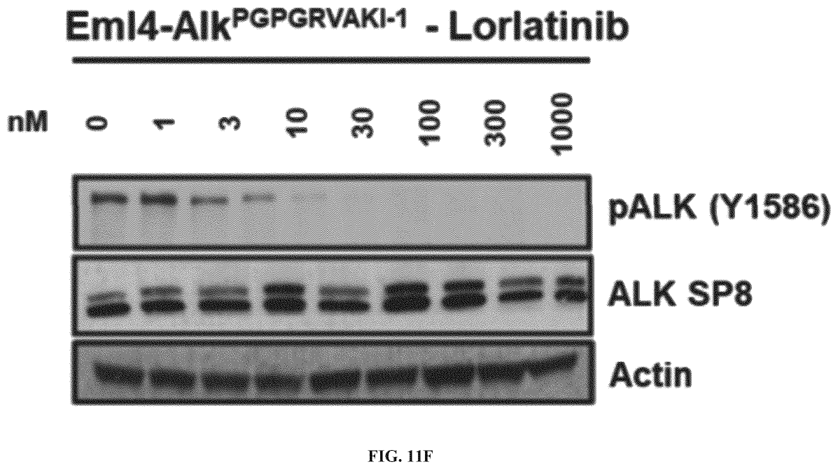

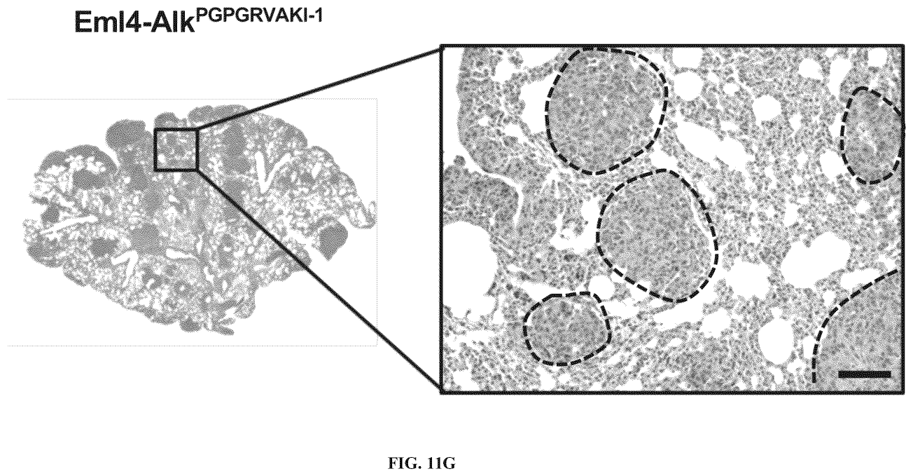

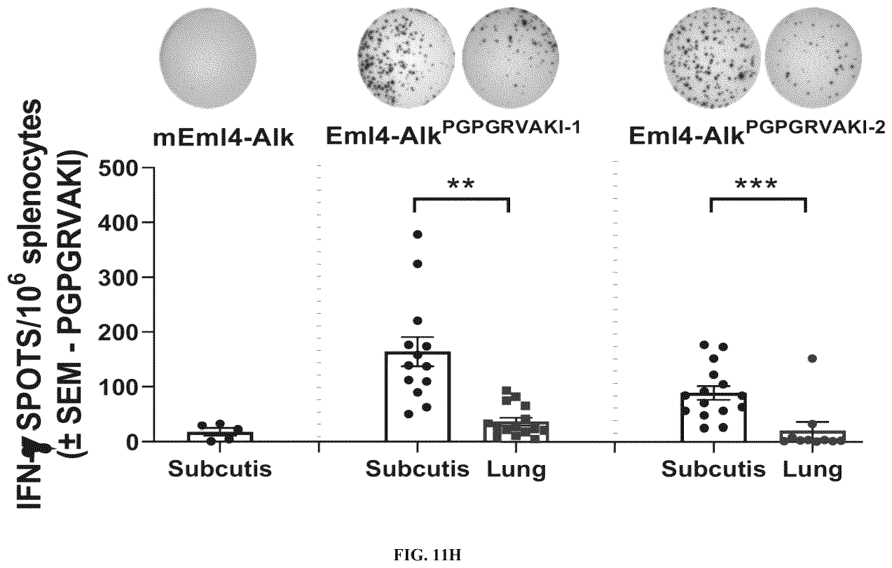

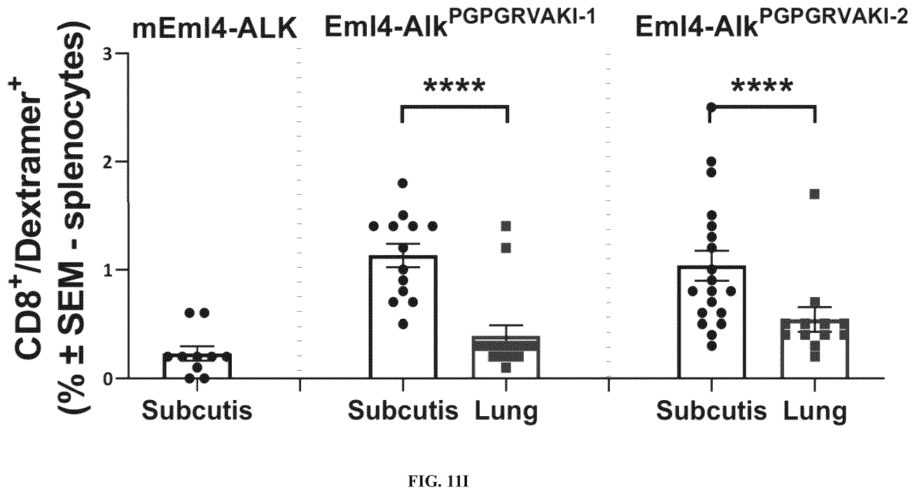

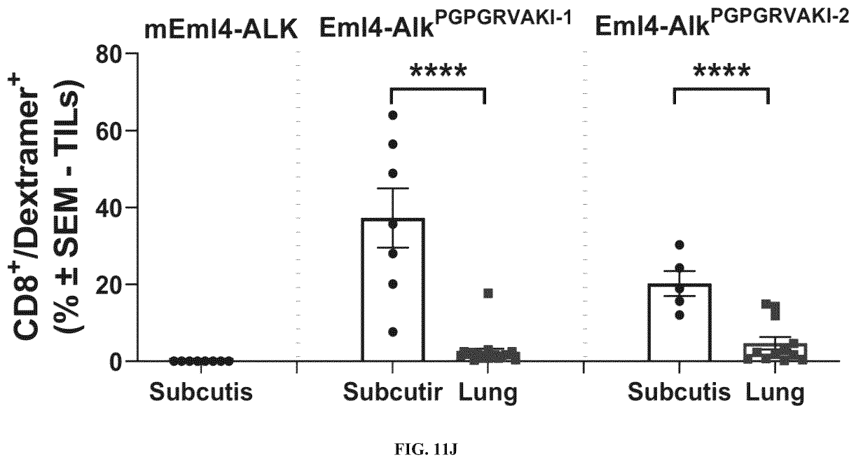

Response To understand why spontaneous anaplastic lymphoma kinase (ALK)-specific CD8⁺ T-cells were insufficient to trigger effective anti-tumor responses, transplantable ALK⁺ lung tumor models were developed by immortalizing cell lines from mouse models. While tumor cell lines were not obtained from hEML4-ALK Tg mice, several tumor lines were immortalized from Ad-EA mice (mEml4-Alk cell lines), in which the EML4-ALK expression was driven by the endogenous Eml4 promoter (FIG. 10A). These immortalized tumor cell lines harbored the Eml4-Alk rearrangement (FIG. 10B) and expressed the EML4-ALK fusion protein (FIG. 10C). The cell lines showed sensitivity to all ALK FDA-approved tyrosine kinase inhibitors (TKIs), lorlatinib being the most effective (lorlatinib IC50=1.17 nM, FIG. 10D), that inhibited EML4-ALK phosphorylation (FIG. 10E). When assessing the in vivo tumorigenicity of two mEml4-Alk cell lines (mEml4-Alk¹ and mEml4-Alk²), both cell lines exhibited comparable tumor growth rates when injected subcutaneously in the flank of immunocompetent BALB/c and immunodeficient NSG mice (FIG. 10F). When injected intravenously into BALB/c mice, both cell lines formed lung tumors with histological features consistent with primary tumors seen in Ad-EA mice (FIG. 10G) and showed marked sensitivity to lorlatinib in vivo (FIG. 10H). Overall, these cell lines represent EML4-ALK-driven lung cancers, sensitive to ALK TKIs, which can be used in immunocompetent mice to study spontaneous and induced anti-ALK immune responses. However, despite the 87% similarity of mouse and human ALK proteins, the corresponding murine ALK sequence differs by two amino-acids from the immunogenic human PGPGRVAKI (SEQ ID NO: 22) peptide identified (FIG. 11A). Since this difference in the amino-acid sequence was predicted to reduce the affinity of the peptide for the H2-Dd allele, the DNA sequence of the mEml4-Alk fusion was edited by CRISPR/Cas9 to modify the coding sequence from PGAGRIAKI (SEQ ID NO: 108) to PGPGRVAKI (SEQ ID NO: 22), and therefore, reconstituting the ALK peptide identified with high affinity for H2-Dd allele (FIG. 11B). By this approach two isogenic cell lines (Eml4-Alk^{PGPGRVAKI-1} and Eml4-Alk^{PGPGRVAKI-2}) were generated that differ only by two amino-acids in the ALK sequence from mEml4-Alk parental cell lines (FIG. 11C). “PGPGRVAKI” is disclosed as SEQ ID NO: 22. Like the parental mElm4-Alk cell lines, Eml4-Alk^{PGPGRVAKI-1} and Eml4-Alk^{PGPGRVAKI-2} cell lines retained sensitivity to ALK FDA-approved TKIs (FIGS. 11D and 11E). “PGPGRVAKI” is disclosed as SEQ ID NO: 22. The phosphorylation of the EML4-ALK fusion protein was efficiently inhibited by lorlatinib (FIG. 11F). When injected intravenously, lung tumors with histologic features like those generated by the parental mEml4-Alk cell line were observed (FIG. 11G).

Next, it was evaluated whether mEml4-Alk^{PGPGRVAKI-1} (“PGPGRVAKI” disclosed as SEQ ID NO: 22) and mEml4-Alk^{PGPGRVAKI-2} cell lines (“PGPGRVAKI” disclosed as SEQ ID NO: 22) spontaneously elicited CD8⁺ T-cell responses to the PGPGRVAKI (SEQ ID NO: 22) peptide. The same number of cells (10⁶ cells) was injected either subcutaneously in the flank or intravenously in syngeneic BALC/c mice. Splenocytes were isolated and ALK-specific CD8⁺ T-cell responses analyzed by IFN-γ-ELISPOT assay and ALK dextramer staining. Eml4-Alk^{PGPGRVAKI-1} (“PGPGRVAKI” disclosed as SEQ ID NO: 22) and Eml4-Alk^{PGPGRVAKI-2} (“PGPGRVAKI” is disclosed as SEQ ID NO: 22) cell lines elicited systemic CD8⁺ T-cell responses specific for the ALK PGPGRVAKI (SEQ ID NO: 22) peptide that were absent in mice injected with the parental mEml4-Alk cell line (FIG. 11H). Surprisingly, the robustness of the systemic anaplastic lymphoma kinase (ALK) responses was significantly higher when tumor grew subcutaneously than in the lung as demonstrated by IFN-γ-ELISPOT assay (FIG. 11H) and ALK dextramer stainings (FIG. 11I). Likewise, the analysis of tumor-infiltrating T lymphocytes (TILs) demonstrated that intratumoral ALK-specific CD8⁺ T-cell responses were higher in flank tumors than in lung tumors (FIG. 11J). Not intending to be bound by theory, these data suggest that the localization of the ALK⁺ tumor determined the amplitude of the spontaneous intratumoral and systemic ALK-specific CD8⁺ T-cell responses.

### Example 4. Tumor Localization Determined the Efficacy of Immune Checkpoint Inhibitors (ICIS)

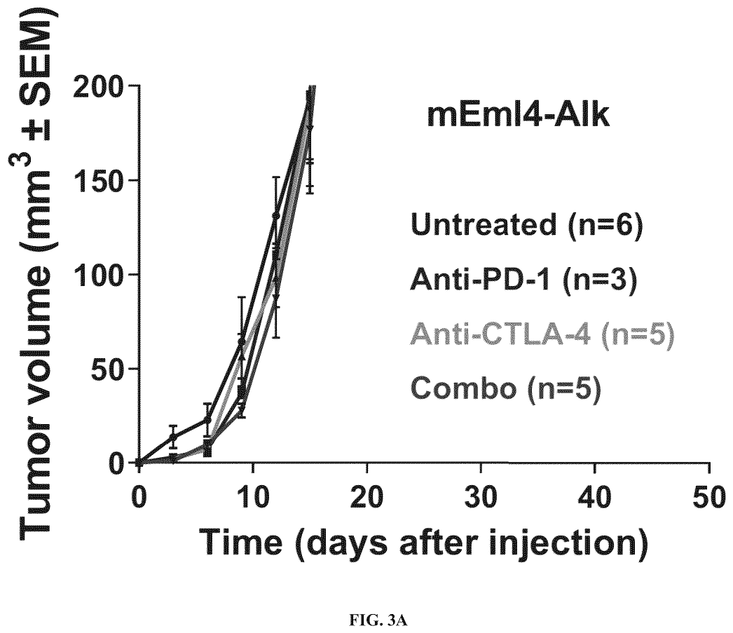

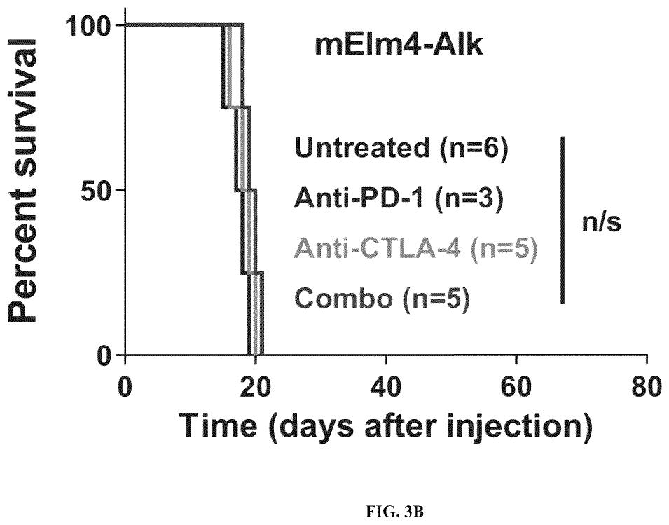

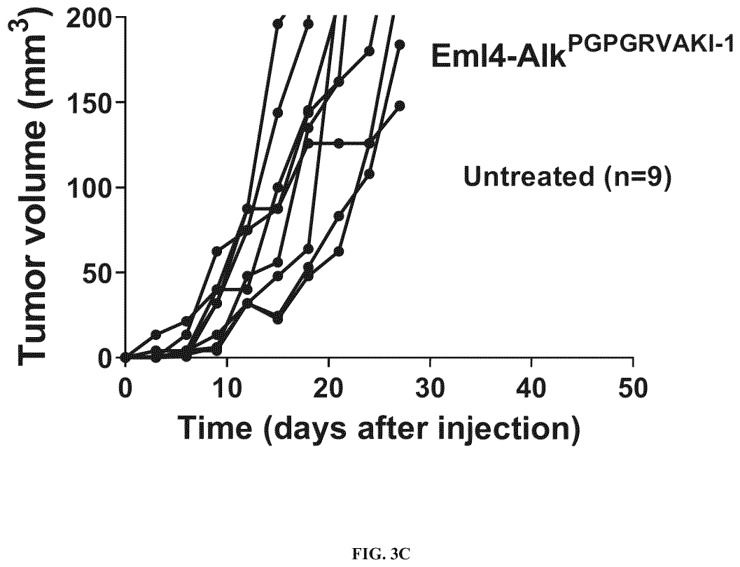

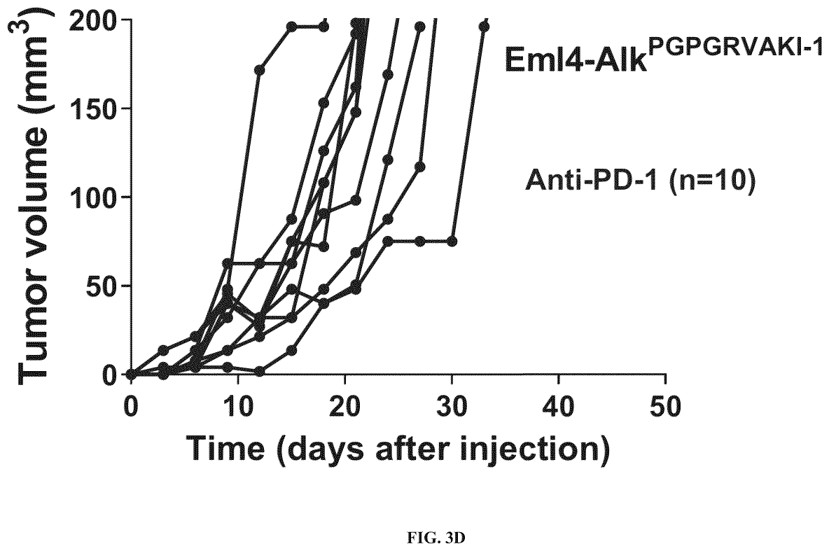

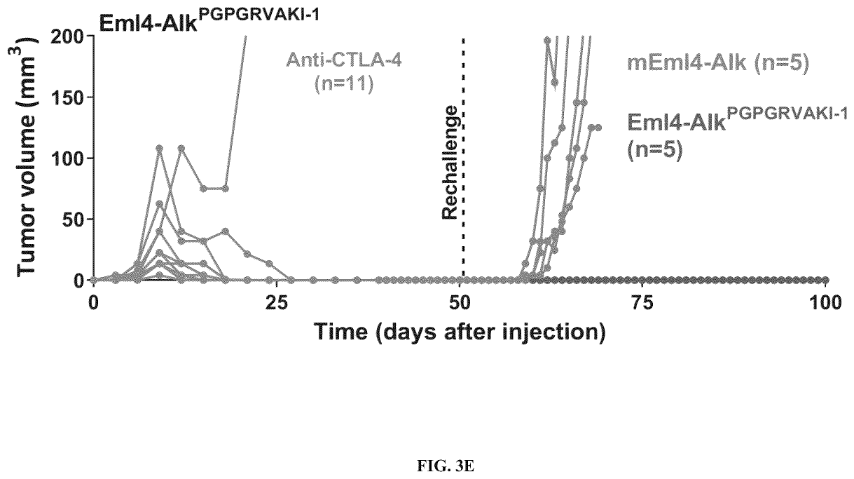

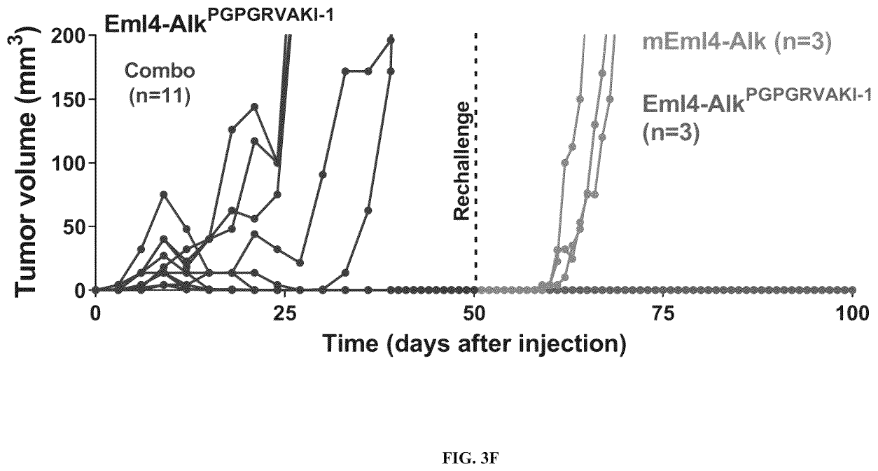

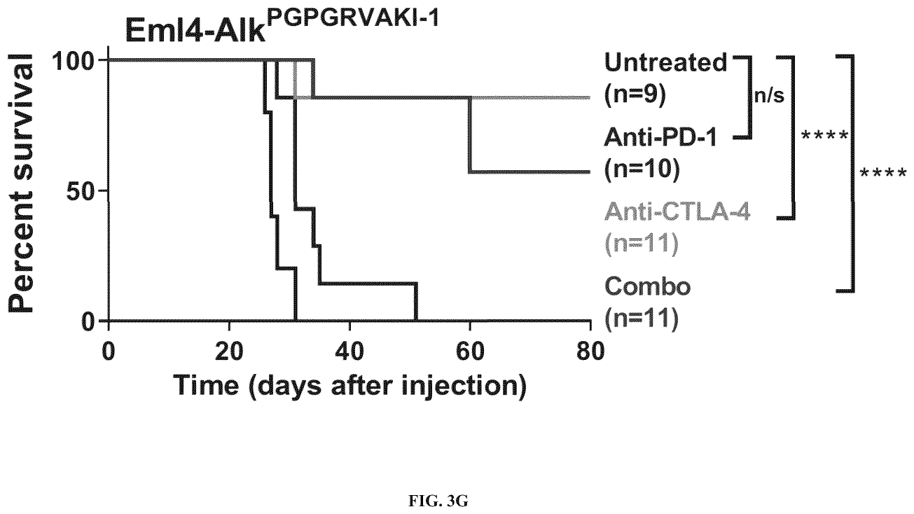

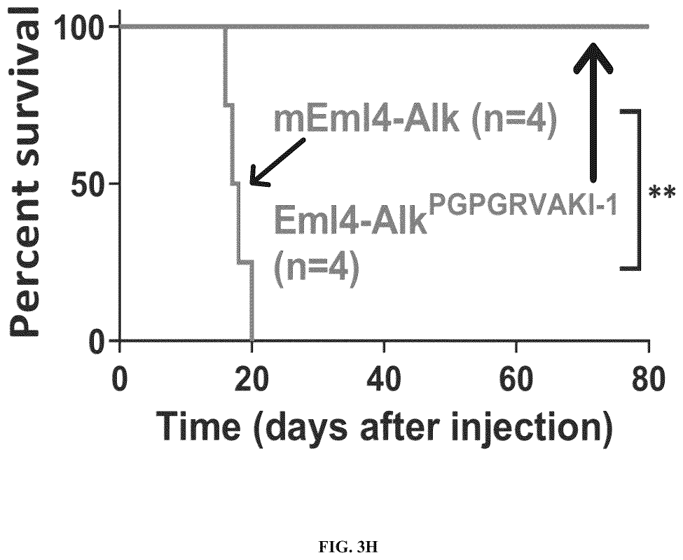

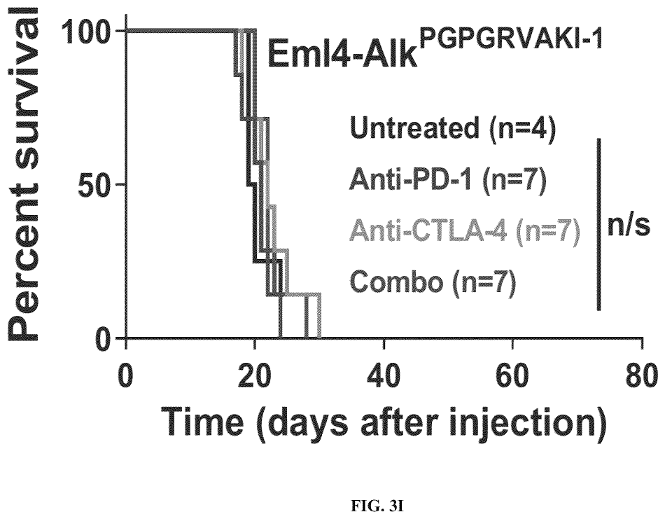

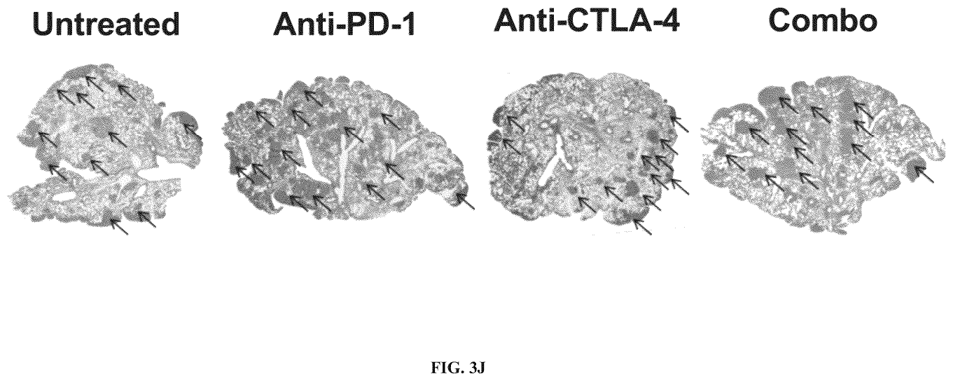

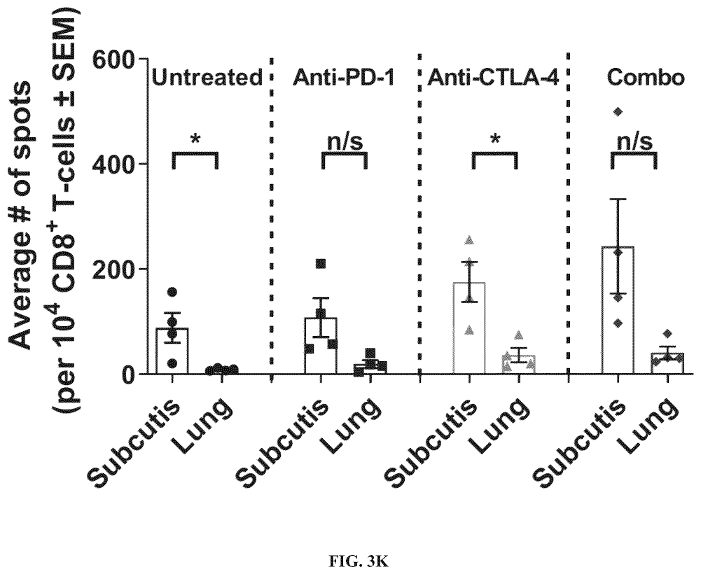

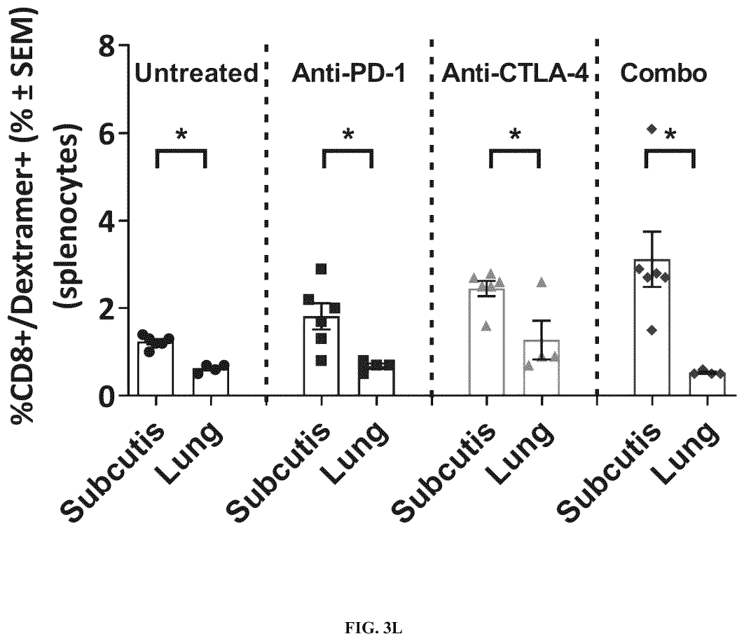

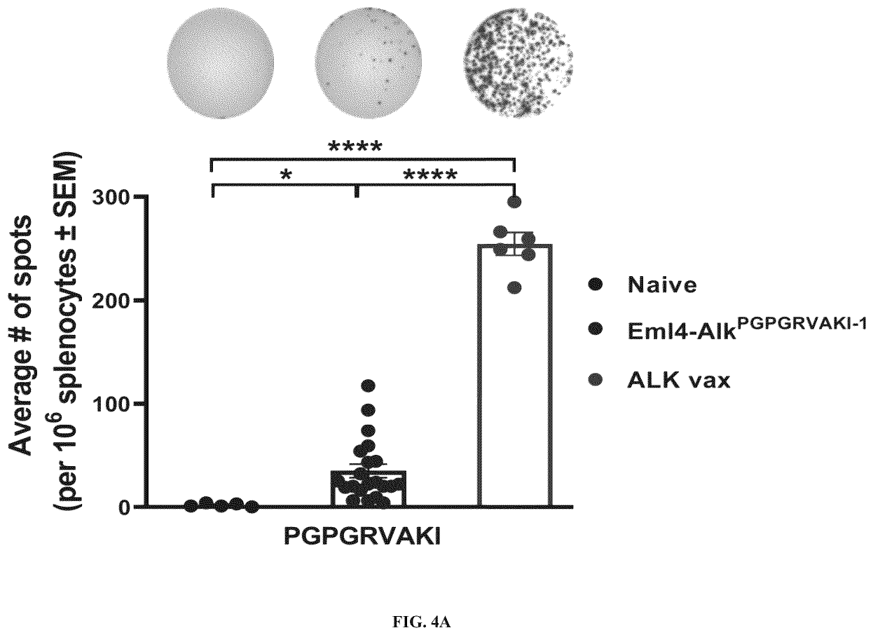

To determine whether these differences in anaplastic lymphoma kinase (ALK)-specific CD8⁺ T-cell responses translated into a different anti-tumor activity, mice were treated with either flank or lung tumors with immune checkpoint inhibitors (ICIs). When injected subcutaneously in the flank, immune checkpoint inhibitor (ICI) treatment did not reduce tumor growth of the parental mEml4-Alk cell line lacking the PGPGRVAKI (SEQ ID NO: 22) peptide (FIG. 3A) and did not extend the overall survival of mice (FIG. 3B), confirming the low immunogenicity of these tumors in the absence of the PGPGRVAKI (SEQ ID NO: 22) peptide. In contrast, Eml4-Alk^{PGPGRVAKI-1} (“PGPGRVAKI” disclosed as SEQ ID NO: 22) flank tumors were rejected when mice were treated either with anti-CTLA-4 alone or in combination with anti-PD1, with only partial impairment of tumor growth with anti-PD1 alone (FIGS. 3C-3F). Accordingly, overall survival was significantly extended in mice with flank tumors treated with anti-CTLA-4 or combination of anti-anti-CTLA-4 with anti-PD1 (FIG. 3G). Tumor-free mice were then rechallenged to assess ALK-specific CD8⁺ T-cell memory response. Tumor-free mice rejected efficiently Eml4-Alk^{PGPGRVAKI-1} (“PGPGRVAKI” disclosed as SEQ ID NO: 22) tumors re-injected subcutaneously in the flank (FIGS. 3E-3F) or intravenously (FIG. 3H) but did not reject mEml4-Alk tumors that lack the PGPGRVAKI (SEQ ID NO: 22) peptide. In contrast to flank tumors, ICI was ineffective against Eml4-Alk^{PGPGRVAKI-1} (“PGPGRVAKI” disclosed as SEQ ID NO: 22) lung tumors as measured by overall survival (FIG. 3I), and lung tumor histology (FIG. 3J). Systemic ALK-specific CD8⁺ T-cell response was analyzed in mice bearing flank or lung tumors. IFN-γ-ELISPOT assay and dextramer staining showed that flank tumors elicited higher ALK-specific CD8⁺ T-cell response when compared to lung tumors (FIGS. 3K and 3L). It was concluded that the robustness of the spontaneous ALK-specific CD8⁺ T-cell response depended on tumor localization and determined the response to immune checkpoint inhibitors (ICIs).

### Example 5. Enhanced Priming of the Anaplastic Lymphoma Kinase (ALK)-Specific Immune Response by Vaccination Lead to Rejection of ALK⁺ Lung Tumors and Prevented Metastatic Spread in Combination with Anaplastic Lymphoma Kinase (ALK) Tyrosine Kinase Inhibitors (TKI)

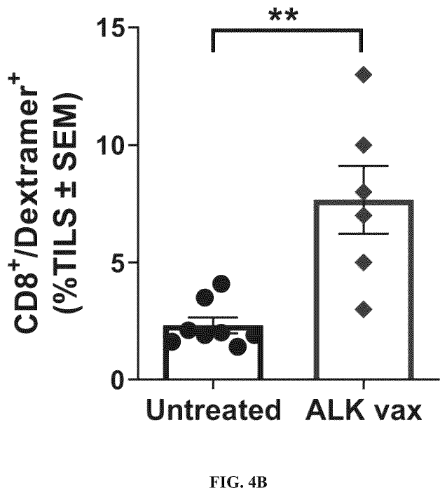

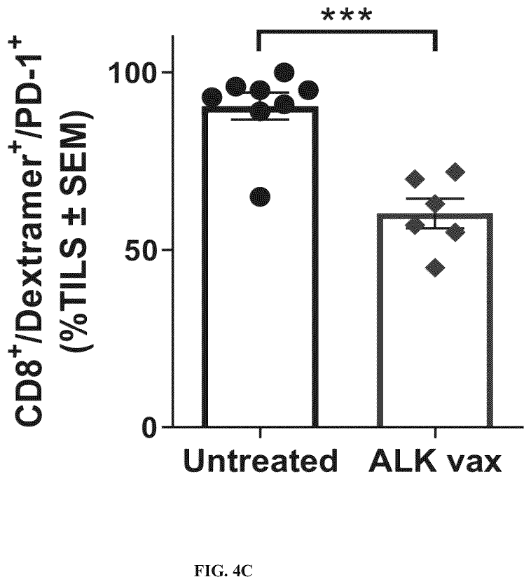

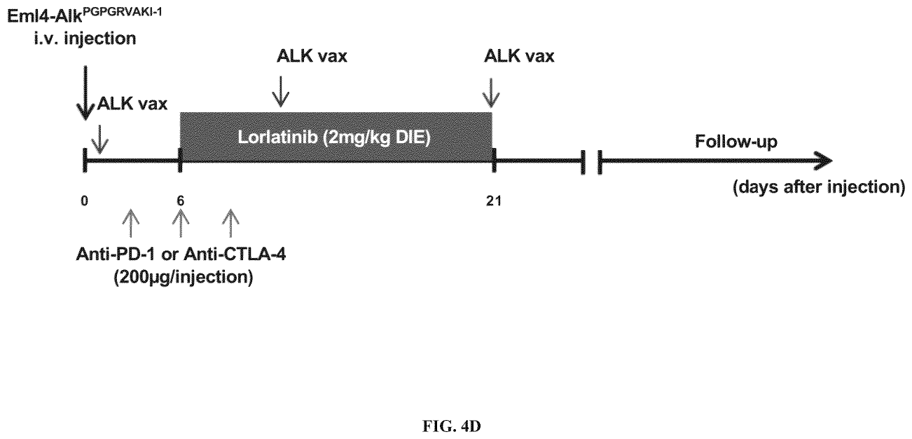

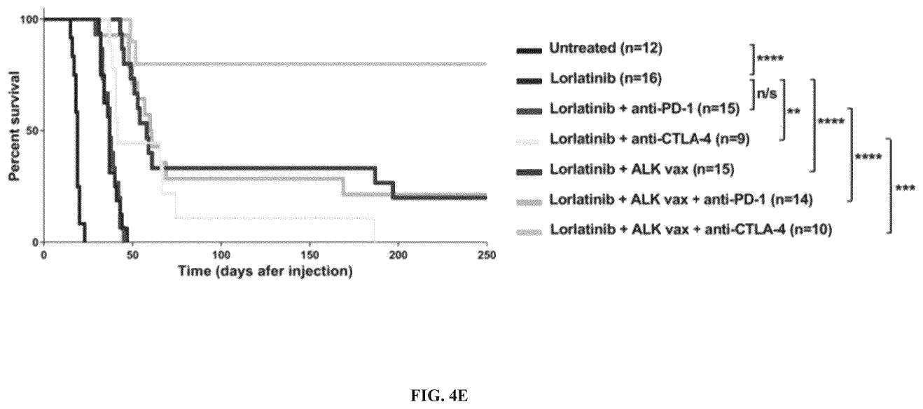

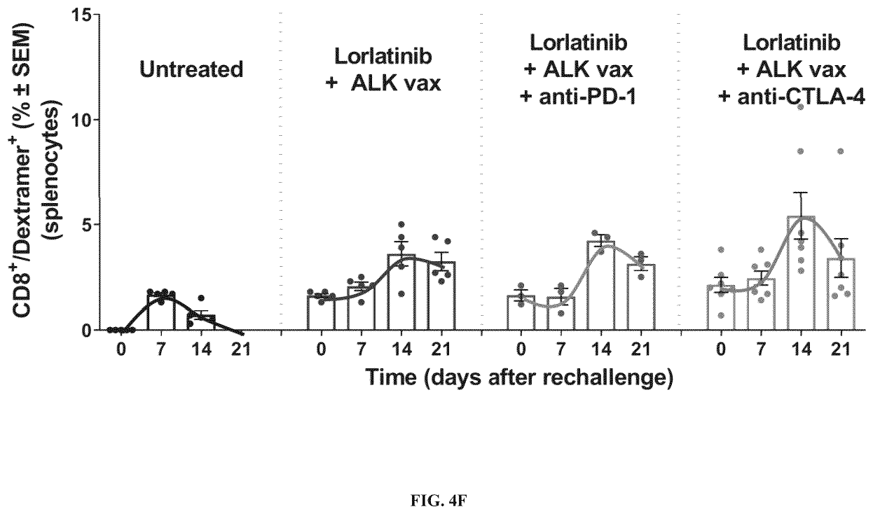

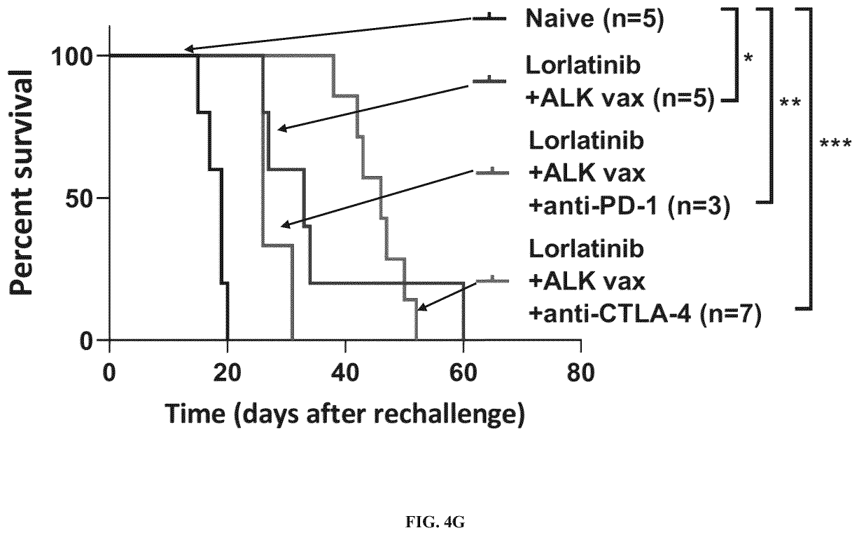

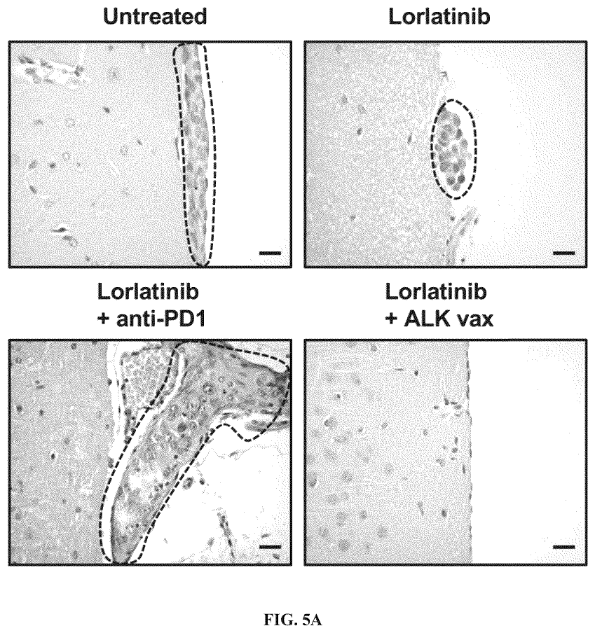

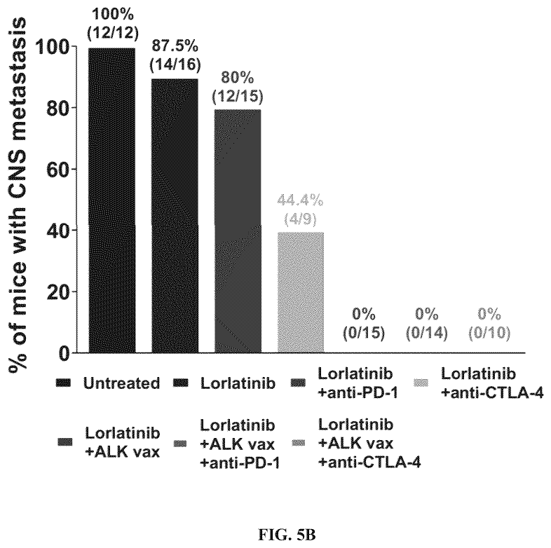

Without intending to be bound by theory, failure of response to immune checkpoint inhibitors (ICIs) in a subset T cell-infiltrated Non-Small Cell Lung Cancer (NSCLC) might be explained by a different trajectory during T cell priming in the mediastinal lymph node compared to tumors injected in the flank. Thus, not intending to be bound by theory, it was reasoned that the failure of ICI to reject ALK+ lung tumors could be secondary to an insufficient priming of spontaneous ALK-specific CD8⁺ T-cell response. Vaccines can revert the poor immunogenicity of some tumors by improving priming of CD8⁺ T-cells for selected antigens. Thus, the spontaneous ALK-specific CD8⁺ T-cell response elicited by ALK+ lung tumors was compared to that by the PGPGRVAKI (SEQ ID NO: 22) peptide vaccine. BALB/c mice were either intravenously injected with Eml4-Alk^{PGPGRVAKI-1} (“PGPGRVAKI” disclosed as SEQ ID NO: 22) or vaccinated with the ALK vaccine, and the systemic ALK-specific CD8⁺ T-cell response was evaluated. By ELISPOT assay, CD8⁺ T-cells from ALK vaccinated mice produced a stronger IFN-γ response than CD8⁺ T-cells primed by ALK⁺ lung tumors when incubated with the PGPGRVAKI (SEQ ID NO: 22) peptide (FIG. 4A). The ALK vaccine also significantly increased the number of ALK-specific tumor-infiltrating T lymphocytes (TILs) when compared with ALK-specific TILs spontaneously induced by ALK+ tumors in non-vaccinated mice (FIG. 4B), and ALK-specific TILs in vaccinated mice showed lower levels of PD-1 expression (FIG. 4C).

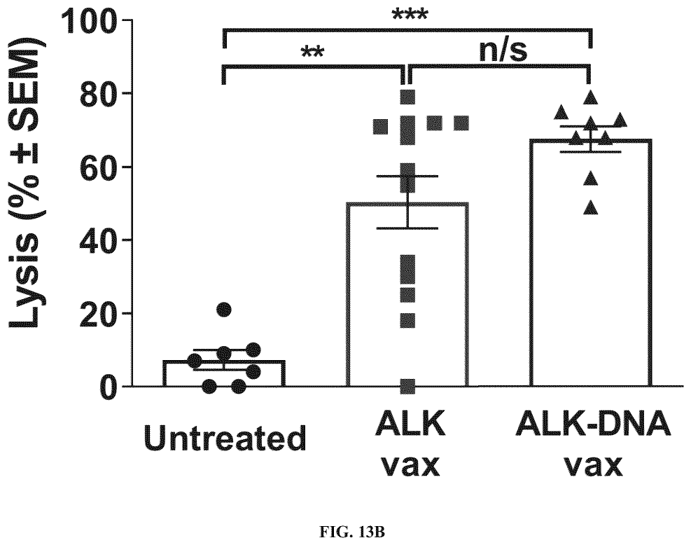

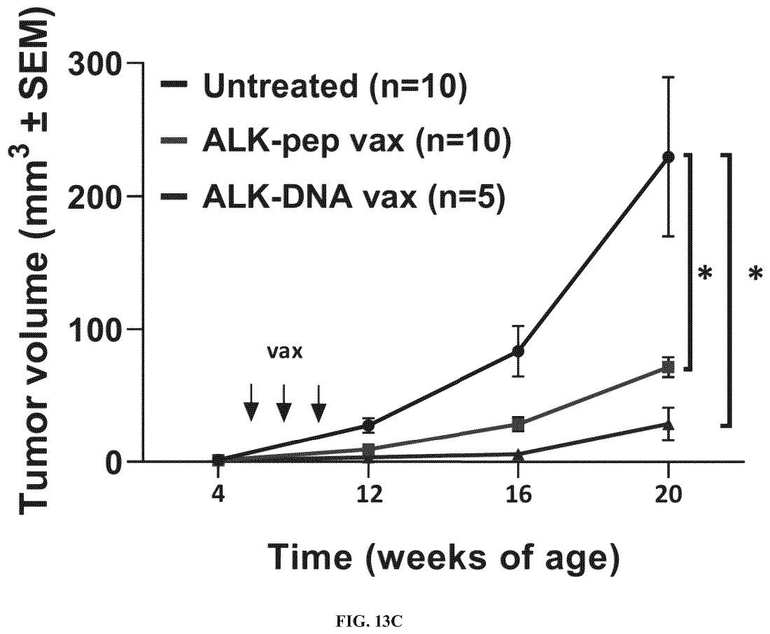

Prompted by these findings, it was tested whether enhanced priming through the ALK vaccine would generate an effective anti-tumoral response. First, the ALK vaccine was tested alone. Because mice injected with Eml4-Alk^{PGPGRVAKI-1} (“PGPGRVAKI” disclosed as SEQ ID NO: 22) cells die too rapidly to test the vaccine alone (FIG. 3K), hEML4-ALK Tg mice were used that have a slower tumor kinetics. Mice were vaccinated with the PGPGRVAKI (SEQ ID NO: 22) peptide or the ALK-DNA and tumor growth was followed by MRI over time (FIG. 13A). Both vaccines induced a strong cytotoxic response against ALK⁺ cells in vivo (FIG. 13B) and impaired tumor growth (FIG. 13C). These data showed that priming of ALK-specific CD8⁺ T-cells by a vaccine generates an immune response that can efficiently target ALK+ tumor cells.

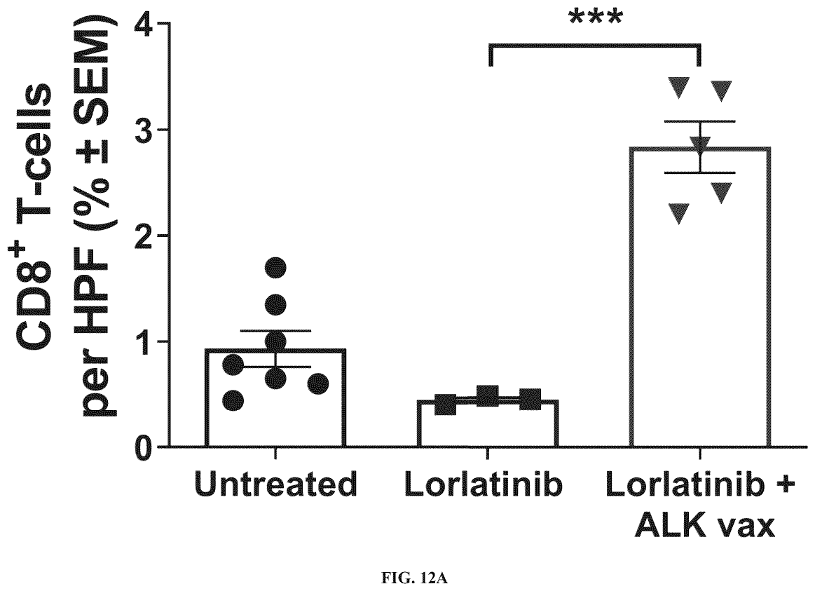

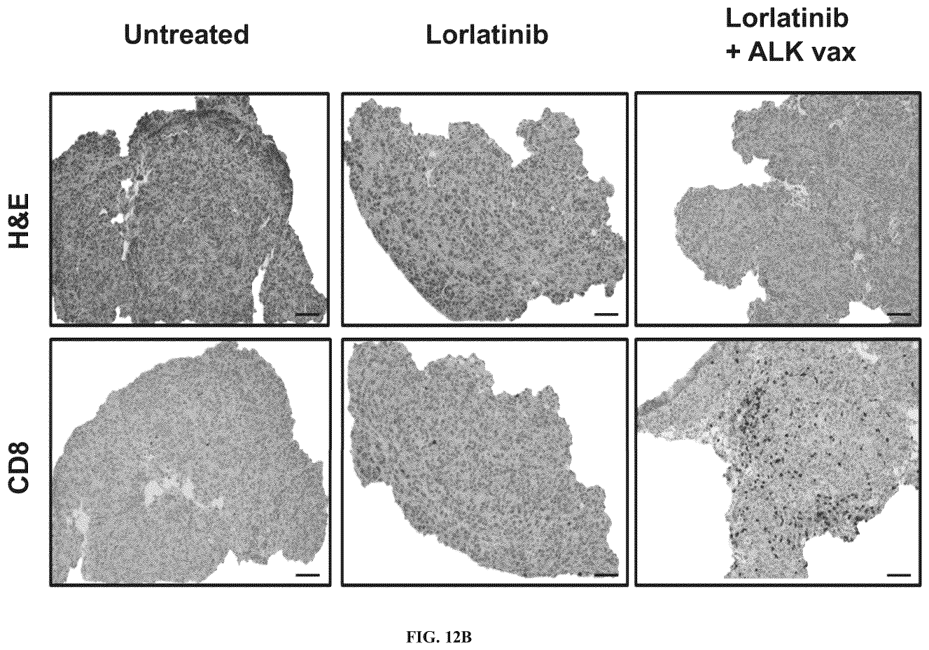

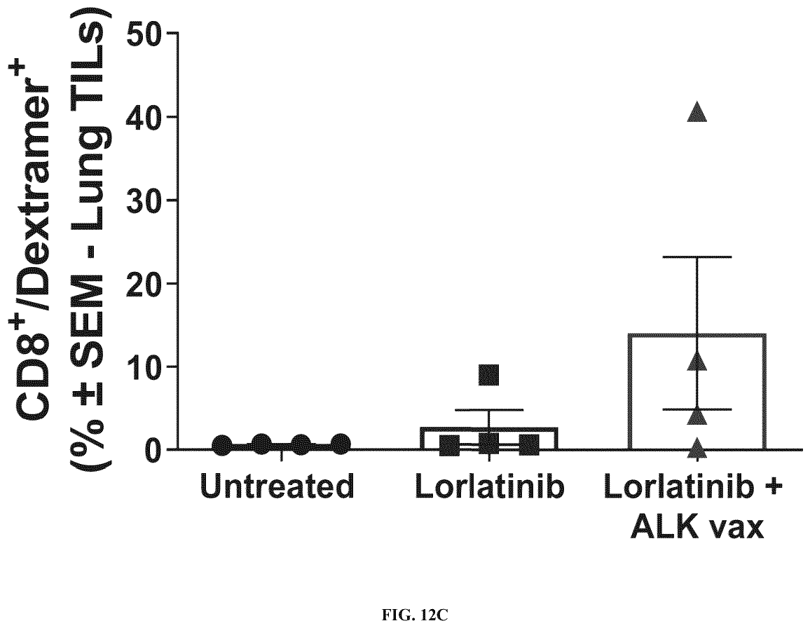

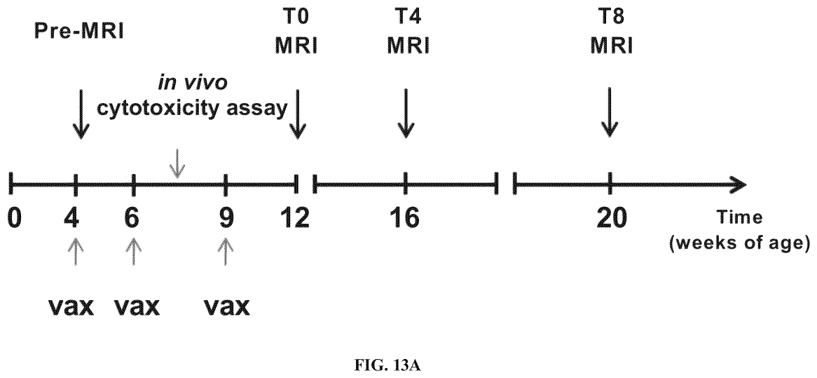

Next, it was asked whether priming of ALK-specific CD8⁺ T-cells by the ALK vaccine would completely eradicate ALK+ lung tumors in combination with ALK TKI treatment. For this therapeutic experiments, hEML4-ALK Tg mice were suboptimal because the constitutive expression of EML4-ALK by the SP-C promoter induced a continuous onset of new tumors. Therefore, the Eml4-Alk^{PGPGRVAKI-1} (“PGPGRVAKI” disclosed as SEQ ID NO: 22) model (FIG. 3K) was used and treated mice with lorlatinib in combination with the ALK vaccine and/or immune checkpoint inhibitors (ICIs) (FIG. 4D). Treatment with lorlatinib extended the overall survival of the mice, but all tumors relapsed upon lorlatinib suspension (FIG. 4E), as in hEML4-ALK Tg or Ad-EA mice (FIGS. 1E-1J). The addition of anti-PD-1 to lorlatinib did not extend survival, while the addition of anti-CTLA-4 produced a modest extension of survival (FIG. 4E). Remarkably, the addition of the ALK vaccine to lorlatinib treatment alone or in combination with ICIs significantly extended the overall survival and achieved a complete cure in a subset of mice (25% cure rate with lorlatinib+ALK vaccine; 25% cure rate with lorlatinib+ALK vaccine+anti-PD-1; 80% cure rate with lorlatinib+ALK vaccine+anti-CTLA-4) (FIG. 4E). Histologic examination of lungs in cured mice did not identify residual tumor cells, which was suggestive of complete tumor eradication. In vaccinated mice, lung tumors that were not eradicated showed increased total CD8⁺ TILs and ALK-dextramer⁺ tumor-infiltrating T lymphocytes (TILs) when compared to untreated mice or mice treated with lorlatinib alone (FIG. 12A-12C). Next, the ALK-specific immune memory was evaluated by re-challenging tumor-free mice (FIG. 4E) with Eml4-Alk^{PGPGRVAKI-1} cells more than 200 days after the last vaccination. Compared to treatment-naive mice, tumor-free mice from all vaccinated groups showed a substantial expansion of systemic ALK dextramer⁺ CD8⁺ T cells upon tumor rechallenge (FIG. 4F) and a significant extension of survival (FIG. 4G). When injected i.v. Eml4-Alk^{PGPGRVAKI-1} (“PGPGRVAKI” disclosed as SEQ ID NO: 22) cells not only form lung tumors but also grow in the brain with tropism for the meningeal space (FIG. 5A), representing a useful model to test whether the treatment with the ALK vaccine prevented the metastatic spread of ALK tumor cells. Metastatic spread to the brain is a negative prognostic factor in ALK+ Non-Small Cell Lung Cancer (NSCLC) patients. Remarkably, the metastatic spread to the brain was completely blocked in all vaccinated mice (FIGS. 5A and 5B). Altogether, these data demonstrated that increasing the robustness of anti-ALK-specific immune responses by a single peptide vaccine was sufficient to achieve therapeutic efficacy by extending survival, eradicating primary ALK⁺ lung tumors, generating an ALK-specific immune memory, and blocking the metastatic spread of ALK⁺ tumor cells.

### Example 6. Tumor Escape in Vaccinated Mice was Associated with Reversible MHC-I Downregulation

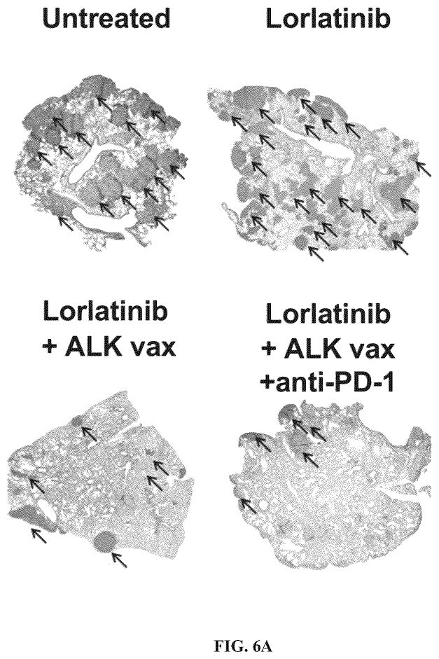

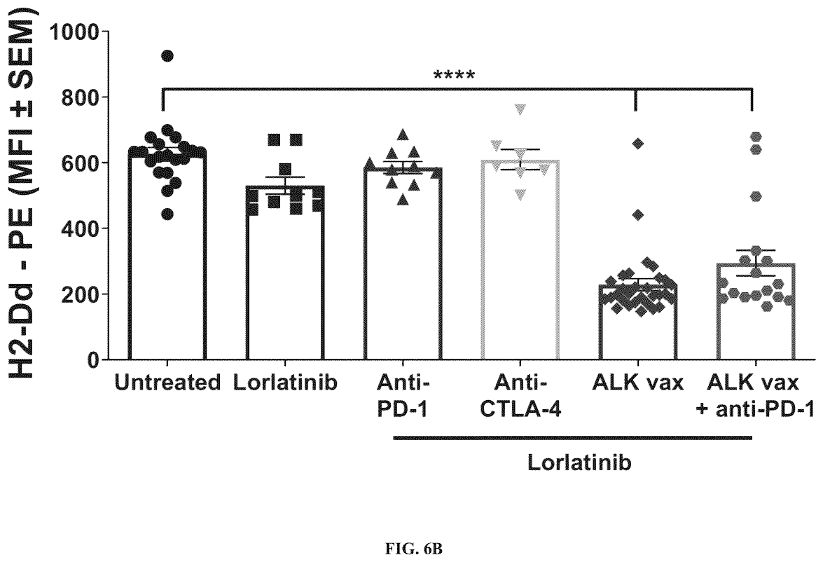

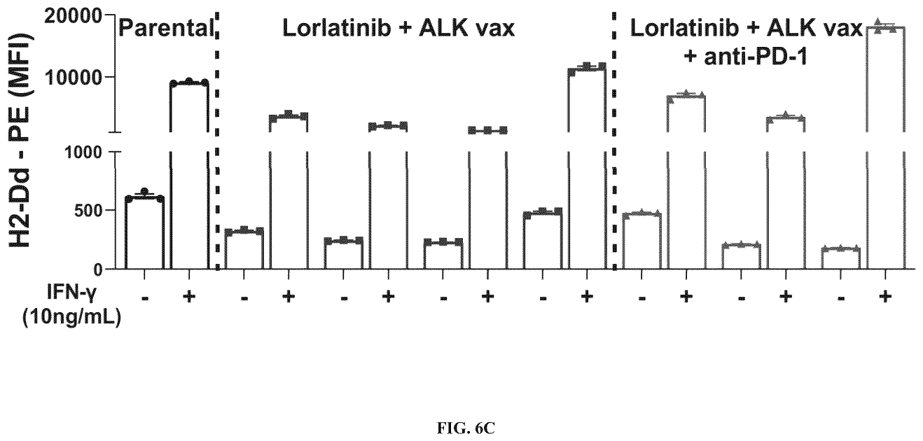

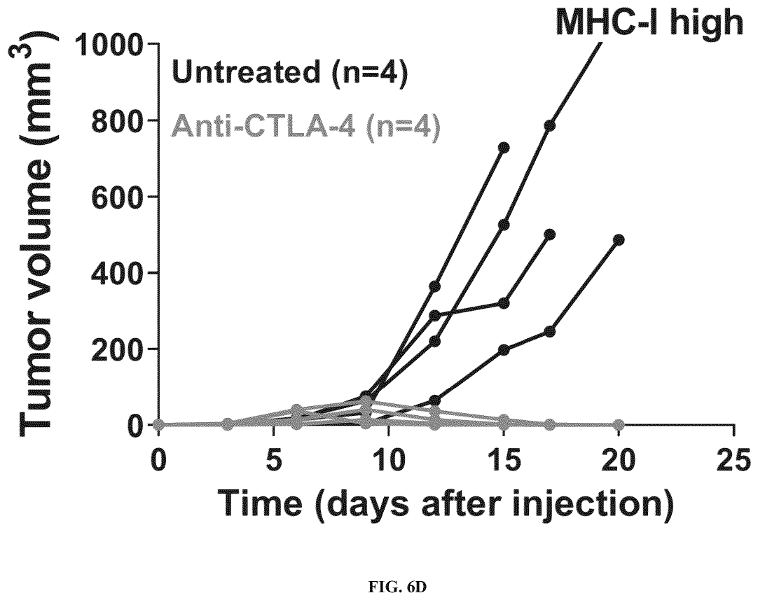

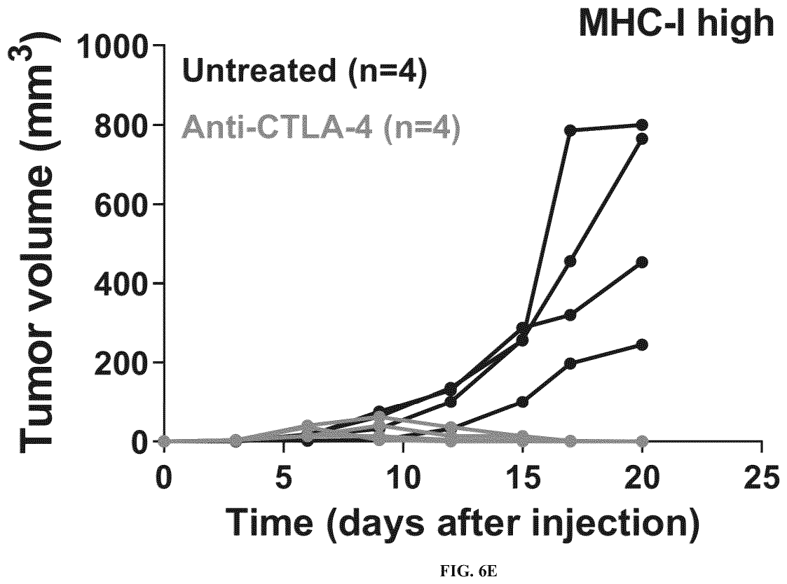

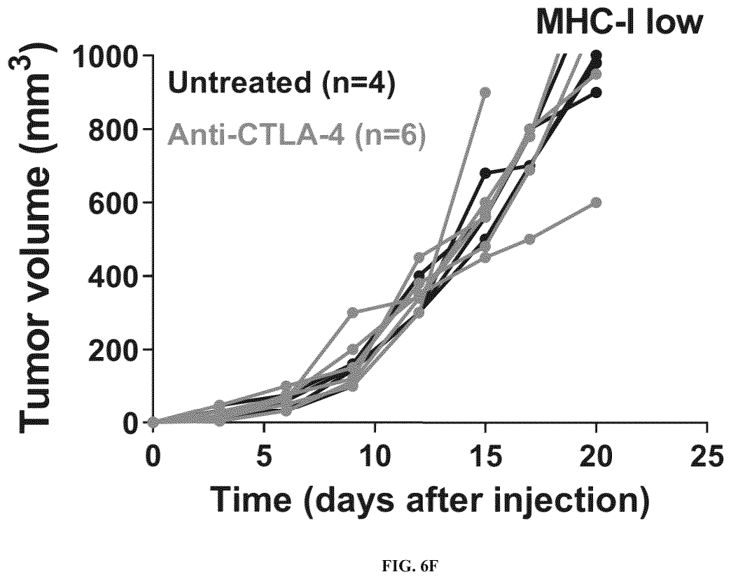

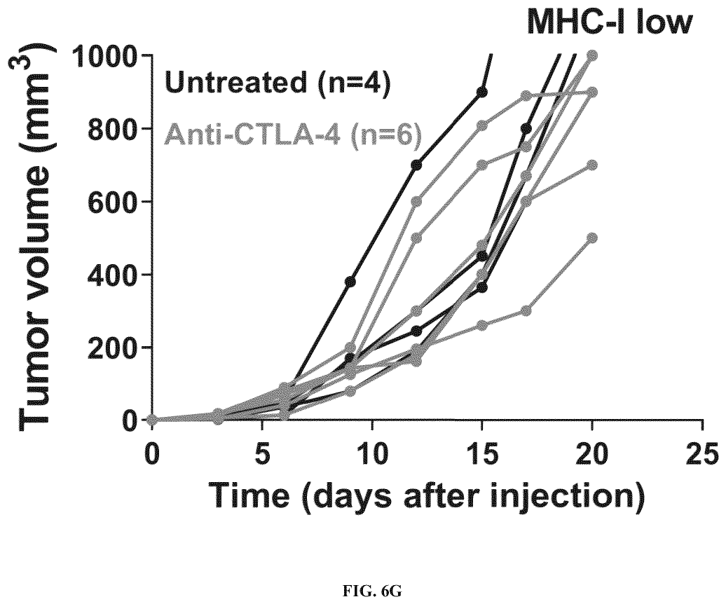

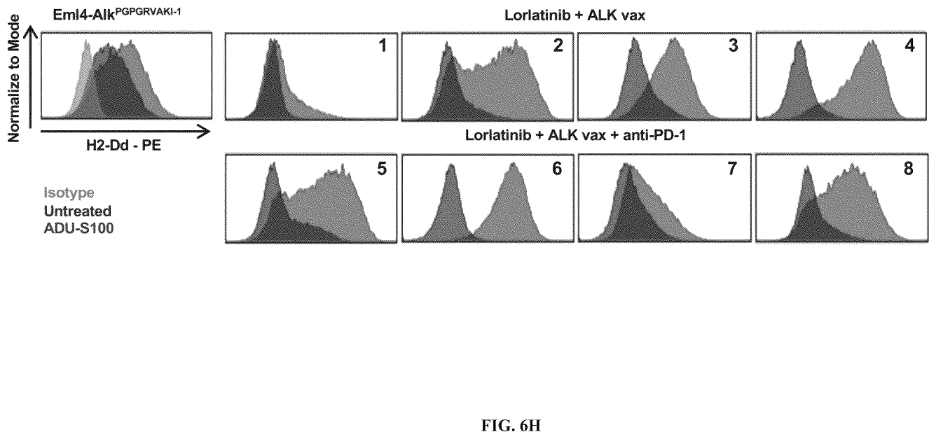

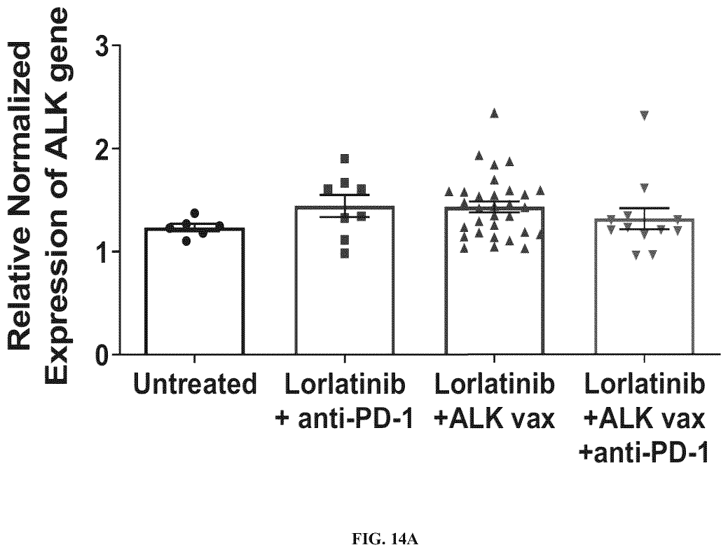

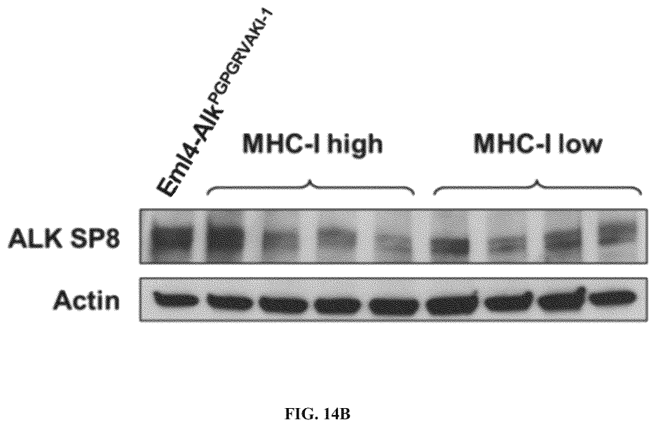

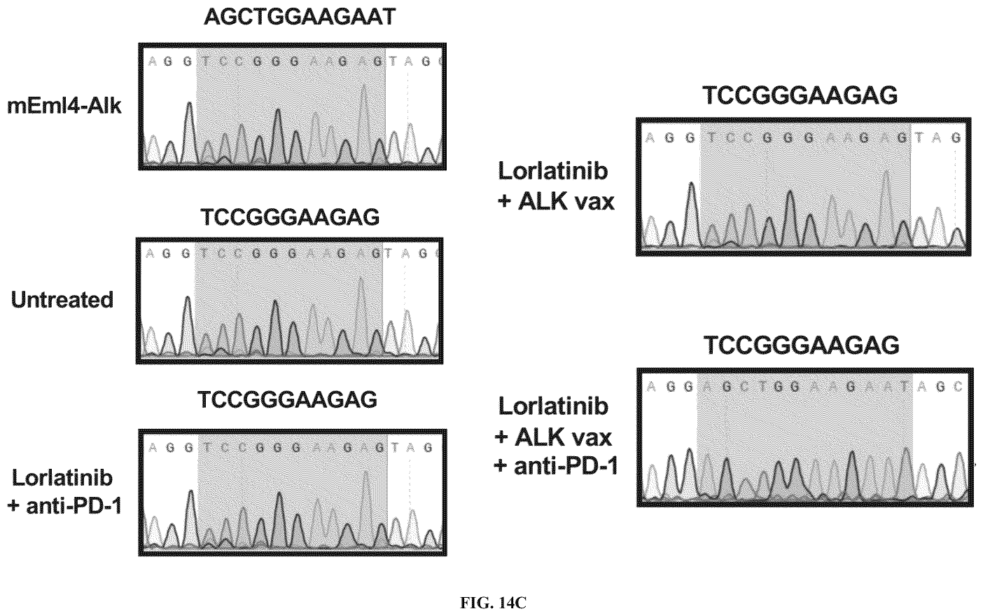

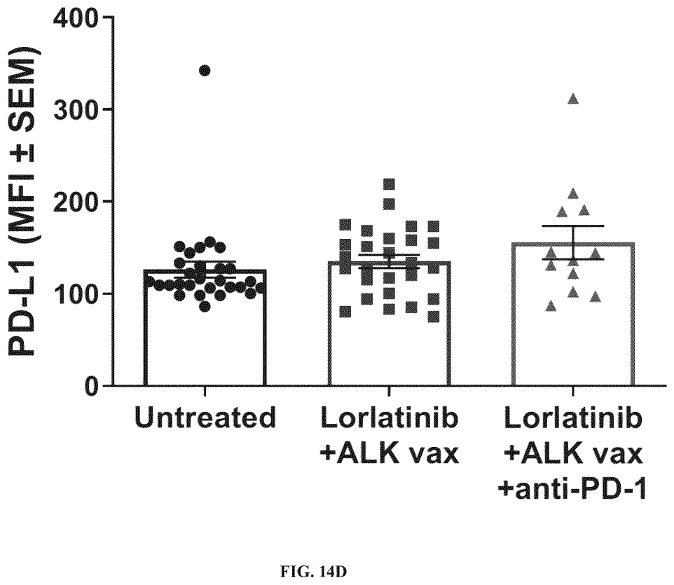

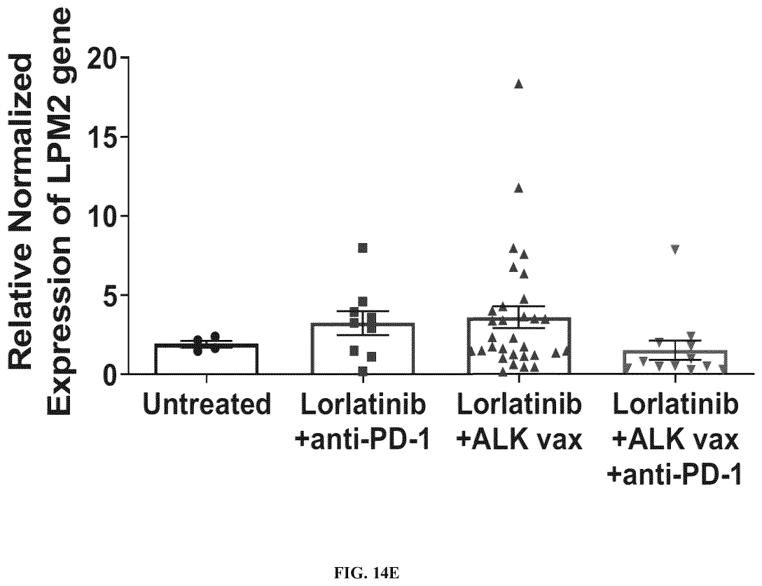

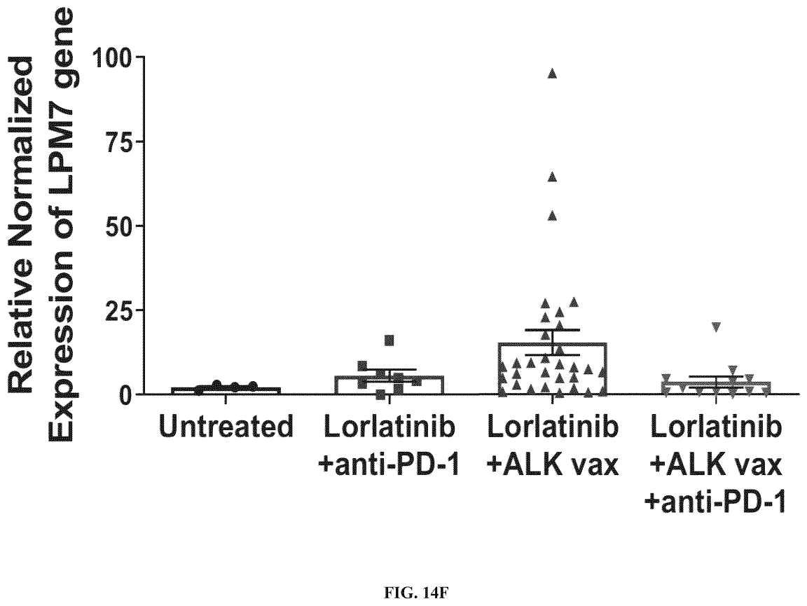

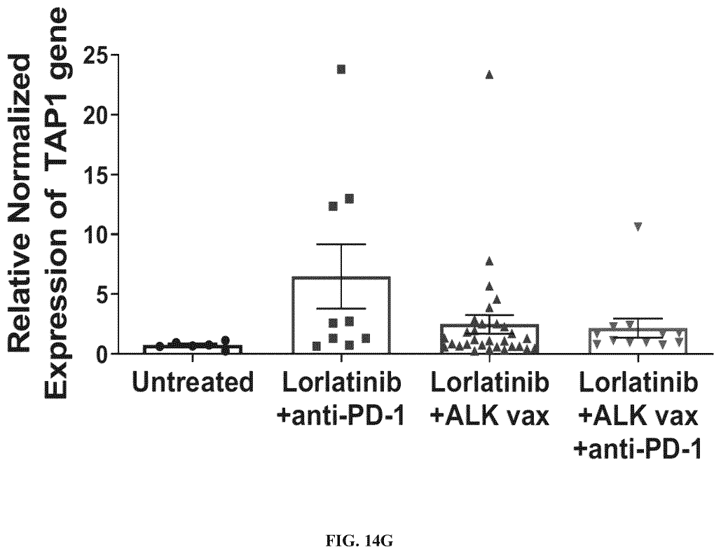

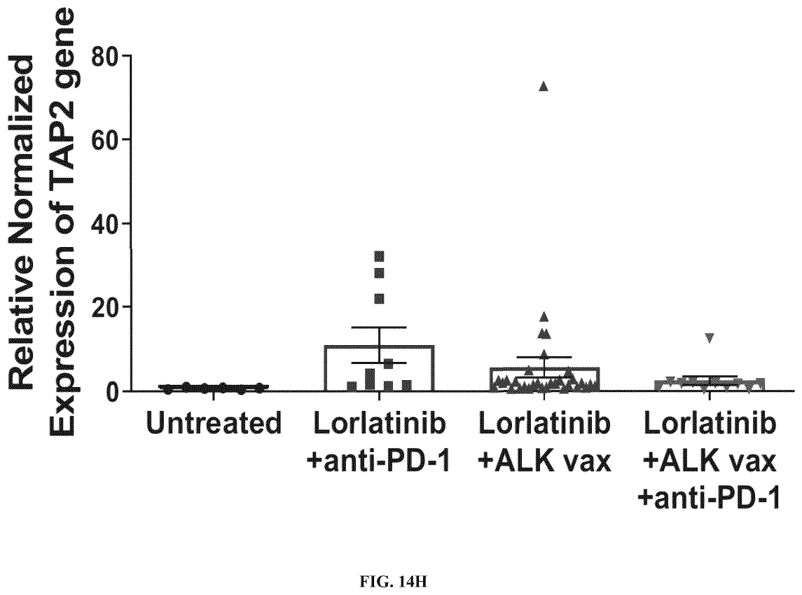

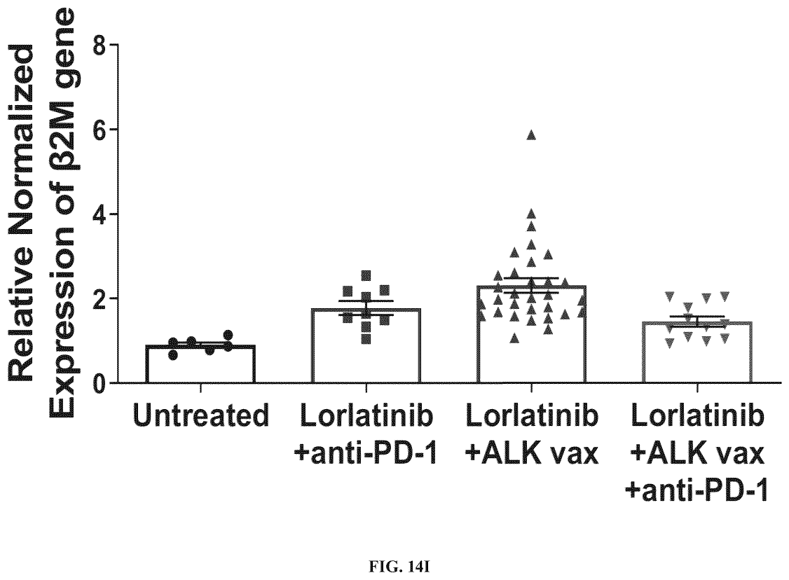

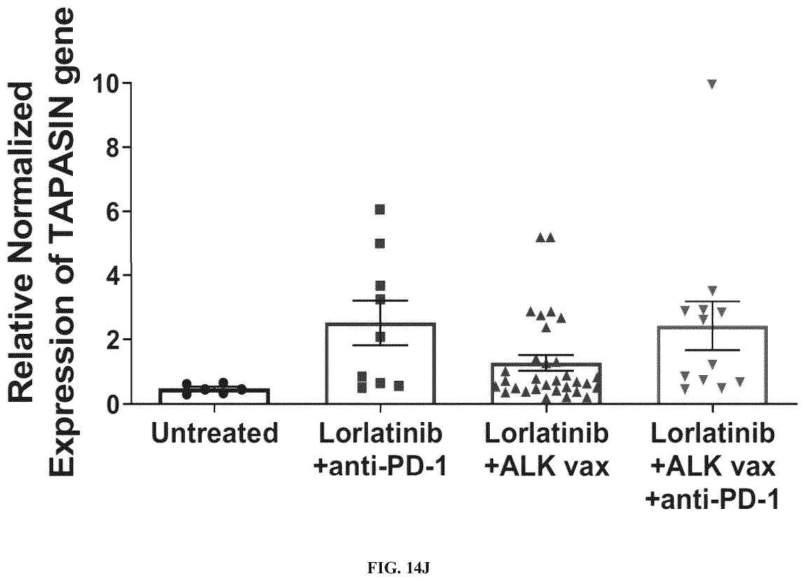

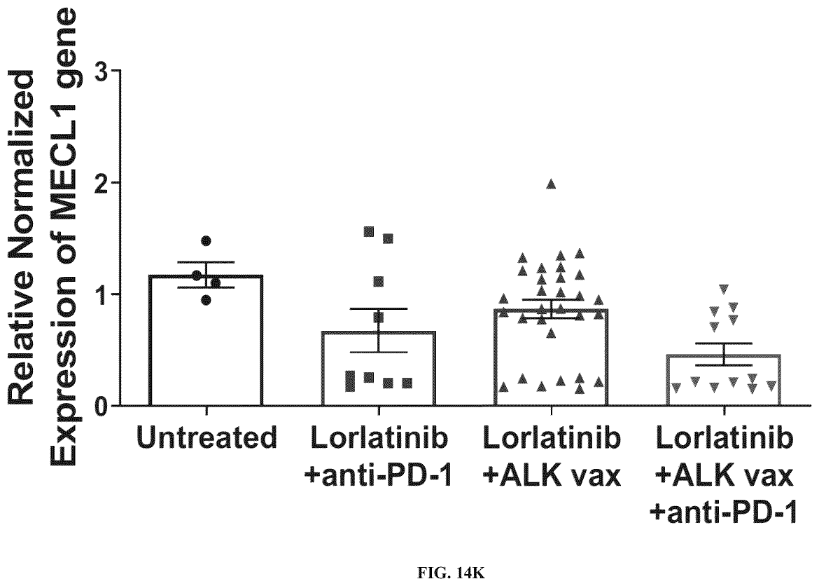

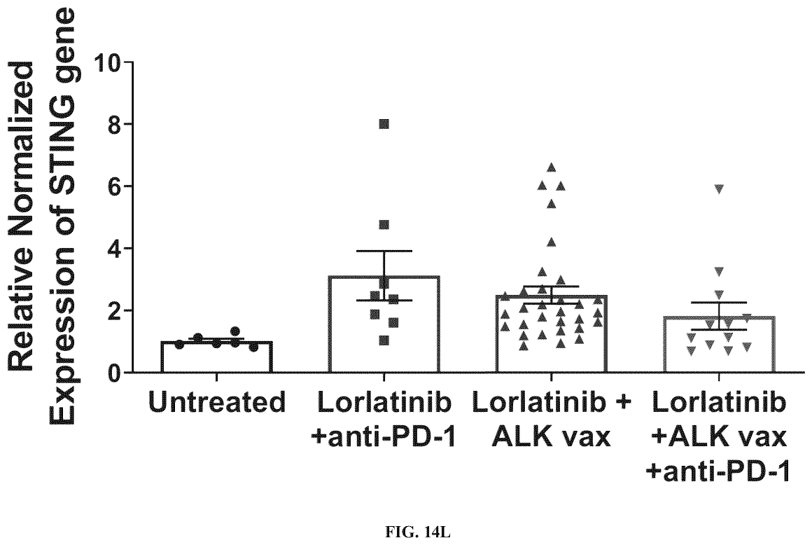

While significantly extending the overall survival in all mice, the addition of the anaplastic lymphoma kinase (ALK) vaccine to lorlatinib failed to cure a substantial portion of mice (FIG. 4E). To uncover the mechanisms that would explain this resistance to treatment, ex vivo escaped tumors were isolated from each treatment group. ALK vaccinated mice showed a reduced number of tumors (FIG. 6A), that however retained the expression of the Eml4-Alk fusion measured by mRNA (FIG. 14A) and EML4-ALK fusion protein (FIG. 14B). Sequencing of the Eml4-Alk mRNA demonstrated that the edited PGPGRVAKI (SEQ ID NO: 22) peptide was still expressed (FIG. 14C), excluding the lack of antigen expression as a mechanism of escape. Also, surface expression of PD-L1 did not show differences among tumors isolated from different treatment groups (FIG. 14D), excluding increased PD-L1 expressing as a resistance mechanism. However, a significant downregulation of H2-Dd expression was observed on the cell surface of tumors isolated from vaccinated mice but not from mice treated with lorlatinib or lorlatinib+immune checkpoint inhibitors (ICIs) (FIG. 6B). Not intending to be bound by theory, H2-Dd downregulation was not due to a genetic deletion nor to decreased expression of any of the genes related to the antigen presentation machinery (Lpm2, Lmp7, Mecl1, Tap1, Tap2, β2M, and Tapasin) (FIGS. 14E-14K), and when stimulated in vitro with IFN-γ, H2-Dd expression was restored in tumor cells (FIG. 6C), suggesting that an epigenetic mechanism might have played a role in H2-Dd downregulation. To understand whether the observed H2-Dd downregulation was sufficient to impair tumor rejection, tumor cells with high H2-Dd isolated from untreated mice or tumor cells with low H2-Dd isolated from ALK vaccinated mice and treated with ICI were injected. Treatment with ICI induced rejection of tumors with high H2-Dd expression (FIGS. 6D-6E), consistent with previous experiments (FIG. 3E), but not of escaped tumors with low H2-Dd expression (FIGS. 6F-6G). Finally, it was asked whether H2-Dd expression could be restored by Stimulator of IFN genes (STING) signaling that can increase MHC-I expression in other models of lung cancer. Tumor cell-intrinsic STING expression was not downregulated in ex vivo tumor cells from all therapeutic groups (FIG. 14L) and in fact, all H2-Dd-downregulated cell lines tested were sensitive to STING agonist ADU-S100, strongly upregulating H2-Dd expression (FIG. 6H). Thus, while not intending to be bound by theory, the reversibility of this phenotype by a STING agonist suggested a potential approach to further increase the ALK vaccine efficacy by reducing the escape of tumor clones with MHC-I downregulated.

### Example 7. Identification of Immunogenic Anaplastic Lymphoma Kinase (ALK) Peptides in Alk⁺ Non-Small Cell Lung Cancer (NSCLC) Patients

Experiments in mice (FIG. 16A) demonstrated that restoring an efficient CD8+ T cell priming by vaccination with an immunogenic ALK peptide achieved therapeutic effect in ALK+ lung tumors that were otherwise poorly immunogenic. To translate these findings into an ALK vaccine for ALK+ Non-Small Cell Lung Cancer (NSCLC) patients, experiments were undertaken to identify immunogenic human ALK peptides in the context of human MHC-I molecules.

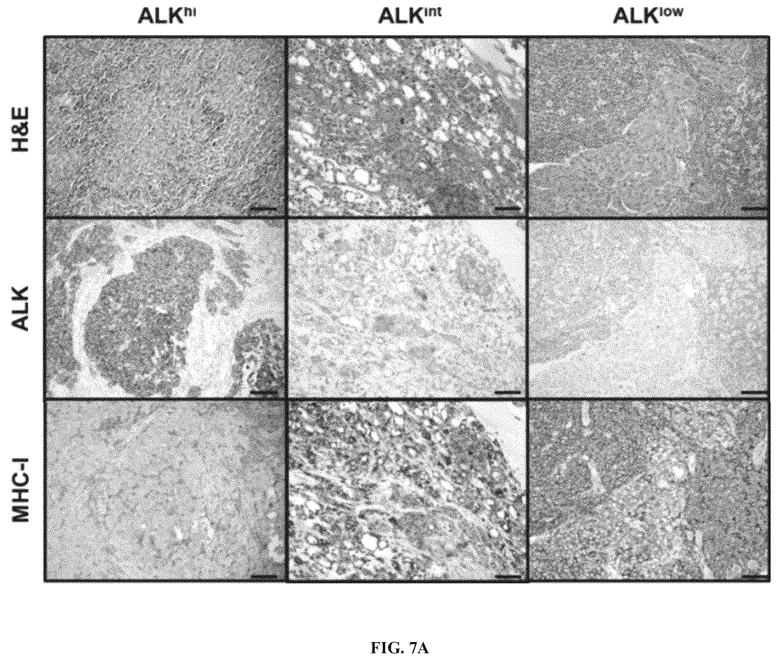

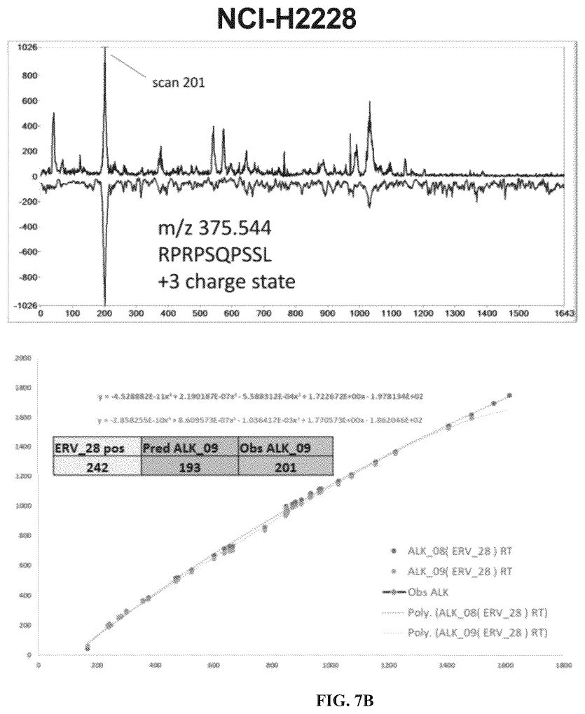

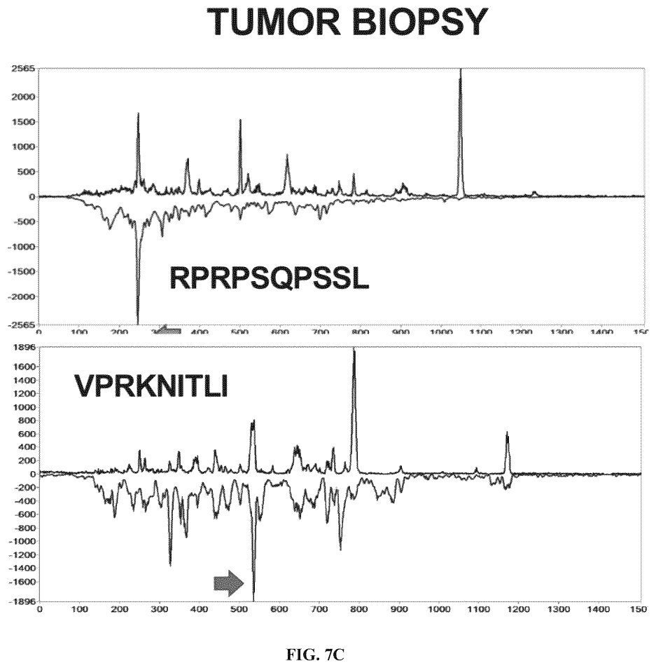

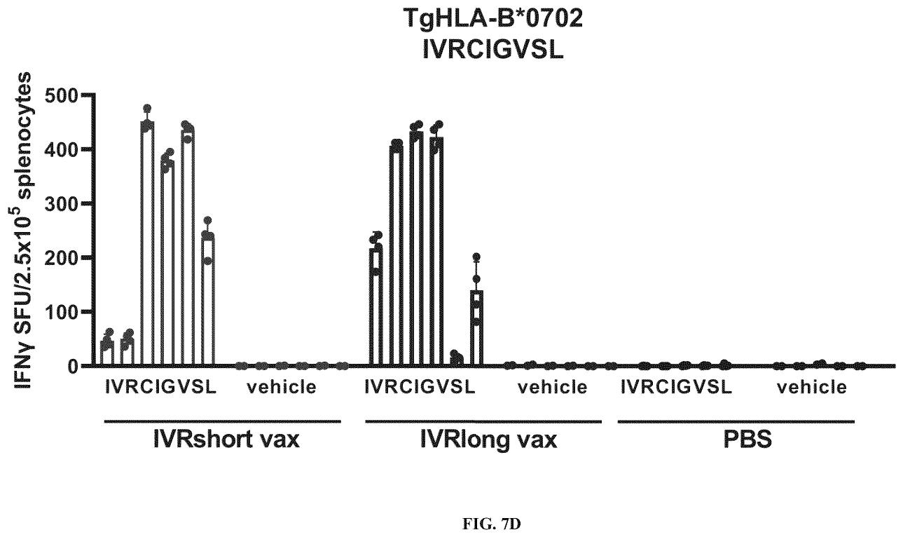

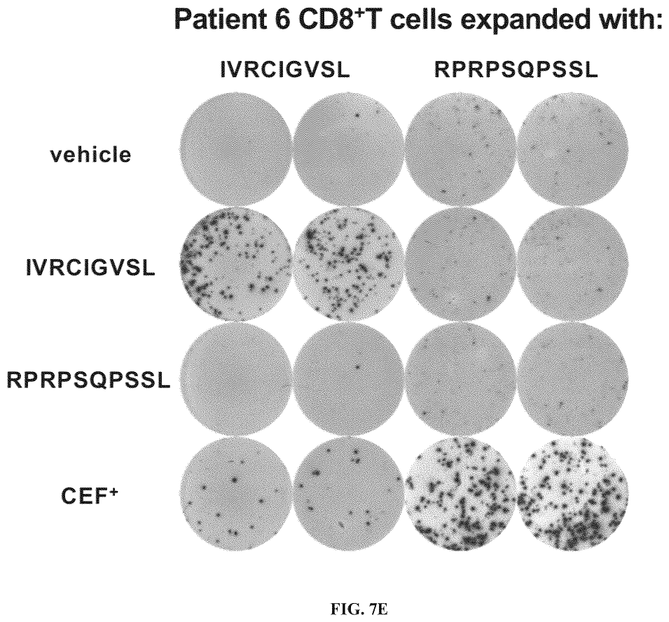

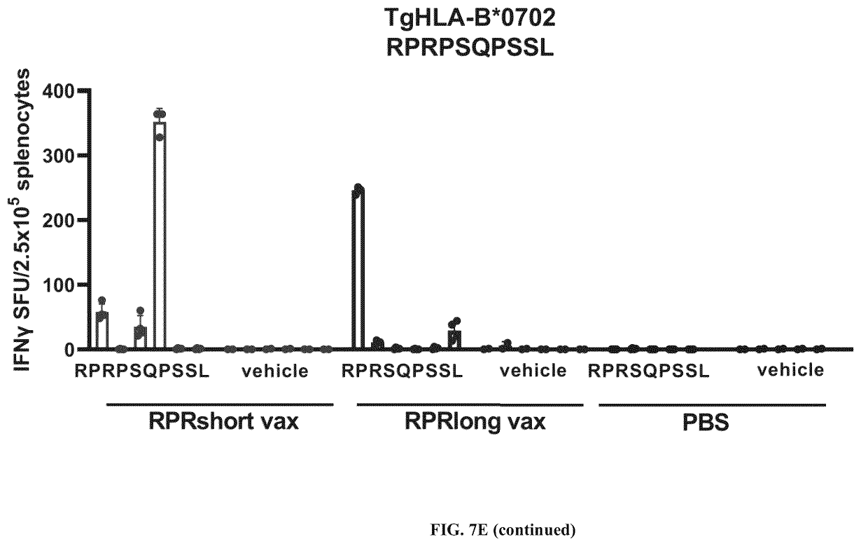

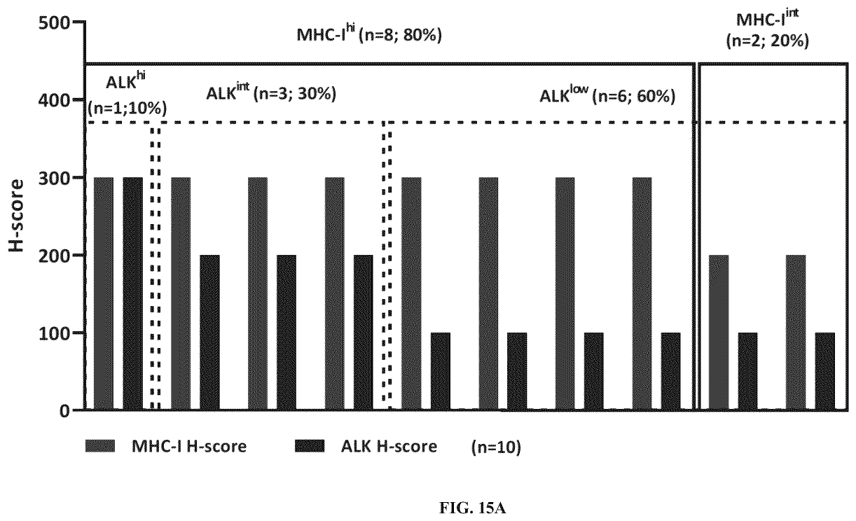

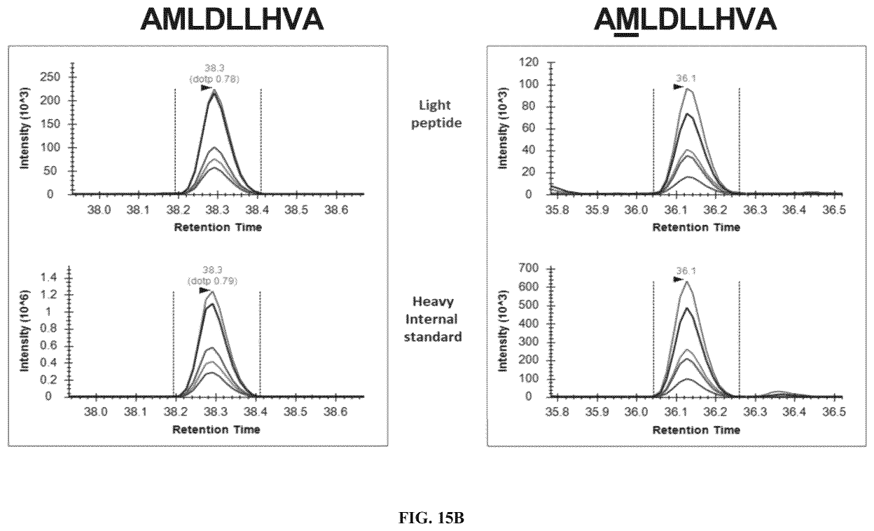

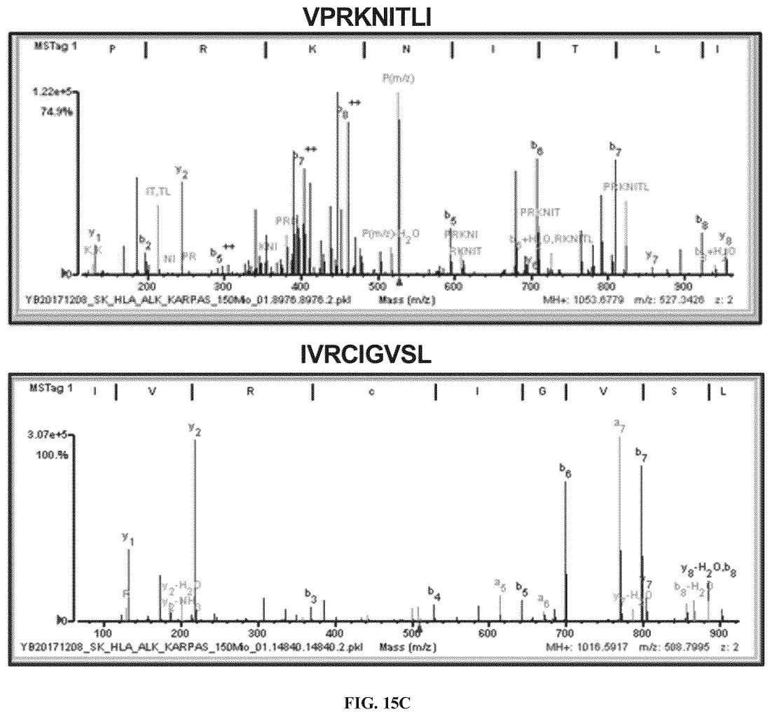

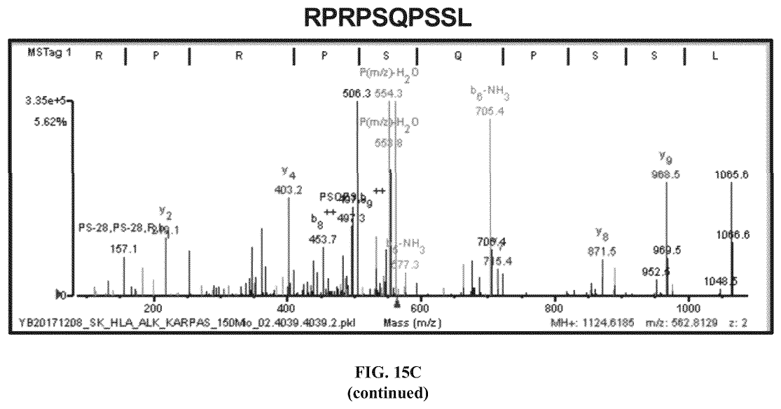

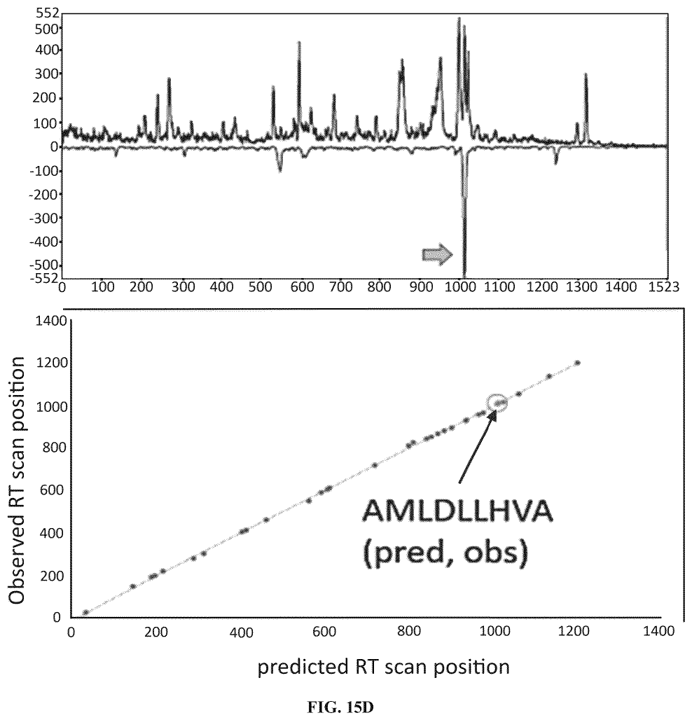

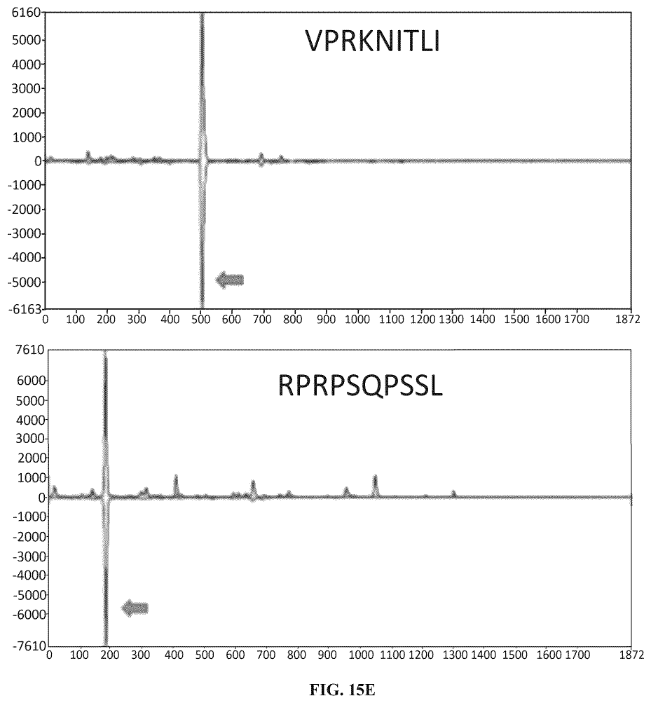

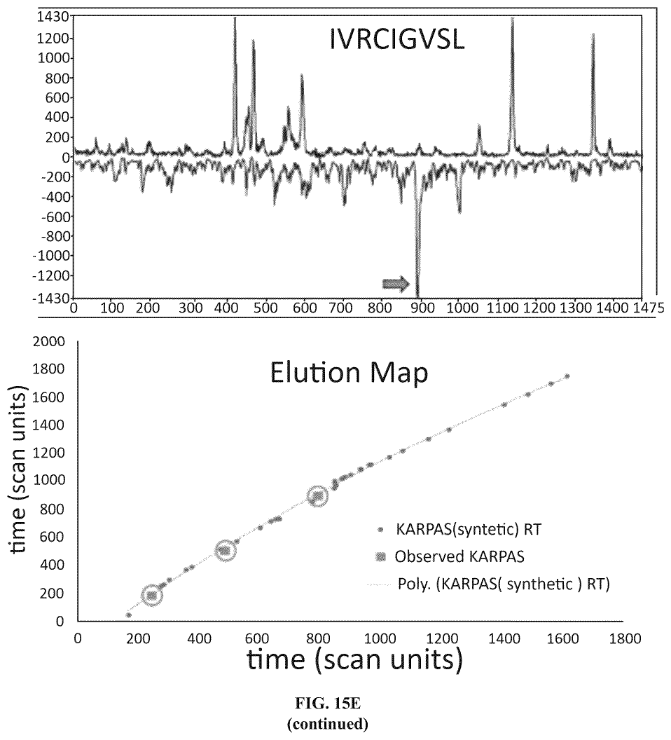

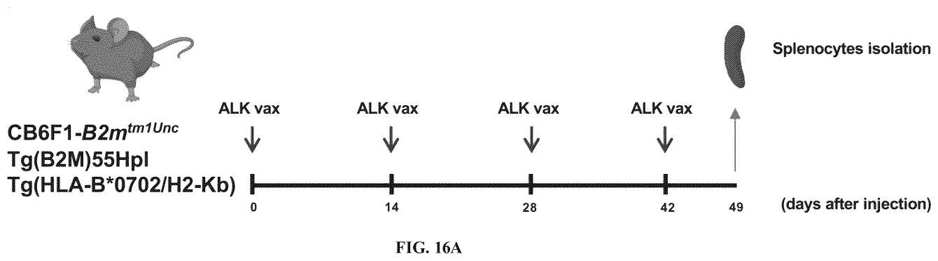

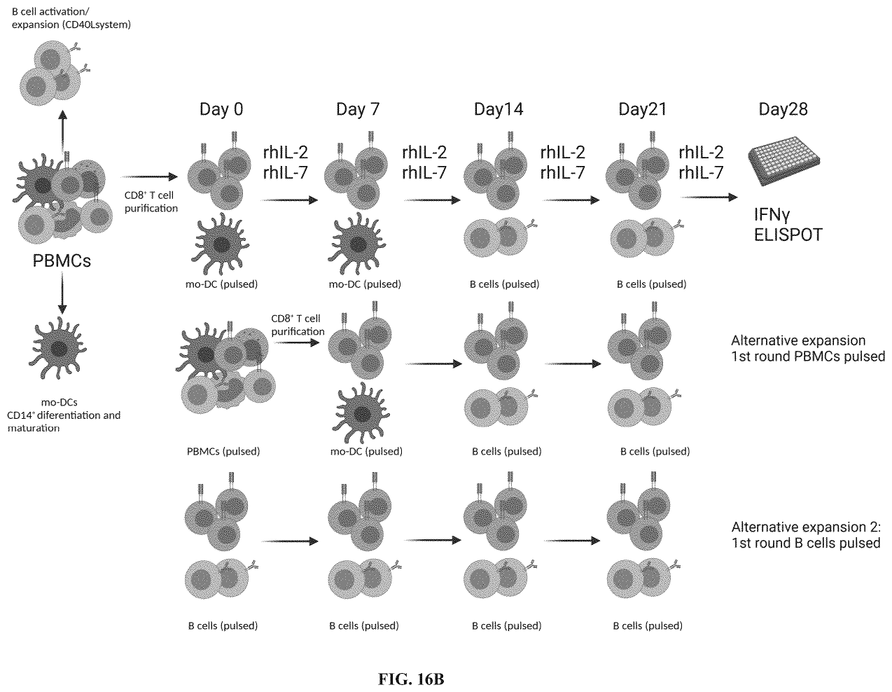

First, it was demonstrated that tumors from ALK⁺ NSCLC patients had a robust and homogenous expression of MHC-I molecules in most tumor cells (FIGS. 7A and 15A). Next, mass spectrometry was used to directly identify ALK peptides presented by human ALK⁺ tumor cell lines expressing HLA A*02:01 or HLA B*07:02 that were among the most frequent MHC-I alleles in a series of 100 NSCLC patients (Table 3). First, two ALK+ lymphoma cell lines DEL and Karpas-299 were used because ALK+ lymphoma is known to be immunogenic and expresses high levels of the ALK fusion. DEL expresses HLA A*02:01 while Karpas-299 expresses HLA B*07:02 (Table 4). The targeted LC-MS/MS analysis of eluted peptides that immunoprecipitated with HLA A*02:01 molecule in DEL cells yielded the AMLDLLHVA (SEQ ID NO: 7) but not the SLAMLDLLHV (SEQ ID NO: 110) peptide, despite both were previously shown to be recognized by CD8⁺ T cell from lymphoma patients (FIG. 15B). For the HLA B*07:02 allele, 3 novel ALK peptides (RPRPSQPSSL (SEQ ID NO: 3), IVRCIGVSL (SEQ ID NO: 4), and VPRKNITLI (SEQ ID NO: 5)) were identified in the discovery LC-MS/MS analysis carried on the pan-HLA eluates from Karpas-299 cells (FIG. 15C). These ALK peptides were unequivocally assigned by the NetMHCpan4.1 algorithm to the HLA-B*07:02 allele present in these cells (Tables 5A-5C). These same peptides were further identified by an independent method of ultra-low flow liquid chromatography-data independent acquisition MS (LC-DIAMS) (Keskin D B, Reinhold B, Lee S Y, Zhang G, Lank S, O'Connor D H, et al. Direct identification of an HPV-16 tumor antigen from cervical cancer biopsy specimens. Frontiers in immunology 2011; 2:75 doi 10.3389/fimmu.2011.00075; Reinherz E L, Keskin D B, Reinhold B. Forward Vaccinology: CTL Targeting Based upon Physical Detection of HLA-Bound Peptides. Frontiers in immunology 2014; 5:418 doi 10.3389/fimmu.2014.00418) in DEL and Karpas-299 cells (FIGS. 15D and 15E), confirming that these four as the only ALK peptides identified in ALK+ lymphoma cells. To validate this discovery in ALK+ lung cancer cells, LC-DIAMS was applied to MHC-I eluates from the NCI-H2228 cell lines that express the EML4-ALK fusion and both HLA-A*02:01 and HLA-B*07:02 alleles (Table 3) as well as to eluates obtained from a tumor biopsy of a HLA-B*07:02 ALK⁺ NSCLC patient. LC-DIAMS promptly identified the RPRPSQPSSL (SEQ ID NO: 3) peptide in NCI-H2228 cells (FIG. 7B) and the RPRPSQPSSL (SEQ ID NO: 3) and VPRKNITLI (SEQ ID NO: 5) peptides in the tumor biopsy (FIG. 7C), while it did not identify the AMLDLLHVA (SEQ ID NO: 7) and the IVRCIGVSL (SEQ ID NO: 4) peptides possibly due to detection limits. Also, peptides were not identified from the EML4-ALK chimeric junction. Thus, four ALK peptides that are processed and presented by the HLA A*02:01 and HLA B*07:02 alleles were discovered and validated in multiple ALK⁺ cell lines and in an ALK⁺ Non-Small Cell Lung Cancer (NSCLC) patient.

Table 5D provides predictions of hEML4-ALK fusion junction peptides binding prediction of hEML4-ALK fusion junction peptides binding to HLA-A*02:01, -B*07:02, and -A*03:01.

TABLE 3
HLA frequencies in a cohort of 100 patients

| HLA A | | HLA B | | HLA C | |
|---|---|---|---|---|---|
| Allele | Frequency | Allele | Frequency | Allele | Frequency |
| *02:01 | 0.43 | *07:02 | 0.18 | *07:01 | 0.27 |
| *01:01 | 0.26 | *08:01 | 0.15 | *04:01 | 0.26 |
| *24:02 | 0.24 | *35:01 | 0.12 | *06:02 | 0.24 |
| *03:01 | 0.22 | *18:01 | 0.1 | *07:02 | 0.22 |
| *11:01 | 0.12 | *44:02 | 0.1 | *03:04 | 0.15 |

TABLE 4
HLA haplotypes from ALK⁺ cell lines

| Cell line | HLA A | HLA B | HLA C |
|---|---|---|---|
| KARPAS-299 | *03:01*11:01 | *07:02*35:01 | *04:01*07:02 |
| DEL | *26:01*02:01 | *14:01*27:05 | *08:02*05:01 |
| NCI-H2228 | *03:XXł*02:01 | *38:01*07:02 | *07:02*12:03 |

łnull allele

TABLE 5A
HLA assignment of peptides identified
by LC-MS/MS in KARPAS-299 cells

| VPRNKNITLI | | |
|---|---|---|
| (SEQ ID NO: 111) | % Rank EL | Bind Level |
| HLA B*07:02 | 0.091 | Strong |
| HLA C*04:01 | 2.700 | |
| HLA B*35:01 | 3.100 | |
| HLA C*07:02 | 8.724 | |
| HLA A*03:01 | 25.286 | |
| HLA A*11:01 | 31.600 | |

TABLE 5B
HLA assignment of peptides identified
by LC-MS/MS in KARPAS-299 cells

| IVRCIGVSL | | |
|---|---|---|
| (SEQ ID NO: 4) | % Rank EL | Bind Level |
| HLA B*07:02 | 0.549 | Weak |
| HLA B*35:01 | 6.989 | |
| HLA C*04:01 | 7.600 | |
| HLA C*07:02 | 9.618 | |
| HLA A*03:01 | 13.074 | |
| HLA A*11:01 | 21.950 | |

TABLE 5C
HLA assignment of peptides identified
by LC-MS/MS in KARPAS-299 cells

| RPRPSQPSSL | | |
|---|---|---|
| (SEQ ID NO: 3) | % Rank EL | Bind Level |
| HLA B*07:02 | 0.001 | Strong |
| HLA C*07:02 | 0.750 | Weak |
| HLA C*04:01 | 1.157 | Weak |
| HLA B*35:01 | 1.830 | Weak |
| HLA A*03:01 | 10.709 | |
| HLA A*11:01 | 13.950 | |

TABLE 5D
Prediction of hEML4-ALK fusion junction peptides binding to HLA-A*02:01,
-B*07:02, and -A*03:01
NCI-H2228 EML4-ALK Variant 3a/b
...VTKTADKHKDVIINQVYRRKHQELQAMQMELQS (SEQ ID NO: 112)... 3a
...VTKTADKHKDVIINQAKMSTREKNSQVYRRKHQELQAMQMELQS (SEQ ID NO: 113)... 3b

| | netMHCpan 4.1 | | | | | |
|---|---|---|---|---|---|---|
| HLA-A*02:01 | % Rank | % Rank | HLAthena | | netMHC 4.0 | |
| Peptide | EL | BA | prank.Msi | prank.MSiC | % Rank | LC-DIAMS |
| VIINQVYRR | 10.92 | 28.06 | 14.98 | 11.65 | 32 | |
| (SEQ ID NO: | | | | | | |
| 114) | | | | | | |
| SQVYRRKHQEL | 22.12 | 41.61 | 22.58 | 19.51 | 70 | |
| (SEQ ID NO: | | | | | | |
| 115) | | | | | | |
| IINQVYRRK | 26.71 | 49.7 | 41.54 | 44.04 | 46 | |
| (SEQ ID NO: | | | | | | |
| 116) | | | | | | |
| REKNSQVYR | 30.91 | 69.29 | 59.04 | 66.45 | 95 | |
| (SEQ ID NO: | | | | | | |
| 117) | | | | | | |
| VIINQVYR | 31.85 | 47.91 | 3.5 | 1.3 | 60 | |
| (SEQ ID NO: | | | | | | |
| 118) | | | | | | |
| | netMHCpan 4.1 | | | | netMHC | |
| HLA-B*07:02 | % Rank | % Rank | HLAthena | | 4.0 | |
| Peptide | EL | BA | prank.Msi | prank.MSiC | % Rank | LC-DIAMS |
| STREKNSQVY | 7.37 | 9.88 | 8.69 | 5.05 | 15 | |
| (SEQ ID NO: | | | | | | |
| 119) | | | | | | |
| SQVYRRKHQEL | 8.49 | 9.13 | 7.74 | 8.72 | 18 | |
| (SEQ ID NO: | | | | | | |
| 115) | | | | | | |
| NQVYRRKHQEL | 11.59 | 13.63 | 13.16 | 16.33 | 18 | |
| (SEQ ID NO: | | | | | | |
| 120) | | | | | | |
| TREKNSQVY | 17.33 | 55.56 | 65.91 | 47.34 | 29 | |
| (SEQ ID NO: | | | | | | |
| 121) | | | | | | |
| KHKDVIINQVY | 22.88 | 71.35 | 10.06 | 12.28 | 60 | |
| (SEQ ID NO: | | | | | | |
| 122) | | | | | | |
| | netMHCpan 4.1 | | | | netMHC | |
| HLA-A*03:01* | % Rank | % Rank | HLAthena | | 4.0 | |
| Peptide | EL | BA | prank.Msi | prank.MSiC | % Rank | LC-DIAMS |
| IINQVYRRK | 0.272 | 0.511 | 0.62 | 0.86 | 0.7 | not detected |
| (SEQ ID NO: | | | | | | |
| 116) | | | | | | |
| VIINQVYRRK | 0.866 | 0.621 | 0.69 | 0.93 | 1 | not detected |
| (SEQ ID NO: | | | | | | |
| 123) | | | | | | |
| STREKNSQVYR | 1.5866 | 3.2285 | 1.92 | 0.84 | 5.5 | |
| (SEQ ID NO: | | | | | | |
| 124) | | | | | | |
| KNSQVYRRK | 2.8246 | 5.1992 | 4.58 | 4.57 | 4.5 | |
| (SEQ ID NO: | | | | | | |
| 125) | | | | | | |
| DVIINQVYRRK | 6.867 | 5.387 | 0.64 | 0.40 | 5.5 | not detected |
| (SEQ ID NO: | | | | | | |
| 126) | | | | | | |

*HLA-A*03:01 allele presents a 14 bp deletion in exon 1 that may affect protein expression in NCI-H2228 cells
Prediction threshold for binder peptides % Rank <1%
Bold: corresponds to hEML4 protein
Bold Underline: corresponds to hALK protein

Transgenic mice vaccinated with the AMLDLLHVA (SEQ ID NO: 7) developed ALK-specific immune responses (Passoni L, Scardino A, Bertazzoli C, Gallo B, Coluccia A M, Lemonnier F A, et al. ALK as a novel lymphoma-associated tumor antigen: identification of 2 HLA-A2.1-restricted CD8+ T-cell epitopes. Blood 2002; 99(6):2100-6). Therefore, experiments were undertaken to demonstrate the immunogenicity of the newly identified ALK peptides presented by the HLA B*07:02 allele. Transgenic mice expressing the human HLA B*07:02 were vaccinated with different peptides containing the core epitopes IVRCIGVSL (SEQ ID NO: 4) or RPRPSQPSSL (SEQ ID NO: 3): IVRCIGVSL (SEQ ID NO: 4) (IVRshort), FNHQNIVRCIGVSL (SEQ ID NO: 1) (IVRlong), RPRPSQPSSL (SEQ ID NO: 3) (RPRshort), GGDLKSFLRETRPRPSQPSSLAM (SEQ ID NO: 2) (RPRlong). ALK-specific CD8+ T cells responses were detected in 12/12 (100%) mice vaccinated with either the IVRshort peptide or the IVRlong peptide and in 6/12 (50%) of mice vaccinated with either the PRPshort peptide or the RPRlong (FIG. 7D). Finally, ALK-specific CD8+ T cells responses were also detected in peripheral blood mononuclear cells (PBMCs) from 3/6 (50%) ALK+ NSCLC patients when stimulated with the IVRCIGVSL (SEQ ID NO: 4) peptide but not with the RPRPSQPSSL (SEQ ID NO: 3) peptide (FIGS. 7E and 16B; Tables 6-7). Overall, four immunogenic ALK peptides in the context of the HLA A*02:01 and B*07:02 alleles were identified by mass spectrometry that can pave the way toward the design of a human ALK vaccine.

TABLE 6
HLA haplotypes and tumor driver mutation from patients
tested for ALK-specific immune responses

| | HLA A | HLA B | HLA C | Tumor driver |
|---|---|---|---|---|
| Patient 1 | *02:01*30:01 | *07:02*13:02 | *07:02*06:02 | ALK |
| Patient 2 | *01:01*02:01 | *07:02*08:01 | *05:01*07:01 | ALK |
| Patient 3 | *02:01*01:01 | *51:01*07:02 | *05:01*07:02 | ALK |
| Patient 4 | *02:01*09:02 | *07:02*44:03 | *07:02*16:01 | ALK |
| Patient 5 | *01:01*02:01 | *07:02*08:01 | *07:01*07:02 | KRASG12V |
| Patient 6 | *03:01*32:01 | *07:02*35:03 | *04:01*07:02 | EGFR del exon19 |
| Patient 7 | *03:01*02:05 | *07:02*50:01 | *07:02*06:02 | KRASG12D |
| Patient 8 | *03:01*80:01 | *07:02*44:03 | *07:02*04:01 | EGFR del exon19 |

TABLE 7
Summary of ELISPOT results and method applied in ALK-specific T cell expansion
Average Spots Forming Units (2 wells)/20 000 CD8⁺ T cells

| | RPRPSQPSSL (SEQ ID | | | | | IVRCIGVSL (SEQ ID | | | | |
|---|---|---|---|---|---|---|---|---|---|---|
| | NO: 3) expanded | | | | | NO: 4) expanded | | | | |
| ELISPOT | vehicle | IVR | RPR | CEF+ | Comments | vehicle | IVR | RPR | CEF+ | Comments |
| Patient 1 | 4 | 5.5 | 4.5 | 170.5 | PBMCs | 5 | 24 | 5.5 | 140 | PBMCs |
| | | | | | pulsed 1st | | | | | pulsed 1st |
| | | | | | round | | | | | round |
| Patient 2 | 1.5 | 1.5 | 0 | 28 | PBMCs | 20.5 | 56 | 27.5 | 114 | B cells |
| | | | | | pulsed 1st | | | | | pulsed 1st |
| | | | | | round | | | | | round |
| Patient 3 | 4.5 | 4 | 3 | 243.5 | PBMCs | 12 | 12 | 6.5 | 87.5 | B cells |
| | | | | | pulsed 1st | | | | | pulsed 1st |
| | | | | | round | | | | | round |
| Patient 4 | 3 | 0 | 2 | 51.5 | PBMCs | No tested | | | | |
| | | | | | pulsed 1st | | | | | |
| | | | | | round | | | | | |
| Patient 5 | 1 | 1 | 1 | 138.5 | PBMCs | No tested | | | | |
| | | | | | pulsed 1st | | | | | |
| | | | | | round | | | | | |
| Patient 6 | 13 | 11.5 | 10 | 195.5 | moDC | 1 | 146 | 1.5 | 16.5 | moDC |
| | | | | | pulsed 1st | | | | | pulsed 1st |
| | | | | | round | | | | | round |
| Patient 7 | 2.5 | 7.5 | 3 | 8 | moDC | 2 | 0.5 | 1 | 381.5 | moDC |
| | | | | | pulsed 1st | | | | | pulsed 1st |
| | | | | | round | | | | | round |
| Patient 8 | 10.5 | 9.5 | 10 | 88 | moDC | 1.5 | 2 | 2 | 108.5 | moDC |
| | | | | | pulsed 1st | | | | | pulsed 1st |
| | | | | | round | | | | | round |

### Example 8. Lack of Detectable Toxicity of the Alk Vaccine

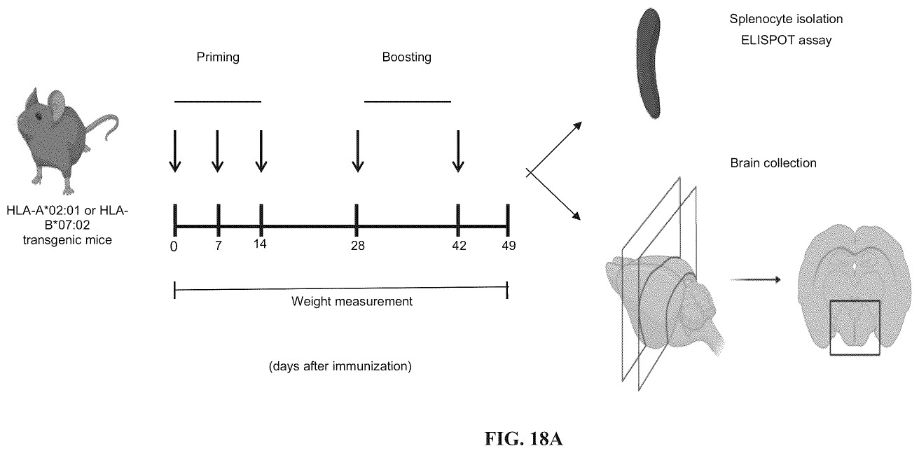

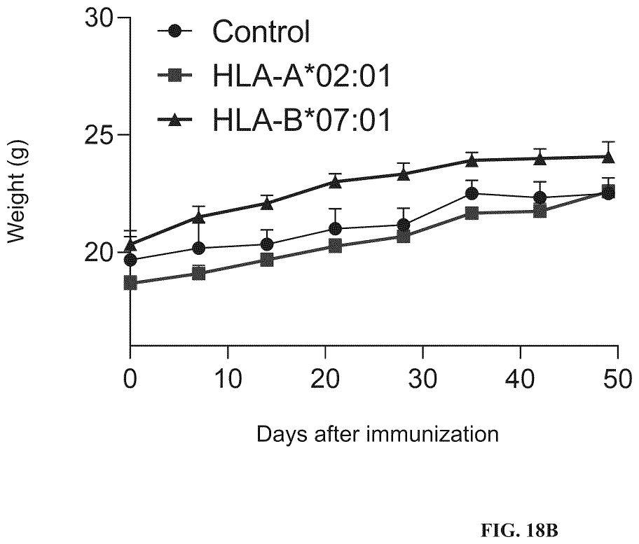

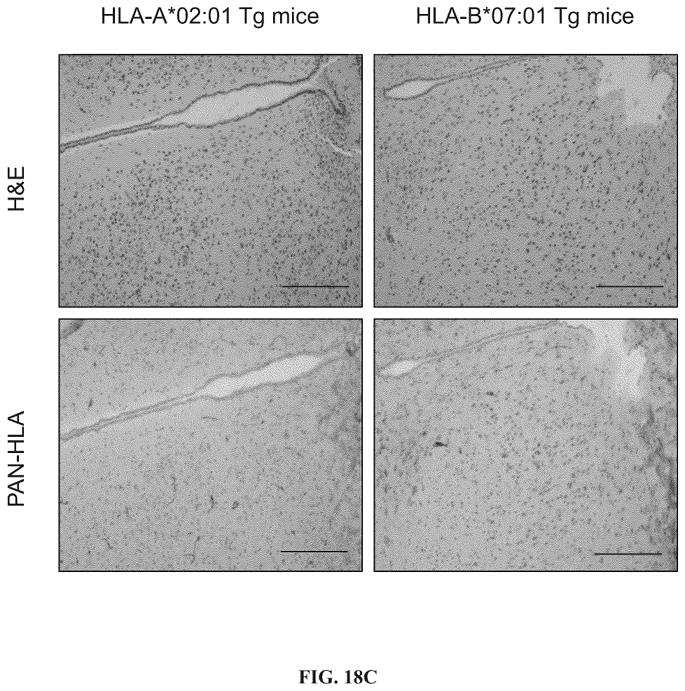

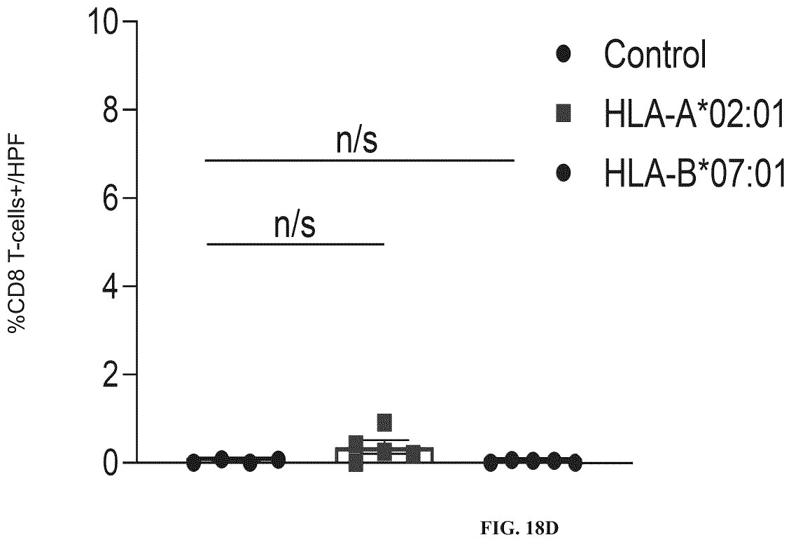

Transgenic mice (HLA-A*02:01 and HLA-1B*07:02) were vaccinated as shown in the schematic in FIG. 18A with either AMLDLLHVA (SEQ ID NO: 7) or IVRCIGVSL (SEQ ID NO: 4) peptides, respectively, and cyclic dinucleotides (CDN) adjuvant. Weight measurements were taken at discrete time points. Next, splenocytes were isolated and assayed and brain tissue was sectioned and imaged (FIG. 18C). Weight gain for the vaccinated mice was the same as the control mice as shown in FIG. 18B. H&E and pan-HLA immunohistochemistry staining in of the hypothalamus region from HLA-A*02:01 and HLA-1B*07:02 transgenic mice showed healthy tissue (FIG. 18C) in the vaccinated mice from FIG. 18A. Not a significant amount of CD8⁺ T cells were found in the hypothalamus region from HLA-A*02:01 and HLA-1B*07:02 transgenic mice (FIG. 18D), which were vaccinated as in FIG. 18A. In sum, this data showed that there was no detectable toxicity found in vaccinated mice.

In the above Examples, immunogenic anaplastic lymphoma kinase (ALK) peptides were identified and ALK-specific CD8⁺ T-cell responses were tracked in mouse models of ALK lung tumors to demonstrate that the poor immunogenicity of ALK-rearranged Non-Small Cell Lung Cancer (NSCLC) can be restored by enhancing the priming of ALK-specific CD8⁺ T-cells by vaccination. Vaccination with one single ALK peptide increased the intratumoral ALK-specific CD8+ T cells, delayed tumor progression extending the overall survival, cured a subset of mice in combination with treatment with the ALK tyrosine kinase inhibitor (TKI) lorlatinib while preventing the metastatic spread of ALK+ tumors cells.

The lack of response of ALK-rearranged Non-Small Cell Lung Cancer (NSCLC) to immune checkpoint inhibitors (ICIs) is still poorly understood. ALK-rearranged NSCLC typically have a low tumor mutational burden (TMB) and low levels of CD8⁺ tumor-infiltrating T lymphocytes (TILs) suggesting a poor immunogenicity that might be due to low numbers of neoantigens capable of inducing functional T-cells responses. Most ALK-rearranged NSCLC express PD-L1, that is considered a predictive factor for ICI response, but it might not reflect the presence of an intratumoral T-cell function but rather represents a direct regulation of PD-L1 expression by the ALK oncogenic activity. Alternatively, ALK-rearranged NSCLC could have a non-favorable tumor microenvironment that is only partially modified by ALK TKI treatment.

Anaplastic lymphoma kinase (ALK) itself is an immunogenic protein that induces spontaneous B- and T-cell responses in patients, including ALK-specific CD8⁺ T-cell responses. Therefore, it is unclear why ICI does not unleash these ALK-specific responses in ALK-rearranged Non-Small Cell Lung Cancer (NSCLC). Through the identification of one CD8⁺ ALK epitope presented in H2-Dd of BALB/c mice, it was found that the ALK-specific CD8⁺ T-cell response generated by ALK tumors localized in the flank is stronger than that generated by tumors in the lung. This difference between flank and lung tumors in the priming of ALK-specific CD8⁺ T-cell translated into a different response to ICI because ICI induced rejection of flank tumors but not lung tumors (FIG. 3). Thus, tumor localization is a critical factor for ALK-specific CD8⁺ T-cell priming.

In the above Examples, it is demonstrated that vaccination with one ALK immunogenic peptide induces stronger systemic CD8⁺ T-cell responses than those spontaneously elicited by ALK+ lung tumors (FIG. 4A), resulting in higher intratumoral CD8⁺ T-cell infiltration (FIG. 4B), and consequently in a superior therapeutic activity (FIG. 4C). Indeed, the advantage of peptide vaccination compared to immune checkpoint inhibitors (ICIs) therapy can be tumor and site dependent. For example, no significant differences in therapeutic activity were observed in models of sarcoma injected subcutaneously in the flank between ICI and neoantigen vaccination. Whether this therapeutic efficacy in the models used in the above Examples is simply due to an increased number of ALK-specific-TILs or to a more functional priming of CD8 T cells by the vaccine compared to spontaneous responses remains to be determined. ALK-specific CD8⁺ lung tumor-infiltrating T lymphocytes (TILs) in vaccinated mice not only were numerically increased but also showed a reduction of PD-1 expression compared to spontaneous ALK-specific TILs (FIG. 4E), suggestive of a less exhausted or dysfunctional phenotype.

A superior antitumoral-activity of anti-CTLA-4 compared to anti-PD-1 was observed not only when administered as monotherapy against subcutaneous tumors (FIG. 3G) but also in combination with the ALK vaccine against lung tumors (FIG. 4E). CTLA-4 treatment also helped to generate a stronger ALK-specific CD8+ T cell memory response that protected mice when they were re-challenged by tumors injected in the flank (FIG. 3E) or in the lungs (FIG. 3H). Likewise, ALK-specific CD8+ T cell memory response was stronger in tumor-free mice that were vaccinated together with anti-CTLA-4 treatment (FIGS. 4E-4F).

Anaplastic lymphoma kinase (ALK)-rearranged Non-Small Cell Lung Cancer (NSCLC) patients have higher incidence of central nervous system (CNS) metastasis compared to other NSCLC. CNS metastases respond well to second and third generation ALK tyrosine kinase inhibitors (TKIs) but remain a poor prognostic indicator. Interventions to control intracranial disease are critical to extend patient survival. It is shown in the above Examples that ALK vaccination, but not the spontaneous immunogenicity of lung tumors, induced an ALK-specific immune response that completely prevented the metastatic spread of ALK⁺ tumor cells to the brain. The prevention of metastatic spread was complete in all mice treated with a combination of ALK TKI and ALK vaccine with any ICI therapy, but a partial protection was also observed in mice treated with ALK TKI and anti-CTLA-4 (FIG. 5A-5B), indicating, without intending to be bound by theory, that prevention of metastatic spread likely correlates with the quantity and quality of ALK-specific CD8⁺ T-cell effector priming.

Loss of MHC Class-I molecules is one mechanism of immune evasion by which the presentation of specific antigens by tumor cells is reduced or lost. Focal HLA allele loss of heterozygosity (LOH) occurs in 40% of NSCLC while selective loss of an HLA allele can be observed as a direct mechanism of tumor immune evasion against specific peptides. In the models used in the above Examples, the ALK protein was not lost in tumors that escaped after treatment with ALK TKI and the ALK vaccine (FIGS. 14A and 14B), which was expected due to the dependency of ALK-rearranged NSCLC on the ALK oncogenic activity. In contrast, while not intending to be bound by theory, lung tumors evaded the immune system by downregulation of the expression of MHC-I molecules (FIG. 6B), while the other components of the antigen presenting machinery were unchanged (FIGS. 14E-14K). MHC-I downregulation, although not complete, was sufficient to impair the rejection of flank tumors treated with ICI (FIGS. 6D-6G). Importantly, MHC-I downregulation was reversible upon treatment with IFN-γ (FIG. 6C) or STING agonist (FIG. 6H), suggesting that a combination treatment with STING agonists (Le Naour J, Zitvogel L, Galluzzi L, Vacchelli E, Kroemer G. Trial watch: STING agonists in cancer therapy. Oncoimmunology 2020; 9(1):1777624 doi 10.1080/2162402X.2020.1777624) during or after ALK vaccination could further delay immune evasion by restoring MHC-I expression and the presentation of ALK antigens.

Based on these findings, the development of a clinical ALK vaccine is attractive given the known toxicities of immune checkpoint inhibitors (ICIs) when associated with anaplastic lymphoma kinase (ALK) treatment with ALK tyrosine kinase inhibitors (TKIs). MHC-I expression was conserved in ALK-rearranged NSCLC (FIGS. 7A and 15A) and ALK expression is not lost in tumors that become resistant to ALK TKI, which, while not intending to be bound by theory, suggests that ALK vaccination can be an approach to enhance immunotherapy against ALK-rearranged NSCLC that develop resistance to ALK TKI. ALK immunogenic peptides were identified in the context of two frequent HLA alleles (HLA A*02:01 and B*07:02) presented by ALK⁺ lymphoma cells, by ALK⁺ lung cancer lines, and in a tumor patient biopsy (FIGS. 7 and 15). In ALK⁺ lung cancer cells, two of the four ALK peptides identified in ALK⁺ lymphoma cells were identified, possibly due to the abundance of ALK protein that is much higher in lymphoma cells. Alternatively, ALK peptides could be processed differently in lymphoma cells compared to lung cancer cells, reflecting a tissue-specific processing of antigens. Vaccination with two of these ALK peptides in HLA transgenic mice induced ALK-specific CD8⁺ T-cells responses (FIG. 7D) and immune responses to the IVRCIGVSL (SEQ ID NO: 4) peptide was detected in NSCLC patients (FIGS. 7D, 14, and 15). The identification of the ALK peptides in human HLAs paves the way for the clinical development of an ALK vaccine and the development of immunotherapies such as transfer of T cells redirected with ALK-T cell receptors.

The following materials and methods were employed in the above examples.

### Patients and Sample Collection

Non-small cell lung cancer (NSCLC) patients, at the Dana-Farber Cancer Institute, consented to an institutional review board (IRB)-approved correlative research protocol that allowed for review of medical records and sample collection. Lung cancer mutation status was determined using standard CLIA-certified clinical assays in the Center for Advanced Molecular Diagnostics at Brigham and Women's Hospital. For each patient, 10 mL of whole blood was collected into K3-EDTA tubes, and peripheral blood mononuclear cells (PBMCs) (peripheral blood mononuclear cells) were isolated with Ficoll-Paque Plus density separation (GE Healthcare), and consequently frozen until use.

### Human and Mouse Cell Lines

Human ALK-rearranged NSCLC cell lines (inv(2)(p21;p23)—NCI-H3122—variant 1, E13;A20; NCI-H2228—variant 3, E6;A20), and human ALK-rearranged ALCL cell line (DEL and Karpas) were obtained from ATCC collection; the murine ASB-XIV cell line, derived from BALB/c mice was purchased from Cell Lines Service (CLS), and the murine KP1233 lung cancer cell line, immortalized from C57BL/6 KRASG12D mice, was kindly gifted by Tyler Jacks (Koch Institute, Cambridge, MA). HEK-293FT packaging cells were used for lentivirus production, and obtained from ATCC collection. NIH-3T3-hCD40Ligand cell line was kindly gifted by Gordon Freeman (Dana Farber Cancer Institute, Boston, MA). All cell lines were passaged for less than 6 months after receipt and resuscitation and maintained either in DMEM (Lonza) (NCI-H3122, NCI-H2228, ASB XIV, KP1233, and HEK-293FT) or in RPMI (Lonza) (DEL and Karpas-299) with 10% fetal bovine serum (FBS—Gibco), 2% penicillin, streptomycin 5 mg/mL (Gibco), and 1% glutamine (Gibco), and were grown at 37° C. in humidified atmosphere with 5% CO₂. NIH-3T3-hCD40L cells were cultivated in DMEM/F12 HEPES (Gibco) 10% FBS, gentamycin (15 μg/mL, Gibco) and G418 (200 μg/mL, Gibco). All cell lines were monitored for mycoplasma by IDEXX BioAnalytics (Impact III PCR profile).

### Generation of Eml4-Alk Murine Cell Lines.

The immortalized murine cell lines mEml4-Alk¹ and mEml4-Alk² were obtained from BALB/c TP53 KO mice infected with adenovirus carrying CRISPR/Cas9 system (sgRNA Eml4 and sgRNA Alk) as described in Maddalo D, Manchado E, Concepcion C P, Bonetti C, Vidigal J A, Han Y C, et al. In vivo engineering of oncogenic chromosomal rearrangements with the CRISPR/Cas9 system. Nature 2014; 516(7531):423-7 doi 10.1038/nature13902; and Blasco R B, Karaca E, Ambrogio C, Cheong T C, Karayol E, Minero V G, et al. Simple and rapid in vivo generation of chromosomal rearrangements using CRISPR/Cas9 technology. Cell reports 2014; 9(4):1219-27 doi 10.1016/j.celrep.2014.10.051. Primary cultures were established using the Lung Dissociation Kit (Miltenyi Biotec) according to the manufacturer's instructions, cultured primarily in 3D culture and finally in 2D culture. The immortalized humanized murine cell lines Eml4-Alk^{PGPGRVAKI-1} (“PGPGRVAKI” disclosed as SEQ ID NO: 22) and Eml4-Alk^{PGPGRVAKI-2} (“PGPGRVAKI” disclosed as SEQ ID NO: 22) were derived from mEml4-Alk¹. To avoid Cas9 off-targets, electroporation of short lifetime recombinant Cas9 protein was performed. Recombinant Cas9 protein was mixed with tracrRNA and crRNA (TTGCTATTCTTCCAGCTCCT (SEQ ID NO: 127)) (IDT) to generate ribonucleoproteins (RNPs). RNPs were then transfected by electroporation into mEml4-Alk¹ together with the ssODN (TATGAAATTAAGAACCCTGTTTTCTTCCCAGGGATATTGCTGCTAGAAACTGTCTGTTGACCTG CCCAGGTCCGGGAAGAGTAGCAAAGATTGGAGACTTTGGGATGGCCCGAGATATCTA (SEQ ID NO: 128), IDT) carrying the edited sequence, using the SE Cell line 4D-Nucleofector X kit S (Lonza) and the program CM-137. After electroporation Scr7 (100 nM, Sigma) was used to inhibit non-homologous end joining and favor homologous recombination. Once recovered from electroporation, single cell clones were generated through consecutive dilutions and validated through DNA and RNA sequencing.

Generation of Ex Vivo Cells Lines from Established Lung Tumors from Eml4-AlkPGPGRVAKI Lines.

Mice lung lobules were harvest and individual lung tumors were isolated and mechanically disaggregated until a single cell suspension was obtained. Consecutively, cells were plated in 6-well plates in DMEM complete medium. After 15 days in culture and at least 3 passages, ex vivo cell lines were established. All murine cell lines were further tested for the presence of the murine and human EML4-ALK rearrangement and passaged for less than 6 months after primary culture. Cells were maintained in DMEM (Lonza) with 10% fetal bovine serum (FBS—Gibco), 2% penicillin, streptomycin 5 mg/mL (Gibco), 1% glutamine (Gibco), 1 mM of sodium pyruvate (Gibco), and 0.5 mM of non-essential amino acids (Gibco), and were grown at 37° C. in humidified atmosphere in 5% of C02. All cell lines were monitored for mycoplasma by IDEXX BioAnalytics (Impact III PCR profile).

### Prediction of ALK Peptides Binding the MHC-I Mouse Alleles

NetMHCpan4.1 and NetMHC4.0 algorithms (services.healthtech.dtu.dk/service.php?NetMHCpan-4.1 and services.healthtech.dtu.dk/service.php?NetMHC-4.0) were used to predict MHC-I binding (H-2K^{d} and H-2D^{d} alleles) for all possible 8- to 11-amino acid long sequences correspond to ALK peptides. Predicted MHC-I binders were selected based on their relative ranking in NetMHCpan4.1 (top 0.5% of ranked peptides were considered strong binders).

### Mice

Mouse strains used include transgenic SP-C-EML4-ALK and NPM-ALK expressing the human EML4-ALK (hEML4-ALK Tg mice) or NPM-ALK, respectively, as described in Voena C, Menotti M, Mastini C, Di Giacomo F, Longo D L, Castella B, et al. Efficacy of a Cancer Vaccine against ALK-Rearranged Lung Tumors. Cancer immunology research 2015; 3(12):1333-43 doi 10.1158/2326-6066.CIR-15-0089. BALB/c TP53KO, WT BALB/c, and NSG mice were purchased from Charles River. CB6F1-B2m^{tm1Unc} Tg(B2M)55Hpl Tg(HLA-B*0702/H2-Kb)B7 mice (HLA-B*07:02 transgenic mice) were purchased from Taconic. B6.Cg-Immp2lTg(HLA-A/H2-D)2Enge/J mice (HLA-A*02:01 transgenic mice) were purchased from the Jackson Laboratory. Ad-EA mice were generated by using CRISPR/Cas9 system to induce Alk rearrangements in vivo as previously described (26). Mice were housed in our specific-pathogen free animal facilities. In all in vivo experiments, 8-12-weeks old females were used and all studies were performed in accordance with procedures approved by either University of Turin, Turin, Italy or ARCH accredited Animal Studies Committee of Boston Children's Hospital, Harvard Medical School, Boston, USA.

### Magnetic Resonance Imaging (MRI)

Anatomical T₂-weighted coronal images were acquired with a respiratory-triggered multislice fast spin echo RARE sequence (TR=4 s, TE=4.5 ms, Rare Factor (RF)=16, FOV=30 mm², Matrix=256×256, slices=16-20, slice thickness=1 mm, 2 averages, providing an in-plane spatial resolution of 117 μm) with a 7T MRI (Bruker Advance III, Ettlingen, Germany) scanner equipped with a quadrature 1H coil. Animals were anesthetized by intraperitoneal injection of a combination of ROMPUN® (Bayer, 5 mg/kg) and ZOLETIL 100® (Laboratoires Virbac, 20 mg/kg); breathing was monitored during acquisition of the MR images with a respiratory sensor (SA Instruments, Inc.). Tumor volumes and numbers of masses calculations were performed by manual segmentation (slice by slice contouring) with ITK-SNAP software (www.itksnap.org).

### ALK Inhibitors

Crizotinib and lorlatinib were kindly gifted by Pfizer. Alectinib, ceritinib, and brigatinib were purchased from Selleckchem. For in vivo treatment, crizotinib and lorlatinib were administrated via oral gavage either once a day (DIE) or twice a day (BID) as indicated. Crizotinib was administrated for short-term treatment (15 days), and lorlatinib treatment was performed either in a short-term (15 days) or prolonged treatment (4 or 8 weeks) as indicated. Crizotinib was administered either at 40 mg/kg BID or higher dose (100 mg/kg DIE). Lorlatinib was administered either at 2 mg/kg B BID or at higher dose (10 mg/kg DIE); vehicle solution: 0.5% Methylcellulose (Sigma-Aldrich), 0.05% Tween-80 (Sigma-Aldrich).

### Immune Checkpoint Inhibitors

When using transgenic NSCLC mouse models, mice were treated intraperitoneally with 300 μg or 200 μg of anti-PD-1 (clone RMP1-14, Bioxcell), anti-PD-L1 (clone 10F.9G2, Bioxcell), and control anti-rat polyclonal IgG, administrated alone or in combination with ALK inhibitors (either crizotinib or lorlatinib) every 3 days or every week for a total of 5 injections. Syngeneic mice models transplanted with tumor cells were treated intraperitoneally with 200 μg of anti-PD-1 and/or anti-CTLA-4 (clone 9D9, Bioxcell), on days 3, 6, and 9 post-tumor transplantation (3 injections/per mouse). When combined with ALK inhibitor lorlatinib and/or vaccination, intraperitoneal injections were performed at day 6 post-tumor injection and synchronized with ALK inhibitor lorlatinib treatment.

### DNA and Peptide ALK Vaccination:

ALK-DNA vaccination was performed as described in Voena C, Menotti M, Mastini C, Di Giacomo F, Longo D L, Castella B, et al. Efficacy of a Cancer Vaccine against ALK-Rearranged Lung Tumors. Cancer immunology research 2015; 3(12):1333-43 doi 10.1158/2326-6066.CIR-15-0089. Briefly, 50 μg of control pDEST (Invitrogen) or pDEST-ALK vectors were diluted in 20 μL 0.9% NaCl with 6 mg/mL polyglutamate and injected on day 1 and day 7 into both tibial muscles of anesthetized BALB/c mice. Electric pulses were applied through two electrodes placed on the skin; two square-wave 25-ms, 375V/cm pulses were generated by a T820 electroporator (BTX) (67). ALK peptides were purchased from Genscript (NJ, USA). Peptide vaccine was prepared by mixing the corresponding peptide (10 μg) with CDN adjuvant (25 μg), according to manufacturer instructions. Mice were vaccinated subcutaneously with 100 μL of peptide vaccine. For amph-vaccination, peptides and CpG (adjuvant) were modified with an amphiphilic (amph) tail. 20 μg of amph-peptides were mixed with 1.24 nmol of amph-CpG were mixed and administered subcutaneously in the base of the tail.

### In Vivo Cytotoxicity Assay

In vivo cytotoxicity assays were performed as described in Voena C, Menotti M, Mastini C, Di Giacomo F, Longo D L, Castella B, et al. Efficacy of a Cancer Vaccine against ALK-Rearranged Lung Tumors. Cancer immunology research 2015; 3(12):1333-43 doi 10.1158/2326-6066.CIR-15-0089. Briefly, vehicle-vaccinated and ALK-vaccinated mice (both peptide and ALK-DNA vaccinated mice) were injected intravenously with 1×10⁷ WT splenocytes mixed with 1×10⁷ NPM-ALK Tg splenocytes labeled with different concentration of CFSE (0.5 μM and 5 μM, respectively). After 72 hours, CFSE⁺ CD4⁺ splenocytes were stained with TRITC-labeled anti-CD4 and analyzed by flow cytometry. ALK directed specific cytotoxicity was calculated as the decrease in ALK+CD4+ T cells (CFSE^{high}) after normalization with the total number of CD4+CFSE+ T cells.

### Tumor Grafting

For syngeneic subcutaneous tumor transplantation, a total of 1×10⁶ immortalized mouse cells were subcutaneously inoculated into the right dorsal flanks of 8-12 week-old BALB/c mice in 100 μL of phosphate-buffered saline (PBS). Subcutaneous tumor-bearing mice were randomized and grouped into different treatment groups when tumors reached 5 mm diameter. Tumor volume was measured by caliper measurements every 3 days in a blinded fashion and calculated according to the following equation: H. W²/2. In accordance with a mouse protocol, maximal tumor diameter was 15 mm (humane endpoint) in one direction, dictating the end of the experiment. For orthotopic syngeneic mouse model, a total of 1×10⁶ immortalized mouse cells were inoculated intravenously into the tail vein of 8-12-week-old BALB/c mice in 100 μL of PBS. Mice were randomized into different treatment groups. In accordance to the mouse protocol, the humane endpoint was reached when mice presented difficulty breathing, lost locomotor activity, lost body weight and/or presented an abnormal coat condition or posture. For the rechallenge study, mice were injected either subcutaneously in the opposite flank or intravenously with 10⁶ immortalized mouse cells in 100 μL of PBS and monitored as described above.

### Tissue, Tumor, Blood Collection and Metastasis Assay

For histologic evaluation, lung lobules were collected, fixed in formalin and embedded in paraffin as described in Voena C, Menotti M, Mastini C, Di Giacomo F, Longo D L, Castella B, et al. Efficacy of a Cancer Vaccine against ALK-Rearranged Lung Tumors. Cancer immunology research 2015; 3(12):1333-43 doi 10.1158/2326-6066.CIR-15-0089. T lymphocytes were quantified by high power field by measuring the number of CD8⁺ T cells among total number of tumor cells. For flow cytometry and/or ex vivo experiments, both subcutaneous tumor and lung lobules were collected and either mechanically disaggregated or dissociated into mouse tumor cell suspensions using the mouse Tumor Disassociation Kit 651 (Cat #130-096-730, Miltenyi Biotec, Bergisch Gladbach, Germany), according to the manufacturer's protocol. After RBC lysis and filtration, cell suspensions were stained for live/dead cells with Zombie Aqua (Zombie Aqua BV510, Biolegend, Cat #423101/423102) and subjected to flow cytometry. Total blood was collected from the venous sinus into a BD VACUTAINER™ 2 mL Blood Collection tube with K₃ EDTA. For the metastatic assay, brains were collected, fixed in formalin, and embedded in paraffin and several tissue sessions were stained for H&E and analyzed for micrometastases.

### Enzyme-Linked Immunosorbent Spot Assay (ELISPOT Assay).

The interferon-γ release enzyme-linked immune absorbent spot (ELISPOT) assay was performed using a commercial kit (Mouse IFN-γ ELISPOT, Mabtech, Stockholm, Sweden) according to the manufacturer's instructions. Briefly, the ELISPOT plate was prepared in sterile conditions and washed with sterile PBS (200 μL/well) for 5 times. Consecutively, the plate was conditioned with fresh DMEM medium (200 μL/well) contained 10% of the same fetal bovine serum used for the splenocytes suspension and incubated for 30 minutes at room temperature. After incubation, the medium was discarded and 2.5×105 splenocytes were plated in each well together with the appropriate stimuli. The plate was incubated over/night at 37° C. in humidified conditions with 5% C02. The day after, cells were discarded, and the plate was washed 5 times with PBS. Biotinylated detection anti-IFN-γ mAb (1 μg/mL) was added into the wells, followed by 2 hours of incubation at room temperature. Successively, and after another wash step, the plate was then incubated for a further 1 hour at room temperature with diluted streptavidin-ALP (1:1000) in PBS-0.5% FCS at 100 μL per well. Finally, the plate was washed again for 5 times with PBS, followed by the addition of substrate solution BCIP/NBT-plus. Tap water was used to stop the reaction when distinct spots appeared. All plates were evaluated by a computer assisted ELISPOT reader (CTL Immunospot analyzer, OH, USA).

### Intracellular Cytokine Staining (ICS)

Mice were bled and 100-200 μL of peripheral blood was lysed with red blood cell ACK lysis buffer. PBMCs were plated in U-bottom 96 well plates with T-cell media (RPMI 10% FBS, Penicillin/Streptomycin, Glutamine, and HEPES 15 mM) and pulsed with 10 nmol of individual peptides. After 2 hrs, Brefeldin A was added (BD Cyotfix/Cytoperm plus kit, BD Pharmigen) and incubated for 4 hrs. PBMCs were then washed with FACs buffer and incubated with Fc blocker (1:100, CD16/CD32 Mouse Fc Block) for 10 min at room temperature before staining with PE-CD4 (1:100; GK1.5, Miltenyi) and FITC-CD8 (1:100; clone 53-6.7, Miltenyi) at 4° C. for 20 min. After washing with FACS buffer, cells were fixed with BD Cytofix/Cytoperm fixation solution for 20 min at 4° C. and washed with BD Perm Wash buffer (BD Cytofix/Cytoperm plus kit, BD Pharmigen) before incubating with APC anti-IFN-γ (1; 50 in BD Perm Wash buffer, BD Pharmigen) for 30 min. at 4° C. Finally, cells were analyzed by flow cytometry once washed with BD Perm Wash Buffer and FACs buffer consecutively.

### H-2D^{d}-PGPGRVAKI (SEQ ID NO: 22) Dextramer Staining and Flow Cytometry

Allophycocyanin (APC)-conjugates H-2D^{d}-PGPGRVAKI (SEQ ID NO: 22) Dextramer reagents were obtained from Immudex (Immudex, Denmark). Briefly, 1×10⁶ cells (either from total splenocytes or total subcutaneous tumor and/or lung disaggregation) were stained with Zombie Aqua (Biolegend, USA) viability marker for 30 minutes at room temperature. After this initial step, cells were treated with 50 nM of dasatinib at room temperature for 30 minutes. Dextramer staining was performed together with mouse Fc block for 20 minutes at room temperature protected from light. Without prior washing, cells were finally stained with mouse FITC-CD8 conjugated (clone G42-8, BD PHARMINGEN™, USA) antibody for 10 minutes at 4° C. After washing step, cells were ready to be acquired. When referred, also PD-1 (clone RMP1-30; BV421-PD-1, BD Pharmingen™, USA) expression was evaluated together with dextramer staining, being added together with FITC-CD8. H2-Dd MHC-I expression was measured on relapsed tumors after treatment. Tumor lungs were isolated and cultured ex vivo until primary cultures were stabilized. Briefly, cells seeded in DMEM complete medium were detached by using cold PBS. Resuspended cells were then stained with APC-anti-H-2Dd (clone 34-1-2S; ThermoFisher) for 20 minutes, washed and resuspended again in PBS. All cells were acquired in a BD Celesta flow cytometer (BD Bioscience, USA) and analyzed by using the FlowJo software (FlowJo LCC, USA).

### Compounds and Treatments

Recombinant IFN-γ (murine IFN-γ [Cat #794 485-MI]was purchased from R&D Systems 795 (Minneapolis, MN) and reconstituted in 1% BSA. Regarding STING agonism experiments, cells were treated with ADU-S100 (50 μM, Cat #CT-ADUS100, ChemieTek, Indianapolis, IN) for 24 h. Flow cytometry analysis of H2-Dd surface expression was performed as a readout for both experiments as described above (PE-H-2D^{d}; 34-2-12, BD Pharmigen).

### Peptide Binding Assay

To generate knock-out the Tap2 gene in ASB-XIV cell line CRISPR/Cas9 technology was used with sgRNAs (ATAGAGGGCACCCTGCGACT (SEQ ID NO: 129) or GAGCACCTCAGTAGTCCGAG (SEQ ID NO: 130)) targeting Tap2 exon II that were cloned into lentiCRISPR v2 (Addgene, #52961). After lentiviral infection and puromycin selection, single-cell clones were obtained through consecutive dilutions and H-2-Dd and H-2K^{d} expression were analyzed by flow cytometry (PE-H-2D^{d}; clone 34-2-12, BD Pharmigen; PE-H-2K^{d}; clone SF1-1.1, BD Pharmigen) to evaluate the downregulation of MHC-I. The H-2K^{d} associated peptide FYIQMCTEL (SEQ ID NO: 131) (IEDB, epitope 18405) was used to validate the ASB-XIV-TAP2KO tool in a binding assay before evaluating PGPGRVAKI (SEQ ID NO: 22) binding. Briefly, cells were incubated with different concentrations of peptide at 26° C. for 16 h and then at 370 for 3 h. Cells were then washed, detached with 2 mM EDTA, stained and analyzed for H-2-Dd and H-2-Kd surface expression by flow cytometry.

### Cell Lysis and Immunoblotting

Cells were lysed in GST buffer (10 mM MgCl2, 150 mM NaCl, 1% NP40, 2% Glycerol, 1 mM EDTA, 25 mM HEPES pH7.5), with protease inhibitors [1 mM phenylmethylsulfonyl fluoride (PMSF), 10 mM NaF, 1 mM Na3VO4, and protease inhibitor cocktail (Amresco)]. The following antibodies were used: anti-pALK (Y1586) (Cell Signaling Technology, USA); anti-ALK SP8 16670 (Abcam, UK) and anti-Actin (Sigma, USA).

### DNA and RNA Extraction, PCR and Quantitative RT-PCR, Sanger Sequencing

DNA and RNA were extracted as described in Voena, et al., “Efficacy of a Cancer Vaccine against ALK-Rearranged Lung Tumors,” Cancer Immunol Res., 3:1333-1343 (2015), PMID: 26419961. PCR reactions were established to detect both genomic DNA and cDNA of peptide7. PCR products were purified using the QIAquickPCR Purification Kit (QIAGEN, USA) and the amplicons were sequenced by GeneScript Company (USA). Sanger sequencing were analyzed with SnapGen software (SnapGen, USA). (Primers: ALK peptide 7 gDNA, For: TATGAAGGCCAGGTGTCTGGAATGC (SEQ ID NO: 132); Rev GACAAACTCCAGAACTTCCTGGTTGC (SEQ ID NO: 133))(Primers: ALK peptide7 cDNA, For: ACCTCGACCATCATGACCGACT (SEQ ID NO: 134); Rev: ACACCTGGCCTTCATACACCTC (SEQ ID NO: 135)). Quantitative real-time (qRT-PCR) was performed using Power SYBR Green PCR Master Mix (Applied Biosystems), according to the manufacturer's instruction. Relative gene expression was measured for the following genes: Alk, Lmp2, Lmp7, Mecl1, Tap1, Tap2, β2m, Tapasin, Sting.

Primers:
Alk,
(SEQ ID NO: 136)
For: GCTGGACCTTCTGCATGTGGC
(SEQ ID NO: 137)
Rev: AGGCTTCAGGGGGCATCCAC;
Lmp2,
(SEQ ID NO: 138)
For: CATGAACCGAGATGGCTCTAGT
(SEQ ID NO: 139)
Rev: TCATCGTAGAATTTTGGCAGCTC;
Lmp7,
(SEQ ID NO: 140)
For: ATGGCGTTACTGGATCTGTGC;
(SEQ ID NO: 141)
Rev: CGCGGAGAAACTGTAGTGTCC;
Mecl1,
(SEQ ID NO: 142)
For: CTTTACTGCCCTTGGCTCTG,
(SEQ ID NO: 143)
Rev: GTGATCACACAGGCATCCAC;
Tap1,
(SEQ ID NO: 144)
For: GGACTTGCCTTGTTCCGAGAG;
(SEQ ID NO: 145)
Rev: GCTGCCACATAACTGATAGCGA;
Tap2,
(SEQ ID NO: 146)
For: CTGGCGGACATGGCTTTACTT;
(SEQ ID NO: 147)
Rev: CTCCCACTTTTAGCAGTCCCC;
β2m,
(SEQ ID NO: 148)
For: TTCTGGTGCTTGTCTCACTGA;
(SEQ ID NO: 149)
Rev: CAGTATGTTCGGCTTCCCATTC,
Tapasin,
(SEQ ID NO: 150)
For: GGCCTGTCTAAGAAACCTGCC;
(SEQ ID NO: 151)
Rev: CCACCTTGAAGTATAGCTTTGGG;
Sting,
(SEQ ID NO: 152)
For: GGTCACCGCTCCAAATATGTA;
(SEQ ID NO: 152)
Rev: GGT CAC CGC TCC AAA TAT GTA.

### Cell Viability Assay

Cell viability assay was performed in all immortalized mouse cell lines by using CellTiter-Glo (Promega, USA) according to the manufacturer's instructions. Briefly, cells were seeded into white-walled 96-well plates (3 wells/sample) in DMEM and incubated using a ten-point dose titration scheme from 1 nM to 1 μM of ALK inhibitors (crizotinib, lorlatinib, alectinib, ceritinib, brigatinib, ensartinib, entrectinib). After 72 h, CellTiter-Glo reagent was added to each well and luminescence output data were taken by GloMax-Multi Detection System (Promega, USA). The correspondent IC50 value for each ALK inhibitor was calculated with GraphPad Prism 9 software (GraphPad, USA).

### Kinetic Growth Assay

Immortalized mouse cell lines were harvested by trypsinization, counted, and plated in triplicate at 1000 cells per well on a 96-well plate. Photomicrographs were taken every hour using an Incucyte live cell imager and culture's confluence was measured using the Incucyte software over a period of 96 hours.

### Dendritic Cells (DCs) Generation

Frozen PBMCs from patients with NSCLC were thawed, resuspended in cold RPMI containing 3% of human AB heat-inactivated serum (Sigma Aldrich), and cultured in a T-175 culture flask for 50 min. at 37° C. in 5% C02 to induce the attachment of CD14⁺ monocytes to the plastic. The remaining floating PBMCs were removed with gentle washes of PBS and warm media. Monocytes were then cultured in RPMI containing 3% of human AB heat-inactivated serum, 2% Penicillin/Streptomycin, 1% Glutamine, and 25 nM HEPES with GM-CSF (120 ng/mL, Preprotech) and IL-4 (70 ng/mL, Preprotech). Fresh GM_CSF and IL-4 were added on days 3 and 5. On day 6, Poly I:C (30 μg/mL, Sigma Aldrich) was added for 24 hours to induce DCs maturation.

### B-Cell Activation and Expansion

B cells were expanded using the CD40 system (68,69). Briefly, NIH-3T3-CD40L cells were irradiated (9600 rads) and plated in a 6 well plate (400.000 cells/well) without Gentamycin. The following day, 8×10⁶ PBMCs were resuspended in 4 mL of IMDM (Glutamine, Hepes, Gibco) containing 10% human AB serum heat-inactivated (Sigma Aldrich), Transferrin (50 μg/ml, Lonza), Insulin (5 μg/ml, BioXtra), Cyclosporine A (5.5×10⁻⁷M, Sigma Aldrich), IL-4 (2 ng/ml, Preprotech) and Gentamycin (15 μg/ml, Gibco), and co-cultured with the irradiated NIH-3T3-CD40L for 5 days. PBMCs were then counted and cultured at the same concentration together with newly irradiated NIH-3T3-CD40L for 3 more days. After 12 days 95% of the cells were CD19⁺ and could be expanded similarly every 3-4 days at a concentration of 10⁶/mL. B cells were always used after 15 days of culture.

### Generation and Expansion of ALK-Specific CD8+ T Cells.

CD8⁺ T cells were purified using magnetic beads (CD8⁺ T cell isolation Kit, Miltenyi) and co-cultured with DCs (20:1; around 10⁶ CD8⁺ T cells:50.000 DCs) in AIM V media (Gibco) with 5% human AB heat-inactivated serum (Sigma Aldrich), 20 units/mL IL-2 (Preprotech) and 10 ng/mL IL-7 (Preprotech). Before co-culture, DCs were pulsed with the 10 μg/mL of the desired peptide in AIM V media without serum at 37° C. in 5% CO₂. Fresh IL-2 and IL-7 were added every 3-4 days. After 7 days, 3-4 million PBMCs CD8⁺ T cells were co-cultured with peptide-pulsed DCs and cytokines (20-40:1 ratio). 3^{rd} and 4^{th} stimulation stimulations were done using 4-5 million CD8⁺ T cells and peptide-pulsed irradiated B cells (ratio 4:1, 3000 rads) and fresh IL2 and IL7 (days 14 and 21). CD8⁺ T-cell responses were then evaluated in an IFN-γ-ELISPOT assay (Mabtech) using peptide-pulsed B cells (1:1 ratio) as target cells. CD8⁺ T cells were purified the day before the ELISPOT assay and rested overnight in media without cytokines. The ELISPOT was performed with FBS heat inactivated as recommended by the manufacturer. When the human material was limited, PBMCs or B0 cells were used in the first round of stimulation.

### Mass Spectrometry

Mass spectrometry was carried out using methods described in Keskin, D. B., et al. “Direct identification of an HPV-16 tumor antigen from cervical cancer biopsy specimens,” Frontiers in immunology 2, 75 (2011); and Reinherz, E. L., Keskin, D. B. & Reinhold, B. “Forward Vaccinology: CTL Targeting Based upon Physical Detection of HLA-Bound Peptides,” Frontiers in immunology 5, 418 (2014), the disclosures of which are incorporated herein by reference in their entireties for all purposes.

### Affinity Purification of Peptide-HLA Complexes and LC-MS/MS Analysis of Pan-HLA Immune Peptidomes

Detailed methods for the affinity purification of HLA complexes and LC-MS/MS analysis are described in Sarkizova, S. et al. A large peptidome dataset improves HLA class I epitope prediction across most of the human population. Nature biotechnology 38, 199-209, doi:10.1038/s41587-019-0322-9 (2020); and Klaeger, S. et al. Optimized Liquid and Gas Phase Fractionation Increases HLA-Peptidome Coverage for Primary Cell and Tissue Samples. Molecular & cellular proteomics: MCP 20, 100133, doi:10.1016/j.mcpro.2021.100133 (2021), the disclosures of which are incorporated herein by reference in their entireties for all purposes.

### Affinity Capture of Peptide-HLA Complexes for LC-DIAMS

Adherent cells were released into suspension by incubation in Non-Enzymatic Cell Dissociation Medium (NECDM; phosphate-buffered saline, pH7.2; 1%, FBS; 10 mM EDTA; 10 mM EGTA) for 1 hour at 37° C. Cells were washed in NECDM (900 g, 5 minutes at 4° C.) and resuspended to 10⁶/mL in NECDM. For each sample, 10⁶ cells were pelleted by centrifugation and the pellet gently resuspended in 1 mL digitonin extraction buffer (0.15 mM digitonin; 75 mM sucrose; 25 mM NaCl; 2.5 mM PIPES pH6.8; 1 mM EDTA; 0.8 mM MgCl₂; protease inhibitor cocktail [cØmplete; Roche]). After incubation on ice for exactly 10 minutes, the permeabilized cells were pelleted by centrifugation (480 g, 10 minutes at 4° C.) and resuspended in Triton X-100/alkylation buffer (0.5% Triton X-100; 75 mM sucrose; 25 mM NaCl; 2.5 mM PIPES pH7.4; 4 mM EDTA; 0.8 mM MgCl₂; protease inhibitor cocktail (cØmplete, Roche); when cysteines were alkylated, iodoacetamide was added to 10 mM final just before use). After incubation for 30 minutes on ice, the nuclei were pelleted by centrifugation (5000 g, 10 minutes at 4° C.) and the clarified supernatant transferred to new tubes (1.5 mL, low protein-binding [Eppendorf]). Further Triton X-100 was added to bring the proportion to 1.5%, together with Protein A-agarose beads (10 μL packed volume) and anti-HLA-A, -B, -C monomorphic determinant (4 μg; clone W6/32, Biolegend) followed by rotation for 3 hours at 4° C. Following centrifugation (900 g for 2 minutes at 4° C. and used in all subsequent steps), the agarose bead pellet was washed 6 times in octyl β-D-glucopyranoside wash buffer (1 mL; 1.75% octyl R-D-glucopyranoside; 400 mM NaCl; 40 mM Tris-HCl, pH7.6; 1 mM EDTA) followed by 2 washes in salt wash buffer (400 mM NaCl; 40 mM Tris-HCl, pH7.6; 1 mM EDTA). After removal of supernatant, pelleted beads with bound immunoprecipitate were stored at −80° C. prior to peptide elution for mass spectrometry.

### Sample Preparation for LC-DIAMS

Beads were washed with 200 μL of 5% acetonitrile (Fisher Chemical, HPLC grade) in water (Pierce™ Water, LC-MS grade Thermo Scientific) 3 times and transferred to a clean 0.5 mL tube (LoBind, Eppendorf) and all fluid aspirated leaving wet beads. 15 μL of 0.2% TFA (Optima LC/MS Fisher Chemical) along with a set of 40 retention time peptides (JPT Peptide Technologies) at 250 attomoles/peptide are added to beads. The bead/acid mixture is set at 65° C. for 5 minutes and then extracted with a C18 tip (Zip Tip, Millipore Sigma). Tip was washed with 0.2% TFA in water 5 times followed with 0.1% formic acid (99+% Thermo Scientific) in water (3 times). Peptides were eluted into 2-3 μL 60% MeOH (Fisher Chemical, HPLC grade) in water and volume was reduced under N₂ stream and 0.1% formic acid added to form 1 μL for loading the trapping column with a He-driven pressure bomb.

### Chromatography for LC-DIAMS

Alkane-modified polystyrene-divinylbenzene monoliths in 20- and 50-micron ID silica capillaries were synthesized in house for analytical and trapping columns, respectively (Gregus, M., Kostas, J. C., Ray, S., Abbatiello, S. E. & Ivanov, A. R. Improved Sensitivity of Ultralow Flow LC-MS-Based Proteomic Profiling of Limited Samples Using Monolithic Capillary Columns and FAIMS Technology. Anal Chem 92, 14702-14712, doi:10.1021/acs.analchem.0c03262 (2020)): 90-minute segmented linear gradients from 0-40% acetonitrile in water (both solvents 0.1% formic acid) were employed with the segmentation varying somewhat depending on the column in use. Flow rates varied with columns but were under 10 nanoliters/minute.

### LC-DIAMS Analysis

A Sciex 6600+ quadrupole-oTOF was used for all experiments. For extracting reference fragmentation patterns from synthetic peptide sets (JPT Peptide Technologies) the instrument was operated in a data dependent acquisition (DDA) mode. The synthetic sets were simple, consisting of orders for 250-400 pooled peptides and were analyzed at a nominal 150 or 300 attomoles per peptide in a 0.5 or 1 μL loading. For elution mapping of the synthetic set and all sample runs the instrument was operated in a data independent acquisition (DIA) mode. The instrument collected a full range mass spectrum followed with 11 MS/MS spectra in which the quadrupole filter was set to transmit an m/z window (the width varies over the 11) such that the union of these windows covered the m/z range of interest. In this way all precursor molecular ions were fragmented, but each fragmentation pattern was embedded in a complex background of other co-selected molecular ions. The sequence of MS and 11 MS/MS collections was repeated through the LC elution.

### MS Data Analysis

Poisson LC-DIAMS is a targeted form of data analysis in contrast to targeted MS data acquisition and analysis. Fragmentation patterns and relative elution positions for all targets were input parameters and acquired from synthetic peptides. A formal treatment of sampling a finite-event stochastic Poisson process (Reinhold, B., Keskin, D. B. & Reinherz, E. L. Molecular detection of targeted major histocompatibility complex I-bound peptides using a probabilistic measure and nanospray MS3 on a hybrid quadrupole-linear ion trap. Anal Chem 82, 9090-9099, doi:10.1021/ac102387t (2010)) generated a Kullback-Leibler cross-entropy measure that was used to identify the elution of a target's fragmentation pattern in an MS/MS window. A chromatogram of this measure was plotted against an extracted ion chromatogram for the target's precursor m/z and displayed as a Poisson plot. Coincident XIC and Poisson peaks for a target were further qualified by their position in the chromatogram. Retention time peptides added to the DIA runs of the synthetic set and sample generated a mapping of target elution positions from the synthetic into sample data. Peak coincidences outside the expected scatter in the elution map were not detections. Further qualification arose in inspecting the fragment XICs. These must be consistent with the precursor ion XIC elution profile and the relative amplitudes of the synthetic pattern given the fragment background and the finite event sampling or shot noise. The mass accuracy of the instrument and calibration must be satisfied. For the 6600+, mass resolution of the precursor m/z is roughly 30,000 while that of the MS/MS spectra is 25,000 with added retention time peptides validating calibration.

### Prediction ALK Peptides Binding the Human MHC-I Alleles

Potential binders to NCI-H2228 HLA-I alleles (A*0201, A*0301, B*0702, B*3801, C*0702 and C*1203) were calculated using netMHC 4.0, netMHCpan 4.1 and HLAthena (with and without peptide context) (Sarkizova, S. et al. A large peptidome dataset improves HLA class I epitope prediction across most of the human population. Nature biotechnology 38, 199-209, doi:10.1038/s41587-019-0322-9 (2020)). The full set of ALK peptides that were predicted, synthesized and for which fragmentation patterns and elution positions could be experimentally established is listed below in Table 8.

TABLE 8
Set of reference hALK-specific peptides employed in LC-DIAMS

| AALADVLRRL | GSWFTEKPTK | LPRFILLEL | RLPGASLL | STPKLIPKVTK |
|---|---|---|---|---|
| (SEQ ID NO: | (SEQ ID NO: | (SEQ ID NO: | (SEQ ID NO: | (SEQ ID NO: |
| 153) | 154) | 155) | 156) | 157) |
| AAPPPLPTT | GSWFTEKPTKK | LPRFILLELM | RLPGASLLL | SVRVPRGPA |
| (SEQ ID NO: | (SEQ ID NO: | (SEQ ID NO: | (SEQ ID NO: | (SEQ ID NO: |
| 158) | 159) | 160) | 161) | 162) |
| AFMEGIFTSK | GVSLQSLPR | LPVKWMPPEAF | RLSALESRV | SWFTEKPTKK |
| (SEQ ID NO: | (SEQ ID NO: | (SEQ ID NO: | (SEQ ID NO: | (SEQ ID NO: |
| 163) | 164) | 165) | 166) | 167) |
| AISEDHVASV | GYMPYPSK | LQSLPRFIL | RPKDPEGVPPL | SYYRKGGZAM |
| (SEQ ID NO: | (SEQ ID NO: | (SEQ ID NO: | (SEQ ID NO: | (SEQ ID NO: |
| 168) | 169) | 170) | 171) | 172) |
| AISEDHVASVK | GYMPYPSKS | LQSPEYKL | RPNFAIIL | TAAEVSVRV |
| (SEQ ID NO: | (SEQ ID NO: | (SEQ ID NO: | (SEQ ID NO: | (SEQ ID NO: |
| 173) | 174) | 175) | 176) | 6) |
| ALADVLRRL | GYQQQGLPL | LQSPEYKLSK | RPNFAIILERI | TANMKEVPL |
| (SEQ ID NO: | (SEQ ID NO: | (SEQ ID NO: | (SEQ ID NO: | (SEQ ID NO: |
| 177) | 48) | 178) | 179) | 90) |
| ALADVLRRLA | HQELQAMQM | LRTSTIMTDY | RPRPSQPSSL | TGRLPGASL |
| (SEQ ID NO: | (SEQ ID NO: | (SEQ ID NO: | (SEQ ID NO: | (SEQ ID NO: |
| 180) | 181) | 182) | 3) | 183) |
| ALADVLRRLAI | HQELQAMQMEL | LSKIPSTPK | RPRPSQPSSLA | TGRLPGASLL |
| (SEQ ID NO: | (SEQ ID NO: | (SEQ ID NO: | (SEQ ID NO: | (SEQ ID NO: |
| 184) | 185) | 186) | 187) | 188) |
| ALPIEYGPLV | HQNIVRZI | LSKLRTSTI | RPSQPSSL | TGRLPGASLLL |
| (SEQ ID NO: | (SEQ ID NO: | (SEQ ID NO: | (SEQ ID NO: | (SEQ ID NO: |
| 189) | 190) | 51) | 191) | 192) |
| AMLDLLHV | HQPEDRPNF | MAGGDLKSF | RPSQPSSLA | TKLSKIPSTPK |
| (SEQ ID NO: | (SEQ ID NO: | (SEQ ID NO: | (SEQ ID NO: | (SEQ ID NO: |
| 193) | 194) | 195) | 196) | 197) |
| AMLDLLHVA | IFSLGYMPY | MARDIYRASY | RPSQPSSLAM | TPKLIPKV |
| (SEQ ID NO: | (SEQ ID NO: | (SEQ ID NO: | (SEQ ID NO: | (SEQ ID NO: |
| 7) | 198) | 199) | 200) | 201) |
| ANMKEVPLFR | IILERIEYZ | MARDIYRASYY | RPSQPSSLAML | TPKLIPKVT |
| (SEQ ID NO: | (SEQ ID NO: | (SEQ ID NO: | (SEQ ID NO: | (SEQ ID NO: |
| 202) | 203) | 204) | 205) | 206) |
| APGAGHYEDTI | IINQVYRRK | MELQSPEYKL | RTSTIMTDY | TQDPDVINTAL |
| (SEQ ID NO: | (SEQ ID NO: | (SEQ ID NO: | (SEQ ID NO: | (SEQ ID NO: |
| 83) | 116) | 65) | 207) | 208) |
| ARDIYRASY | IMTDYNPNY | MKEVPLFRL | SEQDELDFLM | TRPRPSQPSSL |
| (SEQ ID NO: | (SEQ ID NO: | (SEQ ID NO: | (SEQ ID NO: | (SEQ ID NO: |
| 209) | 210) | 211) | 212) | 213) |
| ASYYRKGGZAM | IPSTPKLI | MPNDPSPLQV | SHTSAVSI | TSAVSIAGK |
| (SEQ ID NO: | (SEQ ID NO: | (SEQ ID NO: | (SEQ ID NO: | (SEQ ID NO: |
| 214) | 215) | 216) | 217) | 218) |
| ATAPGAGHY | IPSTPKLIP | MPYPSKSNQEV | SIKRPSPAEK | TSKTDTWSF |
| (SEQ ID NO: | (SEQ ID NO: | (SEQ ID NO: | (SEQ ID NO: | (SEQ ID NO: |
| 219) | 220) | 221) | 222) | 223) |
| ATGRLPGASL | IPSTPKLIPK | MQMELQSPEYK | SISDLKEVPRK | VATGRLPGASL |
| (SEQ ID NO: | (SEQ ID NO: | (SEQ ID NO: | (SEQ ID NO: | (SEQ ID NO: |
| 224) | 225) | 226) | 227) | 228) |
| ATGRLPGASLL | IPSTPKLIPKV | NHFIHRDI | SKIPSTPKL | VAVKTLPEV |
| (SEQ ID NO: | (SEQ ID NO: | (SEQ ID NO: | (SEQ ID NO: | (SEQ ID NO: |
| 229) | 230) | 231) | 232) | 233) |
| AVSIAGKETL | IRGLGHGAF | NHQNIVRZI | SLAMLDLLHV | VIINQVYRRK |
| (SEQ ID NO: | (SEQ ID NO: | (SEQ ID NO: | (SEQ ID NO: | (SEQ ID NO: |
| 234) | 235) | 236) | 110) | 123) |
| DPDVINTAL | ISEDHVASV | NKLSKIPSTPK | SLAMLDLLHVA | VKWMPPEAF |
| (SEQ ID NO: | (SEQ ID NO: | (SEQ ID NO: | (SEQ ID NO: | (SEQ ID NO: |
| 237) | 238) | 239) | 240) | 241) |
| DRPNFAIIL | IVRZIGVSL | NSDDSRNVY | SLDDSISAA | VPLFRLRHF |
| (SEQ ID NO: | (SEQ ID NO: | (SEQ ID NO: | (SEQ ID NO: | (SEQ ID NO: |
| 242) | 243) | 244) | 245) | 77) |
| DVIINQVYRRK | KAALADVLRRL | PDVINTAL | SLGYMPYPSK | VPPNVATGRL |
| (SEQ ID NO: | (SEQ ID NO: | (SEQ ID NO: | (SEQ ID NO: | (SEQ ID NO: |
| 126) | 246) | 247) | 248) | 249) |
| DYNPNYZF | KDPEGVPPLL | PEDRPNFAIIL | SLLLEPSSL | VPRGPAVE |
| (SEQ ID NO: | (SEQ ID NO: | (SEQ ID NO: | (SEQ ID NO: | (SEQ ID NO: |
| 250) | 251) | 252) | 253) | 254) |
| ELQSPEYKL | KEPHDRGNLGL | PHDRGNLGL | SLLLEPSSLTA | VPRGPAVEG |
| (SEQ ID NO: | (SEQ ID NO: | (SEQ ID NO: | (SEQ ID NO: | (SEQ ID NO: |
| 61) | 255) | 256) | 257) | 258) |
| ELQSPEYKLSK | KEVPRKNITLI | PQPSSQPL | SLPRFILLEL | VPRKNITL |
| (SEQ ID NO: | (SEQ ID NO: | (SEQ ID NO: | (SEQ ID NO: | (SEQ ID NO : |
| 259) | 260) | 261) | 262) | 263) |
| EPHDRGNLGL | KHQELQAM | PRFILLEL | SLPRFILLELM | VPRKNITLI |
| (SEQ ID NO: | (SEQ ID NO: | (SEQ ID NO: | (SEQ ID NO: | (SEQ ID NO: |
| 264) | 57) | 265) | 266) | 5) |
| EPSSLTANM | KHQELQAMQM | PRPSQPSSL | SLQSLPRFI | VRZIGVSL |
| (SEQ ID NO: | (SEQ ID NO: | (SEQ ID NO: | (SEQ ID NO: | (SEQ ID NO: |
| 267) | 54) | 268) | 269) | 270) |
| EQDELDFL | KIGDFGMAR | PRPSQPSSLAM | SLQSLPRFIL | VSIAGKETL |
| (SEQ ID NO: | (SEQ ID NO: | (SEQ ID NO: | (SEQ ID NO: | (SEQ ID NO: |
| 271) | 272) | 273) | 274) | 275) |
| EQDELDFLM | KIPSTPKLIPK | PSPQPSSQPL | SLQSLPRFILL | VYEGQVSGM |
| (SEQ ID NO: | (SEQ ID NO: | (SEQ ID NO: | (SEQ ID NO: | (SEQ ID NO: |
| 276) | 277) | 278) | 279) | 280) |
| ESHSNDQSPQI | KLIPKVTKTA | PTYGSWFTEK | SLTANMKEV | YGPLVEEEEKV |
| (SEQ ID NO: | (SEQ ID NO: | (SEQ ID NO: | (SEQ ID NO: | (SEQ ID NO: |
| 281) | 282) | 283) | 284) | 285) |
| FAGKTSSI | KLSKIPSTPK | QDPDVINTAL | SPAAPPPL | YGYQQQGLPL |
| (SEQ ID NO: | (SEQ ID NO: | (SEQ ID NO: | (SEQ ID NO: | (SEQ ID NO: |
| 286) | 287) | 288) | 289) | 49) |
| FGMARDIYRA | KLSKIPSTPKL | QHQPEDRPNF | SPAAPPPLPT | YKLSKLRTSTK |
| (SEQ ID NO: | (SEQ ID NO: | (SEQ ID NO: | (SEQ ID NO: | (SEQ ID NO: |
| 290) | 291) | 292) | 293) | 294) |
| FLMEALII | KPTAAEVSV | QHQPEDRPNFA | SPAAPPPLPTT | YLEENHFIH |
| (SEQ ID NO: | (SEQ ID NO: | (SEQ ID NO: | (SEQ ID NO: | (SEQ ID NO: |
| 295) | 296) | 297) | 298) | 299) |
| FLMEALIIS | KPTAAEVSVRV | QMELQSPEYK | SPAEKSHNSW | YPSKSNQEV |
| (SEQ ID NO: | (SEQ ID NO: | (SEQ ID NO: | (SEQ ID NO: | (SEQ ID NO: |
| 300) | 301) | 302) | 303) | 304) |
| FLMEALIISK | KPTSLWNPT | QPSPRAVI | SPEYKLSKL | YPSKSNQEVL |
| (SEQ ID NO: | (SEQ ID NO: | (SEQ ID NO: | (SEQ ID NO: | (SEQ ID NO: |
| 305) | 306) | 307) | 52) | 308) |
| FMEGIFTSK | KSNQEVLEF | QPSPRAVIPM | SPLQVAVKTL | YRRKHQELQAM |
| (SEQ ID NO: | (SEQ ID NO: | (SEQ ID NO: | (SEQ ID NO: | (SEQ ID NO: |
| 309) | 310) | 311) | 312) | 313) |
| FRLRHFPZ | KTDTWSFGV | QPSSLAML | SPQIRASPS | YYRKGGZAM |
| (SEQ ID NO: | (SEQ ID NO: | (SEQ ID NO: | (SEQ ID NO: | (SEQ ID NO: |
| 314) | 315) | 316) | 317) | 318) |
| FSLGYMPY | KTSSISDLK | QPSSLAMLDL | SPQPSSQPL | YYRKGGZAML |
| (SEQ ID NO: | (SEQ ID NO: | (SEQ ID NO: | (SEQ ID NO: | (SEQ ID NO: |
| 319) | 320) | 321) | 322) | 323) |
| FSLGYMPYPSK | KWMPPEAFM | QPSSQPLQI | SPQPSSQPLQ | ZPGPGRVAKI |
| (SEQ ID NO: | (SEQ ID NO: | (SEQ ID NO: | (SEQ ID NO: | (SEQ ID NO: |
| 324) | 325) | 326) | 327) | 328) |
| FSQSNPPSEL | LAMLDLLHV | QQEDEITVL | SPQPSSQPLQI | ZPGPVYRIM |
| (SEQ ID NO: | (SEQ ID NO: | (SEQ ID NO: | (SEQ ID NO: | (SEQ ID NO: |
| 329) | 330) | 331) | 332) | 333) |
| GHGAFGEVY | LAMLDLLHVA | QQQEDEITV | SPRAVIPM | ZQYLEENHFI |
| (SEQ ID NO: | (SEQ ID NO: | (SEQ ID NO: | (SEQ ID NO: | (SEQ ID NO: |
| 334) | 335) | 336) | 337) | 338) |
| GHYEDTIL | LELMAGGDLK | QQQEDEITVL | SPRAVIPMS | IVRCIGVSL |
| (SEQ ID NO: | (SEQ ID NO: | (SEQ ID NO: | (SEQ ID NO: | (SEQ ID NO: |
| 99) | 339) | 340) | 341) | 4) |
| GMARDIYRA | LEPSSLTANM | QSLPRFILL | SPSPQPSSQPL | |
| (SEQ ID NO: | (SEQ ID NO: | (SEQ ID NO: | (SEQ ID NO: | |
| 342) | 343) | 344) | 345) | |
| GMPNDPSPL | LGYMPYPSK | QSLPRFILLEL | SQSNPPSEL | |
| (SEQ ID NO: | (SEQ ID NO: | (SEQ ID NO: | (SEQ ID NO: | |
| 346) | 347) | 348) | 349) | |
| GMPNDPSPLQV | LIRGLGHGAF | QSNPPSELHK | SQSNPPSELHK | |
| (SEQ ID NO: | (SEQ ID NO: | (SEQ ID NO: | (SEQ ID NO: | |
| 350) | 351) | 352) | 353) | |
| GPAVEGGHVNM | LKAALADVL | QSPEYKLSK | SRNKPTSLW | |
| (SEQ ID NO: | (SEQ ID NO: | (SEQ ID NO: | (SEQ ID NO: | |
| 354) | 355) | 55) | 356) | |
| GRLPGASL | LLLEPSSLTA | QSPEYKLSKL | SSISDLKEV | |
| (SEQ ID NO: | (SEQ ID NO: | (SEQ ID NO: | (SEQ ID NO: | |
| 357) | 358) | 50) | 359) | |
| GRLPGASLL | LMEALIISK | QYLEENHFI | SSLTANMK | |
| (SEQ ID NO: | (SEQ ID NO: | (SEQ ID NO: | (SEQ ID NO: | |
| 360) | 361) | 362) | 363) | |
| GRLPGASLLL | LPGASLLL | RDIYRASYYR | STPKLIPK | |
| (SEQ ID NO: | (SEQ ID NO: | (SEQ ID NO: | (SEQ ID NO: | |
| 364) | 365) | 366) | 367) | |
| GRLPGASLLLE | LPIEYGPL | RKHQELQAM | STPKLIPKV | |
| (SEQ ID NO: | (SEQ ID NO: | (SEQ ID NO: | (SEQ ID NO: | |
| 368) | 369) | 370) | 371) | |

Z denoted alkylated cysteine residue
Underlined peptides were detected in at least one sample

### Statistical Analysis

Kaplan-Meier analyses for survival curves were performed with GraphPad Prism 9, and P values were determined with a log-rank Mantel-Cox test. Paired data were compared with Student t-test.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of some embodiments herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference. The invention may be related to International Patent Application No. PCT/US20/51237, filed 17 Sep. 2020, the entirety of which is incorporated herein by reference for all purposes.

What is claimed is:

1. A method for treating a subject having an anaplastic lymphoma kinase (ALK)-rearranged and/or ALK-positive neoplasia that is resistant to ALK tyrosine kinase inhibitor therapy, the method comprising administering to the subject identified as resistant to ALK tyrosine kinase inhibitor therapy, an ALK peptide and/or a polynucleotide encoding the ALK peptide, alone or in combination with a tyrosine kinase inhibitor (TKI) and/or an immune checkpoint inhibitor (ICI), thereby treating the subject.

2. A method for treating metastasis or inhibiting the development of metastasis in a subject having an anaplastic lymphoma kinase (ALK)-rearranged and/or ALK-positive neoplasia, the method comprising administering to the subject an ALK peptide and/or a polynucleotide encoding the ALK peptide, thereby treating metastasis in the subject.

3. The method of claim 2, further comprising administering to the subject a tyrosine kinase inhibitor (TKI) and/or an immune checkpoint inhibitor (ICI).

4. The method of claim 1, further comprising administering, simultaneously or sequentially, to the subject an effective amount of one or more of an ALK inhibitor, the immune checkpoint inhibitor, and/or the tyrosine kinase inhibitor (TKI).

5. The method of claim 1, wherein the ALK inhibitor or TKI is selected from the group consisting of crizotinib, alectinib, ceritinib, brigatinib, ensartinib, entrectinib, and lorlatinib.

6. The method of claim 1, wherein the neoplasia is selected from the group consisting of non-small cell lung cancer (NSCLC), anaplastic large cell lymphoma (ALCL), neuroblastoma, B-cell lymphoma, thyroid cancer, colon cancer, breast cancer, inflammatory myofibroblastic tumors (IMT), renal carcinoma, esophageal cancer, glioma, glioblastoma, and melanoma.

7. The method of claim 1, wherein the method further comprises administering a STING agonist that comprises ADU-S100.

8. The method of claim 1, wherein the peptide comprises an amino acid sequence that has at least about 95% identity to a sequence listed in any of Tables 1, 2A, 2B, 2C, and/or 7 and/or to an amino acid sequence selected from the group consisting of RPRPSQPSSL (SEQ ID NO: 3); IVRCIGVSL (SEQ ID NO: 4); VPRKNITLI (SEQ ID NO: 5); TAAEVSVRV (SEQ ID NO: 6); AMLDLLHVA (SEQ ID NO: 7); FNHQNIVRCIGVSL (SEQ ID NO: 1); and GGDLKSFLRETRPRPSQPSSLAM (SEQ ID NO: 2).

9. The method of claim 1, wherein the peptide is capable of binding a human leukocyte antigen (HLA) and the subject expresses the LA class I allele.

10. The method of claim 1, wherein the subject expresses the HLA class I allele, wherein the ALK rearrangement is a nucleophosmin-ALK rearrangement (NPM-ALK) or an echinoderm microtubule-associate protein-like 4-ALK rearrangement (EML4-ALK).

11. An isolated anaplastic lymphoma kinase (ALK) peptide capable of generating an immune response against one or more ALK-positive cancers, wherein the ALK peptide comprises a sequence with at least about 85% identity to the amino acid sequence FNHQNIVRCIGVSL (SEQ ID NO: 1) or GGDLKSFLRETRPRPSQPSSLAM (SEQ ID NO: 2).

12. A polynucleotide encoding the ALK peptide of claim 11.

13. An immunogenic composition or vaccine comprising the ALK peptide of claim 11 or a polynucleotide encoding said peptide.

14. A composition comprising an ALK peptide and/or a polynucleotide encoding the ALK peptide, a tyrosine kinase inhibitor (TKI), and/or an immune checkpoint inhibitor (ICI).

15. The composition of claim 14, wherein the ALK peptide and/or polynucleotide encoding the ALK peptide, the TKI, and/or the ICI are formulated together or separately.

16. A kit comprising an agent for administration to a subject with one or more ALK-positive cancers, wherein the agent comprises the isolated ALK peptide selected from the group consisting of RPRPSQPSSL (SEQ ID NO: 3); IVRCIGVSL (SEQ ID NO: 4); VPRKNITLI (SEQ ID NO: 5); TAAEVSVRV (SEQ ID NO: 6); AMLDLLHVA (SEQ ID NO: 7); FNHQNIVRCIGVSL (SEQ ID NO: 1); and GGDLKSFLRETRPRPSQPSSLAM (SEQ ID NO: 2).

17. A method for treating an HLA-B*07:02 subject having an anaplastic lymphoma kinase (ALK)-rearranged Non-Small Cell Lung Cancer (NSCLC) that is resistant to ALK tyrosine kinase inhibitor therapy, the method comprising administering to the subject an ALK peptide consisting of a sequence selected from the group consisting of RPRPSQPSSL (SEQ ID NO: 3); IVRCIGVSL (SEQ ID NO: 4); VPRKNITLI (SEQ ID NO: 5); TAAEVSVRV (SEQ ID NO: 6); AMLDLLHVA (SEQ ID NO: 7); FNHQNIVRCIGVSL (SEQ ID NO: 1); and GGDLKSFLRETRPRPSQPSSLAM (SEQ ID NO: 2), and/or a polynucleotide encoding the ALK peptide, alone or in combination with lorlatinib and/or an immune checkpoint inhibitor (ICI) selected from the group consisting of an anti-PD1 antibody, an anti-PDL1 antibody, and an anti-CTLA-4 antibody, thereby treating the subject.

18. A method for treating metastasis in an HLA-B*07:02 subject having an anaplastic lymphoma kinase (ALK)-rearranged Non-Small Cell Lung Cancer (NSCLC), the method comprising administering to the subject an ALK peptide consisting of a sequence selected from the group consisting of RPRPSQPSSL (SEQ ID NO: 3); IVRCIGVSL (SEQ ID NO: 4); VPRKNITLI (SEQ ID NO: 5); TAAEVSVRV (SEQ ID NO: 6); AMLDLLHVA (SEQ ID NO: 7); FNHQNIVRCIGVSL (SEQ ID NO: 1); and GGDLKSFLRETRPRPSQPSSLAM (SEQ ID NO: 2), and/or a polynucleotide encoding the ALK peptide, alone or in combination with lorlatinib and/or an immune checkpoint inhibitor (ICI) selected from the group consisting of an anti-PD1 antibody, an anti-PDL1 antibody, an anti-CTLA-4 antibody, an anti-TIM3 antibody, an anti-LAG3 antibody, an anti-TIGIT antibody, an anti-VISTA antibody, an anti-ILT2 antibody, an anti-ILT4 antibody, and an anti-KIR3DL3 antibody, thereby treating metastasis in the subject.

19. The method of claim 18, wherein the peptide is administered with ADU-S100.

20. The method of claim 18, wherein the ALK rearrangement is an echinoderm microtubule-associate protein-like 4-ALK rearrangement (EML4-ALK).


SEQUENCE LISTING (385 sequences)

SEQ ID NO: 1 - protein, 14 aa, synthetic construct
FNHQNIVRCIGVSL

SEQ ID NO: 2 - protein, 23 aa, synthetic construct
GGDLKSFLRETRPRPSQPSSLAM

SEQ ID NO: 3 - protein, 10 aa, synthetic construct
RPRPSQPSSL

SEQ ID NO: 4 - protein, 9 aa, synthetic construct
IVRCIGVSL

SEQ ID NO: 5 - protein, 9 aa, synthetic construct
VPRKNITLI

SEQ ID NO: 6 - protein, 9 aa, synthetic construct
TAAEVSVRV

SEQ ID NO: 7 - protein, 9 aa, synthetic construct
AMLDLLHVA

SEQ ID NO: 8 - protein, 1626 aa, Homo sapiens
TASSGGMGAIGLLWLLPLLLSTAAVGSGMGTGQRAGSPAAGPPLQPREPLSYSRLQRKSLAVDFVVPSLFRVYARDLLLPPSSSELKAGRPEARGSLALDCAPLLRLLGPAPGVSWTAGSPAPAEARTLSRVLKGGSVRKLRRAKQLVLELGEEAILEGCVGPPGEAAVGLLQFNLSELFSWWIRQGEGRLRIRLMPEKKASEVGREGRLSAAIRASQPRLLFQIFGTGHSSLESPTNMPSPSPDYFTWNLTWIMKDSFPFLSHRSRYGLECSFDFPCELEYSPPLHDLRNQSWSWRRIPSEEASQMDLLDGPGAERSKEMPRGSFLLLNTSADSKHTILSPWMRSSSEHCTLAVSVHRHLQPSGRYIAQLLPHNEAAREILLMPTPGKHGWTVLQGRIGRPDNPFRVALEYISSGNRSLSAVDFFALKNCSEGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLPLSLILSVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEVSVRVPRGPAVEGGHVNMAFSQSNPPSELHRVHGSRNKPTSLWNPTYGSWFTEKPTKKNNPIAKKEPHERGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 9 - protein, 1620 aa, Homo sapiens [sequence omitted]

SEQ ID NO: 10 - protein, 1620 aa, Homo sapiens [sequence omitted]

SEQ ID NO: 11 - DNA, 4881 bp, Homo sapiens
  source 1..4881: mol_type = genomic DNA
acggcctcctccggcgggatgggagccatcgggctcctgtggctcctgccgctgctgctttccacggcagctgtgggctccgggatggggaccggccagcgcgcgggctccccagctgcggggccgccgctgcagccccgggagccactcagctactcgcgcctgcagaggaagagtctggcagttgacttcgtggtgccctcgctcttccgtgtctacgcccgggacctactgctgccaccatcctcctcggagctgaaggctggcaggcccgaggcccgcggctcgctagctctggactgcgccccgctgctcaggttgctggggccggcgccgggggtctcctggaccgccggttcaccagccccggcagaggcccggacgctgtccagggtgctgaagggcggctccgtgcgcaagctccggcgtgccaagcagttggtgctggagctgggcgaggaggcgatcttggagggttgcgtcgggccccccggggaggcggctgtggggctgctccagttcaatctcagcgagctgttcagttggtggattcgccaaggcgaagggcgactgaggatccgcctgatgcccgagaagaaggcgtcggaagtgggcagagagggaaggctgtccgcggcaattcgcgcctcccagccccgccttctcttccagatcttcgggactggtcatagctccttggaatcaccaacaaacatgccatctccttctcctgattattttacatggaatctcacctggataatgaaagactccttccctttcctgtctcatcgcagccgatatggtctggagtgcagctttgacttcccctgtgagctggagtattcccctccactgcatgacctcaggaaccagagctggtcctggcgccgcatcccctccgaggaggcctcccagatggacttgctggatgggcctggggcagagcgttctaaggagatgcccagaggctcctttctccttctcaacacctcagctgactccaagcacaccatcctgagtccgtggatgaggagcagcagtgagcactgcacactggccgtctcggtgcacaggcacctgcagccctctggaaggtacattgcccagctgctgccccacaacgaggctgcaagagagatcctcctgatgcccactccagggaagcatggttggacagtgctccagggaagaatcgggcgtccagacaacccatttcgagtggccctggaatacatctccagtggaaaccgcagcttgtctgcagtggacttctttgccctgaagaactgcagtgaaggaacatccccaggctccaagatggccctgcagagctccttcacttgttggaatgggacagtcctccagcttgggcaggcctgtgacttccaccaggactgtgcccagggagaagatgagagccagatgtgccggaaactgcctgtgggtttttactgcaactttgaagatggcttctgtggctggacccaaggcacactgtcaccccacactcctcagtggcaggtcaggaccctaaaggatgcccggttccaggaccaccaagaccatgctctattgctcagtaccactgatgtccccgcttctgaaagtgctacagtgaccagtgctacgtttcctgcaccgatcaagagctctccatgtgagctccgaatgtcctggctcattcgtggagtcttgaggggaaacgtgtccttggtgctagtggagaacaaaaccgggaaggagcaaggcaggatggtctggcatgtcgccgcctatgaaggcttgagcctgtggcagtggatggtgttgcctctcctcgatgtgtctgacaggttctggctgcagatggtcgcatggtggggacaaggatccagagccatcgtggcttttgacaatatctccatcagcctggactgctacctcaccattagcggagaggacaagatcctgcagaatacagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaacaagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctga

SEQ ID NO: 12 - DNA, 4863 bp, Homo sapiens
  source 1..4863: mol_type = genomic DNA
atgggagccatcgggctcctgtggctcctgccgctgctgctttccacggcagctgtgggctccgggatggggaccggccagcgcgcgggctccccagctgcggggccgccgctgcagccccgggagccactcagctactcgcgcctgcagaggaagagtctggcagttgacttcgtggtgccctcgctcttccgtgtctacgcccgggacctactgctgccaccatcctcctcggagctgaaggctggcaggcccgaggcccgcggctcgctagctctggactgcgccccgctgctcaggttgctggggccggcgccgggggtctcctggaccgccggttcaccagccccggcagaggcccggacgctgtccagggtgctgaagggcggctccgtgcgcaagctccggcgtgccaagcagttggtgctggagctgggcgaggaggcgatcttggagggttgcgtcgggccccccggggaggcggctgtggggctgctccagttcaatctcagcgagctgttcagttggtggattcgccaaggcgaagggcgactgaggatccgcctgatgcccgagaagaaggcgtcggaagtgggcagagagggaaggctgtccgcggcaattcgcgcctcccagccccgccttctcttccagatcttcgggactggtcatagctccttggaatcaccaacaaacatgccttctccttctcctgattattttacatggaatctcacctggataatgaaagactccttccctttcctgtctcatcgcagccgatatggtctggagtgcagctttgacttcccctgtgagctggagtattcccctccactgcatgacctcaggaaccagagctggtcctggcgccgcatcccctccgaggaggcctcccagatggacttgctggatgggcctggggcagagcgttctaaggagatgcccagaggctcctttctccttctcaacacctcagctgactccaagcacaccatcctgagtccgtggatgaggagcagcagtgagcactgcacactggccgtctcggtgcacaggcacctgcagccctctggaaggtacattgcccagctgctgccccacaacgaggctgcaagagagatcctcctgatgcccactccagggaagcatggttggacagtgctccagggaagaatcgggcgtccagacaacccatttcgagtggccctggaatacatctccagtggaaaccgcagcttgtctgcagtggacttctttgccctgaagaactgcagtgaaggaacatccccaggctccaagatggccctgcagagctccttcacttgttggaatgggacagtcctccagcttgggcaggcctgtgacttccaccaggactgtgcccagggagaagatgagagccagatgtgccggaaactgcctgtgggtttttactgcaactttgaagatggcttctgtggctggacccaaggcacactgtcaccccacactcctcaatggcaggtcaggaccctaaaggatgcccggttccaggaccaccaagaccatgctctattgctcagtaccactgatgtccccgcttctgaaagtgctacagtgaccagtgctacgtttcctgcaccgatcaagagctctccatgtgagctccgaatgtcctggctcattcgtggagtcttgaggggaaacgtgtccttggtgctagtggagaacaaaaccgggaaggagcaaggcaggatggtctggcatgtcgccgcctatgaaggcttgagcctgtggcagtggatggtgttgcctctcctcgatgtgtctgacaggttctggctgcagatggtcgcatggtggggacaaggatccagagccatcgtggcttttgacaatatctccatcagcctggactgctacctcaccattagcggagaggacaagatcctgcagaatacagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggtggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctga

SEQ ID NO: 13 - DNA, 4863 bp, Homo sapiens
  source 1..4863: mol_type = genomic DNA
atgggagccatcgggctcctgtggctcctgccgctgctgctttccacggcagctgtgggctccgggatggggaccggccagcgcgcgggctccccagctgcggggccgccgctgcagccccgggagccactcagctactcgcgcctgcagaggaagagtctggcagttgacttcgtggtgccctcgctcttccgtgtctacgcccgggacctactgctgccaccatcctcctcggagctgaaggctggcaggcccgaggcccgcggctcgctagctctggactgcgccccgctgctcaggttgctggggccggcgccgggggtctcctggaccgccggttcaccagccccggcagaggcccggacgctgtccagggtgctgaagggcggctccgtgcgcaagctccggcgtgccaagcagttggtgctggagctgggcgaggaggcgatcttggagggttgcgtcgggccccccggggaggcggctgtggggctgctccagttcaatctcagcgagctgttcagttggtggattcgccaaggcgaagggcgactgaggatccgcctgatgcccgagaagaaggcgtcggaagtgggcagagagggaaggctgtccgcggcaattcgcgcctcccagccccgccttctcttccagatcttcgggactggtcatagctccttggaatcaccaacaaacatgccttctccttctcctgattattttacatggaatctcacctggataatgaaagactccttccctttcctgtctcatcgcagccgatatggtctggagtgcagctttgacttcccctgtgagctggagtattcccctccactgcatgacctcaggaaccagagctggtcctggcgccgcatcccctccgaggaggcctcccagatggacttgctggatgggcctggggcagagcgttctaaggagatgcccagaggctcctttctccttctcaacacctcagctgactccaagcacaccatcctgagtccgtggatgaggagcagcagtgagcactgcacactggccgtctcggtgcacaggcacctgcagccctctggaaggtacattgcccagctgctgccccacaacgaggctgcaagagagatcctcctgatgcccactccagggaagcatggttggacagtgctccagggaagaatcgggcgtccagacaacccatttcgagtggccctggaatacatctccagtggaaaccgcagcttgtctgcagtggacttctttgccctgaagaactgcagtgaaggaacatccccaggctccaagatggccctgcagagctccttcacttgttggaatgggacagtcctccagcttgggcaggcctgtgacttccaccaggactgtgcccagggagaagatgagagccagatgtgccggaaactgcctgtgggtttttactgcaactttgaagatggcttctgtggctggacccaaggcacactgtcaccccacactcctcaatggcaggtcaggaccctaaaggatgcccggttccaggaccaccaagaccatgctctattgctcagtaccactgatgtccccgcttctgaaagtgctacagtgaccagtgctacgtttcctgcaccgatcaagagctctccatgtgagctccgaatgtcctggctcattcgtggagtcttgaggggaaacgtgtccttggtgctagtggagaacaaaaccgggaaggagcaaggcaggatggtctggcatgtcgccgcctatgaaggcttgagcctgtggcagtggatggtgttgcctctcctcgatgtgtctgacaggttctggctgcagatggtcgcatggtggggacaaggatccagagccatcgtggcttttgacaatatctccatcagcctggactgctacctcaccattagcggagaggacaagatcctgcagaatacagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacacctgccactctcgctgatcctctctgtggtgacctctgccctcgtggccgccctggtcctggctttctccggcatcatgattgtgtaccgccggatgcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctga

SEQ ID NO: 14 - protein, 680 aa, Homo sapiens [sequence omitted]

SEQ ID NO: 15 - protein, 680 aa, Homo sapiens
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEVSVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 16 - DNA, 2043 bp, Homo sapiens
  source 1..2043: mol_type = genomic DNA
atggaagattcgatggacatggacatgagccccctgaggccccagaactatcttttcggttgtgaactaaaggccgacaaagattatcactttaaggtggataatgatgaaaatgagcaccagttatctttaagaacggtcagtttaggggctggtgcaaaggatgagttgcacattgttgaagcagaggcaatgaattacgaaggcagtccaattaaagtaacactggcaactttgaaaatgtctgtacagccaacggtttcccttgggggctttgaaataacaccaccagtggtcttaaggttgaagtgtggttcagggccagtgcatattagtggacagcacttagtagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccacggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaataagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctga

SEQ ID NO: 17 - DNA, 2043 bp, Homo sapiens
  source 1..2043: mol_type = genomic DNA
atggaagattcgatggacatggacatgagccccctgaggccccagaactatcttttcggttgtgaactaaaggccgacaaagattatcactttaaggtggataatgatgaaaatgagcaccagttatctttaagaacggtcagtttaggggctggtgcaaaggatgagttgcacattgttgaagcagaggcaatgaattacgaaggcagtccaattaaagtaacactggcaactttgaaaatgtctgtacagccaacggtttcccttgggggctttgaaataacaccaccagtggtcttaaggttgaagtgtggttcagggccagtgcatattagtggacagcacttagtagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctga

SEQ ID NO: 18 - protein, 1129 aa, Homo sapiens [sequence omitted]

SEQ ID NO: 19 - protein, 1059 aa, Homo sapiens [sequence omitted]

SEQ ID NO: 20 - DNA, 3390 bp, Homo sapiens
  source 1..3390: mol_type = genomic DNA
atggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaggaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaaggtgtatatcaaatcagcaaacaaatcaaagctcatgatggcagtgtgttcacactttgtcagatgagaaatgggatgttattaactggaggagggaaagacagaaaaataattctgtgggatcatgatctgaatcctgaaagagaaatagagtttagtgcttcaagggccaggctgccaggccatgttgcagctgaccacccacctgcagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctga

SEQ ID NO: 21 - DNA, 3180 bp, Homo sapiens
  source 1..3180: mol_type = genomic DNA
atggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaagaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaacaagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctga

SEQ ID NO: 22 - protein, 9 aa, synthetic construct
PGPGRVAKI

SEQ ID NO: 23 - protein, 9 aa, synthetic construct
RPRSQPSSL

SEQ ID NO: 24 - protein, 36 aa, synthetic construct
VYRRKHQELQAMQMELQSPEYKLSKLRTSTIMTDYN

SEQ ID NO: 25 - protein, 36 aa, synthetic construct
KLRTSTIMTDYNPNYCFAGKTSSISDLKEVPRKNIT

SEQ ID NO: 26 - protein, 36 aa, synthetic construct
SDLKEVPRKNITLIRGLGHGAFGEVYEGQVSGMPDN

SEQ ID NO: 27 - protein, 36 aa, synthetic construct
VYEGQVSGMPDNPSPLQVAVKTLPEVCSEQDELDFL

SEQ ID NO: 28 - protein, 36 aa, synthetic construct
EVCSEQDELDFLMEALIISKFNHQNIVRCIGVSLQS

SEQ ID NO: 29 - protein, 35 aa, synthetic construct
NIVRCIGVSLQSLPRFILLELMAGGDLKSFLRETR

SEQ ID NO: 30 - protein, 36 aa, synthetic construct
GDLKSFLRETRPRPSQPSSLAMLDLLHVARDIACGC

SEQ ID NO: 31 - protein, 36 aa, synthetic construct
LLHVARDIACGCQYLEENHFIHRDIAARNCLLTCPG

SEQ ID NO: 32 - protein, 36 aa, synthetic construct
IAARNCLLTCPGPGRVAKIGDFGMARDIYRASYYRK

SEQ ID NO: 33 - protein, 36 aa, synthetic construct
ARDIYRASYYRKGGCAMLPVKWMPPEAFMEGIFTSK

SEQ ID NO: 34 - protein, 36 aa, synthetic construct
PEAFMEGIFTSKTDTWSFGVLLWEIFSLGYMPYPSK

SEQ ID NO: 35 - protein, 36 aa, synthetic construct
IFSLGYMPYPSKSNQEVLEFVTSGGRMDPPKNCPGP

SEQ ID NO: 36 - protein, 36 aa, synthetic construct
GRMDPPKNCPGPVYRIMTQCWQHQPEDRPNFAIILE

SEQ ID NO: 37 - protein, 36 aa, synthetic construct
PEDRPNFAIILERIEYCTQDPDVINTALPIEYGPLV

SEQ ID NO: 38 - protein, 36 aa, synthetic construct
NTALPIEYGPLVEEEEKVPVRPKDPEGVPPLLVSQQ

SEQ ID NO: 39 - protein, 36 aa, synthetic construct
PEGVPPLLVSQQAKREEERSPAAPPPLPTTSSGKAA

SEQ ID NO: 40 - protein, 36 aa, synthetic construct
PPLPTTSSGKAAKKPTAAEISVRVPRGPAVEGGHVN

SEQ ID NO: 41 - protein, 36 aa, synthetic construct
PRGPAVEGGHVNMAFSQSNPPSELHKVHGSRNKPTS

SEQ ID NO: 42 - protein, 36 aa, synthetic construct
HKVHGSRNKPTSLWNPTYGSWFTEKPTKKNNPIAKK

SEQ ID NO: 43 - protein, 36 aa, synthetic construct
KPTKKNNPIAKKEPHDRGNLGLEGSCTVPPNVATGR

SEQ ID NO: 44 - protein, 36 aa, synthetic construct
SCTVPPNVATGRLPGASLLLEPSSLTANMKEVPLFR

SEQ ID NO: 45 - protein, 36 aa, synthetic construct
LTANMKEVPLFRLRHFPCGNVNYGYQQQGLPLEAAT

SEQ ID NO: 46 - protein, 36 aa, synthetic construct
GYQQQGLPLEAATAPGAGHYEDTILKSKNSMNQPGP

SEQ ID NO: 47 - protein, 9 aa, synthetic construct
VYRRKHQEL

SEQ ID NO: 48 - protein, 9 aa, synthetic construct
GYQQQGLPL

SEQ ID NO: 49 - protein, 10 aa, synthetic construct
YGYQQQGLPL

SEQ ID NO: 50 - protein, 10 aa, synthetic construct
QSPEYKLSKL

SEQ ID NO: 51 - protein, 9 aa, synthetic construct
LSKLRTSTI

SEQ ID NO: 52 - protein, 9 aa, synthetic construct
SPEYKLSKL

SEQ ID NO: 53 - protein, 9 aa, synthetic construct
ELQAMQMEL

SEQ ID NO: 54 - protein, 10 aa, synthetic construct
KHQELQAMQM

SEQ ID NO: 55 - protein, 9 aa, synthetic construct
QSPEYKLSK

SEQ ID NO: 56 - protein, 12 aa, synthetic construct
EYKLSKLRTSTI

SEQ ID NO: 57 - protein, 8 aa, synthetic construct
KHQELQAM

SEQ ID NO: 58 - protein, 11 aa, synthetic construct
LQSPEYKLSKL

SEQ ID NO: 59 - protein, 10 aa, synthetic construct
QELQAMQMEL

SEQ ID NO: 60 - protein, 12 aa, synthetic construct
MQMELQSPEYKL

SEQ ID NO: 61 - protein, 9 aa, synthetic construct
ELQSPEYKL

SEQ ID NO: 62 - protein, 12 aa, synthetic construct
KHQELQAMQMEL

SEQ ID NO: 63 - protein, 8 aa, synthetic construct
QSPEYKLS

SEQ ID NO: 64 - protein, 11 aa, synthetic construct
QSPEYKLSKLR

SEQ ID NO: 65 - protein, 10 aa, synthetic construct
MELQSPEYKL

SEQ ID NO: 66 - protein, 8 aa, synthetic construct
SKLRTSTI

SEQ ID NO: 67 - protein, 10 aa, synthetic construct
KLSKLRTSTI

SEQ ID NO: 68 - protein, 8 aa, synthetic construct
YRRKHQEL

SEQ ID NO: 69 - protein, 11 aa, synthetic construct
TCPGPGRVAKI

SEQ ID NO: 70 - protein, 8 aa, synthetic construct
GPGRVAKI

SEQ ID NO: 71 - protein, 10 aa, synthetic construct
VPGPGRVAKI

SEQ ID NO: 72 - protein, 8 aa, synthetic construct
IAARNCLL

SEQ ID NO: 73 - protein, 10 aa, synthetic construct
CPGPGRVAKI

SEQ ID NO: 74 - protein, 11 aa, synthetic construct
GPGRVAKIGDF

SEQ ID NO: 75 - protein, 13 aa, synthetic construct
CPGPGRVAKIGDF

SEQ ID NO: 76 - protein, 12 aa, synthetic construct
LTCPGPGRVAKI

SEQ ID NO: 77 - protein, 9 aa, synthetic construct
VPLFRLRHF

SEQ ID NO: 78 - protein, 9 aa, synthetic construct
ANMKEVPLF

SEQ ID NO: 79 - protein, 9 aa, synthetic construct
NYGYQQQGL

SEQ ID NO: 80 - protein, 11 aa, synthetic construct
NYGYQQQGLPL

SEQ ID NO: 81 - protein, 12 aa, synthetic construct
APGAGHYEDTIL

SEQ ID NO: 82 - protein, 8 aa, synthetic construct
YGYQQQGL

SEQ ID NO: 83 - protein, 11 aa, synthetic construct
APGAGHYEDTI

SEQ ID NO: 84 - protein, 11 aa, synthetic construct
KEVPLFRLRHF

SEQ ID NO: 85 - protein, 8 aa, synthetic construct
YQQQGLPL

SEQ ID NO: 86 - protein, 12 aa, synthetic construct
TAPGAGHYEDTI

SEQ ID NO: 87 - protein, 8 aa, synthetic construct
ANMKEVPL

SEQ ID NO: 88 - protein, 9 aa, synthetic construct
TILKSKNSM

SEQ ID NO: 89 - protein, 13 aa, synthetic construct
HYEDTILKSKNSM

SEQ ID NO: 90 - protein, 9 aa, synthetic construct
TANMKEVPL

SEQ ID NO: 91 - protein, 8 aa, synthetic construct
AGHYDTIL

SEQ ID NO: 92 - protein, 10 aa, synthetic construct
TANMKEVPLD

SEQ ID NO: 93 - protein, 12 aa, synthetic construct
VNYGYQQQGLPL

SEQ ID NO: 94 - protein, 12 aa, synthetic construct
MKEVPLFRLRHF

SEQ ID NO: 95 - protein, 10 aa, synthetic construct
FPCGNVNYGY

SEQ ID NO: 96 - protein, 11 aa, synthetic construct
GYQQQGLPLEA

SEQ ID NO: 97 - protein, 8 aa, synthetic construct
RHFPCGNV

SEQ ID NO: 98 - protein, 11 aa, synthetic construct
YGYQQQWGLPL

SEQ ID NO: 99 - protein, 8 aa, synthetic construct
GHYEDTIL

SEQ ID NO: 100 - protein, 8 aa, synthetic construct
TAPGAGHY

SEQ ID NO: 101 - protein, 13 aa, synthetic construct
TAPGAGHYEDTIL

SEQ ID NO: 102 - protein, 10 aa, synthetic construct
RHFPCGNVNY

SEQ ID NO: 103 - protein, 10 aa, synthetic construct
EVPLFRLRHF

SEQ ID NO: 104 - protein, 8 aa, synthetic construct
KEVPLFRL

SEQ ID NO: 105 - protein, 9 aa, synthetic construct
HYEDTILKS

SEQ ID NO: 106 - protein, 9 aa, synthetic construct
HFPCGNVNY

SEQ ID NO: 107 - protein, 10 aa, synthetic construct
TANMKEVPLF

SEQ ID NO: 108 - protein, 9 aa, synthetic construct
PGAGRIAKI

SEQ ID NO: 109 - protein, 8 aa, synthetic construct
PGPGRVAK

SEQ ID NO: 110 - protein, 10 aa, synthetic construct
SLAMLDLLHV

SEQ ID NO: 111 - protein, 10 aa, synthetic construct
VPRNKNITLI

SEQ ID NO: 112 - protein, 33 aa, synthetic construct
VTKTADKHKDVIINQVYRRKHQELQAMQMELQS

SEQ ID NO: 113 - protein, 44 aa, synthetic construct
VTKTADKHKDVIINQAKMSTREKNSQVYRRKHQELQAMQMELQS

SEQ ID NO: 114 - protein, 9 aa, synthetic construct
VIINQVYRR

SEQ ID NO: 115 - protein, 11 aa, synthetic construct
SQVYRRKHQEL

SEQ ID NO: 116 - protein, 9 aa, synthetic construct
IINQVYRRK

SEQ ID NO: 117 - protein, 9 aa, synthetic construct
REKNSQVYR

SEQ ID NO: 118 - protein, 8 aa, synthetic construct
VIINQVYR

SEQ ID NO: 119 - protein, 10 aa, synthetic construct
STREKNSQVY

SEQ ID NO: 120 - protein, 11 aa, synthetic construct
NQVYRRKHQEL

SEQ ID NO: 121 - protein, 9 aa, synthetic construct
TREKNSQVY

SEQ ID NO: 122 - protein, 11 aa, synthetic construct
KHKDVIINQVY

SEQ ID NO: 123 - protein, 10 aa, synthetic construct
VIINQVYRRK

SEQ ID NO: 124 - protein, 11 aa, synthetic construct
STREKNSQVYR

SEQ ID NO: 125 - protein, 9 aa, synthetic construct
KNSQVYRRK

SEQ ID NO: 126 - protein, 11 aa, synthetic construct
DVIINQVYRRK

SEQ ID NO: 127 - DNA, 20 bp, synthetic construct
  source 1..20: mol_type = other DNA
ttgctattcttccagctcct

SEQ ID NO: 128 - DNA, 121 bp, synthetic construct
  source 1..121: mol_type = other DNA
tatgaaattaagaaccctgttttcttcccagggatattgctgctagaaactgtctgttgacctgcccaggtccgggaagagtagcaaagattggagactttgggatggcccgagatatcta

SEQ ID NO: 129 - DNA, 20 bp, synthetic construct
  source 1..20: mol_type = other DNA
atagagggcaccctgcgact

SEQ ID NO: 130 - DNA, 20 bp, synthetic construct
  source 1..20: mol_type = other DNA
gagcacctcagtagtccgag

SEQ ID NO: 131 - protein, 9 aa, synthetic construct
FYIQMCTEL

SEQ ID NO: 132 - DNA, 25 bp, synthetic construct
  source 1..25: mol_type = other DNA
tatgaaggccaggtgtctggaatgc

SEQ ID NO: 133 - DNA, 26 bp, synthetic construct
  source 1..26: mol_type = other DNA
gacaaactccagaacttcctggttgc

SEQ ID NO: 134 - DNA, 22 bp, synthetic construct
  source 1..22: mol_type = other DNA
acctcgaccatcatgaccgact

SEQ ID NO: 135 - DNA, 22 bp, synthetic construct
  source 1..22: mol_type = other DNA
acacctggccttcatacacctc

SEQ ID NO: 136 - DNA, 21 bp, synthetic construct
  source 1..21: mol_type = other DNA
gctggaccttctgcatgtggc

SEQ ID NO: 137 - DNA, 20 bp, synthetic construct
  source 1..20: mol_type = other DNA
aggcttcagggggcatccac

SEQ ID NO: 138 - DNA, 22 bp, synthetic construct
  source 1..22: mol_type = other DNA
catgaaccgagatggctctagt

SEQ ID NO: 139 - DNA, 23 bp, synthetic construct
  source 1..23: mol_type = other DNA
tcatcgtagaattttggcagctc

SEQ ID NO: 140 - DNA, 21 bp, synthetic construct
  source 1..21: mol_type = other DNA
atggcgttactggatctgtgc

SEQ ID NO: 141 - DNA, 21 bp, synthetic construct
  source 1..21: mol_type = other DNA
cgcggagaaactgtagtgtcc

SEQ ID NO: 142 - DNA, 20 bp, synthetic construct
  source 1..20: mol_type = other DNA
ctttactgcccttggctctg

SEQ ID NO: 143 - DNA, 20 bp, synthetic construct
  source 1..20: mol_type = other DNA
gtgatcacacaggcatccac

SEQ ID NO: 144 - DNA, 21 bp, synthetic construct
  source 1..21: mol_type = other DNA
ggacttgccttgttccgagag

SEQ ID NO: 145 - DNA, 22 bp, synthetic construct
  source 1..22: mol_type = other DNA
gctgccacataactgatagcga

SEQ ID NO: 146 - DNA, 21 bp, synthetic construct
  source 1..21: mol_type = other DNA
ctggcggacatggctttactt

SEQ ID NO: 147 - DNA, 21 bp, synthetic construct
  source 1..21: mol_type = other DNA
ctcccacttttagcagtcccc

SEQ ID NO: 148 - DNA, 21 bp, synthetic construct
  source 1..21: mol_type = other DNA
ttctggtgcttgtctcactga

SEQ ID NO: 149 - DNA, 22 bp, synthetic construct
  source 1..22: mol_type = other DNA
cagtatgttcggcttcccattc

SEQ ID NO: 150 - DNA, 21 bp, synthetic construct
  source 1..21: mol_type = other DNA
ggcctgtctaagaaacctgcc

SEQ ID NO: 151 - DNA, 23 bp, synthetic construct
  source 1..23: mol_type = other DNA
ccaccttgaagtatagctttggg

SEQ ID NO: 152 - DNA, 21 bp, synthetic construct
  source 1..21: mol_type = other DNA
ggtcaccgctccaaatatgta

SEQ ID NO: 153 - protein, 10 aa, synthetic construct
AALADVLRRL

SEQ ID NO: 154 - protein, 10 aa, synthetic construct
GSWFTEKPTK

SEQ ID NO: 155 - protein, 9 aa, synthetic construct
LPRFILLEL

SEQ ID NO: 156 - protein, 8 aa, synthetic construct
RLPGASLL

SEQ ID NO: 157 - protein, 11 aa, synthetic construct
STPKLIPKVTK

SEQ ID NO: 158 - protein, 9 aa, synthetic construct
AAPPPLPTT

SEQ ID NO: 159 - protein, 11 aa, synthetic construct
GSWFTEKPTKK

SEQ ID NO: 160 - protein, 10 aa, synthetic construct
LPRFILLELM

SEQ ID NO: 161 - protein, 9 aa, synthetic construct
RLPGASLLL

SEQ ID NO: 162 - protein, 9 aa, synthetic construct
SVRVPRGPA

SEQ ID NO: 163 - protein, 10 aa, synthetic construct
AFMEGIFTSK

SEQ ID NO: 164 - protein, 9 aa, synthetic construct
GVSLQSLPR

SEQ ID NO: 165 - protein, 11 aa, synthetic construct
LPVKWMPPEAF

SEQ ID NO: 166 - protein, 9 aa, synthetic construct
RLSALESRV

SEQ ID NO: 167 - protein, 10 aa, synthetic construct
SWFTEKPTKK

SEQ ID NO: 168 - protein, 10 aa, synthetic construct
AISEDHVASV

SEQ ID NO: 169 - protein, 8 aa, synthetic construct
GYMPYPSK

SEQ ID NO: 170 - protein, 9 aa, synthetic construct
LQSLPRFIL

SEQ ID NO: 171 - protein, 11 aa, synthetic construct
RPKDPEGVPPL

SEQ ID NO: 172 - protein, 10 aa, synthetic construct
  SITE 8: note = Alkylated cysteine
SYYRKGGCAM

SEQ ID NO: 173 - protein, 11 aa, synthetic construct
AISEDHVASVK

SEQ ID NO: 174 - protein, 9 aa, synthetic construct
GYMPYPSKS

SEQ ID NO: 175 - protein, 8 aa, synthetic construct
LQSPEYKL

SEQ ID NO: 176 - protein, 8 aa, synthetic construct
RPNFAIIL

SEQ ID NO: 177 - protein, 9 aa, synthetic construct
ALADVLRRL

SEQ ID NO: 178 - protein, 10 aa, synthetic construct
LQSPEYKLSK

SEQ ID NO: 179 - protein, 11 aa, synthetic construct
RPNFAIILERI

SEQ ID NO: 180 - protein, 10 aa, synthetic construct
ALADVLRRLA

SEQ ID NO: 181 - protein, 9 aa, synthetic construct
HQELQAMQM

SEQ ID NO: 182 - protein, 10 aa, synthetic construct
LRTSTIMTDY

SEQ ID NO: 183 - protein, 9 aa, synthetic construct
TGRLPGASL

SEQ ID NO: 184 - protein, 11 aa, synthetic construct
ALADVLRRLAI

SEQ ID NO: 185 - protein, 11 aa, synthetic construct
HQELQAMQMEL

SEQ ID NO: 186 - protein, 9 aa, synthetic construct
LSKIPSTPK

SEQ ID NO: 187 - protein, 11 aa, synthetic construct
RPRPSQPSSLA

SEQ ID NO: 188 - protein, 10 aa, synthetic construct
TGRLPGASLL

SEQ ID NO: 189 - protein, 10 aa, synthetic construct
ALPIEYGPLV

SEQ ID NO: 190 - protein, 8 aa, synthetic construct
  SITE 7: note = Alkylated cysteine
HQNIVRCI

SEQ ID NO: 191 - protein, 8 aa, synthetic construct
RPSQPSSL

SEQ ID NO: 192 - protein, 11 aa, synthetic construct
TGRLPGASLLL

SEQ ID NO: 193 - protein, 8 aa, synthetic construct
AMLDLLHV

SEQ ID NO: 194 - protein, 9 aa, synthetic construct
HQPEDRPNF

SEQ ID NO: 195 - protein, 9 aa, synthetic construct
MAGGDLKSF

SEQ ID NO: 196 - protein, 9 aa, synthetic construct
RPSQPSSLA

SEQ ID NO: 197 - protein, 11 aa, synthetic construct
TKLSKIPSTPK

SEQ ID NO: 198 - protein, 9 aa, synthetic construct
IFSLGYMPY

SEQ ID NO: 199 - protein, 10 aa, synthetic construct
MARDIYRASY

SEQ ID NO: 200 - protein, 10 aa, synthetic construct
RPSQPSSLAM

SEQ ID NO: 201 - protein, 8 aa, synthetic construct
TPKLIPKV

SEQ ID NO: 202 - protein, 10 aa, synthetic construct
ANMKEVPLFR

SEQ ID NO: 203 - protein, 9 aa, synthetic construct
  SITE 9: note = Alkylated cysteine
IILERIEYC

SEQ ID NO: 204 - protein, 11 aa, synthetic construct
MARDIYRASYY

SEQ ID NO: 205 - protein, 11 aa, synthetic construct
RPSQPSSLAML

SEQ ID NO: 206 - protein, 9 aa, synthetic construct
TPKLIPKVT

SEQ ID NO: 207 - protein, 9 aa, synthetic construct
RTSTIMTDY

SEQ ID NO: 208 - protein, 11 aa, synthetic construct
TQDPDVINTAL

SEQ ID NO: 209 - protein, 9 aa, synthetic construct
ARDIYRASY

SEQ ID NO: 210 - protein, 9 aa, synthetic construct
IMTDYNPNY

SEQ ID NO: 211 - protein, 9 aa, synthetic construct
MKEVPLFRL

SEQ ID NO: 212 - protein, 10 aa, synthetic construct
SEQDELDFLM

SEQ ID NO: 213 - protein, 11 aa, synthetic construct
TRPRPSQPSSL

SEQ ID NO: 214 - protein, 11 aa, synthetic construct
  SITE 9: note = Alkylated cysteine
ASYYRKGGCAM

SEQ ID NO: 215 - protein, 8 aa, synthetic construct
IPSTPKLI

SEQ ID NO: 216 - protein, 10 aa, synthetic construct
MPNDPSPLQV

SEQ ID NO: 217 - protein, 8 aa, synthetic construct
SHTSAVSI

SEQ ID NO: 218 - protein, 9 aa, synthetic construct
TSAVSIAGK

SEQ ID NO: 219 - protein, 9 aa, synthetic construct
ATAPGAGHY

SEQ ID NO: 220 - protein, 9 aa, synthetic construct
IPSTPKLIP

SEQ ID NO: 221 - protein, 11 aa, synthetic construct
MPYPSKSNQEV

SEQ ID NO: 222 - protein, 10 aa, synthetic construct
SIKRPSPAEK

SEQ ID NO: 223 - protein, 9 aa, synthetic construct
TSKTDTWSF

SEQ ID NO: 224 - protein, 10 aa, synthetic construct
ATGRLPGASL

SEQ ID NO: 225 - protein, 10 aa, synthetic construct
IPSTPKLIPK

SEQ ID NO: 226 - protein, 11 aa, synthetic construct
MQMELQSPEYK

SEQ ID NO: 227 - protein, 11 aa, synthetic construct
SISDLKEVPRK

SEQ ID NO: 228 - protein, 11 aa, synthetic construct
VATGRLPGASL

SEQ ID NO: 229 - protein, 11 aa, synthetic construct
ATGRLPGASLL

SEQ ID NO: 230 - protein, 11 aa, synthetic construct
IPSTPKLIPKV

SEQ ID NO: 231 - protein, 8 aa, synthetic construct
NHFIHRDI

SEQ ID NO: 232 - protein, 9 aa, synthetic construct
SKIPSTPKL

SEQ ID NO: 233 - protein, 9 aa, synthetic construct
VAVKTLPEV

SEQ ID NO: 234 - protein, 10 aa, synthetic construct
AVSIAGKETL

SEQ ID NO: 235 - protein, 9 aa, synthetic construct
IRGLGHGAF

SEQ ID NO: 236 - protein, 9 aa, synthetic construct
  SITE 8: note = Alkylated cysteine
NHQNIVRCI

SEQ ID NO: 237 - protein, 9 aa, synthetic construct
DPDVINTAL

SEQ ID NO: 238 - protein, 9 aa, synthetic construct
ISEDHVASV

SEQ ID NO: 239 - protein, 11 aa, synthetic construct
NKLSKIPSTPK

SEQ ID NO: 240 - protein, 11 aa, synthetic construct
SLAMLDLLHVA

SEQ ID NO: 241 - protein, 9 aa, synthetic construct
VKWMPPEAF

SEQ ID NO: 242 - protein, 9 aa, synthetic construct
DRPNFAIIL

SEQ ID NO: 243 - protein, 9 aa, synthetic construct
  SITE 4: note = Alkylated cysteine
IVRCIGVSL

SEQ ID NO: 244 - protein, 9 aa, synthetic construct
NSDDSRNVY

SEQ ID NO: 245 - protein, 9 aa, synthetic construct
SLDDSISAA

SEQ ID NO: 246 - protein, 11 aa, synthetic construct
KAALADVLRRL

SEQ ID NO: 247 - protein, 8 aa, synthetic construct
PDVINTAL

SEQ ID NO: 248 - protein, 10 aa, synthetic construct
SLGYMPYPSK

SEQ ID NO: 249 - protein, 10 aa, synthetic construct
VPPNVATGRL

SEQ ID NO: 250 - protein, 8 aa, synthetic construct
  SITE 7: note = Alkylated cysteine
DYNPNYCF

SEQ ID NO: 251 - protein, 10 aa, synthetic construct
KDPEGVPPLL

SEQ ID NO: 252 - protein, 11 aa, synthetic construct
PEDRPNFAIIL

SEQ ID NO: 253 - protein, 9 aa, synthetic construct
SLLLEPSSL

SEQ ID NO: 254 - protein, 8 aa, synthetic construct
VPRGPAVE

SEQ ID NO: 255 - protein, 11 aa, synthetic construct
KEPHDRGNLGL

SEQ ID NO: 256 - protein, 9 aa, synthetic construct
PHDRGNLGL

SEQ ID NO: 257 - protein, 11 aa, synthetic construct
SLLLEPSSLTA

SEQ ID NO: 258 - protein, 9 aa, synthetic construct
VPRGPAVEG

SEQ ID NO: 259 - protein, 11 aa, synthetic construct
ELQSPEYKLSK

SEQ ID NO: 260 - protein, 11 aa, synthetic construct
KEVPRKNITLI

SEQ ID NO: 261 - protein, 8 aa, synthetic construct
PQPSSQPL

SEQ ID NO: 262 - protein, 10 aa, synthetic construct
SLPRFILLEL

SEQ ID NO: 263 - protein, 8 aa, synthetic construct
VPRKNITL

SEQ ID NO: 264 - protein, 10 aa, synthetic construct
EPHDRGNLGL

SEQ ID NO: 265 - protein, 8 aa, synthetic construct
PRFILLEL

SEQ ID NO: 266 - protein, 11 aa, synthetic construct
SLPRFILLELM

SEQ ID NO: 267 - protein, 9 aa, synthetic construct
EPSSLTANM

SEQ ID NO: 268 - protein, 9 aa, synthetic construct
PRPSQPSSL

SEQ ID NO: 269 - protein, 9 aa, synthetic construct
SLQSLPRFI

SEQ ID NO: 270 - protein, 8 aa, synthetic construct
  SITE 3: note = Alkylated cysteine
VRCIGVSL

SEQ ID NO: 271 - protein, 8 aa, synthetic construct
EQDELDFL

SEQ ID NO: 272 - protein, 9 aa, synthetic construct
KIGDFGMAR

SEQ ID NO: 273 - protein, 11 aa, synthetic construct
PRPSQPSSLAM

SEQ ID NO: 274 - protein, 10 aa, synthetic construct
SLQSLPRFIL

SEQ ID NO: 275 - protein, 9 aa, synthetic construct
VSIAGKETL

SEQ ID NO: 276 - protein, 9 aa, synthetic construct
EQDELDFLM

SEQ ID NO: 277 - protein, 11 aa, synthetic construct
KIPSTPKLIPK

SEQ ID NO: 278 - protein, 10 aa, synthetic construct
PSPQPSSQPL

SEQ ID NO: 279 - protein, 11 aa, synthetic construct
SLQSLPRFILL

SEQ ID NO: 280 - protein, 9 aa, synthetic construct
VYEGQVSGM

SEQ ID NO: 281 - protein, 11 aa, synthetic construct
ESHSNDQSPQI

SEQ ID NO: 282 - protein, 10 aa, synthetic construct
KLIPKVTKTA

SEQ ID NO: 283 - protein, 10 aa, synthetic construct
PTYGSWFTEK

SEQ ID NO: 284 - protein, 9 aa, synthetic construct
SLTANMKEV

SEQ ID NO: 285 - protein, 11 aa, synthetic construct
YGPLVEEEEKV

SEQ ID NO: 286 - protein, 8 aa, synthetic construct
FAGKTSSI

SEQ ID NO: 287 - protein, 10 aa, synthetic construct
KLSKIPSTPK

SEQ ID NO: 288 - protein, 10 aa, synthetic construct
QDPDVINTAL

SEQ ID NO: 289 - protein, 8 aa, synthetic construct
SPAAPPPL

SEQ ID NO: 290 - protein, 10 aa, synthetic construct
FGMARDIYRA

SEQ ID NO: 291 - protein, 11 aa, synthetic construct
KLSKIPSTPKL

SEQ ID NO: 292 - protein, 10 aa, synthetic construct
QHQPEDRPNF

SEQ ID NO: 293 - protein, 10 aa, synthetic construct
SPAAPPPLPT

SEQ ID NO: 294 - protein, 11 aa, synthetic construct
YKLSKLRTSTK

SEQ ID NO: 295 - protein, 8 aa, synthetic construct
FLMEALII

SEQ ID NO: 296 - protein, 9 aa, synthetic construct
KPTAAEVSV

SEQ ID NO: 297 - protein, 11 aa, synthetic construct
QHQPEDRPNFA

SEQ ID NO: 298 - protein, 11 aa, synthetic construct
SPAAPPPLPTT

SEQ ID NO: 299 - protein, 9 aa, synthetic construct
YLEENHFIH

SEQ ID NO: 300 - protein, 9 aa, synthetic construct
FLMEALIIS

SEQ ID NO: 301 - protein, 11 aa, synthetic construct
KPTAAEVSVRV

SEQ ID NO: 302 - protein, 10 aa, synthetic construct
QMELQSPEYK

SEQ ID NO: 303 - protein, 10 aa, synthetic construct
SPAEKSHNSW

SEQ ID NO: 304 - protein, 9 aa, synthetic construct
YPSKSNQEV

SEQ ID NO: 305 - protein, 10 aa, synthetic construct
FLMEALIISK

SEQ ID NO: 306 - protein, 9 aa, synthetic construct
KPTSLWNPT

SEQ ID NO: 307 - protein, 8 aa, synthetic construct
QPSPRAVI

SEQ ID NO: 308 - protein, 10 aa, synthetic construct
YPSKSNQEVL

SEQ ID NO: 309 - protein, 9 aa, synthetic construct
FMEGIFTSK

SEQ ID NO: 310 - protein, 9 aa, synthetic construct
KSNQEVLEF

SEQ ID NO: 311 - protein, 10 aa, synthetic construct
QPSPRAVIPM

SEQ ID NO: 312 - protein, 10 aa, synthetic construct
SPLQVAVKTL

SEQ ID NO: 313 - protein, 11 aa, synthetic construct
YRRKHQELQAM

SEQ ID NO: 314 - protein, 8 aa, synthetic construct
  SITE 8: note = Alkylated cysteine
FRLRHFPC

SEQ ID NO: 315 - protein, 9 aa, synthetic construct
KTDTWSFGV

SEQ ID NO: 316 - protein, 8 aa, synthetic construct
QPSSLAML

SEQ ID NO: 317 - protein, 9 aa, synthetic construct
SPQIRASPS

SEQ ID NO: 318 - protein, 9 aa, synthetic construct
  SITE 7: note = Alkylated cysteine
YYRKGGCAM

SEQ ID NO: 319 - protein, 8 aa, synthetic construct
FSLGYMPY

SEQ ID NO: 320 - protein, 9 aa, synthetic construct
KTSSISDLK

SEQ ID NO: 321 - protein, 10 aa, synthetic construct
QPSSLAMLDL

SEQ ID NO: 322 - protein, 9 aa, synthetic construct
SPQPSSQPL

SEQ ID NO: 323 - protein, 10 aa, synthetic construct
  SITE 7: note = Alkylated cysteine
YYRKGGCAML

SEQ ID NO: 324 - protein, 11 aa, synthetic construct
FSLGYMPYPSK

SEQ ID NO: 325 - protein, 9 aa, synthetic construct
KWMPPEAFM

SEQ ID NO: 326 - protein, 9 aa, synthetic construct
QPSSQPLQI

SEQ ID NO: 327 - protein, 10 aa, synthetic construct
SPQPSSQPLQ

SEQ ID NO: 328 - protein, 10 aa, synthetic construct
  SITE 1: note = Alkylated cysteine
CPGPGRVAKI

SEQ ID NO: 329 - protein, 10 aa, synthetic construct
FSQSNPPSEL

SEQ ID NO: 330 - protein, 9 aa, synthetic construct
LAMLDLLHV

SEQ ID NO: 331 - protein, 9 aa, synthetic construct
QQEDEITVL

SEQ ID NO: 332 - protein, 11 aa, synthetic construct
SPQPSSQPLQI

SEQ ID NO: 333 - protein, 9 aa, synthetic construct
  SITE 1: note = Alkylated cysteine
CPGPVYRIM

SEQ ID NO: 334 - protein, 9 aa, synthetic construct
GHGAFGEVY

SEQ ID NO: 335 - protein, 10 aa, synthetic construct
LAMLDLLHVA

SEQ ID NO: 336 - protein, 9 aa, synthetic construct
QQQEDEITV

SEQ ID NO: 337 - protein, 8 aa, synthetic construct
SPRAVIPM

SEQ ID NO: 338 - protein, 10 aa, synthetic construct
  SITE 1: note = Alkylated cysteine
CQYLEENHFI

SEQ ID NO: 339 - protein, 10 aa, synthetic construct
LELMAGGDLK

SEQ ID NO: 340 - protein, 10 aa, synthetic construct
QQQEDEITVL

SEQ ID NO: 341 - protein, 9 aa, synthetic construct
SPRAVIPMS

SEQ ID NO: 342 - protein, 9 aa, synthetic construct
GMARDIYRA

SEQ ID NO: 343 - protein, 10 aa, synthetic construct
LEPSSLTANM

SEQ ID NO: 344 - protein, 9 aa, synthetic construct
QSLPRFILL

SEQ ID NO: 345 - protein, 11 aa, synthetic construct
SPSPQPSSQPL

SEQ ID NO: 346 - protein, 9 aa, synthetic construct
GMPNDPSPL

SEQ ID NO: 347 - protein, 9 aa, synthetic construct
LGYMPYPSK

SEQ ID NO: 348 - protein, 11 aa, synthetic construct
QSLPRFILLEL

SEQ ID NO: 349 - protein, 9 aa, synthetic construct
SQSNPPSEL

SEQ ID NO: 350 - protein, 11 aa, synthetic construct
GMPNDPSPLQV

SEQ ID NO: 351 - protein, 10 aa, synthetic construct
LIRGLGHGAF

SEQ ID NO: 352 - protein, 10 aa, synthetic construct
QSNPPSELHK

SEQ ID NO: 353 - protein, 11 aa, synthetic construct
SQSNPPSELHK

SEQ ID NO: 354 - protein, 11 aa, synthetic construct
GPAVEGGHVNM

SEQ ID NO: 355 - protein, 9 aa, synthetic construct
LKAALADVL

SEQ ID NO: 356 - protein, 9 aa, synthetic construct
SRNKPTSLW

SEQ ID NO: 357 - protein, 8 aa, synthetic construct
GRLPGASL

SEQ ID NO: 358 - protein, 10 aa, synthetic construct
LLLEPSSLTA

SEQ ID NO: 359 - protein, 9 aa, synthetic construct
SSISDLKEV

SEQ ID NO: 360 - protein, 9 aa, synthetic construct
GRLPGASLL

SEQ ID NO: 361 - protein, 9 aa, synthetic construct
LMEALIISK

SEQ ID NO: 362 - protein, 9 aa, synthetic construct
QYLEENHFI

SEQ ID NO: 363 - protein, 8 aa, synthetic construct
SSLTANMK

SEQ ID NO: 364 - protein, 10 aa, synthetic construct
GRLPGASLLL

SEQ ID NO: 365 - protein, 8 aa, synthetic construct
LPGASLLL

SEQ ID NO: 366 - protein, 10 aa, synthetic construct
RDIYRASYYR

SEQ ID NO: 367 - protein, 8 aa, synthetic construct
STPKLIPK

SEQ ID NO: 368 - protein, 11 aa, synthetic construct
GRLPGASLLLE

SEQ ID NO: 369 - protein, 8 aa, synthetic construct
LPIEYGPL

SEQ ID NO: 370 - protein, 9 aa, synthetic construct
RKHQELQAM

SEQ ID NO: 371 - protein, 9 aa, synthetic construct
STPKLIPKV

SEQ ID NO: 372 - DNA, 32 bp, synthetic construct
  source 1..32: mol_type = other DNA
cgggaaggacctaaagtgtaccgtcggaagca

SEQ ID NO: 373 - DNA, 27 bp, synthetic construct
  source 1..27: mol_type = other DNA
ccaggagctggaagaatagcaaagatt

SEQ ID NO: 374 - DNA, 27 bp, synthetic construct
  source 1..27: mol_type = other DNA
ccaggccctggaagagtggccaagatt

SEQ ID NO: 375 - DNA, 12 bp, synthetic construct
  source 1..12: mol_type = other DNA
agctggaagaat

SEQ ID NO: 376 - DNA, 31 bp, synthetic construct
  source 1..31: mol_type = other DNA
cctgcccaggagctggaagaatagcaaagat

SEQ ID NO: 377 - DNA, 11 bp, synthetic construct
  source 1..11: mol_type = other DNA
tccgggaagag

SEQ ID NO: 378 - DNA, 31 bp, synthetic construct
  source 1..31: mol_type = other DNA
cctgcccaggtccgggaagagtagcaaagat

SEQ ID NO: 379 - DNA, 18 bp, synthetic construct
  source 1..18: mol_type = other DNA
aggtccgggaagagtagc

SEQ ID NO: 380 - DNA, 17 bp, synthetic construct
  source 1..17: mol_type = other DNA
aggtccgggaagagtag

SEQ ID NO: 381 - DNA, 18 bp, synthetic construct
  source 1..18: mol_type = other DNA
aggagctggaagaatagc

SEQ ID NO: 382 - protein, 5 aa, synthetic construct
PRKNI

SEQ ID NO: 383 - protein, 5 aa, synthetic construct
RKNIT

SEQ ID NO: 384 - protein, 6 aa, synthetic construct
PRKNIT

SEQ ID NO: 385 - protein, 7 aa, synthetic construct
PRKNITL